(19) United States
(12) Patent Application Publication
(10) Pub. No.: US 2022/0002314 A1
(43) Pub. Date: Jan. 6, 2022

(54) PIPERAZINYL AND PIPERIDINYL QUINAZOLIN-4(3H)-ONE DERIVATIVES HAVING ACTIVITY AGAINST PAIN

(71) Applicant: ACONDICIONAMIENTO TARRASENSE, Terrassa (Barcelona) (ES)

(72) Inventors: Ariadna FERNÁNDEZ-DONIS, Barcelona (ES); Jose Luís DÍAZ-FERNÁNDEZ, Manresa (Barcelona) (ES); Carmen ALMANSA-ROSALES, Barcelona (ES); Adriana LORENTE-CRIVILLÉ, BARCELONA (ES)

(73) Assignee: ACONDICIONAMIENTO TARRASENSE, Terrassa (Barcelona) (ES)

(21) Appl. No.: 17/290,747

(22) PCT Filed: Oct. 31, 2019
(86) PCT No.: PCT/EP2019/079846

§ 371 Date: Apr. 30, 2021

(30) Foreign Application Priority Data
Oct. 31, 2018 (EP) 18382772.4

(51) Int. Cl. C07D 498/10; C07D 471/04; C07D 403/06; C07D 239/90; C07D 405/14; C07D 401/14; C07D 403/14; C07D 471/18

(52) U.S. Cl. CPC C07D 498/10; C07D 471/04; C07D 403/06; C07D 471/18; C07D 405/14; C07D 401/14; C07D 403/14; C07D 239/90

(57) ABSTRACT

The present invention relates to piperazinyl and piperidinyl quinazolin-4(3H)-one derivatives having pharmacological activity towards the α2δ subunit, in particular the α2δ-1 subunit, of the voltage-gated calcium channel, in particular having dual pharmacological activity towards both the α2δ subunit, in particular the α2δ-1 subunit, of the voltage-gated calcium channel and the μ-opioid receptor. The present invention also relates to processes of preparation of such compounds, to pharmaceutical compositions comprising them, and to their use in therapy, in particular for the treatment of pain.

### FIELD OF THE INVENTION

The present invention relates to compounds having pharmacological activity towards the α₂δ subunit of the voltage-gated calcium channel. In particular, the present invention relates to compounds having dual pharmacological activity towards both the α₂δ subunit of the voltage-gated calcium channel, and the μ-opioid receptor (MOR or mu-opioid receptor). More particularly, the present invention relates to piperazinyl and piperidinyl quinazolin-4(3H)-one derivatives having this pharmacological activity, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved (Turk, D. C., Wilson, H. D., Cahana, A.; 2011; Lancet, 377; 2226-2235). Pain affects a big portion of the population with an estimated prevalence of 20% and its incidence, particularly in the case of chronic pain, is increasing due to the population ageing. Additionally, pain is clearly related to comorbidities, such as depression, anxiety and insomnia, which lead to important productivity losses and socio-economical burden (Goldberg, D. S., McGee, S. J.; 2011; BMC Public Health; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al., Pharmacol Rev. 2015 67:821-70). The VGCC are assembled through interactions of different subunits, namely α₁ (Caᵥα₁), β (Caᵥβ) α₂δ (Caᵥα₂δ) and γ (Caᵥγ). The α₁ subunits are the key porous forming units of the channel complex, being responsible for the Ca²⁺ conduction and generation of Ca²⁺ influx. The α₂δ, β, and γ subunits are auxiliary, although very important for the regulation of the channel since they increase the expression of the α₁ subunits in the plasma membrane as well as modulate their function, resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N—(Caᵥ2.2), P/Q-(Caᵥ2.1), and R—(Caᵥ2.3) types, depending on the channel forming Caᵥα subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo, Neurotherapeutics. 2009 6:679-92; Zamponi et al., 2015 supra; Neumaier et al., Prog Neurobiol. 2015 129:1-36). A large body of data has clearly indicated that VGCC are implicated in mediating various disease states including pain processing. Drugs interacting with the different calcium channel subtypes and subunits have been developed. Current therapeutic agents include drugs targeting L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of N-type (Caᵥ2.2) calcium channels, has been approved as a treatment of intractable pain. (Perret and Luo, 2009, supra; Vink and Alewood, Br J Pharmacol. 2012 167:970-89).

The Caᵥ1 and Caᵥ2 subfamilies contain an auxiliary α₂δ subunit, which is the therapeutic target of the gabapentinoid drugs of value in certain epilepsies and chronic neuropathic pain. To date, there are four known α₂δ subunits, each encoded by a unique gene and all possessing splice variants. Each α₂δ protein is encoded by a single messenger RNA and is posttranslationally cleaved and then linked by disulfide bonds. Four genes encoding α₂δ subunits have now been cloned. α₂δ-1 was initially cloned from skeletal muscle and shows a fairly ubiquitous distribution. The α₂δ-2 and α₂δ-3 subunits were subsequently cloned from brain. The most recently identified subunit, α₂δ-4, is largely nonneuronal. The human α₂δ-4 protein sequence shares 30, 32 and 61% identity with the human α₂δ-1, α₂δ-2 and α₂δ-3 subunits, respectively. The gene structure of all α₂δ subunits is similar. All α₂δ subunits show several splice variants (Davies et al., Trends Pharmacol Sci. 2007 28:220-8; Dolphin A C, Nat Rev Neurosci. 2012 13:542-55, Biochim Biophys Acta. 2013 1828:1541-9).

The Caᵥα₂δ-1 subunit may play an important role in neuropathic pain development (Perret and Luo, 2009, supra; Vink and Alewood, 2012, supra). Biochemical data have indicated a significant Caᵥα₂δ-1, but not Caᵥα₂δ-2, subunit upregulation in the spinal dorsal horn, and DRG (dorsal root ganglia) after nerve injury that correlates with neuropathic pain development. In addition, blocking axonal transport of injury-induced DRG Caᵥα₂δ-1 subunit to the central presynaptic terminals diminishes tactile allodynia in nerve injured animals, suggesting that elevated DRG Caᵥα₂δ-1 subunit contributes to neuropathic allodynia.

The Caᵥα₂δ-1 subunit (and the Caᵥα₂δ-2, but not Caᵥα₂δ-3 and Caᵥα₂δ-4, subunits) is the binding site for gabapentin which has anti-allodynic/hyperalgesic properties in patients and animal models. Because injury-induced Caᵥα₂δ-1 expression correlates with neuropathic pain development and maintenance, and various calcium channels are known to contribute to spinal synaptic neurotransmission and DRG neuron excitability, injury-induced Caᵥα₂δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα₂δ-1 subunit can block nerve injury-induced Caᵥα₂δ-1 upregulation and prevent the onset of allodynia and reserve established allodynia.

As mentioned above, the α₂δ subunits of VGCC form the binding site for gabapentin and pregabalin, which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα₂δ subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009, supra; Vink and Alewood, 2012, supra, Zamponi et al., 2015, supra).

Thus, the present invention relates to compounds with inhibitory effect towards the α₂δ subunit, in particular the α₂δ-1 subunit, of voltage-gated calcium channels.

As mentioned before, there are few available therapeutic classes for the treatment of pain, and opioids are among the most effective, especially when addressing severe pain states. They act through three different types of opioid receptors (mu, kappa and delta) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al., Nat Biotechnol 2009; 27: 659-666).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al., Lancet Neurol. 2013 November; 12(11):1084-95). In fact, positive synergistic interaction for several compounds, including analgesics, has been described (Schroder et al., J Pharmacol Exp Ther. 2011; 337:312-20. Erratum in: J Pharmacol Exp Ther. 2012; 342:232; Zhang et al., Cell Death Dis. 2014; 5:e1138; Gilron et al., 2013, supra).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, Nat Chem Biol. 2008; 4:682-90).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008, supra).

Thus, in a preferred embodiment, the compounds of the present invention, having inhibitory effects towards the α₂δ subunit, in particular the α₂δ-1 subunit, of voltage-gated calcium channels, additionally inhibit mu opioid receptor. The present invention relates also to the advantages of having dual activity, for μ-receptor and the α₂δ-1 subunit of voltage-gated calcium channels, in the same molecule to treat chronic pain.

In this way, the present invention relates to compounds having a mechanism of action on blocking the α₂δ subunit, in particular the α₂δ-1 subunit, of voltage-gated calcium channels. The present invention also relates to compounds having a complementary dual mechanism of action (μ-receptor agonist and blocker of the α₂δ subunit, in particular the α₂δ-1 subunit, of voltage-gated calcium channels) which implies a better profile of tolerability than the strong opioids (morphine, oxycodone, fentanyl etc) and/or better efficacy and tolerability than gabapentinoids (pregabalin and gabapentin).

Pain is multimodal in nature, since in nearly all pain states several mediators, signaling pathways and molecular mechanisms are implicated. Consequently, monomodal therapies can be complemented with a dual mechanism of action to provide complete pain relief. Currently, combining existing therapies is a common clinical practice and many efforts are directed to assess the best combination of available drugs in clinical studies (Mao, J., Gold, M. S., Backonja, M.; 2011; J. Pain; 12; 157-166).

Accordingly, there is still a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

The authors of the present invention, have found a series of compounds that show pharmacological activity towards the α₂δ subunit, in particular the α₂δ-1 subunit, of the voltage-gated calcium channel, or compounds that show dual pharmacological activity towards both the α₂δ subunit, in particular the α₂δ-1 subunit, of the voltage-gated calcium channel and the μ-opioid receptor (MOR) resulting in an innovative, effective, complementary and alternative solution for the treatment of pain.

In view of the existing results of the currently available therapies and clinical practices, the present invention offers a solution by developing compounds binding to a single target or by combining in a single compound binding to two different targets relevant for the treatment of pain. This was mainly achieved by providing the compounds according to the invention that bind to the α₂δ subunit, in particular the α₂δ-1 subunit, of the voltage-gated calcium channel, or both to the μ-opioid receptor and to the α₂δ subunit, in particular the α₂δ-1 subunit, of the voltage-gated calcium channel.

### SUMMARY OF THE INVENTION

In this invention a family of structurally distinct piperazinyl and piperidinyl quinazolin-4(3H)-one derivatives, encompassed by formula (I), which have a pharmacological activity towards the α₂δ subunit, in particular the α₂δ-1 subunit, of the voltage-gated calcium channel, or which have a dual pharmacological activity towards both the α₂δ subunit, in particular the α₂δ-1 subunit, of the voltage-gated calcium channel and the μ-opioid receptor, were identified thus solving the above problem of identifying alternative or improved pain treatments by offering such compounds.

The main object of the invention is directed to a compound having binding capacity to the α₂δ subunit, in particular the α₂δ-1 subunit, of the voltage-gated calcium channel for use in the treatment of pain.

Another object of the invention is directed to a compound having a dual activity for binding to the α₂δ subunit, in particular the α₂δ-1 subunit, of the voltage-gated calcium channel and the μ-opioid receptor for use in the treatment of pain.

The invention is directed in a main aspect to a compound of general Formula (I),

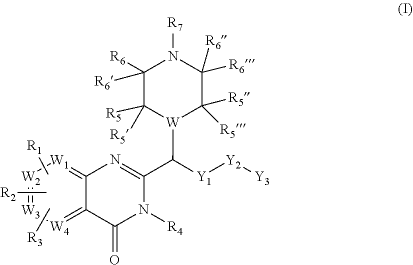

wherein R₁, R₂, R₃, R₄, R₅, R₅′, R₅″, R₅′″, R₆, R₆′, R₆″, R₆′″, R₇, W, w₁, w₂, w₃, w₄, Y₁, Y₂ and Y₃ are as defined below in the detailed description.

A further object of the invention refers to the processes for preparation of compounds of general formula (I).

A still further object of the invention refers to the use of intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct piperazinyl and piperidinyl quinazolin-4(3H)-one derivatives which have primary pharmacological activity towards the α₂δ subunit, in particular the α₂δ-1 subunit, of the voltage-gated calcium channel or which have a dual pharmacological activity towards both the α₂δ subunit, in particular the α₂δ-1 subunit, of the voltage-gated calcium channel and the μ-opioid receptor.

The invention is directed to compounds having primary activity binding to the α₂δ subunit, in particular the α₂δ-1 subunit, of the voltage-gated calcium channel or having a dual activity binding to the α₂δ subunit, in particular the α₂δ-1 subunit, of the voltage-gated calcium channel and the μ-opioid receptor for use in the treatment of pain.

As this invention is aimed at providing a compound or a chemically related series of compounds which act as ligands of the α₂δ subunit, in particular the α₂δ-1 subunit, of the voltage-gated calcium channel or as dual ligands of the α₂δ subunit, in particular the α₂δ-1 subunit, of the voltage-gated calcium channel and the μ-opioid receptor it is a preferred embodiment if the compound has a binding expressed as Kᵢ responding to the following scales:

Kᵢ(μ) is preferably <1000 nM, more preferably <500 nM.

Preferably, when Kᵢ (μ)>500 nM, the following scale has been adopted for representing the binding to the μ-receptor:
+Kᵢ (μ)>500 nM or inhibition ranges between 1% and 50%.

Kᵢ(α₂δ-1) is preferably <10000 nM, more preferably <5000 nM, even more preferably <3000 nM or even more preferably <500 nM.

Preferably, when Kᵢ(α₂δ-1)>5000 nM, the following scale has been adopted for representing the binding to the α₂δ-1 subunit of voltage-gated calcium channels:
+Kᵢ(α₂δ-1)>5000 nM or inhibition ranges between 1% and 50%

The applicant has surprisingly found that the problem of providing a new effective and alternative solution for treating pain and pain related disorders can be solved by using an analgesic approach using ligands binding to the α₂δ subunit, in particular the α₂δ-1 subunit, of the voltage-gated calcium channel or a multimodal balanced analgesic approach combining two different synergistic activities in a single drug (i.e., dual ligands which are bifunctional and bind to μ-opioid receptor and to α₂δ subunit, in particular the α₂δ-1 subunit, of the voltage-gated calcium channel), thereby enhancing through the α₂δ blockade without increasing the undesirable side effects of the μ-opioid activity. This supports the therapeutic value of a dual agent, whereby the α₂δ binding component acts as an intrinsic adjuvant of the MOR binding component.

A dual compound that possess binding to both the μ-opioid receptor and to the α₂δ subunit of the voltage-gated calcium channel shows a highly valuable therapeutic potential by achieving an outstanding analgesia (enhanced in respect to the potency of the opioid component alone) with a reduced side-effect profile (safety margin increased compared to that of the opioid component alone) versus existing opioid therapies.

Advantageously, the compounds according to the present invention would in addition show one or more the following functionalities: blockade of the α₂δ subunit, in particular the α₂δ-1 subunit, of the voltage-gated calcium channel and μ-opioid receptor agonism. It has to be noted, though, that functionalities “antagonism” and “agonism” are also sub-divided in their effect into subfunctionalities like partial agonism or inverse agonism. Accordingly, the functionalities of the compounds should be considered within a relatively broad bandwidth.

An antagonist blocks or dampens agonist-mediated responses. Known subfunctionalities are neutral antagonists or inverse agonists.

An agonist increases the activity of the receptor above its basal level. Known subfunctionalities are full agonists, or partial agonists.

In addition, the two mechanisms complement each other since MOR agonists are only marginally effective in the treatment of neuropathic pain, while the blockers of the α₂δ subunit, in particular the α₂δ-1 subunit, of voltage-gated calcium channels show outstanding effects in preclinical neuropathic pain models. Thus, the α₂δ component, in particular the α₂δ-1 component, adds unique analgesic actions in opioid-resistant pain. Finally, the dual approach has clear advantages over MOR agonists in the treatment of chronic pain as lower and better tolerated doses would be needed based on the potentiation of analgesia but not of the adverse events of MOR agonists.

A further advantage of using designed multiple ligands is a lower risk of drug-drug interactions compared to cocktails or multi-component drugs, thus involving simpler pharmacokinetics and less variability among patients. Additionally, this approach may improve patient compliance and broaden the therapeutic application in relation to monomechanistic drugs, by addressing more complex aetiologies. It is also seen as a way of improving the R&D output obtained using the “one drug-one target” approach, which has been questioned over the last years [Bornot A, Bauer U, Brown A, Firth M, Hellawell C, Engkvist O. Systematic Exploration of Dual-Acting Modulators from a Combined Medicinal Chemistry and Biology Perspective. J. Med. Chem, 56, 1197-1210 (2013)].

In its broader aspect, the present invention is directed to compounds of general Formula (I):

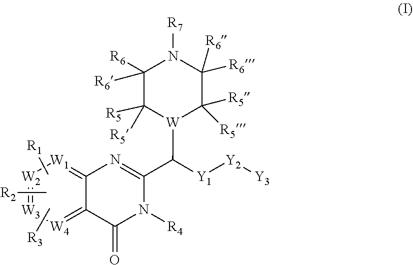

wherein

Y₁ is —C(R_{y}R_{y}′)—;
wherein R_{y} and R_{y}′ are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R_{y} and R_{y}′ form, with the carbon atom to which they are attached, a substituted or unsubstituted cycloalkyl;

Y₂ is —C(R_{y}″R_{y}′″)—;
wherein R_{y}″ and R_{y}′″ are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R_{y}″ and R_{y}′″ form, with the carbon atom to which they are attached, a substituted or unsubstituted cycloalkyl;

Y₃ is —CH₃ or —CH₂CH₃;
alternatively, Y₂ and Y₃ taken together, form a substituted or unsubstituted cycloalkyl;

W is nitrogen or —CR_{w}—; wherein R_{w} is hydrogen or halogen;
alternatively, R_{w} and one of R₅, R₅′, R₅″ or R₅′″ form a double bond;

w1, w2, w3 and w4 are independently selected from the group consisting of nitrogen and carbon;

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R_{8′}, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
n is 0, 1, 2, 3, 4 or 5;
R₈ and R₈′ are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

R₂ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₂₁, —NO₂₁—NR₂₁R₂₁′, —NR₂₁C(O)R₂₁′, —NR₂₁S(O)₂R₂₁′, —S(O)₂NR₂₁R₂₁′, NR₂₁C(O)NR₂₁′R₂₁″, —SR₂₁, —S(O)R₂₁, —S(O)₂R₂₁, —CN, haloalkyl, haloalkoxy, —C(O)OR₂₁, —C(O)NR₂₁R₂₁′, —NR₂₁S(O)₂NR₂₁′R₂₁″ and —C(CH₃)₂OR₂₁;
wherein R₂₁, R₂₁′ and R₂₁″ are independently selected from hydrogen, substituted or unsubstituted C₁₋₆ alkyl, substituted or unsubstituted C₂₋₆ alkenyl and substituted or unsubstituted C₂₋₆ alkynyl;

R₃ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₃₁, —NO₃₁—NR₃₁R₃₁′, —NR₃₁C(O)R₃₁′, —NR₃₁S(O)₃R₃₁′, —S(O)₃NR₃₁R₃₁′, NR₃₁C(O)NR₃₁′R₃₁″, —SR₃₁, —S(O)R₃₁, —S(O)₃R₃₁, —CN, haloalkyl, haloalkoxy, —C(O)OR₃₁, —C(O)NR₃₁R₃₁′, —NR₃₁S(O)₃NR₃₁′R₃₁″ and —C(CH₃)₃OR₃₁;
wherein R₃₁, R₃₁′ and R₃₁″ are independently selected from hydrogen, substituted or unsubstituted C₁₋₆ alkyl, substituted or unsubstituted C₃₋₆ alkenyl and substituted or unsubstituted C₃₋₆ alkynyl;

R₄ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted alkylheterocyclyl, substituted or unsubstituted alkylaryl and substituted or unsubstituted alkylcycloalkyl;

R₅, R₅′, R₅″ and R₅′″ are independently selected from hydrogen, halogen substituted or unsubstituted C₁₋₆ alkyl, substituted or unsubstituted C₂₋₆ alkenyl and substituted or unsubstituted C₂₋₆ alkynyl;
alternatively, R₅ and R₅′ and/or R₅″ and R₅′″ taken together with the carbon atom to which they are attached form a carbonyl group;

R₆, R₆′, R₆″ and R₆′″ are independently selected from hydrogen, substituted or unsubstituted C₁₋₆ alkyl, substituted or unsubstituted C₂₋₆ alkenyl and substituted or unsubstituted C₂₋₆ alkynyl;

R₇ is selected from hydrogen, substituted or unsubstituted C₁₋₆ alkyl, substituted or unsubstituted C₂₋₆ alkenyl and substituted or unsubstituted C₂₋₆ alkynyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a particular embodiment,

w1, w2, w3 and w4 are all carbon.

In a particular embodiment,

one or two of w1, w2, w3 and w4 are nitrogen while the others are carbon.

In a particular embodiment,

one of w1, w2, w3 and w4 is nitrogen while the others are carbon.

In a particular embodiment,

two of w1, w2, w3 and w4 are nitrogen while the others are carbon.

In a particular embodiment, the following proviso applies:

when R₇ is not hydrogen, then one of R₆, R₆′, R₆″ or R₆′″ is not hydrogen.

In a particular embodiment, the following proviso applies:

when R₇ is substituted or unsubstituted C₁₋₆ alkyl, substituted or unsubstituted C₂₋₆ alkenyl or substituted or unsubstituted C₂₋₆ alkynyl, then one of R₆, R₆′, R₆″ or R₆′″ is selected from substituted or unsubstituted C₁₋₆ alkyl, substituted or unsubstituted C₂₋₆ alkenyl and substituted or unsubstituted C₂₋₆ alkynyl.

In a particular embodiment, the following proviso applies:

when R₇ is substituted or unsubstituted C₁₋₆ alkyl, then one of R₆, R₆′, R₆″ or R₆′″ is substituted or unsubstituted C₁₋₆ alkyl.

In a further embodiment the compound according to the invention is a compound of general Formula (I)

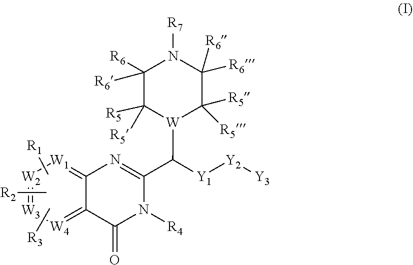

wherein

Y₁ is —C(R_{y}R_{y}′)—;
wherein R_{y} and R_{y}′ are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R_{y} and R_{y}′ form, with the carbon atom to which they are attached, a substituted or unsubstituted cycloalkyl;

Y₂ is —C(R_{y}″R_{y}′″)—;
wherein R_{y}″ and R_{y}′″ are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R_{y}″ and R_{y}′″ form, with the carbon atom to which they are attached, a substituted or unsubstituted cycloalkyl;

Y₃ is —CH₃ or —CH₂CH₃;
alternatively, Y₂ and Y₃ taken together, form a substituted or unsubstituted cycloalkyl;

W is nitrogen or —CR_{w}—; wherein R_{w} is hydrogen or halogen;
alternatively, R_{w} and one of R₅, R₅′, R₅″ or R₅′″ form a double bond;

w1, w2, w3 and w4 are independently selected from the group consisting of nitrogen and carbon;

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R_{8′}, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
wherein
the alkyl, alkenyl or alkynyl defined in R₁, if substituted, is substituted with one or more substituent/s selected from —OR₁₁, halogen, —CN, haloalkyl, haloalkoxy and —NR₁₁R₁₁′;
the cycloalkyl, aryl heterocyclyl, defined in R₁, also in alkylcycloalkyl, alkylaryl and alkylheterocyclyl, if substituted, is substituted with one or more substituent/s selected from ═O, halogen, —R₁₁, —OR₁₁, —NO₂, —(CH₂)ₘNR₁₁R₁₁′, —NR₁₁C(O)R₁₁′, —NR₁₁S(O)₂R₁₁′, —S(O)₂NR₁₁R₁₁′, NR₁₁C(O)NR₁₁′R₁₁″, —SR₁₁, —S(O)R₁₁, —S(O)₂R₁₁, —CN, haloalkyl, haloalkoxy, —C(O)OR₁₁, —C(O)NR₁₁R₁₁′, —OCH₂CH₂OR₁₁, NR₁₁S(O)₂NR₁₁′R₁₁″, —C(CH₃)₂OR₁₁, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
R₁₁, R₁₁′ and R₁₁″ are independently selected from hydrogen, substituted or unsubstituted C₁₋₆ alkyl, substituted or unsubstituted C₂₋₆ alkenyl and substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
m is 0, 1, 2, 3, 4 or 5;
n is 0, 1, 2, 3, 4 or 5;
R₈ and R₈′ are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
wherein
the alkyl, alkenyl or alkynyl defined in R₈ or R₈′, if substituted, is substituted with one or more substituent/s selected from —OR₈₁, halogen, —CN, haloalkyl, haloalkoxy and —NR₈₁R₈₁′;
the cycloalkyl heterocyclyl or aryl defined in R₈ or R₈′, also in alkylcycloalkyl, alkylheterocyclyl and alkylaryl if substituted, is substituted with one or more substituent/s selected from ═O, halogen, —R₈₁, —OR₈₁, —NO₂, —NR₈₁R₈₁′, —NR₈₁C(O)R₈₁′, —NR₈₁S(O)₂R₈₁′, S(O)₂NR₈₁R₈₁′, —NR₈₁C(O)NR₈₁′R₈₁″, —SR₈₁, —S(O)R₈₁, —S(O)₂R₈₁, —CN, haloalkyl, haloalkoxy, —C(O)OR₈₁, —C(O)NR₈₁R₈₁′, —OCH₂CH₂OR₈₁, —NR₈₁S(O)₂NR₈₁′R₈₁″ and —C(CH₃)₂OR₈₁, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
wherein R₈₁, R₈₁′ and R₈₁″ are independently selected from hydrogen, substituted or unsubstituted C₁₋₆ alkyl, substituted or unsubstituted C₂₋₆ alkenyl and substituted or unsubstituted C₂₋₆ alkynyl;

R₂ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₂₁, —NO₂, —NR₂₁R₂₁′, —NR₂₁C(O)R₂₁′, —NR₂₁S(O)₂R₂₁′, —S(O)₂NR₂₁R₂₁′, NR₂₁C(O)NR₂₁′R₂₁″, —SR₂₁, —S(O)R₂₁, —S(O)₂R₂₁, —CN, haloalkyl, haloalkoxy, —C(O)OR₂₁, —C(O)NR₂₁R₂₁′, —NR₂₁S(O)₂NR₂₁′R₂₁″ and —C(CH₃)₂OR₂₁;
wherein R₂₁, R₂₁′ and R₂₁″ are independently selected from hydrogen, substituted or unsubstituted C₁₋₆ alkyl, substituted or unsubstituted C₂₋₆ alkenyl and substituted or unsubstituted C₂₋₆ alkynyl;

R₃ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₃₁, —NO₃₁—NR₃₁R₃₁′, —NR₃₁C(O)R₃₁′, —NR₃₁S(O)₃R₃₁′, —S(O)₃NR₃₁R₃₁′, NR₃₁C(O)NR₃₁′R₃₁″, —SR₃₁, —S(O)R₃₁, —S(O)₃R₃₁, —CN, haloalkyl, haloalkoxy, —C(O)OR₃₁, —C(O)NR₃₁R₃₁′, —NR₃₁S(O)₃NR₃₁′R₃₁″ and —C(CH₃)₃OR₃₁;
wherein R₃₁, R₃₁′ and R₃₁″ are independently selected from hydrogen, substituted or unsubstituted C₁₋₆ alkyl, substituted or unsubstituted C₃₋₆ alkenyl and substituted or unsubstituted C₃₋₆ alkynyl;

R₄ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted alkylheterocyclyl, substituted or unsubstituted alkylaryl and substituted or unsubstituted alkylcycloalkyl;
the alkyl, alkenyl or alkynyl defined in R₄, if substituted, is substituted with one or more substituent/s selected from —OR₄₁, halogen, —CN, —C(O)OR₄₁, haloalkyl, haloalkoxy, —NR₄₁R₄₁′, substituted or unsubstituted cycloalkyl, substituted or unsubstituted heterocyclyl and substituted or unsubstituted aryl;
the cycloalkyl as defined in R₄, also in alkylcycloalkyl, or the heterocyclyl in alkylheterocyclyl, or the aryl in alkylaryl, if substituted and the substitution has not been defined otherwise, it is substituted with one or more substituent/s selected from halogen, —R₄₁, —OR₄₁, —NO₂, —NR₄₁R₄₁′, —NR₄₁C(O)R₄₁′, —NR₄₁S(O)₂R₄₁′, —S(O)₂NR₄₁R₄₁′, —NR₄₁C(O)NR₄₁′R₄₁″, —SR₄₁, —S(O)R₄₁, —S(O)₂R₄₁, —CN, haloalkyl, haloalkoxy, —C(O)OR₄₁, —C(O)NR₄₁R₄₁′, —OCH₂CH₂OR₄₁, —NR₄₁S(O)₂NR₄₁′R₄₁″ and —C(CH₃)₂OR₄₁;
   wherein R₄₁, R₄₁′ and R₄₁″ are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

R₅, R₅′, R₅″ and R₅′″ are independently selected from hydrogen, halogen substituted or unsubstituted C₁₋₆ alkyl, substituted or unsubstituted C₂₋₆ alkenyl and substituted or unsubstituted C₂₋₆ alkynyl;
alternatively, R₅ and R₅′ and/or R₅″ and R₅′″ taken together with the carbon atom to which they are attached form a carbonyl group;

R₆, R₆′, R₆″ and R₆′″ are independently selected from hydrogen, substituted or unsubstituted C₁₋₆ alkyl, substituted or unsubstituted C₂₋₆ alkenyl and substituted or unsubstituted C₂₋₆ alkynyl;
wherein
the alkyl, alkenyl or alkynyl, in R₆, R₆′, R₆″ and R₆′″, if substituted, it is substituted with one or more substituent/s selected from —OR₆₁, —C(O)OR₆₁, halogen, —CN, haloalkyl, haloalkoxy and —NR₆₁R₆₁′;
wherein R₆₁ and R₆₁′ are independently selected from hydrogen, unsubstituted C₁₋₆ alkyl, unsubstituted C₂₋₆ alkenyl, and unsubstituted C₂₋₆ alkynyl;

R₇ is selected from hydrogen, substituted or unsubstituted C₁₋₆ alkyl, substituted or unsubstituted C₂₋₆ alkenyl and substituted or unsubstituted C₂₋₆ alkynyl;

the alkyl, alkenyl or alkynyl, if substituted and the substitution has not been defined otherwise, it is substituted with one or more substituent/s selected from —OR₁₃, halogen, —CN, haloalkyl, haloalkoxy and —NR₁₃R₁₃′;
wherein R₁₃ and R₁₃′ are independently selected from hydrogen, unsubstituted C₁₋₆ alkyl, unsubstituted C₂₋₆ alkenyl, and unsubstituted C₂₋₆ alkynyl;

the aryl, heterocyclyl or cycloalkyl, also in alkylaryl, alkylheterocyclyl or alkylcycloalkyl, if substituted and the substitution has not been defined otherwise, it is substituted with one or more substituent/s selected from halogen, —R₁₄, —OR₁₄, —NO₂, —NR₁₄R₁₄′, —NR₁₄C(O)R₁₄′, —NR₁₄S(O)₂R₁₄′, —S(O)₂NR₁₄R₁₄′, —NR₁₄C(O)NR₁₄′R₁₄″, —SR₁₄, —S(O)R₁₄, —S(O)₂R₁₄, —CN, haloalkyl, haloalkoxy, —C(O)OR₁₄, —C(O)NR₁₄R₁₄′, —OCH₂CH₂OR₁₄, —NR₁₄S(O)₂NR₁₄′R₁₄″ and —C(CH₃)₂OR₁₄;
wherein R₁₄, R₁₄′ and R₁₄″ are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I′)

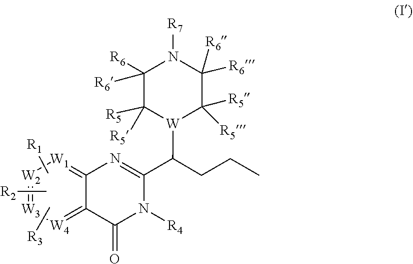

wherein R₂, R₃, R₄, R₅, R₅′, R₅″, R₅′″, R₆, R₆′, R₆″, R₆′″, R₇, W, w₁, w₂, w₃ and w₄ are as defined below in the detailed description,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I²′)

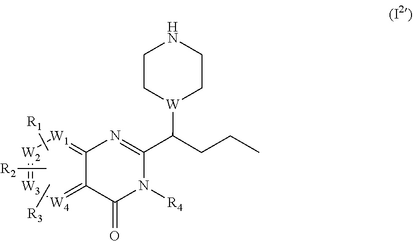

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I³′)

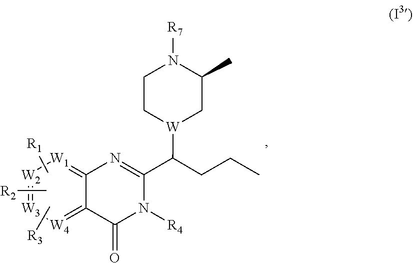

wherein R₁, R₂, R₃, R₄, R₇, W, w₁, w₂, w₃ and w₄ are as defined in the description,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I⁴′)

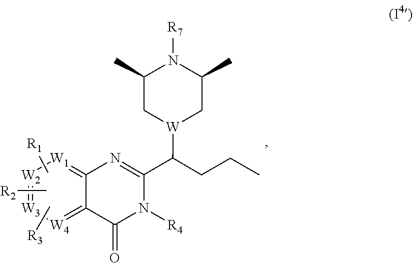

wherein R₁, R₂, R₃, R₄, R₇, W, w₁, w₂, w₃ and w₄ are as defined in the description, optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I⁵′)

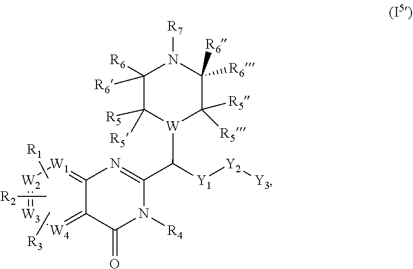

wherein R₂, R₃, R₄, R₅, R₅′, R₅″, R₅′″, R₆, R₆′, R₆″, R₆′″, R₇, W, w₁, w₂, w₃ and w₄ are as defined in the description,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

For clarity purposes, all groups and definitions described in the present description and referring to compounds of general Formula (I), also apply to compounds of general Markush Formulae (I′), (I²′), (I³′), (I⁴′), and (I⁵′), (where applicable), and to all intermediates of synthesis, when those groups are present in the mentioned general Markush formulae, since compounds of general Markush Formulae (I′), (I²′), (I³′), (I⁴′), and (I⁵′), are included within the scope of the larger definition of general Markush Formula (I).

For clarity purposes, the expression e.g. “the cycle in R₈—R₈′”, means the cycle resulting when R₈ and R₈′ form a cycle together with the atom(s) to which they are attached. This cycle can then be substituted or not. This definition is also generally applicable and can be also applied as a definition of any other cycle (preferably cycloalkyls, heterocyclyls or aryls) formed from two different functional groups like e.g. “the cycle in Rᵢ-f_{i′}” means the cycle resulting when Rᵢ and Rᵢ′ form a cycle together with the atom(s) to which they are attached. This cycle can then be substituted or not.

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NRₖR_{k′}, —SRₖ, —S(O)Rₖ, —S(O)₂Rₖ, —ORₖ, —C(O)Rₖ, —C(O)ORₖ, —CN, —C(O)NRₖR_{k′}, haloalkyl, haloalkoxy, being Rₖ represented by R₁₁, R₁₃, R₄₁, R₆₁ or R₈₁ (being R_{k′} represented by R_{11′}, R_{13′}, R_{41′}, R_{61′} or R₈₁); wherein R₁ to R₈₁″ and R_{w}, R_{y}, R_{y′}, R_{y″} and R_{y′″} areas defined in the description, and wherein when different radicals R₁ to R₈₁″ and R_{w}, R_{y}, R_{y′}, R_{y′} and R_{y′″} are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which is substituted with one or more of halogen (F, Cl, Br, I), —NRₖR_{k′}, —ORₖ, —CN, —SRₖ, haloalkyl, haloalkoxy, being Rₖ represented by R₁₁, R₁₃, R₄₁, R₆₁ or R₈₁, (being R_{k′} represented by R_{11′}, R_{13′}, R_{41′}, R_{61′} or R_{81′}) wherein R₁ to R₈₁″ and R_{w}, R_{y}, R_{y′}, R_{y″} and R_{y′″} areas defined in the description, and wherein when different radicals R₁ to R₈₁″ and R_{w}, R_{y}, R_{y′}, R_{y″} and R_{y′″} are present simultaneously in Formula I they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH₂—CHCl₂. Preferably haloalkoxy is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphthyl or anthracenyl, preferably is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic ring contains one or more heteroatoms selected from the group consisting of nitrogen, oxygen and/or sulfur in the ring; preferably is a 5 to 18 membered mono or polycyclic aromatic heterocyclic ring system of one or two rings of which at least one aromatic ring contains one or more heteroatoms selected from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms selected from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, especially is benzodioxane, morpholine, tetrahydropyran, piperidine, oxopyrrolidine and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms selected from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms selected from the group consisting of nitrogen, oxygen and/or sulfur in the ring.

Preferred examples of heterocyclyls include oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane and pyrrolidine.

In the context of this invention oxopyrrolidine is understood as meaning pyrrolidin-2-one.

An N-containing heterocyclyl is a heterocyclic ring system of one or more saturated or unsaturated rings of which at least one ring contains a nitrogen and optionally one or more further heteroatoms selected from the group consisting of nitrogen, oxygen and/or sulfur in the ring; preferably is a heterocyclic ring system of one or two saturated or unsaturated rings of which at least one ring contains a nitrogen and optionally one or more further heteroatoms selected from the group consisting of nitrogen, oxygen and/or sulfur in the ring, more preferably is selected from oxazepam, pyrrolidine, imidazole, oxadiazole, tetrazole, azetidine, pyridine, pyrimidine, piperidine, piperazine, benzimidazole, indazole, benzothiazole, benzodiazole, morpholine, indoline, triazole, isoxazole, pyrazole, pyrrole, pyrazine, pyrrolo[2,3b]pyridine, quinoline, quinolone, isoquinoline, tetrahydrothienopyridine, phthalazine, benzo-1,2,5-thiadiazole, indole, benzotriazole, benzoxazole oxopyrrolidine, carbazole or thiazole.

An heterocyclyl is a heterocyclic ring system of one or more saturated and/or unsaturated rings of which at least one ring contains one or more heteroatoms selected from the group consisting of nitrogen, oxygen and/or sulfur in the ring; preferably is a heterocyclic ring system of one saturated and/or unsaturated ring containing one or more heteroatoms selected from the group consisting of nitrogen, oxygen and/or sulfur in the ring, or a heterocyclic ring system of two saturated and/or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, quinolone, isoquinoline, tetrahydrothienopyridine, phthalazine, benzo-1,2,5-thiadiazole, indole, benzotriazole, benzoxazole oxopyrrolidine, benzodioxolane, benzodioxane, carbazole, oxaspirodecan or thiazole;

In general, such a heterocyclyl may contain between 3 and 32 atoms in the rings (preferably 4 to 20 atoms in the rings, or most preferably 5 to 18 atoms in the rings). Thus, a heterocyclyl may contain between 3 and 12 atoms in the ring (preferably 4 to 10 atoms in the ring, or 5 to 8 atoms in the ring, or 5 to 6 atoms in the ring) in case of a heterocyclyl of one ring. Such a heterocyclyl may also contain between 5 and 22 atoms in both rings together (preferably 6 to 16 atoms in both rings together, or 7 to 12 atoms in both rings together or 8 to 10 atoms in both rings together) in case of a heterocyclyl of two rings. Such a heterocyclyl may also contain between 7 and 32 atoms in the 3 rings together (preferably 10 to 22 atoms in the three rings together, or 12 to 20 atoms in the three rings together or 10 to 18 atoms in the three rings together) in case of a heterocyclyl of three rings. Each ring of the ring system, independently of each other, can be saturated or unsaturated.

In the context of this invention, a cyclic amide is defined as a subgroup of a heterocyclyl (as defined above) formed through the cyclization of a carbon sequence, containing at least the sequence

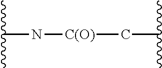

forming part of the cycle. Said cyclic amide may optionally be fused to a ring system. Preferably the cyclic amide is an “indoline-2-one”. A cyclic amide may be substituted or unsubstituted as defined for heterocyclyl above.

In the context of this invention, a cyclic urea is defined as a subgroup of a heterocyclyl (as defined above) formed through the cyclization of a carbon sequence containing at least the sequence

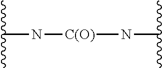

forming part of the cycle. Said cyclic urea may optionally be fused to a ring system. Preferably the cyclic urea is “1H-benzo[d]imidazol-2(3H)-one”. A cyclic urea may be substituted or unsubstituted as defined for heterocyclyl above.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl). More preferably, the “alkyl” in alkylaryl is an unsubstituted alkyl.

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine. More preferably, the “alkyl” in alkylheterocyclyl is an unsubstituted alkyl.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning a cycloalkyl group (see above) being connected to another atom through 1 to 4 (—CH₂—) groups. Most preferably alkylcycloalkyl is —CH₂-cyclopropyl. More preferably, the “alkyl” in alkycycloalkyl is an unsubstituted alkyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —Rₖ, —ORₖ, —CN, —NO₂, —NRₖR_{k′}, —C(O)ORₖ, NRₖC(O)R_{k′}, —C(O)NRₖR_{k′}, —NRₖS(O)₂R_{k′}, ═O, —OCH₂CH₂OH, —NRₖC(O)NR_{k′}R_{k″}, —S(O)₂NRₖR_{k′}, —NRₖS(O)₂NR_{k′}R_{k″}, haloalkyl, haloalkoxy, —SRₖ, —S(O)Rₖ, —S(O)₂Rₖ or C(CH₃)ORₖ, or substituted or unsubstituted alkylcycloalkyl, substituted or unsubstituted alkylaryl, substituted or unsubstituted alkylheterocyclyl, with Rₖ, R_{k′} and R_{k″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆-alkyl; a saturated or unsaturated, linear or branched, substituted or unsubstituted —C(O)—C₁₋₆-alkyl-group; a saturated or unsaturated, linear or branched, substituted or unsubstituted —C(O)—O—C₁₋₆-alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being Rₖ one of R₁₁, R₁₄, R₄₁ or R₈₁, (being Rₖ′ one of R₁₁′, R₁₄′, R₄₁′ or R₈₁′ or R₉₁′, being Rₖ″ one of R_{11″}, R_{14″}, R_{41″} or R_{81″}); wherein R₁ to R₈₁″ and R_{w}, R_{y}, R_{y′}, R_{y″} and R_{y′″} are as defined in the description, and wherein when different radicals R₁ to R₈₁″ and R_{w}, R_{y}, R_{y′}, R_{y″} and R_{y′″} are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alyklaryl, alkylcycloalkyl or alkylheterocyclyl) with one or more of halogen (F, Cl, Br, I), —Rₖ, —ORₖ, —CN, —NO₂, —NRₖR_{k′″}, NRₖC(O)R_{k′}, —NRₖS(O)₂R_{k′}, —S(O)₂NRₖR_{k′}, —NRₖC(O)NR_{k′}R_{k″}, haloalkyl, haloalkoxy, —SRₖ, —S(O)Rₖ or S(O)₂Rₖ, or substituted or unsubstituted alkylcycloalkyl, substituted or unsubstituted alkylaryl, substituted or unsubstituted alkylheterocyclyl, being Rₖ one of R₁₁, R₁₄, R₄₁ or R₈₁, (being R_{k′} one of R₁₁′, R₁₄′, R₄₁′ or R₈₁′; being R_{k″} one of R_{11″}, R_{14″}, R_{41″} or R_{81″}) wherein R₁ to R₈₁″ and R_{w}, R_{y}, R_{y′}, R_{y″} and R_{y′″} are as defined in the description, and wherein when different radicals R₁ to R₈₁″ and R_{w}, R_{y}, R_{y′}, R_{y″} and R_{y′″} are present simultaneously in Formula I they may be identical or different.

In connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl, non-aromatic heterocyclyl or non aromatic alkyl-heterocyclyl with

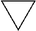

(leading to a spiro structure) and/or with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl, non-aromatic heterocyclyl or non aromatic alkyl-heterocyclyl is spirosubstituted or substituted with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl, non-aromatic heterocyclyl or non aromatic alkyl-heterocyclyl with ═O.

A ring system is an organic system consisting of at least one ring of connected atoms but including also systems in which two or more rings of connected atoms are joined with “joined” meaning that the respective rings are sharing one (like a spiro structure), two or more atoms being a member or members of both joined rings.

The term “polycyclic ring system” means that the ring system is made of two or more rings joined by sharing at least one atom.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO-) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic-especially not caused by the counter-ion) if used appropriately for a treatment especially if used on or applied to humans and/or mammals.

Please note that “or a corresponding salt thereof” does also mean “or a corresponding pharmaceutically acceptable salt thereof”. This does apply to all below described embodiments and uses of “salt” being thus equivalent to “pharmaceutically acceptable salt”.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention. This would especially also apply to the provisos described above so that any mentioning of hydrogen or any “H” in a formula would also cover deuterium or tritium.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a further embodiment the compound according to the invention of general Formula (I) is a compound wherein

R_{y} and R_{y}′ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{y}″ and R_{y}′″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from the group consisting of hydrogen, halogen, substituted or unsubstituted C₁₋₆ alkyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from the group consisting of hydrogen, halogen, substituted or unsubstituted C₁₋₆ alkyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted heterocyclyl, substituted or unsubstituted aryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from the group consisting of hydrogen, halogen, substituted or unsubstituted C₁₋₆ alkyl, —OR₈, —(CH₂)ₙNR₈R₈′, —NR₈C(O)R₈′, —NR₈C(O)OR₈′, —C(O)NR₈R_{8′}, —C(O)OR₈, —OCHR₈R₈′, haloalkyl, haloalkoxy and —CN;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from the group consisting of hydrogen, halogen, substituted or unsubstituted C₁₋₆ alkyl, —OR₈, —(CH₂)ₙNR₈R₈′, —CH(phenyl)-NR₈R₈′, —NR₈C(O)R₈′, —NR₈C(O)OR₈′, —C(O)NR₈R_{8′}, —C(O)OR₈, —OCHR₈R₈′, haloalkyl, haloalkoxy and —CN;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0 or 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 0, 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₈ and R₈′ are independently selected from the group consisting of hydrogen, substituted or unsubstituted C₁₋₆ alkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₈ and R₈′ are independently selected from the group consisting of hydrogen, substituted or unsubstituted C₁₋₆ alkyl, substituted or unsubstituted heterocyclyl, substituted or unsubstituted aryl, substituted or unsubstituted alkylheterocyclyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₈ and R₈′ are independently selected from the group consisting of hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from hydrogen, halogen, substituted or unsubstituted C₁₋₆ alkyl, —OR₂₁, —NO₂, —NR₂₁R₂₁′, —NR₂₁C(O)R₂₁′, —NR₂₁S(O)₂R₂₁′, —S(O)₂NR₂₁R₂₁′, —NR₂₁C(O)NR₂₁′R₂₁″, —SR₂₁, —S(O)R₂₁, —S(O)₂R₂₁, —CN, haloalkyl, haloalkoxy, —C(O)OR₂₁, —C(O)NR₂₁R₂₁′, —NR₂₁S(O)₂NR₂₁′R₂₁″ and —C(CH₃)₂OR₂₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

In a further embodiment the compound according to the invention of general Formula (I) is a compound wherein

R₂ is selected from hydrogen, halogen, substituted or unsubstituted C₁₋₆ alkyl and —OR₂₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is selected from hydrogen, halogen, substituted or unsubstituted C₁₋₆ alkyl, —OR₃₁, —NO₃, —NR₃₁R₃₁′, —NR₃₁C(O)R₃₁′, —NR₃₁S(O)₃R₃₁′, —S(O)₃NR₃₁R₃₁′, —NR₃₁C(O)NR₃₁′R₃₁″, —SR₃₁, —S(O)R₃₁, —S(O)₃R₃₁, —CN, haloalkyl, haloalkoxy, —C(O)OR₃₁, —C(O)NR₃₁R₃₁′, —NR₃₁S(O)₃NR₃₁′R₃₁″ and —C(CH₃)₃OR₃₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is selected from hydrogen, halogen and —OR₃₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
R₄ is selected from substituted or unsubstituted C₁₋₆ alkyl, substituted or unsubstituted cycloalkyl, substituted or unsubstituted alkylheterocyclyl, substituted or unsubstituted alkylaryl and substituted or unsubstituted alkylcycloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
R₄ is selected from substituted or unsubstituted C₁₋₆ alkyl, substituted or unsubstituted alkylheterocyclyl and substituted or unsubstituted alkylcycloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
R₄ is substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₅, R₅′, R₅″ and R₅′″ are independently selected from hydrogen, halogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₅, R₅′, R₅″ and R₅′″ are independently selected from hydrogen and halogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₅ and R₅′ and/or R₅″ and R₅′″ taken together with the carbon atom to which they are attached form a carbonyl group;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₆, R₆′, R₆″ and R₆′″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₆, R₆′, R₆″ and R₆′″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₇ is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁₁, R₁₁′ and R₁₁″ are independently selected from hydrogen, substituted or unsubstituted C₁₋₆ alkyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁₁, R₁₁′ and R₁₁″ are independently selected from hydrogen, substituted or unsubstituted C₁₋₆ alkyl, substituted or unsubstituted aryl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁₁, R₁₁′ and R₁₁″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁₃ and R₁₃′ are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁₄, R₁₄′ and R₁₄″ are independently selected from hydrogen, unsubstituted C₁₋₆ alkyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂₁, R₂₁′ and R₂₁″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃₁, R₃₁′ and R₃₁″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄₁, R₄₁′ and R₄₁″ are independently selected from hydrogen, substituted or unsubstituted C₁₋₆ alkyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄₁, R₄₁′ and R₄₁″ are independently selected from hydrogen, substituted or unsubstituted C₁₋₆ alkyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₆₁ and R₆₁′ are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₈₁, R₈₁′ and R₈₁″ are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl defined in R₁, if substituted, is substituted with one or two substituent/s selected from —OR₁₁, halogen, —CN, haloalkyl, haloalkoxy and —NR₁₁R₁₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₁, if substituted, is substituted with one or more substituent/s selected from —OR₁₁ and halogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl defined in R₁, if substituted, is substituted with one or more substituent/s selected from —OR₁₁ and halogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₁, if substituted, is substituted with one or more substituent/s selected from —OH and fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl defined in R₁, if substituted, is substituted with one or more substituent/s selected from —OH and fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl, aryl heterocyclyl, defined in R₁, also in alkylcycloalkyl, alkylaryl and alkylheterocyclyl, if substituted, is substituted with one or more substituent/s selected from —R₁₁, —OR₁₁, —(CH₂)ₘNR₁₁R₁₁′, —NR₁₁C(O)R₁₁′, substituted or unsubstituted aryl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl, aryl heterocyclyl, defined in R₁, also in alkylcycloalkyl, alkylaryl and alkylheterocyclyl, if substituted, is substituted with one or more substituent/s selected from methyl, OH, —OCH₃, —CH₂NHCH₃, —NH₂, —N(CH₃)₂, —NH(CH₃), —N(CH₃)(benzyl), —N(phenyl)(benzyl), —N(phenyl)(C(O)CH₂CH₃), phenol and phenethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl, aryl heterocyclyl, defined in R₁, also in alkylcycloalkyl, alkylaryl and alkylheterocyclyl, if substituted, is substituted with one or more substituent/s selected from —R₁₁, —OR₁₁, —(CH₂)ₘNR₁₁R₁₁′ and —NR₁₁C(O)R₁₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl, aryl heterocyclyl, defined in R₁, also in alkylcycloalkyl, alkylaryl and alkylheterocyclyl, if substituted, is substituted with one or more substituent/s selected from methyl, OH, —OCH₃, —CH₂NHCH₃, —NH₂, —N(CH₃)₂ and —NH(CH₃);

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₈ or R₈′, if substituted, is substituted with one or more substituent/s selected from —OR₈₁, halogen, —CN, haloalkyl, haloalkoxy and —NR₈₁R₈₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₈ or R₈′, if substituted, is substituted with one or two substituent/s selected from —OR₈₁, halogen, —CN, haloalkyl, haloalkoxy and —NR₈₁R₈₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₈ or R₈′, if substituted, is substituted with one or more —NR₈₁R₈₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₈ or R₈′, if substituted, is substituted with one or more —NH(CH₃);

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl heterocyclyl or aryl defined in R₈ or R₈′, also in alkylcycloalkyl, alkylheterocyclyl and alkylaryl if substituted, is substituted with one or more substituent/s selected from —R₈₁, —OR₈₁, substituted or unsubstituted heterocyclyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl heterocyclyl or aryl defined in R₈ or R₈′, also in alkylcycloalkyl, alkylheterocyclyl and alkylaryl if substituted, is substituted with one or more substituent/s selected from —CH₃, —OCH₃, substituted or unsubstituted pyridine and substituted or unsubstituted benzyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl heterocyclyl or aryl defined in R₈ or R₈′, also in alkylcycloalkyl, alkylheterocyclyl and alkylaryl if substituted, is substituted with one or more substituent/s selected from —R₈₁ and —OR₈₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the cycloalkyl heterocyclyl or aryl defined in R₈ or R₈′, also in alkylcycloalkyl, alkylheterocyclyl and alkylaryl if substituted, is substituted with one or more substituent/s selected from —CH₃ and —OCH₃;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₄, if substituted, is substituted with one or two substituent/s selected from —OR₄₁, halogen, —CN, —C(O)OR₄₁, haloalkyl, haloalkoxy, —NR₄₁R₄₁′, substituted or unsubstituted cycloalkyl, substituted or unsubstituted heterocyclyl and substituted or unsubstituted aryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₄, if substituted, is substituted with one or more substituent/s selected from —OR₄₁, —C(O)OR₄₁ and —NR₄₁R₄₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₄, if substituted, is substituted with one or more substituent/s selected from —OCH₃, —C(O)OH, —C(O)OCH₂CH₃ and —NH(CH₃), —N(CH₃)₂, —N(CH₃)(phenethyl) and —N(CH₃)(CH₂CH₂CH₂-phenyl);

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl defined in R₄, if substituted, is substituted with one or more substituent/s selected from —OCH₃, —C(O)OH, —C(O)OCH₂CH₃ and —NH(CH₃) and —N(CH₃)₂);

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
the cycloalkyl as defined in R₄, also in alkylcycloalkyl, or the heterocyclyl in alkylheterocyclyl, or the aryl in alkylaryl, if substituted and the substitution has not been defined otherwise, it is substituted with one or two substituent/s selected from halogen, —R₄₁, —OR₄₁, —NO₂, —NR₄₁R₄₁′, —NR₄₁C(O)R₄₁′, —NR₄₁S(O)₂R₄₁′, —S(O)₂NR₄₁R₄₁′, —NR₄₁C(O)NR₄₁′R₄₁″, —SR₄₁, —S(O)R₄₁, —S(O)₂R₄₁, —CN, haloalkyl, haloalkoxy, —C(O)OR₄₁, —C(O)NR₄₁R₄₁′, —OCH₂CH₂OR₄₁, —NR₄₁S(O)₂NR₄₁′R₄₁″ and —C(CH₃)₂OR₄₁;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl, in R₆, R₆′, R₆″ and R₆′″, if substituted, it is substituted with one or two substituent/s selected from —OR₆₁, —C(O)OR₆₁, halogen, —CN, haloalkyl, haloalkoxy and —NR₆₁R₆₁′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl in R₆, R₆′, R₆″ and R₆′″, if substituted, it is substituted with one or more substituent/s selected from —OR₆₁, —C(O)OR₆₁ and halogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl in R₆, R₆′, R₆″ and R₆′″, if substituted, it is substituted with one or more substituent/s selected from —OH, —OCH₃, —C(O)OH and fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the alkyl, alkenyl or alkynyl, if substituted and the substitution has not been defined otherwise, it is substituted with one or two substituent/s selected from —OR₁₃, halogen, —CN, haloalkyl, haloalkoxy and —NR₁₃R₁₃′;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the aryl, heterocyclyl or cycloalkyl, also in alkylaryl, alkylheterocyclyl or alkylcycloalkyl, if substituted and the substitution has not been defined otherwise, it is substituted with one or two substituent/s selected from halogen, —R₁₄, —OR₁₄, —NO₂, —NR₁₄R₁₄′, —NR₁₄C(O)R₁₄′, —NR₁₄S(O)₂R₁₄′, —S(O)₂NR₁₄R₁₄′, —NR₁₄C(O)NR₁₄′R₁₄″, —SR₁₄, —S(O)R₁₄, —S(O)₂R₁₄, —CN, haloalkyl, haloalkoxy, —C(O)OR₁₄, —C(O)NR₁₄R₁₄′, —OCH₂CH₂OR₁₄, —NR₁₄S(O)₂NR₁₄′R₁₄″ and —C(CH₃)₂OR₁₄;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the aryl, heterocyclyl or cycloalkyl, also in alkylaryl, alkylheterocyclyl or alkylcycloalkyl, if substituted and the substitution has not been defined otherwise, it is substituted with one or more —OR₁₄;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

the aryl, heterocyclyl or cycloalkyl, also in alkylaryl, alkylheterocyclyl or alkylcycloalkyl, if substituted and the substitution has not been defined otherwise, it is substituted with one or more —OH;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
Y₁ is —C(R_{y}R_{y}′)—; preferably Y₁ is —CH2-;

and/or

R_{y} and R_{y}′ are independently selected from hydrogen, substituted or unsubstituted C₁₋₆ alkyl, substituted or unsubstituted C₂₋₆ alkenyl and substituted or unsubstituted C₂₋₆ alkynyl; preferably R_{y} and R_{y}′ are both hydrogen;

and/or
Y₂ is —C(R_{y}″R_{y}′″)—; preferably Y₂ is —CH₂— or —CH(CH₃)—;

and/or
R_{y}″ and R_{y}′″ are independently selected from hydrogen, substituted or unsubstituted C₁₋₆ alkyl, substituted or unsubstituted C₂₋₆ alkenyl and substituted or unsubstituted C₂₋₆ alkynyl; preferably R_{y}″ and R_{y}′″ are independently selected from hydrogen and substituted or unsubstituted C₁₋₆ alkyl; more preferably R_{y}″ and R_{y}′″ are independently selected from hydrogen and substituted or unsubstituted methyl;

and/or
Y₃ is —CH₃ or —CH₂CH₃;

and/or

Y₂ and Y₃ taken together, form a substituted or unsubstituted cycloalkyl; preferably Y₂ and Y₃ taken together, form a substituted or unsubstituted cyclopropyl;

and/or

W is nitrogen or —CR_{w}—;

and/or

R_{w} is hydrogen or halogen; preferably R_{w} is hydrogen;

and/or
w1, w2, w3 and w4 are independently selected from the group consisting of nitrogen and carbon;

and/or

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₁ is selected from the group consisting of hydrogen, halogen, substituted or unsubstituted C₁₋₆ alkyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted heterocyclyl, substituted or unsubstituted aryl and substituted or unsubstituted alkylheterocyclyl; more preferably R₁ is hydrogen, bromine, fluorine, chlorine, —OH, or a substituted or unsubstituted group selected from methyl, ethyl, —O-methyl, —NH(ethyl), —N(piperidine)(methyl), —NH(piperidine), —NH(CH₂CH₂-Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —N(piperidine)(C(O)-ethyl), —N(ethyl)(C(O)O-isobutyl), —N(benzyl)(C(O)O-isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl;
and/or

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₁ is selected from the group consisting of hydrogen, halogen, substituted or unsubstituted C₁₋₆ alkyl, —OR₈, —(CH₂)ₙNR₈R₈′, —CH(phenyl)-NR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted heterocyclyl, substituted or unsubstituted aryl and substituted or unsubstituted alkylheterocyclyl; more preferably R₁ is hydrogen, bromine, fluorine, chlorine, —OH, or a substituted or unsubstituted group selected from methyl, ethyl, —O-methyl, —NH(ethyl), —N(piperidine)(methyl), —NH(piperidine), —NH(CH₂CH₂-Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —CH(phenyl)-NH(methyl), —N(piperidine)(C(O)-ethyl), —N(ethyl)(C(O)O-isobutyl), —N(benzyl)(C(O)O-isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl;

and/or
n is 0, 1, 2, 3, 4 or 5; preferably n is 0, 1 or 2;

and/or
m is 0, 1, 2, 3, 4 or 5; preferably m is 0 or 1;

and/or

R₂ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₂₁, —NO₂, —NR₂₁R₂₁′, —NR₂₁C(O)R₂₁′, —NR₂₁S(O)₂R₂₁′, —S(O)₂NR₂₁R₂₁′, —NR₂₁C(O)NR₂₁′R₂₁″, —SR₂₁, —S(O)R₂₁, —S(O)₂R₂₁, —CN, haloalkyl, haloalkoxy, —C(O)OR₂₁, —C(O)NR₂₁R₂₁′, —NR₂₁S(O)₂NR₂₁′R₂₁″ and —C(CH₃)₂OR₂₁; preferably R₂ is selected from hydrogen, bromine, fluorine, chlorine or a substituted or unsubstituted group selected from methyl and —O-methyl;

and/or

R₃ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₃₁, —NO₃, —NR₃₁R₃₁′, —NR₃₁C(O)R₃₁′, —NR₃₁S(O)₃R₃₁′, —S(O)₃NR₃₁R₃₁′, —NR₃₁C(O)NR₃₁′R₃₁″, —SR₃₁, —S(O)R₃₁, —S(O)₃R₃₁, —CN, haloalkyl, haloalkoxy, —C(O)OR₃₁, —C(O)NR₃₁R₃₁′, —NR₃₁S(O)₃NR₃₁′R₃₁″ and —C(CH₃)₃OR₃₁; preferably R₃ is selected from hydrogen, bromine, fluorine or substituted or unsubstituted —O-methyl;

and/or

R₄ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted alkylheterocyclyl, substituted or unsubstituted alkylaryl and substituted or unsubstituted alkylcycloalkyl; preferably R₄ is selected from substituted or unsubstituted C₁₋₆ alkyl, substituted or unsubstituted alkylheterocyclyl, and substituted or unsubstituted alkylcycloalkyl; more preferably, R₄ is selected from a substituted or unsubstituted group selected from methyl, ethyl, propyl, —CH₂-cyclopropyl and —CH₂-furan;

and/or

R₅, R₅′, R₅″ and R₅′″ are independently selected from hydrogen, halogen, substituted or unsubstituted C₁₋₆ alkyl, substituted or unsubstituted C₂₋₆ alkenyl and substituted or unsubstituted C₂₋₆ alkynyl; preferably R₅, R₅′, R₅″ and R₅′″ are independently selected from hydrogen and halogen; more preferably R₅, R₅′, R₅″ and R₅′″ are independently selected from hydrogen and fluorine;

and/or

R₅ and R₅′ and/or R₅″ and R₅′″ taken together with the carbon atom to which they are attached form a carbonyl group;

and/or

R₆, R₆′, R₆″ and R₆′″ are independently selected from hydrogen, substituted or unsubstituted C₁₋₆ alkyl, substituted or unsubstituted C₂₋₆ alkenyl and substituted or unsubstituted C₂₋₆ alkynyl; preferably R₆, R₆′, R₆″ and R₆′″ are independently selected from hydrogen and substituted or unsubstituted C₁₋₆ alkyl; more preferably R₆, R₆′, R₆″ and R₆′″ are independently selected from hydrogen and a substituted or unsubstituted group selected from methyl and ethyl;

and/or

R₇ is selected from hydrogen, substituted or unsubstituted C₁₋₆ alkyl, substituted or unsubstituted C₂₋₆ alkenyl and substituted or unsubstituted C₂₋₆ alkynyl; preferably R₇ is selected from hydrogen and substituted or unsubstituted C₁₋₆ alkyl; more preferably R₇ is selected from hydrogen and substituted or unsubstituted methyl;

and/or

R₈ and R₈′ are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₈ and R₈′ are independently selected from the group consisting of hydrogen, substituted or unsubstituted C₁₋₆ alkyl, substituted or unsubstituted heterocyclyl, substituted or unsubstituted aryl, substituted or unsubstituted alkylheterocyclyl and substituted or unsubstituted alkylaryl; more preferably R₈ and R₈′ are independently selected from hydrogen or a substituted or unsubstituted group selected from methyl, ethyl, isobutyl, isopentyl, phenyl, piperidine, benzyl, —CH₂CH₂-oxaspirodecanyl;

and/or

R₁₁, R₁₁′ and R₁₁″ are independently selected from hydrogen, substituted or unsubstituted C₁₋₆ alkyl, substituted or unsubstituted C₂₋₆ alkenyl and substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₁₁, R₁₁′ and R₁₁″ are independently selected from hydrogen, substituted or unsubstituted C₁₋₆ alkyl, substituted or unsubstituted aryl, and substituted or unsubstituted alkylaryl; more preferably R₁₁, R₁₁′ and R₁₁″ are independently selected from hydrogen or a substituted or unsubstituted group selected from methyl, ethyl, phenyl and benzyl;

and/or

R₁₃ and R₁₃′ are independently selected from hydrogen, unsubstituted C₁₋₆ alkyl, unsubstituted C₂₋₆ alkenyl, and unsubstituted C₂₋₆ alkynyl;

and/or

R₁₄, R₁₄′ and R₁₄″ are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl; preferably R₁₄ is hydrogen;

and/or

R₂₁, R₂₁′ and R₂₁″ are independently selected from hydrogen, substituted or unsubstituted C₁₋₆ alkyl, substituted or unsubstituted C₂₋₆ alkenyl and substituted or unsubstituted C₂₋₆ alkynyl; preferably R₂₁ is substituted or unsubstituted C₁₋₆ alkyl; more preferably R₂₁ is substituted or unsubstituted methyl;

and/or

R₃₁, R₃₁′ and R₃₁″ are independently selected from hydrogen, substituted or unsubstituted C₁₋₆ alkyl, substituted or unsubstituted C₃₋₆ alkenyl and substituted or unsubstituted C₃₋₆ alkynyl; preferably R₃₁ is substituted or unsubstituted C₁₋₆ alkyl; more preferably R₃₁ is substituted or unsubstituted methyl;

and/or

R₄₁, R₄₁′ and R₄₁″ are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₄₁, R₄₁′ and R₄₁″ are independently selected from hydrogen, substituted or unsubstituted C₁₋₆ alkyl, and substituted or unsubstituted alkylaryl; more preferably R₄₁, R₄₁′ and R₄₁″ are independently selected from hydrogen and a substituted or unsubstituted group selected from methyl, ethyl, phenethyl and —CH₂CH₂CH₂-phenyl;

and/or

R₆₁ and R₆₁′ are independently selected from hydrogen, unsubstituted C₁₋₆ alkyl, unsubstituted C₂₋₆ alkenyl, and unsubstituted C₂₋₆ alkynyl; preferably R₆₁ and R₆₁′ are independently selected from hydrogen and unsubstituted C₁₋₆ alkyl; more preferably R₆₁ and R₆₁′ are independently selected from hydrogen and unsubstituted methyl;

and/or

R₈₁, R₈₁′ and R₈₁″ are independently selected from hydrogen, substituted or unsubstituted C₁₋₆ alkyl, substituted or unsubstituted C₂₋₆ alkenyl and substituted or unsubstituted C₂₋₆ alkynyl; preferably R₈₁ is substituted or unsubstituted C₁₋₆ alkyl; more preferably R₈₁ is substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

Y₁ is —C(R_{y}R_{y}′)—; preferably Y₁ is —CH2-;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{y} and R_{y}′ are independently selected from hydrogen, substituted or unsubstituted C1-6 alkyl, substituted or unsubstituted C₂₋₆ alkenyl and substituted or unsubstituted C₂₋₆ alkynyl; preferably R_{y} and R_{y}′ are both hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
Y₂ is —C(R_{y}″R_{y}′″)—; preferably Y₂ is —CH₂— or —CH(CH₃)—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R_{y}″ and R_{y}′″ are independently selected from hydrogen, substituted or unsubstituted C₁₋₆ alkyl, substituted or unsubstituted C₂₋₆ alkenyl and substituted or unsubstituted C₂₋₆ alkynyl; preferably R_{y}″ and R_{y}′″ are independently selected from hydrogen and substituted or unsubstituted C₁₋₆ alkyl; more preferably R_{y}″ and R_{y}′″ are independently selected from hydrogen and substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

Y₂ and Y₃ taken together, form a substituted or unsubstituted cycloalkyl; preferably Y₂ and Y₃ taken together, form a substituted or unsubstituted cyclopropyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{w} is hydrogen or halogen; preferably R_{w} is hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₁ is selected from the group consisting of hydrogen, halogen, substituted or unsubstituted C₁₋₆ alkyl, —OR₈, —(CH₂)ₙNR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted heterocyclyl, substituted or unsubstituted aryl and substituted or unsubstituted alkylheterocyclyl; more preferably R₁ is hydrogen, bromine, fluorine, chlorine, —OH, or a substituted or unsubstituted group selected from methyl, ethyl, —O— methyl, —NH(ethyl), —N(piperidine)(methyl), —NH(piperidine), —NH(CH₂CH₂-Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —N(piperidine)(C(O)-ethyl), —N(ethyl)(C(O)O-isobutyl), —N(benzyl)(C(O)O— isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₁ is selected from the group consisting of hydrogen, halogen, substituted or unsubstituted C₁₋₆ alkyl, —OR₈, —(CH₂)ₙNR₈R₈′, —CH(phenyl)-NR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted heterocyclyl, substituted or unsubstituted aryl and substituted or unsubstituted alkylheterocyclyl; more preferably R₁ is hydrogen, bromine, fluorine, chlorine, —OH, or a substituted or unsubstituted group selected from methyl, ethyl, —O-methyl, —NH(ethyl), —N(piperidine)(methyl), —NH(piperidine), —NH(CH₂CH₂-Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —CH(phenyl)-NH(methyl), —N(piperidine)(C(O)-ethyl), —N(ethyl)(C(O)O-isobutyl), —N(benzyl)(C(O)O-isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
n is 0, 1, 2, 3, 4 or 5; preferably n is 0, 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
m is 0, 1, 2, 3, 4 or 5; preferably m is 0 or 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₂ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₂₁, —NO₂, —NR₂₁R₂₁′, —NR₂₁C(O)R₂₁′, —NR₂₁S(O)₂R₂₁′, —S(O)₂NR₂₁R₂₁′, —NR₂₁C(O)NR₂₁′R₂₁″, —SR₂₁, —S(O)R₂₁, —S(O)₂R₂₁, —CN, haloalkyl, haloalkoxy, —C(O)OR₂₁, —C(O)NR₂₁R₂₁′, —NR₂₁S(O)₂NR₂₁′R₂₁″ and —C(CH₃)₂OR₂₁; preferably R₂ is selected from hydrogen, bromine, fluorine, chlorine or a substituted or unsubstituted group selected from methyl and —O-methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₃ is selected from hydrogen, halogen, substituted or unsubstituted C₁₋₆ alkyl, substituted or unsubstituted C₂₋₆ alkenyl, substituted or unsubstituted C₂₋₆ alkynyl, —OR₃₁, —NO₃, —NR₃₁R₃₁′, —NR₃₁C(O)R₃₁′, —NR₃₁S(O)₃R₃₁′, —S(O)₃NR₃₁R₃₁′, —NR₃₁C(O)NR₃₁′R₃₁″, —SR₃₁, —S(O)R₃₁, —S(O)₃R₃₁, —CN, haloalkyl, haloalkoxy, —C(O)OR₃₁, —C(O)NR₃₁R₃₁′, —NR₃₁S(O)₃NR₃₁′R₃₁″ and —C(CH₃)₃OR₃₁; preferably R₃ is selected from hydrogen, bromine, fluorine or substituted or unsubstituted —O-methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₄ is selected from substituted or unsubstituted C1-6 alkyl, substituted or unsubstituted C₂₋₆ alkenyl, substituted or unsubstituted C₂₋₆ alkynyl, substituted or unsubstituted cycloalkyl, substituted or unsubstituted alkylheterocyclyl, substituted or unsubstituted alkylaryl and substituted or unsubstituted alkylcycloalkyl; preferably R₄ is selected from substituted or unsubstituted C₁₋₆ alkyl, substituted or unsubstituted alkylheterocyclyl, and substituted or unsubstituted alkylcycloalkyl; more preferably, R₄ is selected from a substituted or unsubstituted group selected from methyl, ethyl, propyl, —CH₂— cyclopropyl and —CH₂-furan;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₅, R₅′, R₅″ and R₅′″ are independently selected from hydrogen, halogen, substituted or unsubstituted C₁₋₆ alkyl, substituted or unsubstituted C₂₋₆ alkenyl and substituted or unsubstituted C₂₋₆ alkynyl; preferably R₅, R₅′, R₅″ and R₅′″ are independently selected from hydrogen and halogen; more preferably R₅, R₅′, R₅″ and R₅′″ are independently selected from hydrogen and fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆, R₆′, R₆″ and R₆′″ are independently selected from hydrogen, substituted or unsubstituted C₁₋₆ alkyl, substituted or unsubstituted C₂₋₆ alkenyl and substituted or unsubstituted C₂₋₆ alkynyl; preferably R₆, R₆′, R₆″ and R₆′″ are independently selected from hydrogen and substituted or unsubstituted C₁₋₆ alkyl; more preferably R₆, R₆′, R₆″ and R₆′″ are independently selected from hydrogen and a substituted or unsubstituted group selected from methyl and ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆ is selected from hydrogen and substituted or unsubstituted (S)—C₁₋₆ alkyl; more preferably R₆ is selected from hydrogen and a substituted or unsubstituted group selected from (S)-methyl and (S)-ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆ is selected from hydrogen and substituted or unsubstituted (S)—C₁₋₆ alkyl; more preferably R₆ is selected from hydrogen and a substituted or unsubstituted group selected from (S)-methyl and (S)-ethyl; while R₆′, R₆″ and R₆′″ are all hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆′ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆′ is selected from hydrogen and substituted or unsubstituted (S)—C₁₋₆ alkyl; more preferably R₆′ is selected from hydrogen and a substituted or unsubstituted group selected from (S)-methyl and (S)-ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆′ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆′ is selected from hydrogen and substituted or unsubstituted (S)—C₁₋₆ alkyl; more preferably R₆′ is selected from hydrogen and a substituted or unsubstituted group selected from (S)-methyl and (S)-ethyl; while R₆, R₆″ and R₆′″ are all hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆″ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆″ is selected from hydrogen and substituted or unsubstituted (S)—C₁₋₆ alkyl; more preferably R₆″ is selected from hydrogen and a substituted or unsubstituted group selected from (S)-methyl and (S)-ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆″ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆″ is selected from hydrogen and substituted or unsubstituted (S)—C₁₋₆ alkyl; more preferably R₆″ is selected from hydrogen and a substituted or unsubstituted group selected from (S)-methyl and (S)-ethyl; while R₆, R₆′ and R₆′″ are all hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆′″ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆′″ is selected from hydrogen and substituted or unsubstituted (S)—C₁₋₆ alkyl; more preferably R₆′″ is selected from hydrogen and a substituted or unsubstituted group selected from (S)-methyl and (S)-ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₆′″ is selected from hydrogen, substituted or unsubstituted (S)—C₁₋₆ alkyl, substituted or unsubstituted (S)—C₂₋₆ alkenyl and substituted or unsubstituted (S)—C₂₋₆ alkynyl; preferably R₆′″ is selected from hydrogen and substituted or unsubstituted (S)—C₁₋₆ alkyl; more preferably R₆′″ is selected from hydrogen and a substituted or unsubstituted group selected from (S)-methyl and (S)-ethyl; while R₆, R₆′ and R₆″ are all hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₇ is selected from hydrogen, substituted or unsubstituted C₁₋₆ alkyl, substituted or unsubstituted C₂₋₆ alkenyl and substituted or unsubstituted C₂₋₆ alkynyl; preferably R₇ is selected from hydrogen and substituted or unsubstituted C₁₋₆ alkyl; more preferably R₇ is selected from hydrogen and substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₈ and R₈′ are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₈ and R₈′ are independently selected from the group consisting of hydrogen, substituted or unsubstituted C₁₋₆ alkyl, substituted or unsubstituted heterocyclyl, substituted or unsubstituted aryl, substituted or unsubstituted alkylheterocyclyl and substituted or unsubstituted alkylaryl; more preferably R₈ and R₈′ are independently selected from hydrogen or a substituted or unsubstituted group selected from methyl, ethyl, isobutyl, isopentyl, phenyl, piperidine, benzyl, —CH₂CH₂-oxaspirodecanyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₁₁, R₁₁′ and R₁₁″ are independently selected from hydrogen, substituted or unsubstituted C₁₋₆ alkyl, substituted or unsubstituted C₂₋₆ alkenyl and substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₁₁, R₁₁′ and R₁₁″ are independently selected from hydrogen, substituted or unsubstituted C₁₋₆ alkyl, substituted or unsubstituted aryl, and substituted or unsubstituted alkylaryl; more preferably R₁₁, R₁₁′ and R₁₁″ are independently selected from hydrogen or a substituted or unsubstituted group selected from methyl, ethyl, phenyl and benzyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₄, R₁₄′ and R₁₄″ are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl; preferably R₁₄ is hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₂₁, R₂₁′ and R₂₁″ are independently selected from hydrogen, substituted or unsubstituted C₁₋₆ alkyl, substituted or unsubstituted C₂₋₆ alkenyl and substituted or unsubstituted C₂₋₆ alkynyl; preferably R₂₁ is substituted or unsubstituted C₁₋₆ alkyl; more preferably R₂₁ is substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R₃₁, R₃₁′ and R₃₁″ are independently selected from hydrogen, substituted or unsubstituted C₁₋₆ alkyl, substituted or unsubstituted C₃₋₆ alkenyl and substituted or unsubstituted C₃₋₆ alkynyl; preferably R₃₁ is substituted or unsubstituted C₁₋₆ alkyl; more preferably R₃₁ is substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₄₁, R₄₁′ and R₄₁″ are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl; preferably R₄₁, R₄₁′ and R₄₁″ are independently selected from hydrogen, substituted or unsubstituted C₁₋₆ alkyl, and substituted or unsubstituted alkylaryl; more preferably R₄₁, R₄₁′ and R₄₁″ are independently selected from hydrogen and a substituted or unsubstituted group selected from methyl, ethyl, phenethyl and —CH₂CH₂CH₂-phenyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₆₁ and R₆₁′ are independently selected from hydrogen, unsubstituted C₁₋₆ alkyl, unsubstituted C₂₋₆ alkenyl, and unsubstituted C₂₋₆ alkynyl; preferably R₆₁ and R₆₁′ are independently selected from hydrogen and unsubstituted C₁₋₆ alkyl; more preferably R₆₁ and R₆₁′ are independently selected from hydrogen and unsubstituted methyl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₈₁, R₈₁′ and R₈₁″ are independently selected from hydrogen, substituted or unsubstituted C₁₋₆ alkyl, substituted or unsubstituted C₂₋₆ alkenyl and substituted or unsubstituted C₂₋₆ alkynyl; preferably R₈₁ is substituted or unsubstituted C₁₋₆ alkyl; more; preferably R₈₁ is substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{y} and R_{y}′ as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{y} and R_{y}′ as defined in any of the embodiments of the present invention,
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{y}″ and R_{y}′″ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Y₂ and Y₃ as defined in any of the embodiments of the present invention,
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl; more preferably the cycloalkyl is cyclopropyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,
the alkyl in alkylaryl, alkylheterocyclyl, alkylcycloalkyl, haloalkyl or haloalkoxy is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl; more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, octahydro-ethanopyrrolo-pyridine, oxaspirodecane, oxadiazaspiroundecane, indoline-2-one and quinazoline; preferably the heterocyclyl is tetrahydropyrridine, pyridine, piperidine, pyrrole, oxadiazaspiroundecane, octahydro-ethanopyrrolo-pyridine or piperazine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃ as defined in any of the embodiments of the present invention,
the C1-6 alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ as defined in any of the embodiments of the present invention,
the alkyl in alkylaryl, alkylheterocyclyl or alkylcycloalkyl is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl; more preferably the C₁₋₆ alkyl is methyl, ethyl or propyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl; more preferably the cycloalkyl is cyclopropyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, octahydro-ethanopyrrolo-pyridine, oxaspirodecane, oxadiazaspiroundecane, indoline-2-one and quinazoline; preferably the heterocyclyl is furan;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅, R₅′, R₅″ and R₅′″ as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆, R₆′, R₆″ and R₆′″ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₇ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₈ and R₈′ as defined in any of the embodiments of the present invention,
the alkyl in alkylaryl, alkylheterocyclyl or alkylcycloalkyl is C₁₋₆ alkyl like methyl, ethyl, propyl, butyl, pentyl, hexyl, isopropyl, or 2-methylpropyl; more preferably the alkyl is methyl or ethyl;
and/or
the C₁₋₆ alkyl is preferably selected from methyl, ethyl, propyl, butyl, pentyl, hexyl, isopropyl, or 2-methylpropyl; more preferably the C₁₋₆ alkyl is methyl, ethyl, isobutyl, isopentyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, octahydro-ethanopyrrolo-pyridine, oxaspirodecane, oxadiazaspiroundecane, indoline-2-one and quinazoline; preferably the heterocyclyl is oxaspirodecane;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₁, R₁₁′, R₁₁″ and R₁₁′″ as defined in any of the embodiments of the present invention,
the alkyl in alkylaryl, alkylheterocyclyl or alkylcycloalkyl is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, octahydro-ethanopyrrolo-pyridine, oxaspirodecane, oxadiazaspiroundecane, indoline-2-one and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₃ and R₁₃′ as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₄, R₁₄′ and R₁₄″ as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, octahydro-ethanopyrrolo-pyridine, oxaspirodecane, oxadiazaspiroundecane, indoline-2-one and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂₁, R₂₁′ and R₂₁″ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃₁, R₃₁′ and R₃₁″ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄₁, R₄₁′ and R₄₁″ as defined in any of the embodiments of the present invention,
the alkyl in alkylaryl, alkylheterocyclyl or alkylcycloalkyl is C₁₋₆ alkyl like methyl, ethyl, propyl, butyl, pentyl, hexyl, isopropyl, or 2-methylpropyl; more preferably the alkyl is ethyl or propyl;
and/or
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl; more preferably the cycloalkyl is cyclopropyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, octahydro-ethanopyrrolo-pyridine, oxaspirodecane, oxadiazaspiroundecane, indoline-2-one and quinazoline; preferably the heterocyclyl is tetrahydropyrane, pyridine, thiophen or thiazole;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆₁ and R₆₁′ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₈₁, R₈₁′ and R₈₁″ as defined in any of the embodiments of the present invention,
the C₁₋₆ alkyl is preferably selected from methyl, ethyl, propyl, butyl, pentyl, hexyl, isopropyl, or 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

Y₁ is —CH2-;

and/or

R_{y} and R_{y}′ are both hydrogen;

and/or

Y₂ is —CH₂— or —CH(CH₃)—;

and/or

R_{y}″ and R_{y}′″ are independently selected from hydrogen and substituted or unsubstituted methyl;

and/or

Y₃ is —CH₃ or —CH₂CH₃;

and/or

Y₂ and Y₃ taken together, form a substituted or unsubstituted cyclopropyl;

and/or

W is nitrogen or —CR_{w}—;

and/or

R_{w} is hydrogen;

and/or

w1, w2, w3 and w4 are independently selected from the group consisting of nitrogen and carbon;

and/or

R₁ is hydrogen, bromine, fluorine, chlorine, —OH, or a substituted or unsubstituted group selected from methyl, ethyl, —O-methyl, —NH(ethyl), —N(piperidine)(methyl), —NH(piperidine), —NH(CH₂CH₂—Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —N(piperidine)(C(O)-ethyl), —N(ethyl)(C(O)O-isobutyl), —N(benzyl)(C(O)O-isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl;

and/or
R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R₈′, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;

and/or

n is 0, 1 or 2;

and/or

m is 0 or 1;

and/or

R₂ is selected from hydrogen, bromine, fluorine, chlorine or a substituted or unsubstituted group selected from methyl and —O-methyl;

and/or

R₃ is selected from hydrogen, bromine, fluorine or substituted or unsubstituted —O— methyl;

and/or

R₄ is selected from a substituted or unsubstituted group selected from methyl, ethyl, propyl, —CH₂-cyclopropyl and —CH₂-furan;

and/or

R₅, R₅′, R₅″ and R₅′″ are independently selected from hydrogen and fluorine;

and/or

R₆, R₆′, R₆″ and R₆′″ are independently selected from hydrogen and a substituted or unsubstituted group selected from methyl and ethyl;

and/or

R₇ is selected from hydrogen and substituted or unsubstituted methyl; and/or

R₈ and R₈′ are independently selected from hydrogen or a substituted or unsubstituted group selected from methyl, ethyl, isobutyl, isopentyl, phenyl, piperidine, benzyl, —CH₂CH₂-oxaspirodecanyl;

and/or

R₁₁, R₁₁′ and R₁₁″ are independently selected from hydrogen or a substituted or unsubstituted group selected from methyl, ethyl, phenyl and benzyl;

and/or

R₁₄ is hydrogen;

and/or

R₂₁ is substituted or unsubstituted methyl;

and/or

R₃₁ is substituted or unsubstituted methyl;

and/or

R₄₁, R₄₁′ and R₄₁″ are independently selected from hydrogen and a substituted or unsubstituted group selected from methyl, ethyl, phenethyl and —CH₂CH₂CH₂-phenyl;

and/or

R₆₁ and R₆₁′ are independently selected from hydrogen and unsubstituted methyl;

and/or

R₈₁ is substituted or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

Y₁ is —CH2-.

In a preferred embodiment

R_{y} and R_{y}′ are both hydrogen.

In a preferred embodiment

Y₂ is —CH₂— or —CH(CH₃)—.

In a preferred embodiment

R_{y}″ and R_{y}′″ are independently selected from hydrogen and substituted or unsubstituted methyl;

In a preferred embodiment

R_{y}″ is hydrogen or substituted or unsubstituted methyl.

In a preferred embodiment

R_{y}′″ is hydrogen.

In a preferred embodiment

R_{y}″ is hydrogen or substituted or unsubstituted methyl, while R_{y}′″ is hydrogen.

In a preferred embodiment

R_{y}″ is substituted or unsubstituted methyl, while R_{y}′″ is hydrogen.

In a preferred embodiment

R_{y}″ and R_{y}′″ are both hydrogen.

In a preferred embodiment

Y₂ and Y₃ taken together, form a substituted or unsubstituted cyclopropyl.

In a preferred embodiment

W is nitrogen or —CR_{w}—, preferably nitrogen or —CH—.

In a preferred embodiment

R_{w} is hydrogen.

In a preferred embodiment

w1, w2, w3 and w4 are independently selected from the group consisting of nitrogen and carbon.

In a preferred embodiment

w1, w2, w3 and w4 are all carbon.

In a preferred embodiment

w1 is nitrogen, while w2, w3 and w4 are all carbon.

In a preferred embodiment

w2 is nitrogen, while w1, w3 and w4 are all carbon.

In a preferred embodiment

w3 is nitrogen, while w1, w2 and w4 are all carbon.

In a preferred embodiment

w4 is nitrogen, while w1, w2 and w3 are all carbon.

In a preferred embodiment

R₁ is hydrogen, bromine, fluorine, chlorine, —OH, or a substituted or unsubstituted group selected from methyl, ethyl, —O-methyl, —NH(ethyl), —N(piperidine)(methyl), —NH(piperidine), —NH(CH₂CH₂—Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —N(piperidine)(C(O)-ethyl), —N(ethyl)(C(O)O-isobutyl), —N(benzyl)(C(O)O-isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl;

In a preferred embodiment

R₁ is —CH(phenyl)-NH-methyl.

In a preferred embodiment

R₁ is hydrogen, bromine, fluorine, chlorine, —OH, or a substituted or unsubstituted group selected from methyl, ethyl, —O-methyl, —NH(ethyl), —N(piperidine)(methyl), —NH(piperidine), —NH(CH₂CH₂—Oxaspirodecane), —N(methyl)(benzyl), —N(methyl)(ethyl), —CH₂N(methyl)(benzyl), —CH₂N(methyl)(isobutyl), —CH₂N(methyl)(isopentyl), —CH₂CH₂N(methyl)(isopentyl), —CH₂CH₂N(methyl)(benzyl), —CH(phenyl)-NH-methyl, —N(piperidine)(C(O)-ethyl), —N(ethyl)(C(O)O-isobutyl), —N(benzyl)(C(O)O-isobutyl), —C(O)NH(benzyl), —C(O)OH, —C(O)OCH3, —O—CH(phenyl)(methyl), —O—CH(phenyl)(ethyl), —CF3, —O—CF3, —CN, pyridinyl, tetrahydropyridinyl, piperidinyl, pyrrole, oxadiazaspiroundecanyl, octahydro-ethanopyrrolo-pyridinyl phenyl, —CH2-piperidinyl and —CH2-piperazinyl.

In a preferred embodiment

n is 0, 1 or 2.

In a preferred embodiment

m is 0 or 1.

In a preferred embodiment

R₂ is selected from hydrogen, bromine, fluorine, chlorine or a substituted or unsubstituted group selected from methyl and —O-methyl.

In a preferred embodiment

R₃ is selected from hydrogen, bromine, fluorine or substituted or unsubstituted —O— methyl.

In a preferred embodiment

R₄ is selected from a substituted or unsubstituted group selected from methyl, ethyl, propyl, —CH₂-cyclopropyl and —CH₂-furan.

In a preferred embodiment

R₅, R₈′, R₈″ and R₈′″ are independently selected from hydrogen and fluorine.

In a preferred embodiment

R₅ is hydrogen or fluorine.

In a preferred embodiment

R₅′ is hydrogen or fluorine.

In a preferred embodiment

R₅″ is hydrogen.

In a preferred embodiment

R₅′″ is hydrogen.

In a preferred embodiment

R₅, R₅′, R₅″ and R₅′″ are all hydrogen.

In a preferred embodiment

R₅ and R₅′ are hydrogen or fluorine, while R₅″ and R₅′″ are both hydrogen.

In a preferred embodiment

R₅ and R₅′ are both fluorine, while R₅″ and R₅′″ are both hydrogen.

In a preferred embodiment

R₅ and R₅′ are both fluorine, while R₅″ and R₅′″ are both hydrogen and W is —CH—.

In a preferred embodiment

R₅, R₅′ and R₅″ are all hydrogen, while R₅′″ and R_{w} form a double bond.

In a preferred embodiment

R₆, R₆′, R₆″ and R₆′″ are independently selected from hydrogen and a substituted or unsubstituted group selected from methyl and ethyl.

In a preferred embodiment

R₆ is hydrogen or a substituted or unsubstituted group selected from methyl and ethyl.

In a preferred embodiment

R₆ is substituted or unsubstituted methyl.

In a preferred embodiment

R₆′ is hydrogen.

In a preferred embodiment

R₆″ is hydrogen or substituted or unsubstituted methyl.

In a preferred embodiment

R₆″ is substituted or unsubstituted methyl.

In a preferred embodiment

R₆″ is hydrogen or a substituted or unsubstituted group selected from methyl and ethyl.

In a preferred embodiment

R₆′″ is hydrogen.

In a preferred embodiment

R₆ is hydrogen, methyl or ethyl, while R₆′ is hydrogen.

In a preferred embodiment

R₆″ is hydrogen or substituted or unsubstituted methyl, while R₆′″ is hydrogen.

In a preferred embodiment

R₆, R₆′, R₆″ and R₆′″ are all hydrogen.

In a preferred embodiment

R₆ is substituted or unsubstituted methyl, while R₆′, R₆″ and R₆′″ are all hydrogen.

In a preferred embodiment

R₆″ is substituted or unsubstituted methyl, while R₆, R₆′ and R₆′″ are all hydrogen.

In a preferred embodiment

R₆ is substituted or unsubstituted (S)-methyl, while R₆′, R₆″ and R₆′″ are all hydrogen.

In a preferred embodiment

R₆″ is substituted or unsubstituted (S)-methyl, while R₆, R₆′ and R₆′″ are all hydrogen.

In a preferred embodiment

R₆ is substituted or unsubstituted ethyl, while R₆′, R₆″ and R₆′″ are all hydrogen.

In a preferred embodiment

R₆″ is substituted or unsubstituted ethyl, while R₆, R₆′ and R₆′″ are all hydrogen.

In a preferred embodiment

R₆ is substituted or unsubstituted (S)-ethyl, while R₆′, R₆″ and R₆′″ are all hydrogen.

In a preferred embodiment

R₆″ is substituted or unsubstituted (S)-ethyl, while R₆, R₆′ and R₆′″ are all hydrogen.

In a preferred embodiment

R₆ and R₆″ are both substituted or unsubstituted methyl.

In a preferred embodiment

R₆ and R₆″ are both substituted or unsubstituted methyl, while R₆′ and R₆′″ are both hydrogen.

In a preferred embodiment

R₆ is substituted or unsubstituted (S)-methyl, and W is carbon, while R₆′, R₆″ and R₆′″ are all hydrogen.

In a preferred embodiment

R₆″ is substituted or unsubstituted (S)-methyl, and W is carbon, while R₆, R₆′ and R₆′″ are all hydrogen.

In a preferred embodiment

R₇ is hydrogen or substituted or unsubstituted methyl.

In a preferred embodiment

R₈ and R₈′ are independently selected from hydrogen and a substituted or unsubstituted group selected from methyl, ethyl, isobutyl, isopentyl, phenyl, piperidine, benzyl and —CH₂CH₂-oxaspirodecanyl.

In a preferred embodiment

R₈ is hydrogen or a substituted or unsubstituted group selected from methyl, ethyl, isobutyl, isopentyl, phenyl, piperidine, benzyl and —CH₂CH₂-oxaspirodecanyl.

In a preferred embodiment

R₈′ is hydrogen or a substituted or unsubstituted group selected from methyl, ethyl and isobutyl.

In a preferred embodiment

R₈ is hydrogen or a substituted or unsubstituted group selected from methyl, ethyl, isobutyl, isopentyl, phenyl, piperidine, benzyl and —CH₂CH₂-oxaspirodecanyl, while R₈′ is hydrogen or a substituted or unsubstituted group selected from methyl, ethyl and isobutyl.

In a preferred embodiment

R₈ is a substituted or unsubstituted group selected from ethyl, piperidine, benzyl, and —CH₂CH₂-oxaspirodecanyl, while R₈′ is hydrogen.

In a preferred embodiment

R₈ is hydrogen or a substituted or unsubstituted group selected from ethyl, isobutyl, isopentyl, phenyl, piperidine and benzyl, while R₈′ is substituted or unsubstituted methyl.

In a preferred embodiment

R₈ is substituted or unsubstituted group selected from isobutyl, phenyl and piperidine, while R₈′ is substituted or unsubstituted ethyl.

In a preferred embodiment

R₈ is substituted or unsubstituted benzyl, while R₈′ is substituted or unsubstituted isobutyl.

In a preferred embodiment

R₈ is substituted or unsubstituted methyl, while R₈′ is hydrogen.

In a preferred embodiment

R₁₁, R₁₁′ and R₁₁″ are independently selected from hydrogen or a substituted or unsubstituted group selected from methyl, ethyl, phenyl and benzyl.

In a preferred embodiment

R₁₁ is hydrogen or a substituted or unsubstituted group selected from methyl, phenyl and benzyl.

In a preferred embodiment

R₁₁′ is hydrogen or a substituted or unsubstituted group selected from methyl and ethyl.

In a preferred embodiment

R₁₁ is hydrogen or a substituted or unsubstituted group selected from methyl, phenyl and benzyl, while R₁₁′ is hydrogen or a substituted or unsubstituted group selected from methyl and ethyl.

In a preferred embodiment

R₁₁ is substituted or unsubstituted group selected from methyl, while R₁₁′ is hydrogen or substituted or unsubstituted methyl.

In a preferred embodiment

R₁₁ is substituted or unsubstituted phenyl, while R₁₁′ is substituted or unsubstituted ethyl.

In a preferred embodiment

R₁₁ is substituted or unsubstituted benzyl, while R₁₁′ is substituted or unsubstituted methyl.

In a preferred embodiment

R₁₁ and R₁₁′ are both hydrogen.

In a preferred embodiment

R₁₄ is hydrogen.

In a preferred embodiment

R₂₁ is substituted or unsubstituted methyl.

In a preferred embodiment

R₃₁ is substituted or unsubstituted methyl.

In a preferred embodiment

R₄₁, R₄₁′ and R₄₁″ are independently selected from hydrogen and a substituted or unsubstituted group selected from methyl, ethyl, phenethyl and —CH₂CH₂CH₂-phenyl.

In a preferred embodiment

R₄₁ is hydrogen or a substituted or unsubstituted group selected from methyl, ethyl, phenethyl and —CH₂CH₂CH₂-phenyl.

In a preferred embodiment

R₄₁′ is hydrogen or substituted or unsubstituted methyl.

In a preferred embodiment

R₄₁ is a substituted or unsubstituted group selected from methyl, phenethyl and —CH₂CH₂CH₂-phenyl, while R₄₁′ is hydrogen or substituted or unsubstituted methyl.

In a preferred embodiment

R₄₁ is substituted or unsubstituted methyl, while R₄₁′ is hydrogen or substituted or unsubstituted methyl.

In a preferred embodiment

R₄₁ is substituted or unsubstituted methyl, while R₄₁′ is hydrogen.

In a preferred embodiment

R₄₁ is substituted or unsubstituted methyl, while R₄₁′ is substituted or unsubstituted methyl.

In a preferred embodiment

R₄₁ is substituted or unsubstituted phenethyl, while R₄₁′ is substituted or unsubstituted methyl.

In a preferred embodiment

R₄₁ is substituted or unsubstituted —CH₂CH₂CH₂-phenyl, while R₄₁′ is substituted or unsubstituted methyl.

In a preferred embodiment

R₆₁ and R₆₁′ are independently selected from hydrogen and unsubstituted methyl.

In a preferred embodiment

R₈₁ is substituted or unsubstituted methyl.

In an embodiment of the compound according to the invention of general Formula (I),

the halogen is fluorine, chlorine, iodine or bromine; preferably fluorine, chlorine, or bromine,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF₃;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF₃;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

the alkyl, alkenyl or alkynyl defined in R₁, if substituted, is substituted with one or more substituent/s selected from —OR₁₁, halogen, —CN, haloalkyl, haloalkoxy and —NR₁₁R₁₁′; preferably the alkyl, alkenyl or alkynyl defined in R₁, if substituted, is substituted with one or more substituent/s selected from —OR₁₁ and halogen; more preferably the alkyl, alkenyl or alkynyl defined in R₁, if substituted, is substituted with one or more substituent/s selected from —OR₁₁ and halogen

In a preferred further embodiment, the compounds of the general Formula (I) are selected from

| EX | CHEMICAL NAME |
|---|---|
| 1 | 6-bromo-2-(1-((3S,5R)-3,5-dimethylpiperazin-1-yl)butyl)-3-ethylquinazolin-4(3H)-one |
| 2 | 2-(1-((3S,5R)-3,5-dimethylpiperazin-1-yl)butyl)-3-methylquinazolin-4(3H)-one |
| 3 | 2-(1-((3S,5R)-3,5-dimethylpiperazin-1-yl)butyl)-3-(2-(methylamino)ethyl)quinazolin- |
| | 4(3H)-one |
| 4 | 2-(1-((3S,5R)-3,5-dimethylpiperazin-1-yl)butyl)-3-ethyl-7-methylquinazolin-4(3H)-one |
| 5 | 8-bromo-2-(1-((3S,5R)-3,5-dimethylpiperazin-1-yl)butyl)-3-ethyl-7- |
| | methoxyquinazolin-4(3H)-one |
| 6 | 2-(1-((3S,5R)-3,5-dimethylpiperazin-1-yl)butyl)-3-ethyl-7-methoxyquinazolin-4(3H)-one |
| 7 | methyl 2-(1-((3S,5R)-3,5-dimethylpiperazin-1-yl)butyl)-3-ethyl-4-oxo-3,4- |
| | dihydroquinazoline-7-carboxylate |
| 8 | methyl 2-(1-((3S,5R)-3,5-dimethylpiperazin-1-yl)butyl)-3-methyl-4-oxo-3,4- |
| | dihydroquinazoline-7-carboxylate |
| 9 | 7-bromo-2-(1-((3S,5R)-3,5-dimethylpiperazin-1-yl)butyl)-3-ethylquinazolin-4(3H)-one |
| 10 | 2-(1-((3S,5R)-3,5-dimethylpiperazin-1-yl)butyl)-7-hydroxy-3-methylquinazolin- |
| | 4(3H)-one |
| 11 | 2-(1-((3S,5R-3,5-dimethylpiperazin-1-yl)butyl)-3-ethyl-7-fluoroquinazolin-4(3H)-one |
| 12 | 2-(1-((3S,5R)-3,5-dimethylpiperazin-1-yl)butyl)-3-ethyl-6-hydroxyquinazolin-4(3H)-one |
| 13 | 8-bromo-2-(1-((3S,5R)-3,5-dimethylpiperazin-1-yl)butyl)-3-ethylquinazolin-4(3H)-one |
| 14 | 5-bromo-2-(1-((3S,5R)-3,5-dimethylpiperazin-1-yl)butyl)-3-ethylquinazolin-4(3H)-one |
| 15 | 2-(1-((3S,5R)-3,5-dimethylpiperazin-1-yl)butyl)-3-ethyl-5-hydroxyquinazolin-4(3H)-one |
| 16 | 6-bromo-2-(1-((3S,5R)-3,5-dimethylpiperazin-1-yl)butyl)-3-methylquinazolin-4(3H)-one |
| 17 | 7-bromo-2-(1-((3S,5R)-3,5-dimethylpiperazin-1-yl)butyl)-3-methylquinazolin-4(3H)-one |
| 18 | 6-bromo-2-(1-((3S,5R)-3,5-dimethylpiperazin-1-yl)butyl)-3-ethyl-7-fluoroquinazolin- |
| | 4(3H)-one |
| 19 | 6-bromo-2-(1-((3S,5R)-3,5-dimethylpiperazin-1-yl)butyl)-3-ethyl-7- |
| | methylquinazolin-4(3H)-one |
| 20 | 6-bromo-2-(1-((3S,5R)-3,5-dimethylpiperazin-1-yl)butyl)-3-ethyl-8- |
| | methylquinazolin-4(3H)-one |
| 21 | 6-bromo-2-(1-((3S,5R)-3,5-dimethylpiperazin-1-yl)butyl)-3-ethyl-5- |
| | methylquinazolin-4(3H)-one |
| 22 | 6-bromo-2-(1-((3S,5R)-3,5-dimethylpiperazin-1-yl)butyl)-3-ethyl-5-fluoroquinazolin- |
| | 4(3H)-one |
| 23 | 6-bromo-5-chloro-2-(1-((3S,5R)-3,5-dimethylpiperazin-1-yl)butyl)-3- |
| | ethylquinazolin-4(3H)-one |
| 24 | 2-(1-((3S,5R)-3,5-dimethylpiperazin-1-yl)butyl)-3-ethyl-6-(ethylamino)quinazolin- |
| | 4(3H)-one |
| 25 | 2-(1-((3S,5R)-3,5-dimethylpiperazin-1-yl)butyl)-3-ethyl-7-(ethylamino)quinazolin- |
| | 4(3H)-one |
| 26 | tert-butyl (2-(1-((3S,5R)-3,5-dimethylpiperazin-1-yl)butyl)-3-ethyl-4-oxo-3,4- |
| | dihydroquinazolin-7-yl)(ethyl)carbamate |
| 27 | 2-(1-((3S,5R)-3,5-dimethylpiperazin-1-yl)butyl)-3-ethylpyrido[3,2-d]pyrimidin-4(3H)-one |
| 28 | 6-bromo-2-(1-((3S,5R)-3,5-dimethylpiperazin-1-yl)butyl)-3-ethylpyrido[2,3- |
| | d]pyrimidin-4(3H)-one |
| 29 | 6-bromo-2-((R)-1-((3S,5R)-3,5-dimethylpiperazin-1-yl)butyl)-3-ethylquinazolin- |
| | 4(3H)-one |
| 30 | 6-bromo-2-((S)-1-((3S,5R)-3,5-dimethylpiperazin-1-yl)butyl)-3-ethylquinazolin- |
| | 4(3H)-one |
| 31 | -((S)-1-((3S,5R)-3,5-dimethylpiperazin-1-yl)butyl)-3-methylquinazolin-4(3H)-one |
| 32 | 2-((R)-1-((3S,5R)-3,5-dimethylpiperazin-1-yl)butyl)-3-methylquinazolin-4(3H)-one |
| 33 | 2-((S)-1-((3S,5R)-3,5-dimethylpiperazin-1-yl)butyl)-3-methylquinazolin-4(3H)-one |
| 34 | 2-((R)-1-((3S,5R)-3,5-dimethylpiperazin-1-yl)butyl)-3-ethylquinazolin-4(3H)-one |
| 35 | 2-((R)-1-((3S,5R)-3,5-dimethylpiperazin-1-yl)butyl)-3-ethyl-6-hydroxyquinazolin- |
| | 4(3H)-one |
| 36 | 2-((S)-1-((3S,5R)-3,5-dimethylpiperazin-1-yl)butyl)-3-ethyl-6-hydroxyquinazolin- |
| | 4(3H)-one |
| 37 | (S)-3-ethyl-8-fluoro-6-methoxy-2-(1-(piperazin-1-yl)butyl)quinazolin-4(3H)-one |
| 38 | (R)-3-ethyl-8-fluoro-6-methoxy-2-(1-(piperazin-1-yl)butyl)quinazolin-4(3H)-one |
| 39 | (S)-5-bromo-3-ethyl-8-fluoro-6-methoxy-2-(1-(piperazin-1-yl)butyl)quinazolin- |
| | 4(3H)-one |
| 40 | (R)-5-bromo-3-ethyl-8-fluoro-6-methoxy-2-(1-(piperazin-1-yl)butyl)quinazolin- |
| | 4(3H)-one |
| 41 | 6-bromo-3-methyl-2-((R)-1-((S)-3-methylpiperazin-1-yl)butyl)quinazolin-4(3H)-one |
| 42 | 6-bromo-3-methyl-2-((S)-1-((S)-3-methylpiperazin-1-yl)butyl)quinazolin-4(3H)-one |
| 43 | 3-ethyl-2-((R)-1-((S)-3-methylpiperazin-1-yl)butyl)quinazolin-4(3H)-one |
| 44 | 3-ethyl-2-((S)-1-((S)-3-methylpiperazin-1-yl)butyl)quinazolin-4(3H)-one |
| 45 | 3-ethyl-8-fluoro-6-methoxy-2-((S)-1-((S)-3-methylpiperazin-1-yl)butyl)quinazolin- |
| | 4(3H)-one |
| 46 | 3-ethyl-8-fluoro-6-methoxy-2-((R)-1-((S)-3-methylpiperazin-1-yl)butyl)quinazolin- |
| | 4(3H)-one |
| 47 | 6-bromo-3-ethyl-8-fluoro-2-((R)-1-((R)-3-methylpiperazin-1-yl)butyl)quinazolin- |
| | 4(3H)-one |
| 48 | 6-bromo-3-ethyl-8-fluoro-2-((S)-1-((R)-3-methylpiperazin-1-yl)butyl)quinazolin- |
| | 4(3H)-one |
| 49 | 6,7-dichloro-3-ethyl-2-((R)-1-((S)-3-methylpiperazin-1-yl)butyl)quinazolin-4(3H)-one |
| 50 | 6,7-dichloro-3-ethyl-2-((S)-1-((S)-3-methylpiperazin-1-yl)butyl)quinazolin-4(3H)-one |
| 51 | 6-bromo-3-ethyl-8-fluoro-2-((R)-1-((S)-3-methylpiperazin-1-yl)butyl)quinazolin- |
| | 4(3H)-one |
| 52 | 6-bromo-3-ethyl-8-fluoro-2-((S)-1-((S)-3-methylpiperazin-1-yl)butyl)quinazolin- |
| | 4(3H)-one |
| 53 | 6-chloro-3-ethyl-7-fluoro-2-((R)-1-((S)-3-methylpiperazin-1-yl)butyl)quinazolin- |
| | 4(3H)-one |
| 54 | 6-chloro-3-ethyl-7-fluoro-2-((S)-1-((S)-3-methylpiperazin-1-yl)butyl)quinazolin- |
| | 4(3H)-one |
| 55 | 6-bromo-3-ethyl-2-((R)-1-((S)-3-(fluoromethyl)piperazin-1-yl)butyl)quinazolin- |
| | 4(3H)-one |
| 56 | 6-bromo-3-ethyl-2-((S)-1-((R)-3-(fluoromethyl)piperazin-1-yl)butyl)quinazolin- |
| | 4(3H)-one |
| 57 | 6-bromo-3-ethyl-2-((S)-1-((S)-3-(fluoromethyl)piperazin-1-yl)butyl)quinazolin- |
| | 4(3H)-one |
| 58 | 6-bromo-3-ethyl-2-((R)-1-((S)-3-(hydroxymethyl)piperazin-1-yl)butyl)quinazolin- |
| | 4(3H)-one |
| 59 | 6-bromo-3-ethyl-2-((S)-1-((S)-3-(hydroxymethyl)piperazin-1-yl)butyl)quinazolin- |
| | 4(3H)-one |
| 60 | 6-bromo-3-ethyl-2-((R)-1-((R)-3-(hydroxymethyl)piperazin-1-yl)butyl)quinazolin- |
| | 4(3H)-one |
| 61 | 6-bromo-3-ethyl-2-((S)-1-((R)-3-(hydroxymethyl)piperazin-1-yl)butyl)quinazolin- |
| | 4(3H)-one |
| 62 | 6-bromo-7-fluoro-3-methyl-2-((R)-1-((S)-3-methylpiperazin-1-yl)butyl)quinazolin- |
| | 4(3H)-one |
| 63 | 6-bromo-7-fluoro-3-methyl-2-((S)-1-((S)-3-methylpiperazin-1-yl)butyl)quinazolin- |
| | 4(3H)-one |
| 64 | 3-ethyl-2-((R)-1-((R)-3-methylpiperazin-1-yl)butyl)pyrido[4,3-d]pyrimidin-4(3H)-one |
| 65 | 3-ethyl-2-((S)-1-((R)-3-methylpiperazin-1-yl)butyl)pyrido[4,3-d]pyrimidin-4(3H)-one |
| 66 | 3-ethyl-2-((R)-1-((S)-3-methylpiperazin-1-yl)butyl)pyrido[4,3-d]pyrimidin-4(3H)-one |
| 67 | 3-ethyl-2-((S)-1-((S)-3-methylpiperazin-1-yl)butyl)pyrido[4,3-d]pyrimidin-4(3H)-one |
| 68 | 6-bromo-3-ethyl-2-((R)-1-((S)-3-methylpiperazin-1-yl)butyl)pyrido[2,3-d]pyrimidin- |
| | 4(3H)-one |
| 69 | 6-bromo-3-ethyl-2-((S)-1-((S)-3-methylpiperazin-1-yl)butyl)pyrido[2,3-d]pyrimidin- |
| | 4(3H)-one |
| 70 | 3-ethyl-2-((R)-1-((S)-3-methylpiperazin-1-yl)butyl)pyrido[3,2-d]pyrimidin-4(3H)-one |
| 71 | 3-ethyl-2-((S)-1-((S)-3-methylpiperazin-1-yl)butyl)pyrido[3,2-d]pyrimidin-4(3H)-one |
| 72 | 6-bromo-2-((R)-1-((S)-3-methylpiperazin-1-yl)butyl)-3-propylpyrido[2,3-d]pyrimidin- |
| | 4(3H)-one |
| 73 | 6-bromo-2-((S)-1-((S)-3-methylpiperazin-1-yl)butyl)-3-propylpyrido[2,3-d]pyrimidin- |
| | 4(3H)-one |
| 74 | 6-bromo-3-(cyclopropylmethyl)-2-((S)-1-((S)-3-methylpiperazin-1- |
| | yl)butyl)pyrido[2,3-d]pyrimidin-4(3H)-one |
| 75 | 6-bromo-3-(cyclopropylmethyl)-2-((R)-1-((S)-3-methylpiperazin-1- |
| | yl)butyl)pyrido[2,3-d]pyrimidin-4(3H)-one |
| 76 | 6-chloro-3-ethyl-2-((R)-1-((S)-3-methylpiperazin-1-yl)butyl)pyrido[2,3-d]pyrimidin- |
| | 4(3H)-one |
| 77 | 3-ethyl-2-((S)-1-((S)-3-methylpiperazin-1-yl)butyl)-7-(trifluoromethyl)pyrido[2,3- |
| | d]pyrimidin-4(3H)-one |
| 78 | 3-ethyl-2-((R)-1-((S)-3-methylpiperazin-1-yl)butyl)-7-(trifluoromethyl)pyrido[2,3- |
| | d]pyrimidin-4(3H)-one |
| 79 | 6-bromo-3-ethyl-2-(1-(piperazin-1-yl)butyl)quinazolin-4(3H)-one. |
| 80 | 3-methyl-2-(1-(piperazin-1-yl)butyl)quinazolin-4(3H)-one |
| 81 | 7-bromo-3-ethyl-2-(1-(piperazin-1-yl)butyl)quinazolin-4(3H)-one |
| 82 | 6-bromo-2-(1-(piperazin-1-yl)butyl)-3-propylquinazolin-4(3H)-one |
| 83 | 6-bromo-3-ethyl-7-fluoro-2-(1-(piperazin-1-yl)butyl)quinazolin-4(3H)-one |
| 84 | 6-bromo-3-ethyl-5-methyl-2-(1-(piperazin-1-yl)butyl)quinazolin-4(3H)-one |
| 85 | 6-bromo-3-ethyl-7-methyl-2-(1-(piperazin-1-yl)butyl)quinazolin-4(3H)-one |
| 86 | 3-ethyl-6,7-difluoro-2-(1-(piperazin-1-yl)butyl)quinazolin-4(3H)-one |
| 87 | 6-bromo-3-ethyl-8-fluoro-2-(1-(piperazin-1-yl)butyl)quinazolin-4(3H)-one |
| 88 | 6-bromo-3-ethyl-5-fluoro-2-(1-(piperazin-1-yl)butyl)quinazolin-4(3H)-one |
| 89 | 6-bromo-3-ethyl-8-methyl-2-(1-(piperazin-1-yl)butyl)quinazolin-4(3H)-one |
| 90 | 8-chloro-3-ethyl-6-fluoro-2-(1-(piperazin-1-yl)butyl)quinazolin-4(3H)-one |
| 91 | 6-bromo-5-chloro-3-ethyl-2-(1-(piperazin-1-yl)butyl)quinazolin-4(3H)-one |
| 92 | 3-ethyl-2-(1-(piperazin-1-yl)butyl)pyrido[3,2-d]pyrimidin-4(3H)-one |
| 93 | 6-bromo-3-ethyl-2-(1-(piperazin-1-yl)butyl)pyrido[2,3-d]pyrimidin-4(3H)-one |
| 94 | 3-ethyl-2-(1-(piperazin-1-yl)butyl)pyrido[2,3-d]pyrimidin-4(3H)-one |
| 95 | (S)-6-bromo-3-ethyl-8-fluoro-2-(1-(piperazin-1-yl)butyl)quinazolin-4(3H)-one |
| 96 | (R)-6-bromo-3-ethyl-8-fluoro-2-(1-(piperazin-1-yl)butyl)quinazolin-4(3H)-one |
| 97 | 6-bromo-3-ethyl-2-((R)-1-((S)-3-methylpiperazin-1-yl)butyl)quinazolin-4(3H)-one |
| 98 | 6-bromo-3-ethyl-2-((R)-1-((R)-3-methylpiperazin-1-yl)butyl)quinazolin-4(3H)-one |
| 99 | 6-bromo-3-ethyl-2-((S)-1-((S)-3-methylpiperazin-1-yl)butyl)quinazolin-4(3H)-one |
| 100 | 6-bromo-3-ethyl-2-((S)-1-((R)-3-methylpiperazin-1-yl)butyl)quinazolin-4(3H)-one |
| 101 | 6-chloro-3-ethyl-2-((R)-1-((R)-3-methylpiperazin-1-yl)butyl)quinazolin-4(3H)-one |
| 102 | 6-chloro-3-ethyl-2-((S)-1-((R)-3-methylpiperazin-1-yl)butyl)quinazolin-4(3H)-one |
| 103 | 6-chloro-3-ethyl-2-((R)-1-((S)-3-methylpiperazin-1-yl)butyl)quinazolin-4(3H)-one |
| 104 | 6-chloro-3-ethyl-2-((S)-1-((S)-3-methylpiperazin-1-yl)butyl)quinazolin-4(3H)-one |
| 105 | 6-bromo-3-ethyl-7-fluoro-2-((S)-1-((S)-3-methylpiperazin-1-yl)butyl)quinazolin- |
| | 4(3H)-one |
| 106 | 6-bromo-3-ethyl-7-fluoro-2-((R)-1-((S)-3-methylpiperazin-1-yl)butyl)quinazolin- |
| | 4(3H)-one |
| 107 | 3-((S)-4-((S)-1-(6-bromo-3-ethyl-4-oxo-3,4-dihydroquinazolin-2-yl)butyl)piperazin- |
| | 2-yl)propanoic acid |
| 108 | 3-((S)-4-((R)-1-(6-bromo-3-ethyl-4-oxo-3,4-dihydroquinazolin-2-yl)butyl)piperazin- |
| | 2-yl)propanoic acid |
| 109 | 6-bromo-2-((R)-1-((R)-3,4-dimethylpiperazin-1-yl)butyl)-3-ethylquinazolin-4(3H)-one |
| 110 | 6-bromo-2-((S)-1-((R)-3,4-dimethylpiperazin-1-yl)butyl)-3-ethylquinazolin-4(3H)-one |
| 111 | 6-bromo-2-((S)-1-((S)-3,4-dimethylpiperazin-1-yl)butyl)-3-ethylquinazolin-4(3H)-one |
| 112 | 6-bromo-2-((R)-1-((S)-3,4-dimethylpiperazin-1-yl)butyl)-3-ethylquinazolin-4(3H)-one |
| 113 | 2-(1-((3S,5R)-3,5-dimethylpiperazin-1-yl)butyl)-3-(2-methoxyethyl)quinazolin- |
| | 4(3H)-one |
| 114 | 2-(1-((3S,5R)-3,5-dimethylpiperazin-1-yl)-3-methylbutyl)-3-(2- |
| | methoxyethyl)quinazolin-4(3H)-one |
| 115 | 2-(1-((3S,5R)-3,5-dimethylpiperazin-1-yl)pentyl)-3-(2-methoxyethyl)quinazolin- |
| | 4(3H)-one |
| 116 | 2-(2-cyclopropyl-1-((3S,5R)-3,5-dimethylpiperazin-1-yl)ethyl)-3-(2- |
| | methoxyethyl)quinazolin-4(3H)-one |
| 117 | 3-(2-methoxyethyl)-2-(1-(piperazin-1-yl)butyl)quinazolin-4(3H)-one |
| 118 | 2-(1-((3S,5R)-3,5-dimethylpiperazin-1-yl)butyl)-3-(furan-2-ylmethyl)quinazolin- |
| | 4(3H)-one |
| 119 | 2-(1-((3S,5R)-3,5-dimethylpiperazin-1-yl)butyl)-3-(2-methoxyethyl)-6- |
| | methylquinazolin-4(3H)-one |
| 120 | 2-(1-((3S,5R)-3,5-dimethylpiperazin-1-yl)butyl)-6-methoxy-3-(2- |
| | methoxyethyl)quinazolin-4(3H)-one |
| 121 | 2-(1-((3S,5R)-3,5-dimethylpiperazin-1-yl)butyl)-3-(2-methoxyethyl)pyrido[3,4- |
| | d]pyrimidin-4(3H)-one |
| 122 | ethyl 3-(6-bromo-2-(1-((3S,5R)-3,5-dimethylpiperazin-1-yl)butyl)-4-oxoquinazolin- |
| | 3(4H)-yl)propanoate |
| 123 | 2-((R)-1-((3S,5R)-3,5-dimethylpiperazin-1-yl)butyl)-3-(2-methoxyethyl)quinazolin- |
| | 4(3H)-one |
| 124 | 2-((S)-1-((3S,5R)-3,5-dimethylpiperazin-1-yl)butyl)-3-(2-methoxyethyl)quinazolin- |
| | 4(3H)-one |
| 125 | 3-(6-bromo-2-(1-((3S,5R)-3,5-dimethylpiperazin-1-yl)butyl)-4-oxoquinazolin-3(4H)- |
| | yl)propanoic acid |
| 126 | 2-(6-bromo-2-(1-((3S,5R)-3,5-dimethylpiperazin-1-yl)butyl)-4-oxoquinazolin-3(4H)- |
| | yl)acetic acid |
| 127 | 2-(1-((3S,5R)-3,5-dimethylpiperazin-1-yl)butyl)-3-(2-(methylamino)ethyl)quinazolin- |
| | 4(3H)-one |
| 128 | 3-(2-(dimethylamino)ethyl)-2-(1-((3S,5R)-3,5-dimethylpiperazin-1- |
| | yl)butyl)quinazolin-4(3H)-one |
| 129 | 2-(1-((3S,5R)-3,5-dimethylpiperazin-1-yl)butyl)-3-(2- |
| | (methyl(phenethyl)amino)ethyl)quinazolin-4(3H)-one |
| 130 | 2-(1-((3S,5R)-3,5-dimethylpiperazin-1-yl)butyl)-3-(2-(methyl(3- |
| | phenylpropyl)amino)ethyl)quinazolin-4(3H)-one |
| 131 | 3-(2-(dimethylamino)ethyl)-2-(1-((3S,5R)-3,4,5-trimethylpiperazin-1- |
| | yl)butyl)quinazolin-4(3H)-one |
| 132 | 2-(1-((3S,5R)-3,5-dimethylpiperazin-1-yl)butyl)-3-ethyl-6-(pyridin-4-yl)quinazolin- |
| | 4(3H)-one |
| 133 | 2-(1-((3S,5R)-3,5-dimethylpiperazin-1-yl)butyl)-3-ethyl-7-(pyridin-4-yl)quinazolin- |
| | 4(3H)-one |
| 134 | 2-(1-((3S,5R)-3,5-dimethylpiperazin-1-yl)butyl)-3-ethyl-7-(3- |
| | hydroxyphenyl)quinazolin-4(3H)-one |
| 135 | 2-(1-((3S,5R)-3,5-dimethylpiperazin-1-yl)butyl)-3-ethyl-7-(2-methoxypyridin-4- |
| | yl)quinazolin-4(3H)-one |
| 136 | 2-(1-((3S,5R)-3,5-dimethylpiperazin-1-yl)butyl)-3-methyl-7-(2- |
| | ((methylamino)methyl)pyridin-4-yl)quinazolin-4(3H)-one |
| 137 | 2-(1-((3S,5R-3,5-dimethylpiperazin-1-yl)butyl)-3-ethyl-7-(pyrrolidin-1-yl)quinazolin- |
| | 4(3H)-one |
| 138 | 2-(1-((3S,5R)-3,5-dimethylpiperazin-1-yl)butyl)-3-ethyl-6-(pyrrolidin-1-yl)quinazolin- |
| | 4(3H)-one |
| 139 | 6-(4-(dimethylamino)piperidin-1-yl)-2-(1-((3S,5R)-3,5-dimethylpiperazin-1-yl)butyl)- |
| | 3-ethylquinazolin-4(3H)-one |
| 140 | 2-(1-((3S,5R)-3,5-dimethylpiperazin-1-yl)butyl)-3-ethyl-6-(4- |
| | (methylamino)piperidin-1-yl)quinazolin-4(3H)-one |
| 141 | 6-(4-aminopiperidin-1-yl)-2-(1-((3S,5R)-3,5-dimethylpiperazin-1-yl)butyl)-3- |
| | ethylquinazolin-4(3H)-one |
| 142 | 2-(1-((3S,5R)-3,5-dimethylpiperazin-1-yl)butyl)-3-ethyl-6-((1-methylpiperidin-4- |
| | yl)amino)quinazolin-4(3H)-one |
| 143 | 2-(1-((3S,5R)-3,5-dimethylpiperazin-1-yl)butyl)-3-ethyl-6-(methyl(1- |
| | methylpiperidin-4-yl)amino)quinazolin-4(3H)-one |
| 144 | 6-(benzyl(methyl)amino)-2-(1-((3S,5R)-3,5-dimethylpiperazin-1-yl)butyl)-3- |
| | ethylquinazolin-4(3H)-one |
| 145 | 2-(1-((3S,5R)-3,5-dimethylpiperazin-1-yl)butyl)-3-ethyl-6-((2- |
| | (methylamino)ethyl)amino)quinazolin-4(3H)-one |
| 146 | 2-(1-((3S,5R)-3,5-dimethylpiperazin-1-yl)butyl)-3-ethyl-6-(methyl(2- |
| | (methylamino)ethyl)amino)quinazolin-4(3H)-one |
| 147 | 2-(1-((3S,5R)-3,5-dimethylpiperazin-1-yl)butyl)-3-ethyl-7-((2- |
| | (methylamino)ethyl)amino)quinazolin-4(3H)-one |
| 148 | 2-(1-((3S,5R)-3,5-dimethylpiperazin-1-yl)butyl)-3-ethyl-6-(4-(2-hydroxyphenyl)-5,6- |
| | dihydropyridin-1(2H)-yl)quinazolin-4(3H)-one |
| 149 | 2-(1-((3S,5R)-3,5-dimethylpiperazin-1-yl)butyl)-3-ethyl-6-((3S,3aR,7aR)-3- |
| | phenylhexahydro-4,7-ethanopyrrolo[3,2-b]pyridin-1(2H)-yl)quinazolin-4(3H)-one |
| 150 | 6-((1-benzylpiperidin-4-yl)amino)-2-(1-((3S,5R)-3,5-dimethylpiperazin-1-yl)butyl)-3- |
| | ethylquinazolin-4(3H)-one |
| 151 | 6-(4-(benzyl(methyl)amino)piperidin-1-yl)-2-(1-((3S,5R)-3,5-dimethylpiperazin-1- |
| | yl)butyl)-3-ethylquinazolin-4(3H)-one |
| 152 | 2-((S)-1-((3S,5R)-3,5-dimethylpiperazin-1-yl)butyl)-3-ethyl-6-((S)-2-methyl-1-oxa- |
| | 4,9-diazaspiro[5.5]undecan-4-yl)quinazolin-4(3H)-one |
| 153 | 2-((R)-1-((3S,5R)-3,5-dimethylpiperazin-1-yl)butyl)-3-ethyl-6-((S)-2-methyl-1-oxa- |
| | 4,9-diazaspiro[5.5]undecan-4-yl)quinazolin-4(3H)-one |
| 154 | 2-((S)-1-((3S,5R)-3,5-dimethylpiperazin-1-yl)butyl)-3-ethyl-6-((R)-2-methyl-9- |
| | phenethyl-1-oxa-4,9-diazaspiro[5.5]undecan-4-yl)quinazolin-4(3H)-one |
| 155 | 2-((S)-1-((3S,5R)-3,5-dimethylpiperazin-1-yl)butyl)-3-ethyl-6-((S)-2-methyl-9- |
| | phenethyl-1-oxa-4,9-diazaspiro[5.5]undecan-4-yl)quinazolin-4(3H)-one |
| 156 | 2-((R)-1-((3S,5R)-3,5-dimethylpiperazin-1-yl)butyl)-3-ethyl-6-((S)-2-methyl-9- |
| | phenethyl-1-oxa-4,9-diazaspiro[5.5]undecan-4-yl)quinazolin-4(3H)-one |
| 157 | 2-((R)-1-((3S,5R)-3,5-dimethylpiperazin-1-yl)butyl)-3-ethyl-6-((H)-2-methyl-9- |
| | phenethyl-1-oxa-4,9-diazaspiro[5.5]undecan-4-yl)quinazolin-4(3H)-one |
| 158 | 2-((S)-1-((3S,5R)-3,5-dimethylpiperazin-1-yl)butyl)-3-ethyl-6-((2-((R)-9-(pyridin-2- |
| | yl)-6-oxaspiro[4.5]decan-9-yl)ethyl)amino)quinazolin-4(3H)-one |
| 159 | 2-((R)-1-((3S,5R)-3,5-dimethylpiperazin-1-yl)butyl)-3-ethyl-6-((2-((S)-9-(pyridin-2- |
| | yl)-6-oxaspiro[4.5]decan-9-yl)ethyl)amino)quinazolin-4(3H)-one |
| 160 | 2-((R)-1-((3S,5R)-3,5-dimethylpiperazin-1-yl)butyl)-3-ethyl-6-((2-((R)-9-(pyridin-2- |
| | yl)-6-oxaspiro[4.5]decan-9-yl)ethyl)amino)quinazolin-4(3H)-one |
| 161 | 2-((S)-1-((3S,5R)-3,5-dimethylpiperazin-1-yl)butyl)-3-ethyl-6-((2-((S)-9-(pyridin-2- |
| | yl)-6-oxaspiro[4.5]decan-9-yl)ethyl)amino)quinazolin-4(3H)-one |
| 162 | tert-Butyl (8-bromo-2-(1-((3S,5R)-3,5-dimethylpiperazin-1-yl)butyl)-3-ethyl-4-oxo- |
| | 3,4-dihydroquinazolin-7-yl)(3-methoxybenzyl)carbamate |
| 163 | N-(2-(1-((3S,5R)-3,5-dimethylpiperazin-1-yl)butyl)-3-ethyl-4-oxo-3,4- |
| | dihydroquinazolin-6-yl)-N-(1-methylpiperidin-4-yl)propionamide. |
| 164 | 2-((S)-1-((3S,5R)-3,5-dimethylpiperazin-1-yl)butyl)-3-methyl-7-((R)-3- |
| | (methylamino)-1-phenylpropoxy)quinazolin-4(3H)-one |
| 165 | 2-((R)-1-((3S,5R)-3,5-dimethylpiperazin-1-yl)butyl)-3-methyl-7-((R)-3- |
| | (methylamino)-1-phenylpropoxy)quinazolin-4(3H)-one |
| 166 | 2-((S)-1-((3S,5R)-3,5-dimethylpiperazin-1-yl)butyl)-3-methyl-7-((S)-3- |
| | (methylamino)-1-phenylpropoxy)quinazolin-4(3H)-one |
| 167 | 2-((R)-1-((3S,5R)-3,5-dimethylpiperazin-1-yl)butyl)-3-methyl-7-((S)-3- |
| | (methylamino)-1-phenylpropoxy)quinazolin-4(3H)-one |
| 168 | 2-((S)-1-((3S,5R)-3,5-dimethylpiperazin-1-yl)butyl)-3-methyl-7-((S)-2- |
| | (methylamino)-1-phenylethoxy)quinazolin-4(3H)-one |
| 169 | 2-((R)-1-((3S,5R)-3,5-dimethylpiperazin-1-yl)butyl)-3-methyl-7-((R)-2- |
| | (methylamino)-1-phenylethoxy)quinazolin-4(3H)-one |
| 170 | 2-((R)-1-((3S,5R)-3,5-dimethylpiperazin-1-yl)butyl)-3-methyl-7-((S)-2- |
| | (methylamino)-1-phenylethoxy)quinazolin-4(3H)-one |
| 171 | 2-((S)-1-((3S,5R)-3,5-dimethylpiperazin-1-yl)butyl)-3-methyl-7-((R)-2- |
| | (methylamino)-1-phenylethoxy)quinazolin-4(3H)-one |
| 172 | 2-(1-((3S,5R)-3,5-dimethylpiperazin-1-yl)butyl)-7-(hydroxymethyl)-3- |
| | methylquinazolin-4(3H)-one |
| 173 | 2-(1-((3S,5R)-3,5-dimethylpiperazin-1-yl)butyl)-3-ethyl-6-(2- |
| | hydroxyethyl)quinazolin-4(3H)-one |
| 174 | 6-(2-(benzyl(methyl)amino)ethyl)-2-(1-((3S,5R)-3,5-dimethylpiperazin-1-yl)butyl)-3- |
| | ethylquinazolin-4(3H)-one |
| 175 | 2-(1-((3S,5R)-3,5-dimethylpiperazin-1-yl)butyl)-3-ethyl-6-(2- |
| | (isopentyl(methyl)amino)ethyl)quinazolin-4(3H)-one |
| 176 | 2-(1-((3S,5R)-3,5-dimethylpiperazin-1-yl)butyl)-3-methyl-4-oxo-3,4- |
| | dihydroquinazoline-7-carbonitrile |
| 177 | 2-(1-((3S,5R)-3,5-dimethylpiperazin-1-yl)butyl)-3-ethyl-4-oxo-3,4- |
| | dihydroquinazoline-6-carbonitrile |
| 178 | 2-(1-((3S,5R)-3,5-dimethylpiperazin-1-yl)butyl)-3-methyl-4-oxo-3,4- |
| | dihydroquinazoline-7-carboxylic acid |
| 179 | 2-(1-((3S,5R)-3,5-dimethylpiperazin-1-yl)butyl)-3-ethyl-4-oxo-3,4- |
| | dihydroquinazoline-7-carboxylic acid |
| 180 | N-benzyl-2-(1-((3S,5R)-3,5-dimethylpiperazin-1-yl)butyl)-3-ethyl-4-oxo-3,4- |
| | dihydroquinazoline-7-carboxamide |
| 181 | N-(1-((2-(1-((3S,5R)-3,5-dimethylpiperazin-1-yl)butyl)-3-ethyl-4-oxo-3,4- |
| | dihydroquinazolin-6-yl)methyl)piperidin-4-yl)-N-phenylpropionamide |
| 182 | N-(1-((2-(1-((3S,5R)-3,5-dimethylpiperazin-1-yl)butyl)-3-methyl-4-oxo-3,4- |
| | dihydroquinazolin-7-yl)methyl)piperidin-4-yl)-N-phenylpropionamide |
| 183 | 6-((benzyl(methyl)amino)methyl)-2-(1-((3S,5R)-3,5-dimethylpiperazin-1-yl)butyl)-3- |
| | ethylquinazolin-4(3H)-one |
| 184 | 2-(1-((3S,5R)-3,5-dimethylpiperazin-1-yl)butyl)-3-ethyl-6- |
| | ((isobutyl(methyl)amino)methyl)quinazolin-4(3H)-one |
| 185 | 2-(1-((3S,5R)-3,5-dimethylpiperazin-1-yl)butyl)-3-ethyl-6- |
| | ((isopentyl(methyl)amino)methyl)quinazolin-4(3H)-one |
| 186 | 2-(1-((3S,5R)-3,5-dimethylpiperazin-1-yl)butyl)-3-methyl-7-((4-methylpiperazin-1- |
| | yl)methyl)quinazolin-4(3H)-one |
| 187 | 6-bromo-3-ethyl-2-(1-(piperidin-4-yl)butyl)quinazolin-4(3H)-one |
| 188 | 6-bromo-3-ethyl-2-(1-(1,2,3,6-tetrahydropyridin-4-yl)butyl)quinazolin-4(3H)-one |
| 189 | 6-bromo-2-((S)-1-((R)-3,3-difluoropiperidin-4-yl)butyl)-3-ethylquinazolin-4(3H)-one |
| 190 | 6-bromo-3-ethyl-2-((R)-1-((2S,4S)-2-methylpiperidin-4-yl)butyl)quinazolin-4(3H)-one |
| 191 | 6-bromo-3-ethyl-2-((S)-1-((2S,4S)-2-methylpiperidin-4-yl)butyl)quinazolin-4(3H)-one |
| 192 | 6-bromo-3-ethyl-2-((R)-1-((2S,4R)-2-methylpiperidin-4-yl)butyl)quinazolin-4(3H)-one |
| 193 | 6-bromo-3-ethyl-2-((S)-1-((2S,4R)-2-methylpiperidin-4-yl)butyl)quinazolin-4(3H)-one |
| 194 | 6-bromo-2-((S)-1-((S)-3,3-difluoropiperidin-4-yl)butyl)-3-ethylquinazolin-4(3H)-one |
| 195 | 6-bromo-2-((R)-1-((R)-3,3-difluoropiperidin-4-yl)butyl)-3-ethylquinazolin-4(3H)-one |
| 196 | 6-bromo-2-((S)-1-((R)-3,3-difluoropiperidin-4-yl)butyl)-3-ethylquinazolin-4(3H)-one |
| 197 | 6-bromo-2-((R)-1-((S)-3,3-difluoropiperidin-4-yl)butyl)-3-ethylquinazolin-4(3H)-one |
| 198 | 6-chloro-3-methyl-2-((R)-1-((S)-3-methylpiperazin-1-yl)butyl)quinazolin-4(3H)-one |
| 199 | (R)-6-chloro-3-ethyl-8-fluoro-2-(1-(piperazin-1-yl)butyl)quinazolin-4(3H)-one |
| 200 | (R)-3-ethyl-6,8-difluoro-2-(1-(piperazin-1-yl)butyl)quinazolin-4(3H)-one |
| 201 | 3-ethyl-6,8-difluoro-2-((R)-1-((S)-3-methylpiperazin-1-yl)butyl)quinazolin-4(3H)-one |
| 202 | 6-fluoro-2-((R)-1-((S)-3-methylpiperazin-1-yl)butyl)-3-propylquinazolin-4(3H)-one |
| 203 | 3-ethyl-8-fluoro-2-((R)-1-((S)-3-methylpiperazin-1-yl)butyl)quinazolin-4(3H)-one |
| 204 | 3-ethyl-2-((R)-1-((S)-3-methylpiperazin-1-yl)butyl)-6-(trifluoromethoxy)quinazolin- |
| | 4(3H)-one |
| 205 | 6-fluoro-3-methyl-2-((R)-1-((S)-3-methylpiperazin-1-yl)butyl)quinazolin-4(3H)-one |
| 206 | 6,7-dichloro-3-methyl-2-((R)-1-((S)-3-methylpiperazin-1-yl)butyl)quinazolin-4(3H)-one |
| 207 | 3-ethyl-2-((R)-1-((S)-3-methylpiperazin-1-yl)butyl)-6-(trifluoromethyl)quinazolin- |
| | 4(3H)-one |
| 208 | 6-bromo-3-ethyl-7-fluoro-2-((R)-1-((R)-3-methylpiperazin-1-yl)butyl)quinazolin- |
| | 4(3H)-one |
| 209 | 6-bromo-3-ethyl-7-fluoro-2-((S)-1-((R)-3-methylpiperazin-1-yl)butyl)quinazolin- |
| | 4(3H)-one |
| 210 | 3-ethyl-7-fluoro-6-methoxy-2-((R)-1-((R)-3-methylpiperazin-1-yl)butyl)quinazolin- |
| | 4(3H)-one |
| 211 | 3-ethyl-6-methoxy-2-((R)-1-((R)-3-methylpiperazin-1-yl)butyl)quinazolin-4(3H)-one |
| 212 | 6-bromo-3-ethyl-2-((R)-1-((S)-3-ethylpiperazin-1-yl)butyl)quinazolin-4(3H)-one |
| 213 | 3-ethyl-7-fluoro-6-methoxy-2-((R)-1-((S)-3-methylpiperazin-1-yl)butyl)quinazolin- |
| | 4(3H)-one |
| 214 | 3-ethyl-6-fluoro-2-((R)-1-((S)-3-methylpiperazin-1-yl)butyl)quinazolin-4(3H)-one |
| 215 | 3-ethyl-6-methoxy-2-((R)-1-((S)-3-methylpiperazin-1-yl)butyl)quinazolin-4(3H)-one |
| 216 | 3-ethyl-6,7-difluoro-2-((R)-1-((S)-3-methylpiperazin-1-yl)butyl)quinazolin-4(3H)-one |
| 217 | 6-chloro-3-ethyl-8-fluoro-2-((R)-1-((S)-3-methylpiperazin-1-yl)butyl)quinazolin- |
| | 4(3H)-one |
| 218 | 3-ethyl-5,6-difluoro-2-((R)-1-((S)-3-methylpiperazin-1-yl)butyl)quinazolin-4(3H)-one |
| 219 | 6-bromo-3-ethyl-2-((R)-1-((R)-3-(methoxymethyl)piperazin-1-yl)butyl)quinazolin- |
| | 4(3H)-one |
| 220 | 6-bromo-3-ethyl-2-((S)-1-((R)-3-(methoxymethyl)piperazin-1-yl)butyl)quinazolin- |
| | 4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | CHEMICAL NAME |
|---|---|
| 221 | 6-chloro-7-fluoro-3-methyl-2-((R)-1-((S)-3-methylpiperazin-1- |
| | yl)butyl)quinazolin-4(3H)-one |
| 222 | 5,6-difluoro-3-methyl-2-((R)-1-((S)-3-methylpiperazin-1-yl)butyl)quinazolin- |
| | 4(3H)-one |
| 223 | 6-chloro-8-fluoro-3-methyl-2-((R)-1-((S)-3-methylpiperazin-1- |
| | yl)butyl)quinazolin-4(3H)-one |
| 224 | (R)-6-bromo-2-(1-(3,3-dimethylpiperazin-1-yl)butyl)-3-ethylquinazolin-4(3H)-one |
| 225 | 6,8-difluoro-3-methyl-2-((R)-1-((S)-3-methylpiperazin-1-yl)butyl)quinazolin- |
| | 4(3H)-one |
| 226 | 3-ethyl-5,6,8-trifluoro-2-((R)-1-((S)-3-methylpiperazin-1-yl)butyl)quinazolin- |
| | 4(3H)-one |
| 227 | 6-Bromo-2-((S)-1-((3S,5S)-3,5-dimethylpiperazin-1-yl)butyl)-3-ethylquinazolin- |
| | 4(3H)-one |
| 228 | 6-Bromo-2-((R)-1-((3S,5S)-3,5-dimethylpiperazin-1-yl)butyl)-3-ethylquinazolin- |
| | 4(3H)-one |
| 229 | 6-Chloro-3-ethyl-2-((R)-1-((S)-3-(fluoromethyl)piperazin-1-yl)butyl)quinazolin- |
| | 4(3H)-one |
| 230 | 6-chloro-3-ethyl-2-((R)-1-((R)-3-(fluoromethyl)piperazin-1-yl)butyl)quinazolin- |
| | 4(3H)-one |
| 231 | 6-chloro-3-ethyl-2-((S)-1-((R)-3-(fluoromethyl)piperazin-1-yl)butyl)quinazolin- |
| | 4(3H)-one |
| 232 | 6-chloro-3-ethyl-2-((S)-1-((S)-3-(fluoromethyl)piperazin-1-yl)butyl)quinazolin- |
| | 4(3H)-one. |
| 233 | 3-Ethyl-2-((R)-1-((R)-3-methylpiperazin-1-yl)butyl)-6-(trifluoromethyl)pyrido[3,4- |
| | d]pyrimidin-4(3H)-one |
| 234 | 3-ethyl-2-((R)-1-((S)-3-methylpiperazin-1-yl)butyl)-6-(trifluoromethyl)pyrido[3,4- |
| | d]pyrimidin-4(3H)-one |
| 235 | 3-ethyl-2-((S)-1-((S)-3-methylpiperazin-1-yl)butyl)-6-(trifluoromethyl)pyrido[3,4- |
| | d]pyrimidin-4(3H)-one |
| 236 | 2-((R)-1-((3S,5R)-3,5-Dimethylpiperazin-1-yl)butyl)-3-ethyl-6- |
| | ((methylamino)(phenyl)methyl)quinazolin-4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | CHEMICAL NAME |
|---|---|
| 41 | 6-bromo-3-methyl-2-((R)-1-((S)-3-methylpiperazin-1-yl)butyl)quinazolin-4(3H)-one |
| 42 | 6-bromo-3-methyl-2-((S)-1-((S)-3-methylpiperazin-1-yl)butyl)quinazolin-4(3H)-one |
| 43 | 3-ethyl-2-((R)-1-((S)-3-methylpiperazin-1-yl)butyl)quinazolin-4(3H)-one |
| 44 | 3-ethyl-2-((S)-1-((S)-3-methylpiperazin-1-yl)butyl)quinazolin-4(3H)-one |
| 45 | 3-ethyl-8-fluoro-6-methoxy-2-((S)-1-((S)-3-methylpiperazin-1- |
| | yl)butyl)quinazolin-4(3H)-one |
| 46 | 3-ethyl-8-fluoro-6-methoxy-2-((R)-1-((S)-3-methylpiperazin-1- |
| | yl)butyl)quinazolin-4(3H)-one |
| 49 | 6,7-dichloro-3-ethyl-2-((R)-1-((S)-3-methylpiperazin-1-yl)butyl)quinazolin-4(3H)-one |
| 50 | 6,7-dichloro-3-ethyl-2-((S)-1-((S)-3-methylpiperazin-1-yl)butyl)quinazolin-4(3H)-one |
| 51 | 6-bromo-3-ethyl-8-fluoro-2-((R)-1-((S)-3-methylpiperazin-1-yl)butyl)quinazolin- |
| | 4(3H)-one |
| 52 | 6-bromo-3-ethyl-8-fluoro-2-((S)-1-((S)-3-methylpiperazin-1-yl)butyl)quinazolin- |
| | 4(3H)-one |
| 53 | 6-chloro-3-ethyl-7-fluoro-2-((R)-1-((S)-3-methylpiperazin-1-yl)butyl)quinazolin- |
| | 4(3H)-one |
| 54 | 6-chloro-3-ethyl-7-fluoro-2-((S)-1-((S)-3-methylpiperazin-1-yl)butyl)quinazolin- |
| | 4(3H)-one |
| 55 | 6-bromo-3-ethyl-2-((R)-1-((S)-3-(fluoromethyl)piperazin-1-yl)butyl)quinazolin- |
| | 4(3H)-one |
| 58 | 6-bromo-3-ethyl-2-((R)-1-((S)-3-(hydroxymethyl)piperazin-1-yl)butyl)quinazolin- |
| | 4(3H)-one |
| 59 | 6-bromo-3-ethyl-2-((S)-1-((S)-3-(hydroxymethyl)piperazin-1-yl)butyl)quinazolin- |
| | 4(3H)-one |
| 62 | 6-bromo-7-fluoro-3-methyl-2-((R)-1-((S)-3-methylpiperazin-1- |
| | yl)butyl)quinazolin-4(3H)-one |
| 63 | 6-bromo-7-fluoro-3-methyl-2-((S)-1-((S)-3-methylpiperazin-1- |
| | yl)butyl)quinazolin-4(3H)-one |
| 66 | 3-ethyl-2-((R)-1-((S)-3-methylpiperazin-1-yl)butyl)pyrido[4,3-d]pyrimidin-4(3H)-one |
| 67 | 3-ethyl-2-((S)-1-((S)-3-methylpiperazin-1-yl)butyl)pyrido[4,3-d]pyrimidin-4(3H)-one |
| 68 | 6-bromo-3-ethyl-2-((R)-1-((S)-3-methylpiperazin-1-yl)butyl)pyrido[2,3- |
| | d]pyrimidin-4(3H)-one |
| 69 | 6-bromo-3-ethyl-2-((S)-1-((S)-3-methylpiperazin-1-yl)butyl)pyrido[2,3- |
| | d]pyrimidin-4(3H)-one |
| 70 | 3-ethyl-2-((R)-1-((S)-3-methylpiperazin-1-yl)butyl)pyrido[3,2-d]pyrimidin-4(3H)-one |
| 71 | 3-ethyl-2-((S)-1-((S)-3-methylpiperazin-1-yl)butyl)pyrido[3,2-d]pyrimidin-4(3H)-one |
| 72 | 6-bromo-2-((R)-1-((S)-3-methylpiperazin-1-yl)butyl)-3-propylpyrido[2,3- |
| | d]pyrimidin-4(3H)-one |
| 73 | 6-bromo-2-((S)-1-((S)-3-methylpiperazin-1-yl)butyl)-3-propylpyrido[2,3- |
| | d]pyrimidin-4(3H)-one |
| 74 | 6-bromo-3-(cyclopropylmethyl)-2-((S)-1-((S)-3-methylpiperazin-1- |
| | yl)butyl)pyrido[2,3-d]pyrimidin-4(3H)-one |
| 75 | 6-bromo-3-(cyclopropylmethyl)-2-((R)-1-((S)-3-methylpiperazin-1- |
| | yl)butyl)pyrido[2,3-d]pyrimidin-4(3H)-one |
| 76 | 6-chloro-3-ethyl-2-((R)-1-((S)-3-methylpiperazin-1-yl)butyl)pyrido[2,3- |
| | d]pyrimidin-4(3H)-one |
| 77 | 3-ethyl-2-((S)-1-((S)-3-methylpiperazin-1-yl)butyl)-7-(trifluoromethyl)pyrido[2,3- |
| | d]pyrimidin-4(3H)-one |
| 78 | 3-ethyl-2-((R)-1-((S)-3-methylpiperazin-1-yl)butyl)-7-(trifluoromethyl)pyrido[2,3- |
| | d]pyrimidin-4(3H)-one |
| 97 | 6-bromo-3-ethyl-2-((R)-1-((S)-3-methylpiperazin-1-yl)butyl)quinazolin-4(3H)-one |
| 99 | 6-bromo-3-ethyl-2-((S)-1-((S)-3-methylpiperazin-1-yl)butyl)quinazolin-4(3H)-one |
| 103 | 6-chloro-3-ethyl-2-((R)-1-((S)-3-methylpiperazin-1-yl)butyl)quinazolin-4(3H)-one |
| 104 | 6-chloro-3-ethyl-2-((S)-1-((S)-3-methylpiperazin-1-yl)butyl)quinazolin-4(3H)-one |
| 105 | 6-bromo-3-ethyl-7-fluoro-2-((S)-1-((S)-3-methylpiperazin-1-yl)butyl)quinazolin- |
| | 4(3H)-one |
| 106 | 6-bromo-3-ethyl-7-fluoro-2-((R)-1-((S)-3-methylpiperazin-1-yl)butyl)quinazolin- |
| | 4(3H)-one |
| 107 | 3-((S)-4-((S)-1-(6-bromo-3-ethyl-4-oxo-3,4-dihydroquinazolin-2- |
| | yl)butyl)piperazin-2-yl)propanoic acid |
| 108 | 3-((S)-4-((R)-1-(6-bromo-3-ethyl-4-oxo-3,4-dihydroquinazolin-2- |
| | yl)butyl)piperazin-2-yl)propanoic acid |
| 111 | 6-bromo-2-((S)-1-((S)-3,4-dimethylpiperazin-1-yl)butyl)-3-ethylquinazolin-4(3H)-one |
| 112 | 6-bromo-2-((R)-1-((S)-3,4-dimethylpiperazin-1-yl)butyl)-3-ethylquinazolin- |
| | 4(3H)-one |
| 190 | 6-bromo-3-ethyl-2-((R)-1-((2S,4S)-2-methylpiperidin-4-yl)butyl)quinazolin- |
| | 4(3H)-one |
| 191 | 6-bromo-3-ethyl-2-((S)-1-((2S,4S)-2-methylpiperidin-4-yl)butyl)quinazolin- |
| | 4(3H)-one |
| 192 | 6-bromo-3-ethyl-2-((R)-1-((2S,4R)-2-methylpiperidin-4-yl)butyl)quinazolin- |
| | 4(3H)-one |
| 193 | 6-bromo-3-ethyl-2-((S)-1-((2S,4R)-2-methylpiperidin-4-yl)butyl)quinazolin- |
| | 4(3H)-one |
| 198 | 6-chloro-3-methyl-2-((R)-1-((S)-3-methylpiperazin-1-yl)butyl)quinazolin-4(3H)-one |
| 201 | 3-ethyl-6,8-difluoro-2-((R)-1-((S)-3-methylpiperazin-1-yl)butyl)quinazolin-4(3H)-one |
| 202 | 6-fluoro-2-((R)-1-((S)-3-methylpiperazin-1-yl)butyl)-3-propylquinazolin-4(3H)-one |
| 203 | 3-ethyl-8-fluoro-2-((R)-1-((S)-3-methylpiperazin-1-yl)butyl)quinazolin-4(3H)-one |
| 204 | 3-ethyl-2-((R)-1-((S)-3-methylpiperazin-1-yl)butyl)-6- |
| | (trifluoromethoxy)quinazolin-4(3H)-one |
| 205 | 6-fluoro-3-methyl-2-((R)-1-((S)-3-methylpiperazin-1-yl)butyl)quinazolin-4(3H)-one |
| 206 | 6,7-dichloro-3-methyl-2-((R)-1-((S)-3-methylpiperazin-1-yl)butyl)quinazolin- |
| | 4(3H)-one |
| 207 | 3-ethyl-2-((R)-1-((S)-3-methylpiperazin-1-yl)butyl)-6-(trifluoromethyl)quinazolin- |
| | 4(3H)-one |
| 212 | 6-bromo-3-ethyl-2-((R)-1-((S)-3-ethylpiperazin-1-yl)butyl)quinazolin-4(3H)-one |
| 213 | 3-ethyl-7-fluoro-6-methoxy-2-((R)-1-((S)-3-methylpiperazin-1- |
| | yl)butyl)quinazolin-4(3H)-one |
| 214 | 3-ethyl-6-fluoro-2-((R)-1-((S)-3-methylpiperazin-1-yl)butyl)quinazolin-4(3H)-one |
| 215 | 3-ethyl-6-methoxy-2-((R)-1-((S)-3-methylpiperazin-1-yl)butyl)quinazolin-4(3H)-one |
| 216 | 3-ethyl-6,7-difluoro-2-((R)-1-((S)-3-methylpiperazin-1-yl)butyl)quinazolin-4(3H)-one |
| 217 | 6-chloro-3-ethyl-8-fluoro-2-((R)-1-((S)-3-methylpiperazin-1-yl)butyl)quinazolin- |
| | 4(3H)-one |
| 218 | 3-ethyl-5,6-difluoro-2-((R)-1-((S)-3-methylpiperazin-1-yl)butyl)quinazolin-4(3H)-one |
| 219 | 6-bromo-3-ethyl-2-((R)-1-((R)-3-(methoxymethyl)piperazin-1- |
| | yl)butyl)quinazolin-4(3H)-one |
| 220 | 6-bromo-3-ethyl-2-((S)-1-((R)-3-(methoxymethyl)piperazin-1- |
| | yl)butyl)quinazolin-4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | CHEMICAL NAME |
|---|---|
| 41 | 6-bromo-3-methyl-2-((R)-1-((S)-3-methylpiperazin-1-yl)butyl)quinazolin-4(3H)-one |
| 42 | 6-bromo-3-methyl-2-((S)-1-((S)-3-methylpiperazin-1-yl)butyl)quinazolin-4(3H)-one |
| 43 | 3-ethyl-2-((R)-1-((S)-3-methylpiperazin-1-yl)butyl)quinazolin-4(3H)-one |
| 44 | 3-ethyl-2-((S)-1-((S)-3-methylpiperazin-1-yl)butyl)quinazolin-4(3H)-one |
| 45 | 3-ethyl-8-fluoro-6-methoxy-2-((S)-1-((S)-3-methylpiperazin-1-yl)butyl)quinazolin- |
| | 4(3H)-one |
| 46 | 3-ethyl-8-fluoro-6-methoxy-2-((R)-1-((S)-3-methylpiperazin-1-yl)butyl)quinazolin- |
| | 4(3H)-one |
| 49 | 6,7-dichloro-3-ethyl-2-((R)-1-((S)-3-methylpiperazin-1-yl)butyl)quinazolin-4(3H)-one |
| 50 | 6,7-dichloro-3-ethyl-2-((S)-1-((S)-3-methylpiperazin-1-yl)butyl)quinazolin-4(3H)-one |
| 51 | 6-bromo-3-ethyl-8-fluoro-2-((R)-1-((S)-3-methylpiperazin-1-yl)butyl)quinazolin- |
| | 4(3H)-one |
| 52 | 6-bromo-3-ethyl-8-fluoro-2-((S)-1-((S)-3-methylpiperazin-1-yl)butyl)quinazolin- |
| | 4(3H)-one |
| 53 | 6-chloro-3-ethyl-7-fluoro-2-((R)-1-((S)-3-methylpiperazin-1-yl)butyl)quinazolin- |
| | 4(3H)-one |
| 54 | 6-chloro-3-ethyl-7-fluoro-2-((S)-1-((S)-3-methylpiperazin-1-yl)butyl)quinazolin- |
| | 4(3H)-one |
| 62 | 6-bromo-7-fluoro-3-methyl-2-((R)-1-((S)-3-methylpiperazin-1-yl)butyl)quinazolin- |
| | 4(3H)-one |
| 63 | 6-bromo-7-fluoro-3-methyl-2-((S)-1-((S)-3-methylpiperazin-1-yl)butyl)quinazolin- |
| | 4(3H)-one |
| 66 | 3-ethyl-2-((R)-1-((S)-3-methylpiperazin-1-yl)butyl)pyrido[4,3-d]pyrimidin-4(3H)-one |
| 67 | 3-ethyl-2-((S)-1-((S)-3-methylpiperazin-1-yl)butyl)pyrido[4,3-d]pyrimidin-4(3H)-one |
| 68 | 6-bromo-3-ethyl-2-((R)-1-((S)-3-methylpiperazin-1-yl)butyl)pyrido[2,3-d]pyrimidin- |
| | 4(3H)-one |
| 69 | 6-bromo-3-ethyl-2-((S)-1-((S)-3-methylpiperazin-1-yl)butyl)pyrido[2,3-d]pyrimidin- |
| | 4(3H)-one |
| 70 | 3-ethyl-2-((R)-1-((S)-3-methylpiperazin-1-yl)butyl)pyrido[3,2-d]pyrimidin-4(3H)-one |
| 71 | 3-ethyl-2-((S)-1-((S)-3-methylpiperazin-1-yl)butyl)pyrido[3,2-d]pyrimidin-4(3H)-one |
| 72 | 6-bromo-2-((R)-1-((S)-3-methylpiperazin-1-yl)butyl)-3-propylpyrido[2,3-d]pyrimidin- |
| | 4(3H)-one |
| 73 | 6-bromo-2-((S)-1-((S)-3-methylpiperazin-1-yl)butyl)-3-propylpyrido[2,3-d]pyrimidin- |
| | 4(3H)-one |
| 74 | 6-bromo-3-(cyclopropylmethyl)-2-((S)-1-((S)-3-methylpiperazin-1- |
| | yl)butyl)pyrido[2,3-d]pyrimidin-4(3H)-one |
| 75 | 6-bromo-3-(cyclopropylmethyl)-2-((R)-1-((S)-3-methylpiperazin-1- |
| | yl)butyl)pyrido[2,3-d]pyrimidin-4(3H)-one |
| 76 | 6-chloro-3-ethyl-2-((R)-1-((S)-3-methylpiperazin-1-yl)butyl)pyrido[2,3-d]pyrimidin- |
| | 4(3H)-one |
| 77 | 3-ethyl-2-((S)-1-((S)-3-methylpiperazin-1-yl)butyl)-7-(trifluoromethyl)pyrido[2,3- |
| | d]pyrimidin-4(3H)-one |
| 78 | 3-ethyl-2-((R)-1-((S)-3-methylpiperazin-1-yl)butyl)-7-(trifluoromethyl)pyrido[2,3- |
| | d]pyrimidin-4(3H)-one |
| 97 | 6-bromo-3-ethyl-2-((R)-1-((S)-3-methylpiperazin-1-yl)butyl)quinazolin-4(3H)-one |
| 99 | 6-bromo-3-ethyl-2-((S)-1-((S)-3-methylpiperazin-1-yl)butyl)quinazolin-4(3H)-one |
| 103 | 6-chloro-3-ethyl-2-((R)-1-((S)-3-methylpiperazin-1-yl)butyl)quinazolin-4(3H)-one |
| 104 | 6-chloro-3-ethyl-2-((S)-1-((S)-3-methylpiperazin-1-yl)butyl)quinazolin-4(3H)-one |
| 105 | 6-bromo-3-ethyl-7-fluoro-2-((S)-1-((S)-3-methylpiperazin-1-yl)butyl)quinazolin- |
| | 4(3H)-one |
| 106 | 6-bromo-3-ethyl-7-fluoro-2-((R)-1-((S)-3-methylpiperazin-1-yl)butyl)quinazolin- |
| | 4(3H)-one |
| 111 | 6-bromo-2-((S)-1-((S)-3,4-dimethylpiperazin-1-yl)butyl)-3-ethylquinazolin-4(3H)-one |
| 112 | 6-bromo-2-((R)-1-((S)-3,4-dimethylpiperazin-1-yl)butyl)-3-ethylquinazolin-4(3H)-one |
| 190 | 6-bromo-3-ethyl-2-((R)-1-((2S,4S)-2-methylpiperidin-4-yl)butyl)quinazolin-4(3H)-one |
| 191 | 6-bromo-3-ethyl-2-((S)-1-((2S,4S)-2-methylpiperidin-4-yl)butyl)quinazolin-4(3H)-one |
| 192 | 6-bromo-3-ethyl-2-((R)-1-((2S,4R)-2-methylpiperidin-4-yl)butyl)quinazolin-4(3H)-one |
| 193 | 6-bromo-3-ethyl-2-((S)-1-((2S,4R)-2-methylpiperidin-4-yl)butyl)quinazolin-4(3H)-one |
| 198 | 6-chloro-3-methyl-2-((R)-1-((S)-3-methylpiperazin-1-yl)butyl)quinazolin-4(3H)-one |
| 201 | 3-ethyl-6,8-difluoro-2-((R)-1-((S)-3-methylpiperazin-1-yl)butyl)quinazolin-4(3H)-one |
| 202 | 6-fluoro-2-((R)-1-((S)-3-methylpiperazin-1-yl)butyl)-3-propylquinazolin-4(3H)-one |
| 203 | 3-ethyl-8-fluoro-2-((R)-1-((S)-3-methylpiperazin-1-yl)butyl)quinazolin-4(3R)-one |
| 204 | 3-ethyl-2-((R)-1-((S)-3-methylpiperazin-1-yl)butyl)-6-(trifluoromethoxy)quinazolin- |
| | 4(3H)-one |
| 205 | 6-fluoro-3-methyl-2-((R)-1-((S)-3-methylpiperazin-1-yl)butyl)quinazolin-4(3H)-one |
| 206 | 6,7-dichloro-3-methyl-2-((R)-1-((S)-3-methylpiperazin-1-yl)butyl)quinazolin-4(3H)-one |
| 207 | 3-ethyl-2-((R)-1-((S)-3-methylpiperazin-1-yl)butyl)-6-(trifluoromethyl)quinazolin- |
| | 4(3H)-one |
| 213 | 3-ethyl-7-fluoro-6-methoxy-2-((R)-1-((S)-3-methylpiperazin-1-yl)butyl)quinazolin- |
| | 4(3H)-one |
| 214 | 3-ethyl-6-fluoro-2-((R)-1-((S)-3-methylpiperazin-1-yl)butyl)quinazolin-4(3H)-one |
| 215 | 3-ethyl-6-methoxy-2-((R)-1-((S)-3-methylpiperazin-1-yl)butyl)quinazolin-4(3H)-one |
| 216 | 3-ethyl-6,7-difluoro-2-((R)-1-((S)-3-methylpiperazin-1-yl)butyl)quinazolin-4(3H)-one |
| 217 | 6-chloro-3-ethyl-8-fluoro-2-((R)-1-((S)-3-methylpiperazin-1-yl)butyl)quinazolin- |
| | 4(3H)-one |
| 218 | 3-ethyl-5,6-difluoro-2-((R)-1-((S)-3-methylpiperazin-1-yl)butyl)quinazolin-4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | CHEMICAL NAME |
|---|---|
| 222 | 5,6-difluoro-3-methyl-2-((R)-1-((S)-3-methylpiperazin-1-yl)butyl)quinazolin- |
| | 4(3H)-one |
| 227 | 6-Bromo-2-((S)-1-((3S,5S)-3,5-dimethylpiperazin-1-yl)butyl)-3-ethylquinazolin- |
| | 4(3H)-one |
| 229 | 6-Chloro-3-ethyl-2-((R)-1-((S)-3-(fluoromethyl)piperazin-1-yl)butyl)quinazolin- |
| | 4(3H)-one |
| 230 | 6-chloro-3-ethyl-2-((R)-1-((R)-3-(fluoromethyl)piperazin-1-yl)butyl)quinazolin- |
| | 4(3H)-one |
| 231 | 6-chloro-3-ethyl-2-((S)-1-((R)-3-(fluoromethyl)piperazin-1-yl)butyl)quinazolin- |
| | 4(3H)-one |
| 232 | 6-chloro-3-ethyl-2-((S)-1-((S)-3-(fluoromethyl)piperazin-1-yl)butyl)quinazolin- |
| | 4(3H)-one. |
| 233 | 3-Ethyl-2-((R)-1-((R)-3-methylpiperazin-1-yl)butyl)-6- |
| | (trifluoromethyl)pyrido[3,4-d]pyrimidin-4(3H)-one |
| 234 | 3-ethyl-2-((R)-1-((S)-3-methylpiperazin-1-yl)butyl)-6-(trifluoromethyl)pyrido[3,4- |
| | d]pyrimidin-4(3H)-one |
| 235 | 3-ethyl-2-((S)-1-((S)-3-methylpiperazin-1-yl)butyl)-6-(trifluoromethyl)pyrido[3,4- |
| | d]pyrimidin-4(3H)-one |
| 236 | 2-((R)-1-((3S,5R)-3,5-Dimethylpiperazin-1-yl)butyl)-3-ethyl-6- |
| | ((methylamino)(phenyl)methyl)quinazolin-4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | CHEMICAL NAME |
|---|---|
| 221 | 6-chloro-7-fluoro-3-methyl-2-((R)-1-((S)-3-methylpiperazin-1- |
| | yl)butyl)quinazolin-4(3H)-one |
| 223 | 6-chloro-8-fluoro-3-methyl-2-((R)-1-((S)-3-methylpiperazin-1- |
| | yl)butyl)quinazolin-4(3H)-one |
| 224 | (R)-6-bromo-2-(1-(3,3-dimethylpiperazin-1-yl)butyl)-3-ethylquinazolin-4(3H)-one |
| 225 | 6,8-difluoro-3-methyl-2-((R)-1-((S)-3-methylpiperazin-1-yl)butyl)quinazolin- |
| | 4(3H)-one |
| 226 | 3-ethyl-5,6,8-trifluoro-2-((R)-1-((S)-3-methylpiperazin-1-yl)butyl)quinazolin- |
| | 4(3H)-one |
| 228 | 6-Bromo-2-((R)-1-((3S,5S)-3,5-dimethylpiperazin-1-yl)butyl)-3- |
| | ethylquinazolin-4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | CHEMICAL NAME |
|---|---|
| 1 | 6-bromo-2-(1-((3S,5R)-3,5-dimethylpiperazin-1-yl)butyl)-3-ethylquinazolin-4(3H)-one |
| 2 | 2-(1-((3S,5R)-3,5-dimethylpiperazin-1-yl)butyl)-3-methylquinazolin-4(3H)-one |
| 3 | 2-(1-((3S,5R)-3,5-dimethylpiperazin-1-yl)butyl)-3-(2-(methylamino)ethyl)quinazolin- |
| | 4(3H)-one |
| 4 | 2-(1-((3S,5R)-3,5-dimethylpiperazin-1-yl)butyl)-3-ethyl-7-methylquinazolin-4(3H)-one |
| 5 | 8-bromo-2-(1-((3S,5R)-3,5-dimethylpiperazin-1-yl)butyl)-3-ethyl-7-methoxyquinazolin- |
| | 4(3H)-one |
| 6 | 2-(1-((3S,5R)-3,5-dimethylpiperazin-1-yl)butyl)-3-ethyl-7-methoxyquinazolin-4(3H)-one |
| 7 | methyl 2-(1-((3S,5R)-3,5-dimethylpiperazin-1-yl)butyl)-3-ethyl-4-oxo-3,4- |
| | dihydroquinazoline-7-carboxylate |
| 8 | methyl 2-(1-((3S,5R)-3,5-dimethylpiperazin-1-yl)butyl)-3-methyl-4-oxo-3,4- |
| | dihydroquinazoline-7-carboxylate |
| 9 | 7-bromo-2-(1-((3S,5R)-3,5-dimethylpiperazin-1-yl)butyl)-3-ethylquinazolin-4(3H)-one |
| 10 | 2-(1-((3S,5R)-3,5-dimethylpiperazin-1-yl)butyl)-7-hydroxy-3-methylquinazolin-4(3H)-one |
| 11 | 2-(1-((3S,5R)-3,5-dimethylpiperazin-1-yl)butyl)-3-ethyl-7-fluoroquinazolin-4(3H)-one |
| 12 | 2-(1-((3S,5R)-3,5-dimethylpiperazin-1-yl)butyl)-3-ethyl-6-hydroxyquinazolin-4(3H)-one |
| 13 | 8-bromo-2-(1-((3S,5R)-3,5-dimethylpiperazin-1-yl)butyl)-3-ethylquinazolin-4(3H)-one |
| 14 | 5-bromo-2-(1-((3S,5R)-3,5-dimethylpiperazin-1-yl)butyl)-3-ethylquinazolin-4(3H)-one |
| 15 | 2-(1-((3S,5R)-3,5-dimethylpiperazin-1-yl)butyl)-3-ethyl-5-hydroxyquinazolin-4(3H)-one |
| 16 | 6-bromo-2-(1-((3S,5R)-3,5-dimethylpiperazin-1-yl)butyl)-3-methylquinazolin-4(3H)-one |
| 17 | 7-bromo-2-(1-((3S,5R)-3,5-dimethylpiperazin-1-yl)butyl)-3-methylquinazolin-4(3H)-one |
| 18 | 6-bromo-2-(1-((3S,5R)-3,5-dimethylpiperazin-1-yl)butyl)-3-ethyl-7-fluoroquinazolin- |
| | 4(3H)-one |
| 19 | 6-bromo-2-(1-((3S,5R)-3,5-dimethylpiperazin-1-yl)butyl)-3-ethyl-7-methylquinazolin- |
| | 4(3H)-one |
| 20 | 6-bromo-2-(1-((3S,5R)-3,5-dimethylpiperazin-1-yl)butyl)-3-ethyl-8-methylquinazolin- |
| | 4(3H)-one |
| 21 | 6-bromo-2-(1-((3S,5R)-3,5-dimethylpiperazin-1-yl)butyl)-3-ethyl-5-methylquinazolin- |
| | 4(3H)-one |
| 22 | 6-bromo-2-(1-((3S,5R)-3,5-dimethylpiperazin-1-yl)butyl)-3-ethyl-5-fluoroquinazolin- |
| | 4(3H)-one |
| 23 | 6-bromo-5-chloro-2-(1-((3S,5R)-3,5-dimethylpiperazin-1-yl)butyl)-3-ethylquinazolin- |
| | 4(3H)-one |
| 24 | 2-(1-((3S,5R)-3,5-dimethylpiperazin-1-yl)butyl)-3-ethyl-6-(ethylamino)quinazolin- |
| | 4(3H)-one |
| 25 | 2-(1-((3S,5R)-3,5-dimethylpiperazin-1-yl)butyl)-3-ethyl-7-(ethylamino)quinazolin- |
| | 4(3H)-one |
| 26 | tert-butyl (2-(1-((3S,5R)-3,5-dimethylpiperazin-1-yl)butyl)-3-ethyl-4-oxo-3,4- |
| | dihydroquinazolin-7-yl)(ethyl)carbamate |
| 27 | 2-(1-((3S,5R)-3,5-dimethylpiperazin-1-yl)butyl)-3-ethylpyrido[3,2-d]pyrimidin-4(3H)-one |
| 28 | 6-bromo-2-(1-((3S,5R)-3,5-dimethylpiperazin-1-yl)butyl)-3-ethylpyrido[2,3-d]pyrimidin- |
| | 4(3H)-one |
| 29 | 6-bromo-2-((R)-1-((3S,5R)-3,5-dimethylpiperazin-1-yl)butyl)-3-ethylquinazolin-4(3H)-one |
| 30 | 6-bromo-2-((S)-1-((3S,5R)-3,5-dimethylpiperazin-1-yl)butyl)-3-ethylquinazolin-4(3H)-one |
| 31 | 2-((S)-1-((3S,5R)-3,5-dimethylpiperazin-1-yl)butyl)-3-methylquinazolin-4(3H)-one |
| 32 | 2-((R)-1-((3S,5R)-3,5-dimethylpiperazin-1-yl)butyl)-3-methylquinazolin-4(3H)-one |
| 33 | 2-((S)-1-((3S,5R)-3,5-dimethylpiperazin-1-yl)butyl)-3-methylquinazolin-4(3H)-one |
| 34 | 2-((R)-1-((3S,5R)-3,5-dimethylpiperazin-1-yl)butyl)-3-ethylquinazolin-4(3H)-one |
| 35 | 2-((R)-1-((3S,5R)-3,5-dimethylpiperazin-1-yl)butyl)-3-ethyl-6-hydroxyquinazolin-4(3H)-one |
| 36 | 2-((S)-1-((3S,5R)-3,5-dimethylpiperazin-1-yl)butyl)-3-ethyl-6-hydroxyquinazolin-4(3H)-one |
| 113 | 2-(1-((3S,5R)-3,5-dimethylpiperazin-1-yl)butyl)-3-(2-methoxyethyl)quinazolin-4(3H)-one |
| 114 | 2-(1-((3S,5R)-3,5-dimethylpiperazin-1-yl)-3-methylbutyl)-3-(2- |
| | methoxyethyl)quinazolin-4(3H)-one |
| 115 | 2-(1-((3S,5R)-3,5-dimethylpiperazin-1-yl)pentyl)-3-(2-methoxyethyl)quinazolin-4(3H)-one |
| 116 | 2-(2-cyclopropyl-1-((3S,5R)-3,5-dimethylpiperazin-1-yl)ethyl)-3-(2- |
| | methoxyethyl)quinazolin-4(3H)-one |
| 118 | 2-(1-((3S,5R)-3,5-dimethylpiperazin-1-yl)butyl)-3-(furan-2-ylmethyl)quinazolin-4(3H)-one |
| 119 | 2-(1-((3S,5R)-3,5-dimethylpiperazin-1-yl)butyl)-3-(2-methoxyethyl)-6- |
| | methylquinazolin-4(3H)-one |
| 120 | 2-(1-((3S,5R)-3,5-dimethylpiperazin-1-yl)butyl)-6-methoxy-3-(2- |
| | methoxyethyl)quinazolin-4(3H)-one |
| 121 | 2-(1-((3S,5R)-3,5-dimethylpiperazin-1-yl)butyl)-3-(2-methoxyethyl)pyrido[3,4- |
| | d]pyrimidin-4(3H)-one |
| 122 | ethyl 3-(6-bromo-2-(1-((3S,5R)-3,5-dimethylpiperazin-1-yl)butyl)-4-oxoquinazolin- |
| | 3(4H)-yl)propanoate |
| 123 | 2-((R)-1-((3S,5R)-3,5-dimethylpiperazin-1-yl)butyl)-3-(2-methoxyethyl)quinazolin- |
| | 4(3H)-one |
| 124 | 2-((S)-1-((3S,5R)-3,5-dimethylpiperazin-1-yl)butyl)-3-(2-methoxyethyl)quinazolin- |
| | 4(3H)-one |
| 125 | 3-(6-bromo-2-(1-((3S,5R)-3,5-dimethylpiperazin-1-yl)butyl)-4-oxoquinazolin-3(4H)- |
| | yl)propanoic acid |
| 126 | 2-(6-bromo-2-(1-((3S,5R)-3,5-dimethylpiperazin-1-yl)butyl)-4-oxoquinazolin-3(4H)- |
| | yl)acetic acid |
| 127 | 2-(1-((3S,5R)-3,5-dimethylpiperazin-1-yl)butyl)-3-(2-(methylamino)ethyl)quinazolin- |
| | 4(3H)-one |
| 128 | 3-(2-(dimethylamino)ethyl)-2-(1-((3S,5R)-3,5-dimethylpiperazin-1-yl)butyl)quinazolin- |
| | 4(3H)-one |
| 129 | 2-(1-((3S,5R)-3,5-dimethylpiperazin-1-yl)butyl)-3-(2- |
| | (methyl(phenethyl)amino)ethyl)quinazolin-4(3H)-one |
| 130 | 2-(1-((3S,5R)-3,5-dimethylpiperazin-1-yl)butyl)-3-(2-(methyl(3- |
| | phenylpropyl)amino)ethyl)quinazolin-4(3H)-one |
| 131 | 3-(2-(dimethylamino)ethyl)-2-(1-((3S,5R)-3,4,5-trimethylpiperazin-1- |
| | yl)butyl)quinazolin-4(3H)-one |
| 132 | 2-(1-((3S,5R)-3,5-dimethylpiperazin-1-yl)butyl)-3-ethyl-6-(pyridin-4-yl)quinazolin- |
| | 4(3H)-one |
| 133 | 2-(1-((3S,5R)-3,5-dimethylpiperazin-1-yl)butyl)-3-ethyl-7-(pyridin-4-yl)quinazolin- |
| | 4(3H)-one |
| 134 | 2-(1-((3S,5R)-3,5-dimethylpiperazin-1-yl)butyl)-3-ethyl-7-(3-hydroxyphenyl)quinazolin- |
| | 4(3H)-one |
| 135 | 2-(1-((3S,5R)-3,5-dimethylpiperazin-1-yl)butyl)-3-ethyl-7-(2-methoxypyridin-4- |
| | yl)quinazolin-4(3H)-one |
| 136 | 2-(1-((3S,5R)-3,5-dimethylpiperazin-1-yl)butyl)-3-methyl-7-(2- |
| | ((methylamino)methyl)pyridin-4-yl)quinazolin-4(3H)-one |
| 137 | 2-(1-((3S,5R)-3,5-dimethylpiperazin-1-yl)butyl)-3-ethyl-7-(pyrrolidin-1-yl)quinazolin- |
| | 4(3H)-one |
| 138 | 2-(1-((3S,5R)-3,5-dimethylpiperazin-1-yl)butyl)-3-ethyl-6-(pyrrolidin-1-yl)quinazolin- |
| | 4(3H)-one |
| 139 | 6-(4-(dimethylamino)piperidin-1-yl)-2-(1-((3S,5R)-3,5-dimethylpiperazin-1-yl)butyl)-3- |
| | ethylquinazolin-4(3H)-one |
| 140 | 2-(1-((3S,5R)-3,5-dimethylpiperazin-1-yl)butyl)-3-ethyl-6-(4-(methylamino)piperidin-1- |
| | yl)quinazolin-4(3H)-one |
| 141 | 6-(4-aminopiperidin-1-yl)-2-(1-((3S,5R)-3,5-dimethylpiperazin-1-yl)butyl)-3- |
| | ethylquinazolin-4(3H)-one |
| 142 | 2-(1-((3S,5R)-3,5-dimethylpiperazin-1-yl)butyl)-3-ethyl-6-((1-methylpiperidin-4- |
| | yl)amino)quinazolin-4(3H)-one |
| 143 | 2-(1-((3S,5R)-3,5-dimethylpiperazin-1-yl)butyl)-3-ethyl-6-(methyl(1-methylpiperidin-4- |
| | yl)amino)quinazolin-4(3H)-one |
| 144 | 6-(benzyl(methyl)amino)-2-(1-((3S,5R)-3,5-dimethylpiperazin-1-yl)butyl)-3- |
| | ethylquinazolin-4(3H)-one |
| 145 | 2-(1-((3S,5R)-3,5-dimethylpiperazin-1-yl)butyl)-3-ethyl-6-((2- |
| | (methylamino)ethyl)amino)quinazolin-4(3H)-one |
| 146 | 2-(1-((3S,5R)-3,5-dimethylpiperazin-1-yl)butyl)-3-ethyl-6-(methyl(2- |
| | (methylamino)ethyl)amino)quinazolin-4(3H)-one |
| 147 | 2-(1-((3S,5R)-3,5-dimethylpiperazin-1-yl)butyl)-3-ethyl-7-((2- |
| | (methylamino)ethyl)amino)quinazolin-4(3H)-one |
| 148 | 2-(1-((3S,5R)-3,5-dimethylpiperazin-1-yl)butyl)-3-ethyl-6-(4-(2-hydroxyphenyl)-5,6- |
| | dihydropyridin-1(2H)-yl)quinazolin-4(3H)-one |
| 149 | 2-(1-((3S,5R)-3,5-dimethylpiperazin-1-yl)butyl)-3-ethyl-6-((3S,3aR,7aR)-3- |
| | phenylhexahydro-4,7-ethanopyrrolo[3,2-b]pyridin-1(2H)-yl)quinazolin-4(3H)-one |
| 150 | 6-((1-benzylpiperidin-4-yl)amino)-2-(1-((3S,5R)-3,5-dimethylpiperazin-1-yl)butyl)-3- |
| | ethylquinazolin-4(3H)-one |
| 151 | 6-(4-(benzyl(methyl)amino)piperidin-1-yl)-2-(1-((3S,5R)-3,5-dimethylpiperazin-1- |
| | yl)butyl)-3-ethylquinazolin-4(3H)-one |
| 152 | 2-((S)-1-((3S,5R)-3,5-dimethylpiperazin-1-yl)butyl)-3-ethyl-6-((S)-2-methyl-1-oxa-4,9- |
| | diazaspiro[5.5]undecan-4-yl)quinazolin-4(3H)-one |
| 153 | 2-((R)-1-((3S,5R)-3,5-dimethylpiperazin-1-yl)butyl)-3-ethyl-6-((S)-2-methyl-1-oxa-4,9- |
| | diazaspiro[5.5]undecan-4-yl)quinazolin-4(3H)-one |
| 154 | 2-((S)-1-((3S,5R)-3,5-dimethylpiperazin-1-yl)butyl)-3-ethyl-6-((R)-2-methyl-9- |
| | phenethyl-1-oxa-4,9-diazaspiro[5.5]undecan-4-yl)quinazolin-4(3H)-one |
| 155 | 2-((S)-1-((3S,5R)-3,5-dimethylpiperazin-1-yl)butyl)-3-ethyl-6-((S)-2-methyl-9- |
| | phenethyl-1-oxa-4,9-diazaspiro[5.5]undecan-4-yl)quinazolin-4(3H)-one |
| 156 | 2-((R)-1-((3S,5R)-3,5-dimethylpiperazin-1-yl)butyl)-3-ethyl-6-((S)-2-methyl-9- |
| | phenethyl-1-oxa-4,9-diazaspiro[5.5]undecan-4-yl)quinazolin-4(3H)-one |
| 157 | 2-((R)-1-((3S,5R)-3,5-dimethylpiperazin-1-yl)butyl)-3-ethyl-6-((R)-2-methyl-9- |
| | phenethyl-1-oxa-4,9-diazaspiro[5.5]undecan-4-yl)quinazolin-4(3H)-one |
| 158 | 2-((S)-1-((3S,5R)-3,5-dimethylpiperazin-1-yl)butyl)-3-ethyl-6-((2-((R)-9-(pyridin-2-yl)- |
| | 6-oxaspiro[4.5]decan-9-yl)ethyl)amino)quinazolin-4(3H)-one |
| 159 | 2-((R)-1-((3S,5R)-3,5-dimethylpiperazin-1-yl)butyl)-3-ethyl-6-((2-((S)-9-(pyridin-2-yl)- |
| | 6-oxaspiro[4.5]decan-9-yl)ethyl)amino)quinazolin-4(3H)-one |
| 160 | 2-((R)-1-((3S,5R)-3,5-dimethylpiperazin-1-yl)butyl)-3-ethyl-6-((2-((R)-9-(pyridin-2-yl)- |
| | 6-oxaspiro[4.5]decan-9-yl)ethyl)amino)quinazolin-4(3H)-one |
| 161 | 2-((S)-1-((3S,5R)-3,5-dimethylpiperazin-1-yl)butyl)-3-ethyl-6-((2-((S)-9-(pyridin-2-yl)-6- |
| | oxaspiro[4.5]decan-9-yl)ethyl)amino)quinazolin-4(3H)-one |
| 162 | tert-Butyl (8-bromo-2-(1-((3S,5R)-3,5-dimethylpiperazin-1-yl)butyl)-3-ethyl-4-oxo-3,4- |
| | dihydroquinazolin-7-yl)(3-methoxybenzyl)carbamate |
| 163 | N-(2-(1-((3S,5R)-3,5-dimethylpiperazin-1-yl)butyl)-3-ethyl-4-oxo-3,4- |
| | dihydroquinazolin-6-yl)-N-(1-methylpiperidin-4-yl)propionamide. |
| 164 | 2-((S)-1-((3S,5R)-3,5-dimethylpiperazin-1-yl)butyl)-3-methyl-7-((R)-3-(methylamino)- |
| | 1-phenylpropoxy)quinazolin-4(3H)-one |
| 165 | 2-((R)-1-((3S,5R)-3,5-dimethylpiperazin-1-yl)butyl)-3-methyl-7-((R)-3-(methylamino)- |
| | 1-phenylpropoxy)quinazolin-4(3H)-one |
| 166 | 2-((S)-1-((3S,5R)-3,5-dimethylpiperazin-1-yl)butyl)-3-methyl-7-((S)-3-(methylamino)- |
| | 1-phenylpropoxy)quinazolin-4(3H)-one |
| 167 | 2-((R)-1-((3S,5R)-3,5-dimethylpiperazin-1-yl)butyl)-3-methyl-7-((S)-3-(methylamino)- |
| | 1-phenylpropoxy)quinazolin-4(3H)-one |
| 168 | 2-((S)-1-((3S,5R)-3,5-dimethylpiperazin-1-yl)butyl)-3-methyl-7-((S)-2-(methylamino)- |
| | 1-phenylethoxy)quinazolin-4(3H)-one |
| 169 | 2-((R)-1-((3S,5R)-3,5-dimethylpiperazin-1-yl)butyl)-3-methyl-7-((R)-2-(methylamino)- |
| | 1-phenylethoxy)quinazolin-4(3H)-one |
| 170 | 2-((R)-1-((3S,5R)-3,5-dimethylpiperazin-1-yl)butyl)-3-methyl-7-((S)-2-(methylamino)- |
| | 1-phenylethoxy)quinazolin-4(3H)-one |
| 171 | 2-((S)-1-((3S,5R))-3,5-dimethylpiperazin-1-yl)butyl)-3-methyl-7-((R)-2-(methylamino)- |
| | 1-phenylethoxy)quinazolin-4(3H)-one |
| 172 | 2-(1-((3S,5R)-3,5-dimethylpiperazin-1-yl)butyl)-7-(hydroxymethyl)-3-methylquinazolin- |
| | 4(3H)-one |
| 173 | 2-(1-((3S,5R)-3,5-dimethylpiperazin-1-yl)butyl)-3-ethyl-6-(2-hydroxyethyl)quinazolin- |
| | 4(3H)-one |
| 174 | 6-(2-(benzyl(methyl)amino)ethyl)-2-(1-((3S,5R)-3,5-dimethylpiperazin-1-yl)butyl)-3- |
| | ethylquinazolin-4(3H)-one |
| 175 | 2-(1-((3S,5R)-3,5-dimethylpiperazin-1-yl)butyl)-3-ethyl-6-(2- |
| | (isopentyl(methyl)amino)ethyl)quinazolin-4(3H)-one |
| 176 | 2-(1-((3S,5R)-3,5-dimethylpiperazin-1-yl)butyl)-3-methyl-4-oxo-3,4- |
| | dihydroquinazoline-7-carbonitrile |
| 177 | 2-(1-((3S,5R)-3,5-dimethylpiperazin-1-yl)butyl)-3-ethyl-4-oxo-3,4-dihydroquinazoline- |
| | 6-carbonitrile |
| 178 | 2-(1-((3S,5R)-3,5-dimethylpiperazin-1-yl)butyl)-3-methyl-4-oxo-3,4- |
| | dihydroquinazoline-7-carboxylic acid |
| 179 | 2-(1-((3S,5R)-3,5-dimethylpiperazin-1-yl)butyl)-3-ethyl-4-oxo-3,4-dihydroquinazoline- |
| | 7-carboxylic acid |
| 180 | N-benzyl-2-(1-((3S,5R)-3,5-dimethylpiperazin-1-yl)butyl)-3-ethyl-4-oxo-3,4- |
| | dihydroquinazoline-7-carboxamide |
| 181 | N-(1-((2-(1-((3S,5R)-3,5-dimethylpiperazin-1-yl)butyl)-3-ethyl-4-oxo-3,4- |
| | dihydroquinazolin-6-yl)methyl)piperidin-4-yl)-N-phenylpropionamide |
| 182 | N-(1-((2-(1-((3S,5R)-3,5-dimethylpiperazin-1-yl)butyl)-3-methyl-4-oxo-3,4- |
| | dihydroquinazolin-7-yl)methyl)piperidin-4-yl)-N-phenylpropionamide |
| 183 | 6-((benzyl(methyl)amino)methyl)-2-(1-((3S,5R)-3,5-dimethylpiperazin-1-yl)butyl)-3- |
| | ethylquinazolin-4(3H)-one |
| 184 | 2-(1-((3S,5R)-3,5-dimethylpiperazin-1-yl)butyl)-3-ethyl-6- |
| | ((isobutyl(methyl)amino)methyl)quinazolin-4(3H)-one |
| 185 | 2-(1-((3S,5R)-3,5-dimethylpiperazin-1-yl)butyl)-3-ethyl-6- |
| | ((isopentyl(methyl)amino)methyl)quinazolin-4(3H)-one |
| 186 | 2-(1-((3S,5R)-3,5-dimethylpiperazin-1-yl)butyl)-3-methyl-7-((4-methylpiperazin-1- |
| | yl)methyl)quinazolin-4(3H)-one |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compounds are selected which act as ligands of the α2δ subunit, particularly the α2δ-1 subunit, of the voltage-gated calcium channel. In a very preferred embodiment, the compounds are selected which act as dual ligands of the α2δ subunit, particularly the α2δ-1 subunit, of the voltage-gated calcium channel and the μ-opioid receptor and especially compounds which have a binding expressed as Kᵢ responding to the following scales:

Kᵢ(μ) is preferably <1000 nM, more preferably <500 nM, even more preferably <100 nM.

Preferably, when Kᵢ (μ)>500 nM, the following scale has been adopted for representing the binding to the μ-receptor:
+Kᵢ (μ)>500 nM or inhibition ranges between 1% and 50%.

Kᵢ(α2δ1) is preferably <10000 nM, more preferably <5000 nM, even more preferably <500 nM.

Preferably, when Kᵢ(α₂δ-1)>5000 nM, the following scale has been adopted for representing the binding to the α₂δ-1 subunit of voltage-gated calcium channels:
+Kᵢ(α₂δ-1)>5000 nM or inhibition ranges between 1% and 50%.

In the following the phrase “compound of the invention” is used. This is to be understood as any compound according to the invention as described above according to general Formula (I), (I^{2′}), (I^{3′}), (I^{4′}) and (I^{5′}).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

For the sake of clarity the expression “a compound according to Formula (I), wherein e.g. R₁, R₂, R₃, R₄, R₅, R₅′, R₅″, R₅′″, R₆, R₆′, R₆″, R₆′″, R₇, Y₁, Y₂, Y₃, W, w₁, w₂, w₃ and w₄ are as defined below in the detailed description” would (just like the expression “a compound of Formula (I) as defined in any one of claims e.g. 1 to 8” found in the claims) refer to “a compound according to Formula (I)”, wherein the definitions of the respective substituents R₁ etc. (also from the cited claims) are applied. In addition, this would also mean, though (especially in regards to the claims) that also one or more disclaimers or provisos defined in the description (or used in any of the cited claims like e.g. claim 1) would be applicable to define the respective compound. Thus, a disclaimer or a proviso found in e.g. claim 1 would be also used to define the compound “of Formula (I) as defined in any one of the corresponding related claims e.g. 1 to 8”.

In general the processes are described below in the experimental part. The starting materials are commercially available or can be prepared by conventional methods.

A preferred embodiment of the invention is a process for the production of a compound according to Formula (I), wherein, if not defined otherwise, R₁, R₂, R₃, R₄, R₅, R₅′, R₅″, R₅′″, R₆, R₆′, R₆″, R₆′″, R₇, Y₁, Y₂, Y₃, W, w₁, w₂, w₃ and w₄ have the meanings defined in the description. LG represents a leaving group (such as chloro, bromo, iodo, mesylate, tosylate, nosylate or triflate).

In a particular embodiment there is a process for the production of a compound according to Formula (I), wherein W is —CH—, said process comprises the alkylation of a compound of formula XIV

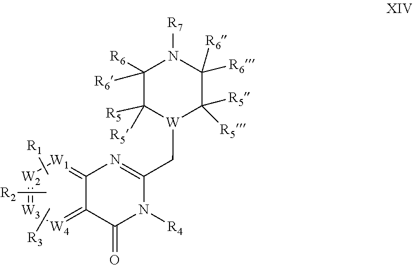

with a compound of formula XV,

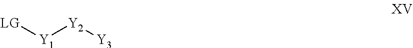

using a suitable base, such as lithium bis(trimethylsilyl)amide, in a suitable solvent, such as tetrahydrofuran at a suitable temperature, such as room temperature, wherein R₁, R₂, R₃, R₄, R₅, R₅′, R₅″, R₅′″, R₆, R₆′, R₆″, R₆′″, R₇, Y₁, Y₂, Y₃, w₁, w₂, w₃ and w₄ have the meanings as defined in the description, and LG is a leaving group.

In another particular embodiment there is a process for the production of a compound according to Formula (I), wherein W is nitrogen, said process comprises reacting a compound of formula VIII

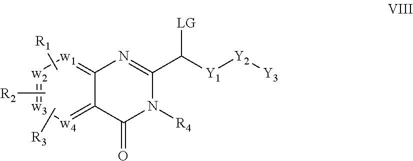

with a suitable amine of formula IX,

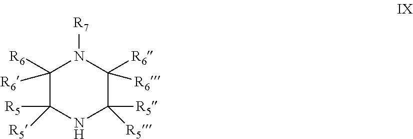

in a suitable solvent, such as acetonitrile or dimethylformamide, in the presence of a base such as triethylamine, K₂CO₃ or N,N-diisopropylethylamine, at a suitable temperature comprised between room temperature and the reflux temperature, preferably heating, wherein R₁, R₂, R₃, R₄, R₅, R₅′, R₅″, R₅′″, R₆, R₆′, R₆″, R₆′″, R₇, Y₁, Y₂, Y₃, w₁, w₂, w₃ and w₄ have the meanings as defined in the description, and LG is a leaving group.

In a particular embodiment there is a process for the production of a compound according to Formula (I),
a) wherein W is —CH—, said process comprises the alkylation of a compound of formula XIV

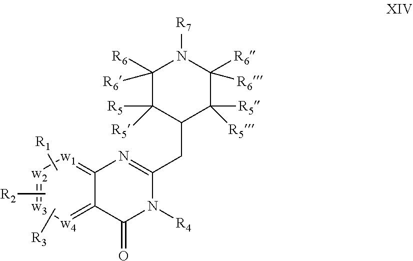

with a compound of formula XV,

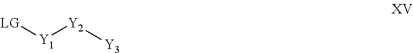

using a suitable base, such as lithium bis(trimethylsilyl)amide, in a suitable solvent, such as tetrahydrofuran at a suitable temperature, such as room temperature, wherein R₁, R₂, R₃, R₄, R₅, R₅′, R₅″, R₅′″, R₆, R₆′, R₆″, R₆′″, R₇, Y₁, Y₂, Y₃, w₁, w₂, w₃ and w₄ have the meanings as defined in the description, and LG is a leaving group,

or
b) wherein W is nitrogen, said process comprises reacting a compound of formula VIII

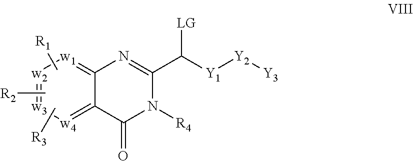

with a suitable amine of formula IX,

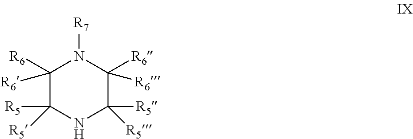

in a suitable solvent, such as acetonitrile or dimethylformamide, in the presence of a base such as triethylamine, K₂CO₃ or N,N-diisopropylethylamine, at a suitable temperature comprised between room temperature and the reflux temperature, preferably heating, wherein R₁, R₂, R₃, R₄, R₅, R₅′, R₅″, R₅′″, R₆, R₆′, R₆″, R₆′″, R₇, Y₁, Y₂, Y₃, w₁, w₂, w₃ and w₄ have the meanings as defined in the description, and LG is a leaving group.

In a particular embodiment there is a process for the production of a compound according to Formula (I), by the reduction reaction of a carbonyl derivative with a suitable reductive reagent, preferably sodium borohydride, in an organic solvent, preferably MeOH, to afford a hydroxyl compound.

In a particular embodiment there is a process for the production of a compound according to Formula (I), by deprotection reaction of a compound of formula I that contains an amine protecting group such as a carbamate, preferably tert-butoxy carbonyl, by any suitable method, such as treatment with an acid, preferably HCl or trifluoroacetic acid in an appropriate solvent such as 1,4-dioxane, DCM, ethyl acetate or a mixture of an organic solvent and water.

In a particular embodiment there is a process for the production of a compound according to Formula (I), by reductive amination reaction of a compound of formula I that contains an amino group with an aldehyde, preferably carried out with a reductive reagent, preferably sodium triacetoxyborohydride, in an organic solvent, preferably DCE, in the presence of an organic base, preferably DIPEA or TEA. Alternatively, the reaction can be carried out in the presence of an acid, preferably acetic acid.

In a particular embodiment there is a process for the production of a compound according to Formula (I), by reaction of a compound of formula I that contains an amino group with an alkylating reagent, in the presence of a base, preferably DIPEA or K₂CO₃, in an organic solvent, preferably acetonitrile, at suitable temperature, such as in the range of 0-120° C.

In a particular embodiment there is a process for the production of a compound according to Formula (I), by reaction of a compound of formula I that contains an amino group with a vinyl derivative, in an organic solvent, preferably 2-methoxyethanol, at suitable temperature, such as in the range of 20-140° C.

A particular embodiment of the invention refers to the use of a compound of Formula (IIa),

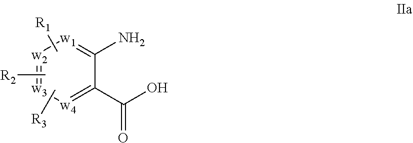

wherein R₁, R₂, R₃, w₁, w₂, w₃ and w₄ have the meanings as defined in the description, for the preparation of compounds of Formula (I).

A particular embodiment of the invention refers to the use of a compound of Formula (IIb),

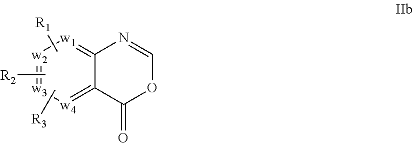

wherein R₁, R₂, R₃, w₁, w₂, w₃ and w₄ have the meanings as defined in the description, for the preparation of compounds of Formula (I).

A particular embodiment of the invention refers to the use of a compound of Formula (III),

H₂N—R₄ III

wherein R₄ has the meaning as defined in the description for the preparation of compounds of Formula (I).

A particular embodiment of the invention refers to the use of a compound of Formula (IV),

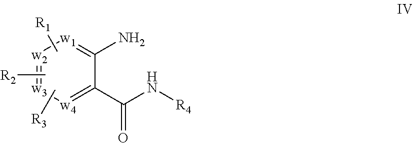

wherein R₁, R₂, R₃, R₄, w₁, w₂, w₃ and w₄ have the meanings as defined in the description, for the preparation of compounds of Formula (I).

A particular embodiment of the invention refers to the use of a compound of Formula (V),

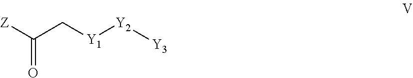

wherein Y₁, Y₂ and Y₃ have the meanings as defined in the description, and Z represents OH or a halogen for the preparation of compounds of Formula (I).

A particular embodiment of the invention refers to the use of a compound of Formula (VI),

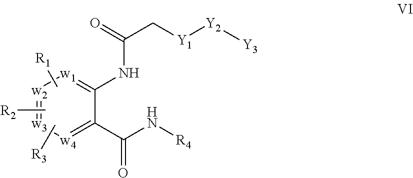

wherein R₁, R₂, R₃, R₄, w₁, w₂, w₃, w₄, Y₁, Y₂ and Y₃ have the meanings as defined in the description, for the preparation of compounds of Formula (I).

A particular embodiment of the invention refers to the use of a compound of Formula (VII),

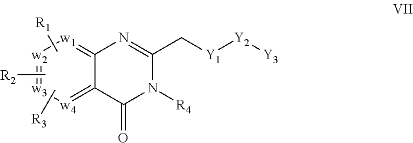

wherein R₁, R₂, R₃, R₄, w₁, w₂, w₃, w₄, Y₁, Y₂ and Y₃ have the meanings as defined in the description, for the preparation of compounds of Formula (I).

A particular embodiment of the invention refers to the use of a compound of Formula (VIII),

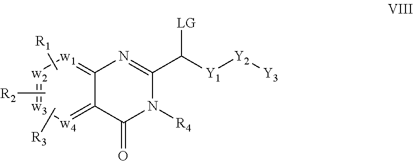

wherein R₁, R₂, R₃, R₄, w₁, w₂, w₃, w₄, Y₁, Y₂ and Y₃ have the meanings as defined in the description, and LG is a leaving group, for the preparation of compounds of Formula (I).

A particular embodiment of the invention refers to the use of a compound of Formula (IX),

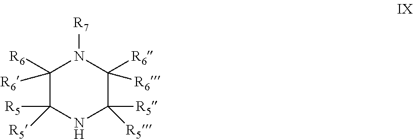

wherein R₅, R₅′, R₅″, R₅′″, R₆, R₆′, R₆″, R₆′″ and R₇ have the meanings as defined in the description, for the preparation of compounds of Formula (I).

A particular embodiment of the invention refers to the use of a compound of Formula (XII),

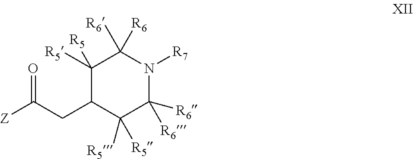

wherein R₅, R₅′, R₅″, R₅′″, R₆, R₆′, R₆″, R₆′″ and R₇ have the meanings as defined in the description, and Z represents OH or halogen, for the preparation of compounds of Formula (I).

A particular embodiment of the invention refers to the use of a compound of Formula (XIII),

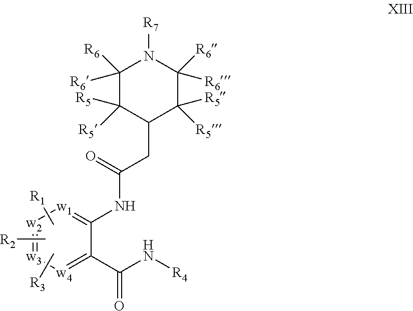

wherein R₁, R₂, R₃, R₄, R₅, R₅′, R₅″, R₅′″, R₆, R₆′, R₆″, R₆′″, R₇, w₁, w₂, w₃ and w₄ have the meanings as defined in the description, for the preparation of compounds of Formula (I).

A particular embodiment of the invention refers to the use of a compound of Formula (XIV),

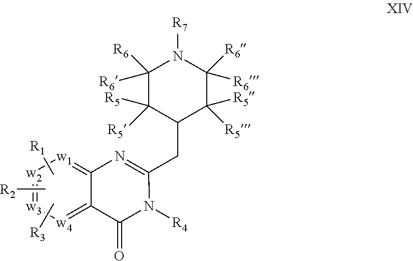

wherein R₁, R₂, R₃, R₄, R₅, R₅′, R₅″, R₅′″, R₆, R₆′, R₆″, R₆′″, R₇, w₁, w₂, w₃ and w₄ have the meanings as defined in the description, for the preparation of compounds of Formula (I).

A particular embodiment of the invention refers to the use of a compound of Formula (XV),

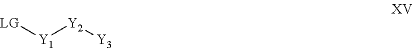

wherein Y₁, Y₂, and Y₃, have the meanings as defined in the description, and LG represents a leaving group, for the preparation of compounds of Formula (I).

A particular embodiment of the invention refers to the use of a compound of Formula (XVI),

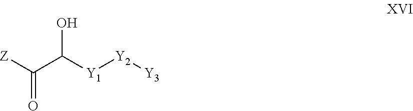

wherein Y₁, Y₂, and Y₃ have the meanings as defined in the description, and Z represents OH or halogen, for the preparation of compounds of Formula (I).

A particular embodiment of the invention refers to the use of a compound of Formula (XVII),

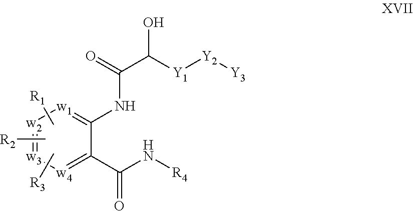

wherein R₁, R₂, R₃, R₄, w₁, w₂, w₃, w₄ Y₁, Y₂, and Y₃ have the meanings as defined in the description, for the preparation of compounds of Formula (I).

A particular embodiment of the invention refers to the use of a compound of Formula (XVIII),

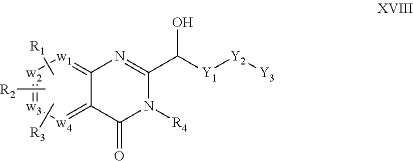

wherein R₁, R₂, R₃, R₄, w₁, w₂, w₃, w₄ Y₁, Y₂, and Y₃ have the meanings as defined in the description, for the preparation of compounds of Formula (I).

A particular embodiment of the invention refers to the use of a compound of Formula (IIa), (IIb), (III), (IV), (V), (VI), (VII), (VIII), (IX), (XII), (XIII), (XIV), (XV), (XVI), (XVII) or (XVIII)

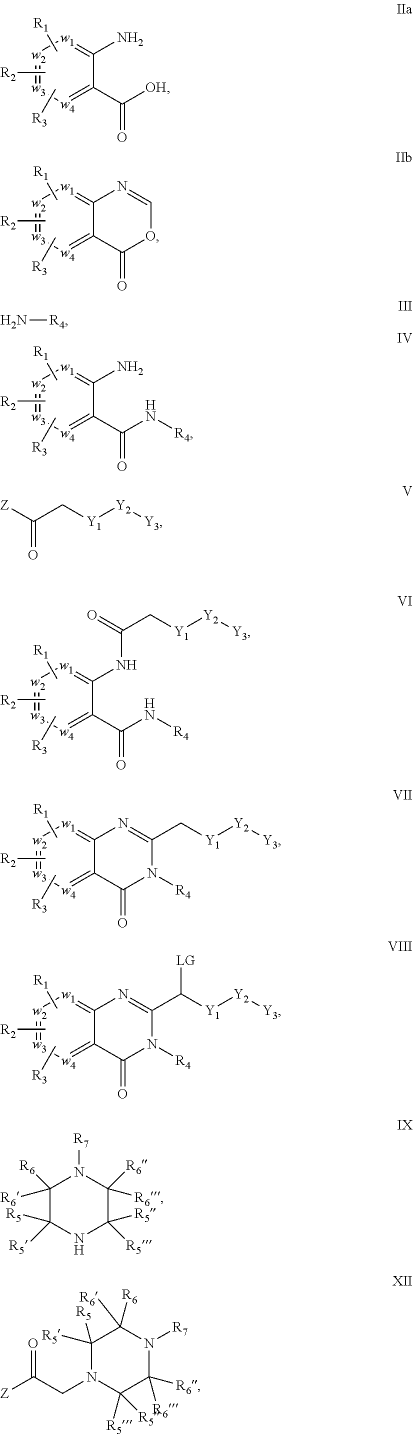

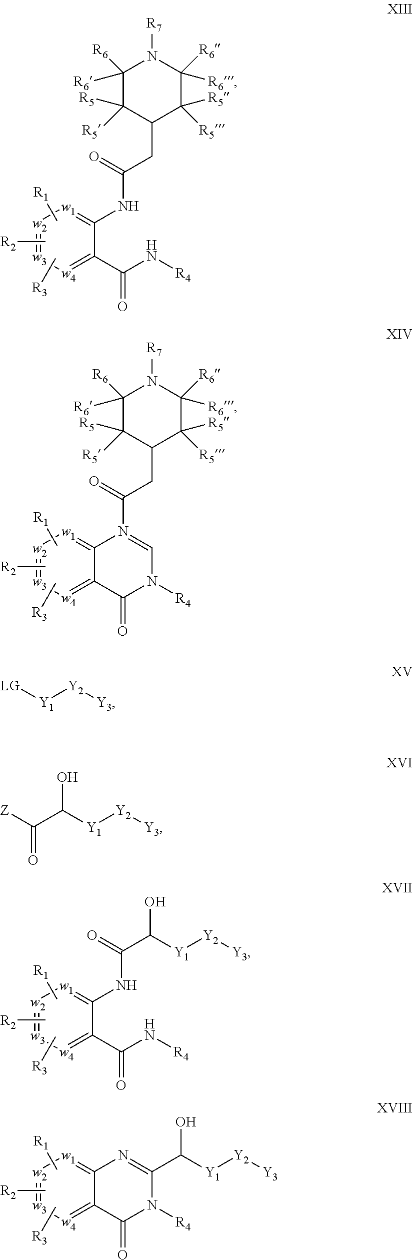

wherein R₁, R₂, R₃, R₄, R₅, R₅′, R₅″, R₅′″, R₆, R₆′, R₆″, R₆′″, R₇, Y₁, Y₂, Y₃, W, w₁, w₂, w₃ and w₄ have the meanings as defined in the description, Z represents OH or a halogen and LG represents a leaving group (such as chloro, bromo, iodo, mesylate, tosylate, nosylate or triflate), for the preparation of compounds of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or steroisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the present invention.

General Experimental Part

Synthesis Description

The compounds of formula I may be prepared by a four to five step process as described in Scheme 1,

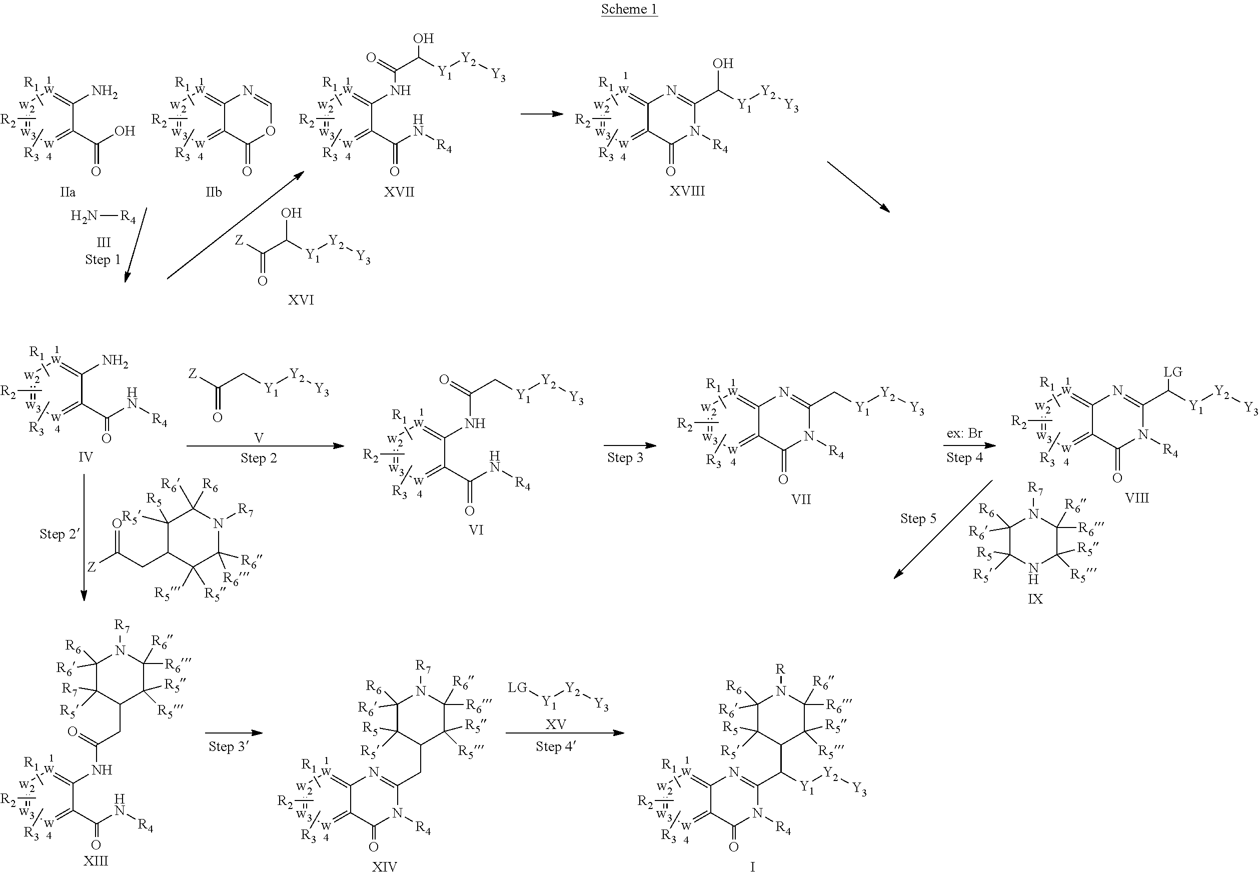

wherein R₁, R₂, R₃, R₄, R₅₋₅′″, R₆₋₆′″, W, w₁, w₂, w₃, w₄, Y₁, Y₂ and Y₃ have the meanings as defined in claim 1, LG represents a leaving group (such as chloro, bromo, iodo, mesylate, tosylate, nosylate or triflate) and Z represents OH or a halogen atom.

The process can be carried out as described below:

Step 1: A compound of formula IV can be prepared by treating an acid of formula IIa with a suitable amine of formula III in the presence of a suitable coupling agent, such as 1-[bis(dimethylamino)methylene]-1H-1,2,3-triazolo[4,5-b]pyridinium 3-oxid hexafluorophosphate, in the presence of a base such as triethylamine, in a suitable solvent, such as dimethylformamide, at a suitable temperature, preferably at room temperature. Alternatively, an oxazine derivative of formula IIb may be used as starting material, in which case the reaction with the amine of formula III is performed in acetonitrile, at a suitable temperature, such as heating.

Step 2: A compound of formula VI can be prepared by treating a compound of formula IV with a suitable acid derivative of formula V. When Z is a halogen atom the reaction may be carried out in the presence of a base, such as triethylamine, in a suitable solvent, such as dichloromethane, at a suitable temperature, such as room temperature. When Z is OH the reaction can be carried out using similar conditions to those described in step 1.

Step 3: A compound of formula VII can be prepared by treating a compound of formula VI with a suitable halogen such as iodine, in the presence of a base, such as hexamethyldisilazane, in a suitable solvent, such as dichloromethane, at a suitable temperature, preferably room temperature. Alternatively, the reaction may be carried out using a strong base, such as lithium hydroxide in a suitable solvent, such as ethylene glycol, at a suitable temperature, such as heating.

Step 4: A compound of formula VIII, where LG represents a leaving group, such as a halogen atom, can be prepared by reacting a compound of formula VII with a suitable halogenating agent, such as bromine in the presence of a suitable base such as sodium acetate, in a suitable solvent, such as acetic acid, at a suitable temperature, preferably heating.

Alternatively, a compound of formula VIII can be prepared by converting the hydroxyl group of a compound of formula XVIII into a leaving group. For instance, it can be converted to a triflate group by using triflic anhydride in the presence of a suitable base, such as 2,6-lutidine, at a suitable temperature such as between −78° C. and room temperature. A compound of formula XVIII may be obtained from a compound of formula XVII using the conditions described in Step 3. In turn, XVII may be prepared by coupling a compound of formula IV with an acid derivative of formula XVI using the conditions described in Step 2.

Step 5: A compound of formula I, in which W is nitrogen, can be prepared by reacting a compound of formula VIII with a suitable amine of formula IX, in a suitable solvent, such as acetonitrile or dimethylformamide, in the presence of a base such as triethylamine, K₂CO₃ or N,N-diisopropylethylamine, at a suitable temperature comprised between room temperature and the reflux temperature, preferably heating. Alternatively, the reactions can be carried out under microwave heating and optionally using an activating agent such as sodium iodide or potassium iodide.

Alternatively, a compound of formula I, in which W is a carbon atom, may be prepared by reacting a compound of formula IV with a compound of formula XII under the conditions used in Step 2 (Step 2′), to give a compound of formula XIII. This may be followed by cyclization under the conditions used in Step 3 (Step 3′) and final alkylation of a compound of formula XIV with a compound of formula XV, using a suitable base, such as lithium bis(trimethylsilyl)amide, in a suitable solvent, such as tetrahydrofuran at a suitable temperature, such as room temperature (Step 4′).

In addition, certain compounds of the present invention can also be obtained by functional group interconversion over compounds of formula I or any of the intermediates shown in Scheme 1. The following conversions are examples of transformations that may be carried out:
An ester group may be converted to a carboxylic acid group by reaction with sodium or lithium hydroxide in a suitable solvent, such as methanol or ethanol, at a suitable temperature, such as heating.
An amino group can be alkylated using an aldehyde in reductive amination conditions, in the presence of a suitable reducing agent, such as sodium borohydride and a base, such as potassium carbonate, in a suitable solvent, such as methanol, at a suitable temperature, preferably room temperature. Alternatively, an amino group may be alkylated using a suitable alkylating agent, such as an alkyl halide, in the presence of a base, such as triethylamine or sodium hydride, in a suitable solvent, such as ethanol or dimethylformamide, at a suitable temperature, preferably heating.
An aromatic halogen atom, ie a bromine atom, may be converted to an aryl group by reaction with a suitable arylboronic acid derivative, in the presence of a Pd catalyst such as tetrakis(triphenylphosphine)palladium(0), in a suitable solvent, such as mixtures of dimethoxyethane-water, in the presence of a base, such as potassium carbonate, at a suitable temperature, such as heating.
An aromatic halogen atom, ie a bromine atom, may be converted to an amino derivative by reaction with a suitable amine under Buchwald-Hartwig conditions, using a Pd catalyst such as tris(dibenzylideneacetone)dipalladium(0) or palladium acetate, and a suitable ligand, preferably a phosphine ligand such as BINAP or XPhos, using a suitable base such as sodium tert-butoxide or cesium carbonate, in a suitable solvent such as tert-butanol, toluene or 1,4-dioxane, at a suitable temperature, preferably heating.
A nitro group can be reduced to an amino group using a suitable reducing agent, such as tin(II) chloride, in a suitable solvent such as methanol, at a suitable temperature, preferably room temperature.
An amino group can be acylated to give an amide derivative under the conditions described above in steps 1 or 2.
An acid derivative may be converted to an amide derivative by reaction with and amine derivative under the conditions described above in steps 1 or 2.
An ester group can be reduced to a hydroxyl group using a suitable reducing agent, such as lithium borohydride, in a suitable solvent such as methanol or thetrrahydrofuran, at a suitable temperature, preferably room temperature.
An aromatic halogen atom, ie a bromine atom, may be converted to an alkyl derivative by reaction with a suitable potassium trifluoroborate derivative, using a Pd catalyst, such as palladium acetate or bis(di-tert-butyl(4-dimethylaminophenyl)phosphine)dichloropalladium(II) and a suitable base, such as cesium carbonate, in a suitable solvent such as toluene-water mixtures, at a suitable temperature, preferably heating and optionally under microwave irradiation
An aromatic halogen atom, ie a bromine atom, may be converted to a carbonitrile by reaction with zinc cyanide in the presence of a Pd catalyst such as tetrakis(triphenylphosphine)palladium(0), in a suitable solvent, such as dimethylformamide, at a suitable temperature, such as heating.

In some of the processes described above it may be necessary to protect the reactive or labile groups present with suitable protecting groups, such as for example Boc (tert-butoxycarbonyl), Teoc (2-(trimethylsilyl)ethoxycarbonyl) or benzyl for the protection of amino groups, and common silyl protecting groups for the protection of the hydroxyl group. The procedures for the introduction and removal of these protecting groups are well known in the art and can be found thoroughly described in the literature.

In addition, a compound of formula I can be obtained in enantiopure form by resolution of a racemic compound of formula I either by chiral preparative HPLC or by crystallization of a diastereomeric salt or co-crystal. Alternatively, the resolution step can be carried out at a previous stage, using any suitable intermediate.

The compounds of formula IIa, IIb, III, V, IX, XII, XV and XVI used in the methods disclosed above are commercially available or can be synthesized following common procedures described in the literature and exemplified in the synthesis of some intermediates.

### Examples

The following abbreviations are used in the examples:

ACN: acetonitrile

Aq: aqueous

Anh: anhydrous

Chx: cyclohexane

DCM: dichloromethane

DIPEA: N,N-diisopropylethylamine

DME: dimethoxyethane

DMF dimethylformamide

Eq: equivalent/s

Et₂O: diethyl ether

EtOAc; ethyl acetate

h: hours

HATU: (1-[bis(dimethylamino)methylene]-1H-1,2,3-triazolo[4,5-b]pyridinium 3-oxid hexafluorophosphate)

HMDS: hexamethyldisilazane

HPLC: high performance liquid chromatography

LiHMDS: lithium bis(trimethylsilyl)amide

MeOH: methanol

MS: mass spectrometry

Min: minutes

Pd(dppf)FeCl₂: [1,1′-Bis(diphenylphosphino)ferrocene]dichloropalladium(II)

PdCl₂(bpy): (2,2′-Bipyridine)dichloropalladium(II)

Quant: quantitative

Rt.: retention time

r.t.: room temperature

Sat: saturated

Sol: solution

TBAF: tetrabutylammonium fluoride

TEA: triethylamine

TFA: trifluoroacetic acid

THF: tetrahydrofuran

Wt: weight

XPhos: 2-dicyclohexylphosphino-2′,4′,6′-triisopropylbiphenyl

The following methods were used to determine the HPLC-MS spectra:

Method A

Column Aquity UPLC BEH C18 2.1×50 mm, 1.7 μm, flow rate 0.61 mL/min; A: NH₄HCO₃ 10 mM, B: ACN, C: MeOH+0.1% formic acid; gradient 0.3 min 98% A, 98% A to 0:95:5 A:B:C in 2.7 min; 0:95:5 A:B:C to 100% B in 0.1 min; isocratic 2 min 100% B.

Method B

Column Aquity UPLC BEH C18 2.1×50 mm, 1.7 μm, flow rate 0.61 mL/min; A: NH₄HCO₃ 10 mM, B: ACN; gradient 0.3 min 98% A, 98% A to 100% B in 2.65 min; isocratic 2.05 min 100% B.

Method C

Column Acquity UPLC BEH C18 2.1×50 mm, 1.7 μm; flow rate 0.61 mL/min; A: NH₄HCO₃ 10 mM; B: ACN; gradient: 0.3 min in 98% A, 98% A to 5% A in 2.52 min, isocratic 1.02 min 5% A.

Method D

Column Acquity UPLC BEH C18 2.1×50 mm, 1.7 μm; flow rate 0.60 mL/min; A: NH₄HCO₃ 10 mM; B: ACN; Gradient: 0.3 min in 90% A, 90% A to 5% A in 2.7 min, isocratic 0.7 min 5% A.

Method E

Column Acquity UPLC BEH C18 2.1×50 mm, 1.7 μm; flow rate 0.60 mL/min; A: HCO₂NH₄ 10 mM; B: ACN; Gradient: 0.3 min in 90% A, 90% A to 5% A in 2.7 min, isocratic 0.7 min 5% A.

Method F

Column Aquity UPLC BEH C18 2.1×50 mm, 1.7 μm, flow rate 0.60 mL/min; A: NH₄HCO₃ 10 mM, B: ACN; gradient: 0.3 min in 90% A, 90% A to 5% A in 2.7 min, isocratic 2 min 5% A.

Method G

Column Aquity UPLC BEH C18 2.1×50 mm, 1.7 μm, flow rate 0.60 mL/min; A: NH₄HCO₃10 mM pH 10.6, B: ACN; gradient: 0.3 min in 90% A, 90% A to 5% A in 2.7 min, isocratic 2 min 5% A.

Method H

Column Zodiac C-18 4.6×50 mm, 3 μm, flow rate 0.6 mL/min: A: 0.1% HCOOH in H₂O, B: ACN; gradient 90% A to 50% A in 1 min, 50% A to 10% A in 2 min isocratic 2 min 10% A.

Method I

Column Acquity UPLC BEH C18 2.1×50 mm, 1.7 μm; flow rate 0.60 mL/min; A: NH₄HCO₃ 10 mM pH 10.6; B: ACN; Gradient: 0.3 min in 90% A, 90% A to 5% A in 2.7 min, isocratic 0.7 min 5% A.

### SYNTHESIS OF EXAMPLES

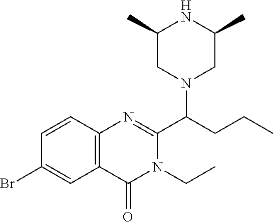 [SMILES: CCCC(c1nc2ccc(Br)cc2c(=O)n1CC)N1C[C@@H](C)N[C@@H](C)C1]

### Example 1. 6-Bromo-2-(1-((3S,5R)-3,5-dimethylpiperazin-1-yl)butyl)-3-ethylquinazolin-4(3H)-one

### Step a. 2-Amino-5-bromo-N-ethylbenzamide

To a solution of 2-amino-5-bromobenzoic acid (10 g, 46.3 mmol) in anh DMF (200 mL) under argon atmosphere, TEA (13 mL, 92.6 mmol) and HATU (21.1 g, 55 mmol) were added and the reaction mixture was stirred at 0° C. for 10 min. Then, ethylamine (2 M in THF, 35 mL, 69.4 mmol) was added dropwise and the reaction mixture was allowed to reach r.t. and stirred overnight. The reaction crude was diluted with EtOAc:Et₂O (300 mL, 1:1) and washed with aq NaHCO₃ sat sol. The organic layer was dried over anh Na₂SO₄, filtered and concentrated to dryness to give the title compound (10.8 g, Yield: 85%).

### Step b. 5-Bromo-N-ethyl-2-pentanamidobenzamide

To a solution of the compound obtained in step a (10.7 g, 44.1 mmol) in anh DCM (200 mL) under argon atmosphere, TEA (9.23 mL, 66.2 mmol) was added dropwise and the mixture was stirred for 10 min. The solution was cooled at 0° C., pentanoyl chloride (6 mL, 48.5 mmol) was added dropwise and the reaction mixture was allowed to reach r.t. and stirred overnight. The resulting mixture was diluted with DCM and washed with aq NaHCO₃ sat sol. The organic layer was dried over anh Na₂SO₄ and filtered and the solvent was removed under vacuum to give the title compound (13.3 g, Yield: 82%).

### Step c. 6-Bromo-2-butyl-3-ethylquinazolin-4(3H)-one

To a solution of the compound obtained in step b (13.3 g, 40.7 mmol) in anh DCM (150 mL), iodine (20.7 g, 81.4 mmol) was added portion wise and the mixture was stirred until full solution was observed. The solution was cooled at 0° C., HMDS (34 mL, 26.3 mmol) was added dropwise and the reaction mixture was allowed to reach r.t. and stirred overnight. DCM was added and the reaction mixture was washed with a 5% Na₂S₂O₃ sol. The organic layer was dried over Na₂SO₄, filtered and solvent was removed under vacuum to give the title compound (12.5 g, Yield: 89%).

### Step d. 6-Bromo-2-(1-bromobutyl)-3-ethylquinazolin-4(3H)-one

To a solution of the compound obtained in step c (12.5 g, 40.5 mmol) in acetic acid (125 mL), NaOAc (4 g, 48.6 mmol) was added portion wise and the reaction was stirred for 15 min at r.t. Bromine (3.1 mL, 60.7 mmol) was added dropwise and the reaction mixture was heated at 50° C. for 3 h. The mixture was concentrated under vacuum and the residue was dissolved in EtOAc and washed twice with 10% NaHSO₃ aq sol and brine. The organic layer was dried over anh Na₂SO₄ and the solvent was removed under vacuum. The crude product was purified by flash chromatography, silica gel, gradient Chx to Chx:EtOAc (9:1) to give the title compound (12.2 g, Yield: 78%).

### Step e. Title Compound

To a solution of the compound obtained in step d (3.0 g, 7.7 mmol) in ACN (180 mL), TEA (4.3 mL, 30.9 mmol) and KI (128 mg, 0.77 mmol) were added and the reaction mixture was stirred at r.t. for 20 min. (2R,6S)-2,6-Dimethylpiperazine (2.2 g, 19.3 mmol) was added portion wise, the mixture was heated at 90° C. and stirred overnight. The mixture was concentrated under vacuum, the crude product was dissolved in EtOAc and washed with aq NaHCO₃ sat sol. The organic layer was dried over anh Na₂SO₄, filtered and concentrated to dryness. The crude product was purified by flash chromatography, silica gel, gradient Chx to Chx:EtOAc (4:1) to give the title compound (2.1 g, Yield: 64%).

HPLC-MS (C) Rt, 2.04 my; ESI+-MS m/z: 421.3 (M+1).

This method was used for the preparation of examples 2-28 using suitable starting materials:

| | | CHEMICAL | Rt | MS | HPLC |
|---|---|---|---|---|---|
| STRUCTURE | EX | NAME | (min) | (M + H) | Method |
| 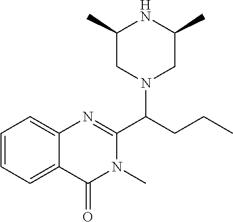 [SMILES: CCCC(c1nc2ccccc2c(=O)n1C)N1C[C@@H](C)N[C@@H](C)C1] | 2 | 2-(1-((3S,5R)-3,5- dimethylpiperazin-1- yl)butyl)-3- methylquinazolin- 4(3H)-one | 1.58 | 329.1 | C |
| 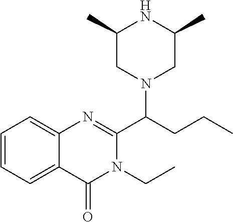 [SMILES: CCCC(c1nc2ccccc2c(=O)n1CC)N1C[C@@H](C)N[C@@H](C)C1] | 3 | 2-(1-((3S,5R)-3,5- dimethylpiperazin-1- yl)butyl)-3-(2- (methylamino)ethyl) quinazolin-4(3H)-one | 1.72 | 343.2 | C |
| 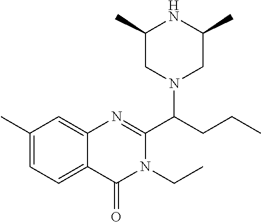 [SMILES: CCCC(c1nc2cc(C)ccc2c(=O)n1CC)N1C[C@@H](C)N[C@@H](C)C1] | 4 | 2-(1-((3S,5R)-3,5- dimethylpiperazin-1- yl)butyl)-3-ethyl-7- methylquinazolin- 4(3H)-one | 1.90 | 357.3 | C |
| 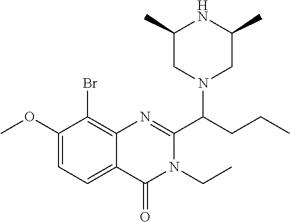 [SMILES: CCCC(c1nc2c(Br)c(OC)ccc2c(=O)n1CC)N1C[C@@H](C)N[C@@H](C)C1] | 5 | 8-bromo-2-(1- ((3S,5R)-3,5- dimethylpiperazin-1- yl)butyl)-3-ethyl-7- methoxyquinazolin- 4(3H)-one | 1.98 | 451.1 | C |
| 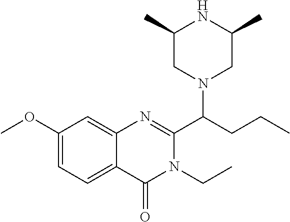 [SMILES: CCCC(c1nc2cc(OC)ccc2c(=O)n1CC)N1C[C@@H](C)N[C@@H](C)C1] | 6 | 2-(1-((3S,5R)-3,5- dimethylpiperazin-1- yl)butyl)-3-ethyl-7- methoxyquinazolin- 4(3H)-one | 1.79 | 373.2 | C |
| 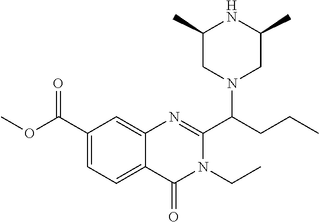 [SMILES: CCCC(c1nc2cc(C(=O)OC)ccc2c(=O)n1CC)N1C[C@@H](C)N[C@@H](C)C1] | 7 | methyl 2-(1-((3S,5R)- 3,5-dimethylpiperazin- 1-yl)butyl)-3-ethyl-4- oxo-3,4- dihydroquinazoline-7- carboxylate | 1.81 | 401.3 | C |
| 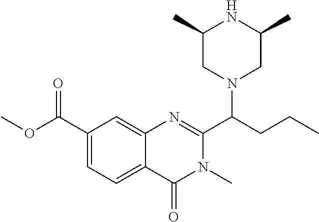 [SMILES: CCCC(c1nc2cc(C(=O)OC)ccc2c(=O)n1C)N1C[C@@H](C)N[C@@H](C)C1] | 8 | methyl 2-(1-((3S,5R)- 3,5-dimethylpiperazin- 1-yl)butyl)-3-methyl-4- oxo-3,4- dihydroquinazoline-7- carboxylate | 1.70 | 387.3 | F |
| 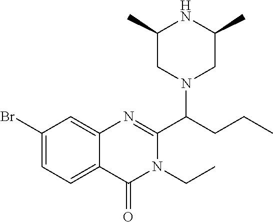 [SMILES: CCCC(c1nc2cc(Br)ccc2c(=O)n1CC)N1C[C@@H](C)N[C@@H](C)C1] | 9 | 7-bromo-2-(1- ((3S,5R)-3,5- dimethylpiperazin-1- yl)butyl)-3- ethylquinazolin-4(3H)- one | 2.14 | 421.1 | C |
| 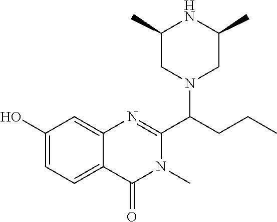 [SMILES: CCCC(c1nc2cc(O)ccc2c(=O)n1C)N1C[C@@H](C)N[C@@H](C)C1] | 10 | 2-(1-((3S,5R)-3,5- dimethylpiperazin-1- yl)butyl)-7-hydroxy-3- methylquinazolin- 4(3H)-one | 1.20 | 345.3 | C |
| 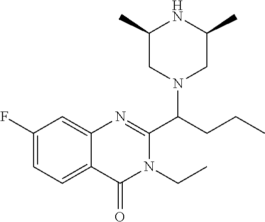 [SMILES: CCCC(c1nc2cc(F)ccc2c(=O)n1CC)N1C[C@@H](C)N[C@@H](C)C1] | 11 | 2-(1-((3S,5R)-3,5- dimethylpiperazin-1- yl)butyl)-3-ethyl-7- fluoroquinazolin- 4(3H)-one | 1.83 | 361.3 | C |
| 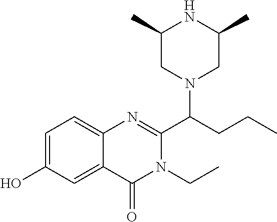 [SMILES: CCCC(c1nc2ccc(O)cc2c(=O)n1CC)N1C[C@@H](C)N[C@@H](C)C1] | 12 | 2-(1-((3S,5R)-3,5- dimethylpiperazin-1- yl)butyl)-3-ethyl-6- hydroxyquinazolin- 4(3H)-one | 1.45 | 359.2 | F |
| 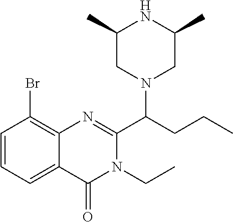 [SMILES: CCCC(c1nc2c(Br)cccc2c(=O)n1CC)N1C[C@@H](C)N[C@@H](C)C1] | 13 | 8-bromo-2-(1- ((3S,5R)-3,5- dimethylpiperazin-1- yl)butyl)-3- ethylquinazolin-4(3H)- one | 2.11 | 421.1 | F |
| 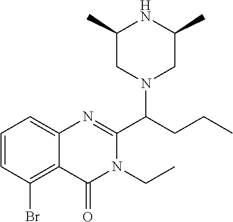 [SMILES: CCCC(c1nc2cccc(Br)c2c(=O)n1CC)N1C[C@@H](C)N[C@@H](C)C1] | 14 | 5-bromo-2-(1- ((3S,5R)-3,5- dimethylpiperazin-1- yl)butyl)-3- ethylquinazolin-4(3H)- one | 2.05 | 421.1 | F |
| 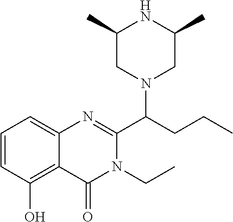 [SMILES: CCCC(c1nc2cccc(O)c2c(=O)n1CC)N1C[C@@H](C)N[C@@H](C)C1] | 15 | 2-(1-((3S,5R)-3,5- dimethylpiperazin-1- yl)butyl)-3-ethyl-5- hydroxyquinazolin- 4(3H)-one | 2.04 | 359.3 | F |
| 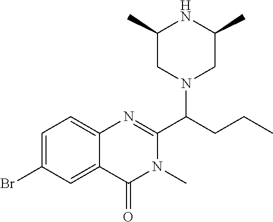 [SMILES: CCCC(c1nc2ccc(Br)cc2c(=O)n1C)N1C[C@@H](C)N[C@@H](C)C1] | 16 | 6-bromo-2-(1- ((3S,5R)-3,5- dimethylpiperazin-1- yl)butyl)-3- methylquinazolin- 4(3H)-one | 1.97 | 407.1 | F |
| 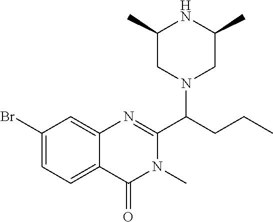 [SMILES: CCCC(c1nc2cc(Br)ccc2c(=O)n1C)N1C[C@@H](C)N[C@@H](C)C1] | 17 | 7-bromo-2-(1- ((3S,5R)-3,5- dimethylpiperazin-1- yl)butyl)-3- methylquinazolin- 4(3H)-one | 2.26 | 407.2 | H |
| 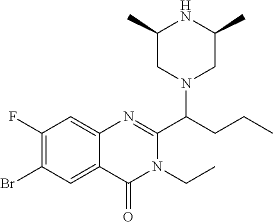 [SMILES: CCCC(c1nc2cc(F)c(Br)cc2c(=O)n1CC)N1C[C@@H](C)N[C@@H](C)C1] | 18 | 6-bromo-2-(1- ((3S,5R)-3,5- dimethylpiperazin-1- yl)butyl)-3-ethyl-7- fluoroquinazolin- 4(3H)-one | 2.34 | 439.1 | A |
| 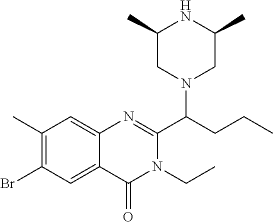 [SMILES: CCCC(c1nc2cc(C)c(Br)cc2c(=O)n1CC)N1C[C@@H](C)N[C@@H](C)C1] | 19 | 6-bromo-2-(1- ((3S,5R)-3,5- dimethylpiperazin-1- yl)butyl)-3-ethyl-7- methylquinazolin- 4(3H)-one | 2.43 | 435.2 | A |
| 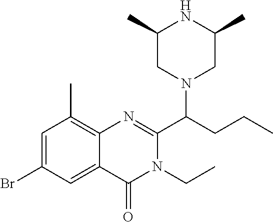 [SMILES: CCCC(c1nc2c(C)cc(Br)cc2c(=O)n1CC)N1C[C@@H](C)N[C@@H](C)C1] | 20 | 6-bromo-2-(1- ((3S,5R)-3,5- dimethylpiperazin-1- yl)butyl)-3-ethyl-8- methylquinazolin- 4(3H)-one | 2.54 | 435.2 | A |
| 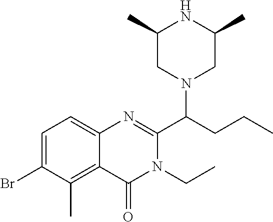 [SMILES: CCCC(c1nc2ccc(Br)c(C)c2c(=O)n1CC)N1C[C@@H](C)N[C@@H](C)C1] | 21 | 6-bromo-2-(1- ((3S,5R)-3,5- dimethylpiperazin-1- yl)butyl)-3-ethyl-5- methylquinazolin- 4(3H)-one | 2.59 | 435.2 | A |
| 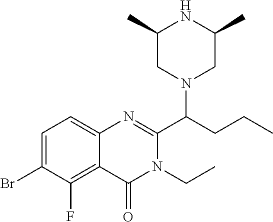 [SMILES: CCCC(c1nc2ccc(Br)c(F)c2c(=O)n1CC)N1C[C@@H](C)N[C@@H](C)C1] | 22 | 6-bromo-2-(1- ((3S,5R)-3,5- dimethylpiperazin-1- yl)butyl)-3-ethyl-5- fluoroquinazolin- 4(3H)-one | 2.20 | 439.2 | A |
| 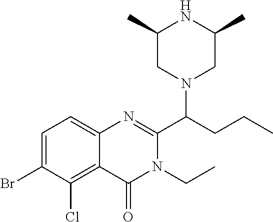 [SMILES: CCCC(c1nc2ccc(Br)c(Cl)c2c(=O)n1CC)N1C[C@@H](C)N[C@@H](C)C1] | 23 | 6-bromo-5-chloro-2-(1- ((3S,5R)-3,5- dimethylpiperazin-1- yl)butyl)-3- ethylquinazolin-4(3H)- one | 2.32 | 455.2 | A |
| 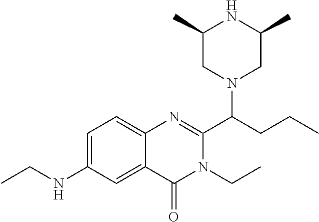 [SMILES: CCCC(c1nc2ccc(NCC)cc2c(=O)n1CC)N1C[C@@H](C)N[C@@H](C)C1] | 24 | 2-(1-((3S,5R)-3,5- dimethylpiperazin-1- yl)butyl)-3-ethyl-6- (ethylamino)quinazolin- 4(3H)-one | 1.71 | 386.3 | C |
| 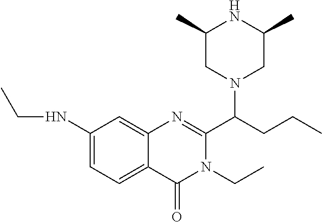 [SMILES: CCCC(c1nc2cc(NCC)ccc2c(=O)n1CC)N1C[C@@H](C)N[C@@H](C)C1] | 25 | 2-(1-((3S,5R)-3,5- dimethylpiperazin-1- yl)butyl)-3-ethyl-7- (ethylamino)quinazolin- 4(3H)-one | 2.08 | 386.2 | D |
| 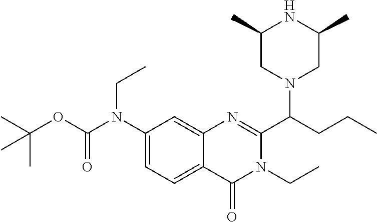 [SMILES: CCCC(c1nc2cc(N(CC)C(=O)OC(C)(C)C)ccc2c(=O)n1CC)N1C[C@@H](C)N[C@@H](C)C1] | 26 | tert-butyl (2-(1- ((3S,5R)-3,5- dimethylpiperazin-1- yl)butyl)-3-ethyl-4-oxo- 3,4-dihydroquinazolin- 7-yl)(ethyl)carbamate | 2.17 | 486.3 | C |
| 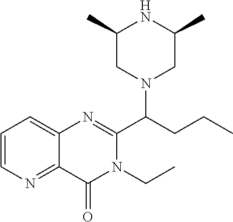 [SMILES: CCCC(c1nc2cccnc2c(=O)n1CC)N1C[C@@H](C)N[C@@H](C)C1] | 27 | 2-(1-((3S,5R)-3,5- dimethylpiperazin-1- yl)butyl)-3- ethylpyrido[3,2- d]pyrimidin-4(3H)-one | 1.48 | 344.2 | A |
| 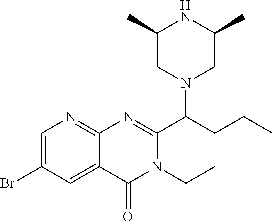 [SMILES: CCCC(c1nc2ncc(Br)cc2c(=O)n1CC)N1C[C@@H](C)N[C@@H](C)C1] | 28 | 6-bromo-2-(1- ((3S,5R)-3,5- dimethylpiperazin-1- yl)butyl)-3- ethylpyrido[2,3- d]pyrimidin-4(3H)-one | 1.86 | 422.2 | A |

### Examples 29 and 30. 6-Bromo-2-((R)-1-((3S,5R)-3,5-dimethyl piperazin-1-yl)butyl)-3-ethylquinazolin-4(3H)-one and 6-bromo-2-((S)-1-((3S,5R)-3,5-dimethylpiperazin-1-yl)butyl)-3-ethylquinazolin-4(3H)-one

Starting from the compound obtained in example 1, a chiral preparative SFC separation [column: Chiralpak IG (4.6×250) mm, 5p, temperature: ambient; flow: 3 mL/min, CO₂/0.2% TEA in MeOH (80:20)] was carried out to give the title compounds.

### Examples 31 and 32. 2-((S)-1-((3S,5R)-3,5-dimethylpiperazin-1-yl)butyl)-3-methylquinazolin-4(3H)-one and 2-((R)-1-((3S,5R)-3,5-dimethylpiperazin-1-yl)butyl)-3-methylquinazolin-4(3H)-one

Starting from the compound obtained in example 2, a chiral preparative HPLC separation [column: Chiralpak IC, temperature: ambient; flow: 12 mL/min, eluent n-Heptane/(EtOH+0.33% DEA) 85/15 v/v; Rt₁: 20.9′, Rt₂: 24.7′] was carried out to give the title compounds.

### Examples 33 and 34. 2-((S)-1-((3S,5R)-3,5-Dimethylpiperazin-1-yl)butyl)-3-ethylquinazolin-4(3H)-one and 2-((R)-1-((3S,5R)-3,5-dimethylpiperazin-1-yl)butyl)-3-ethylquinazolin-4(3H)-one

Starting from the compound obtained in example 3, a chiral preparative HPLC separation [column: Chiralpak IC, temperature: ambient; flow: 12 mL/min, eluent n-Heptane/(IPA+0.33% DEA) 95/5 v/v; Rt₁: 31.7′, Rt₂: 35.9′] was carried out to give the title compounds.

### Examples 35 and 36. 2-((R)-1-((3S,5R)-3,5-Dimethylpiperazin-1-yl)butyl)-3-ethyl-6-hydroxyquinazolin-4(3H)-one and 2-((S)-1-((3S,5R)-3,5-dimethylpiperazin-1-yl)butyl)-3-ethyl-6-hydroxyquinazolin-4(3H)-one

Starting from the compound obtained in example 12, a chiral preparative HPLC separation [column: Chiralpak AD-H, temperature: ambient; flow: 13 mL/min, eluent n-Heptane/(IPA+0.33% DEA) 70/30 v/v; Rt₁: 5.6′, Rt₂: 9.6′] was carried out to give the title compounds.

The following compounds were obtained using the same method described in Example 1, but directly separating the enantiomeric or diastereomeric mixtures using chiral HPLC:

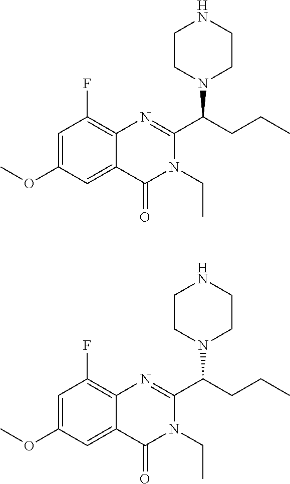

### Examples 37 and 38. (S)-3-Ethyl-8-fluoro-6-methoxy-2-(1-(piperazin-1-yl)butyl)quinazolin-4(3H)-one and (R)-3-ethyl-8-fluoro-6-methoxy-2-(1-(piperazin-1-yl)butyl)quinazolin-4(3H)-one

Starting from 3-ethyl-8-fluoro-6-methoxy-2-(1-(piperazin-1-yl)butyl)quinazolin-4(3H)-one, a chiral preparative HPLC separation [column: Chiralpak OD-H, temperature: ambient; flow: 12 mL/min, eluent n-Heptane/(EtOH+0.33% DEA) 90/10 v/v; Rt₁: 13.7′, Rt₂: 15.8′] was carried out to give the title compounds.

HPLC-MS (A) Rt, 1.79 min; ESI+-MS m/z: 363.2 (M+1).

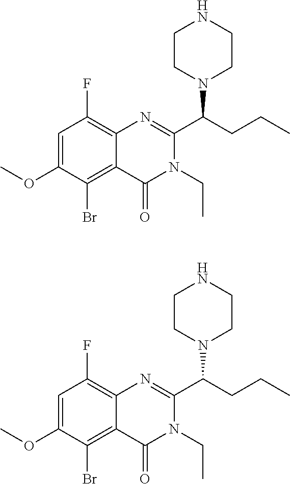

### Examples 39 and 40. (S)-5-Bromo-3-ethyl-8-fluoro-6-methoxy-2-(1-(piperazin-1-yl)butyl)quinazolin-4(3H)-one and (R)-5-bromo-3-ethyl-8-fluoro-6-methoxy-2-(1-(piperazin-1-yl)butyl)quinazolin-4(3H)-one

Starting from 5-bromo-3-ethyl-8-fluoro-6-methoxy-2-(1-(piperazin-1-yl)butyl)quinazolin-4(3H)-one, a chiral preparative HPLC separation [column: Chiralpak OD-H, temperature: ambient; flow: 12 mL/min, eluent n-Heptane/(EtOH+0.33% DEA) 90/10 v/v; Rt₁: 21.5′, Rt₂: 25.2′] was carried out to give the title compounds.

HPLC-MS (A) Rt, 1.85 min; ESI+-MS m/z: 441.1 (M+1).

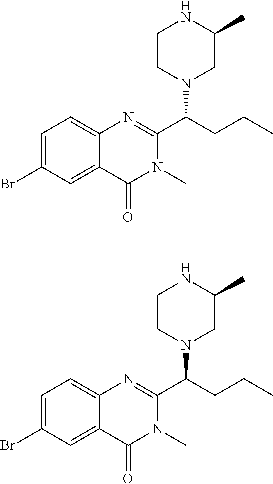 [SMILES: CCC[C@H](c1nc2ccc(Br)cc2c(=O)n1C)N1CCN[C@@H](C)C1 ; CCC[C@@H](c1nc2ccc(Br)cc2c(=O)n1C)N1CCN[C@@H](C)C1]

### Examples 41 and 42. 6-Bromo-3-methyl-2-((R)-1-((S)-3-methylpiperazin-1-yl)butyl)quinazolin-4(3H)-one and 6-bromo-3-methyl-2-((S)-1-((S)-3-methylpiperazin-1-yl)butyl)quinazolin-4(3H)-one

Starting from 6-bromo-3-methyl-2-(1-((S)-3-methylpiperazin-1-yl)butyl)quinazolin-4(3H)-one, a chiral preparative HPLC separation [column: Chiralpak AD-H, temperature: ambient; flow: 13 mL/min, eluent n-Heptane/(EtOH+0.33% DEA) 90/10 v/v; Rt₁: 10.9′, Rt₂: 16.1′] was carried out to give the title compounds.

HPLC-MS (B) Rt, 1.96 min; ESI+-MS m/z: 393.0 (M+1).

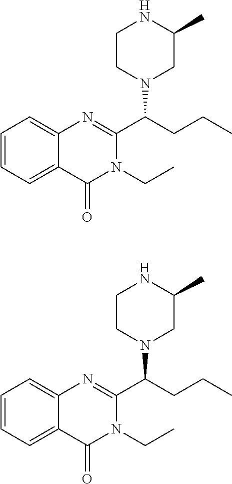 [SMILES: CCC[C@H](c1nc2ccccc2c(=O)n1CC)N1CCN[C@@H](C)C1 ; CCC[C@@H](c1nc2ccccc2c(=O)n1CC)N1CCN[C@@H](C)C1]

### Examples 43 and 44. 3-Ethyl-2-((R)-1-((S)-3-methylpiperazin-1-yl)butyl)quinazolin-4(3H)-one and 3-ethyl-2-((S)-1-((S)-3-methylpiperazin-1-yl)butyl)quinazolin-4(3H)-one

Starting from 3-ethyl-2-(1-((S)-3-methylpiperazin-1-yl)butyl)quinazolin-4(3H)-one, a chiral preparative HPLC separation [column: Chiralpak AD-H, temperature: ambient; flow: 13 mL/min, eluent n-Heptane/(EtOH+0.33% DEA) 90/10 v/v; Rt₁: 7.8′, Rt₂: 21.4′] was carried out to give the title compounds.

HPLC-MS (B) Rt, 1.76 min; ESI+-MS m/z: 329.0 (M+1).

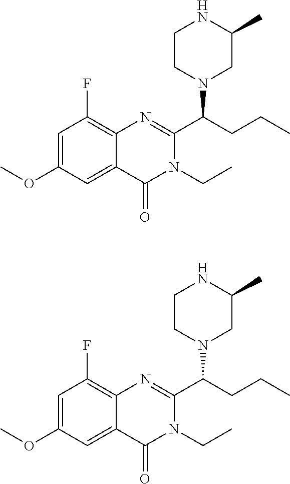

### Examples 45 and 46. 3-Ethyl-8-fluoro-6-methoxy-2-((S)-1-((S)-3-methylpiperazin-1-yl)butyl)quinazolin-4(3H)-one and 3-ethyl-8-fluoro-6-methoxy-2-((R)-1-((S)-3-methylpiperazin-1-yl)butyl)quinazolin-4(3H)-one

Starting from 3-ethyl-8-fluoro-6-methoxy-2-(1-((S)-3-methylpiperazin-1-yl)butyl)quinazolin-4(3H)-one, a chiral preparative HPLC separation [column: Chiralpak OD-H, temperature: ambient; flow: 13 mL/min, eluent n-Heptane/(EtOH+0.33% DEA) 95/5 v/v; Rt₁: 11.8′, Rt₂: 14.2′] was carried out to give the title compounds.

HPLC-MS (A) Rt, 1.89 min; ESI+-MS m/z: 377.3 (M+1).

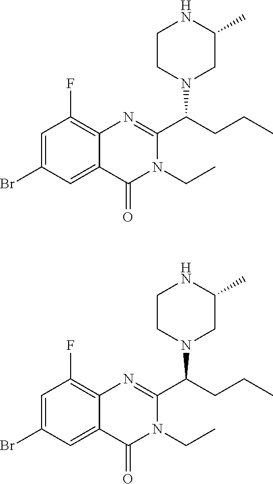 [SMILES: CCC[C@H](c1nc2c(F)cc(Br)cc2c(=O)n1CC)N1CCN[C@H](C)C1 ; CCC[C@@H](c1nc2c(F)cc(Br)cc2c(=O)n1CC)N1CCN[C@H](C)C1]

### Examples 47 and 48. 6-Bromo-3-ethyl-8-fluoro-2-((R)-1-((R)-3-methylpiperazin-1-yl)butyl)quinazolin-4(3H)-one and 6-bromo-3-ethyl-8-fluoro-2-((S)-1-((R)-3-methylpiperazin-1-yl)butyl)quinazolin-4(3H)-one

Starting from 6-bromo-3-ethyl-8-fluoro-2-(1-((R)-3-methylpiperazin-1-yl)butyl)quinazolin-4(3H)-one, a chiral preparative HPLC separation [column: Chiralpak IA, temperature: ambient; flow: 12 mL/min, eluent n-Heptane/(IPA+0.33% DEA) 70/30 v/v; Rt₁: 6.1′, Rt₂: 16.3′] was carried out to give the title compounds.

HPLC-MS (A) Rt, 2.10 min; ESI+-MS m/z: 425.2 (M+1).

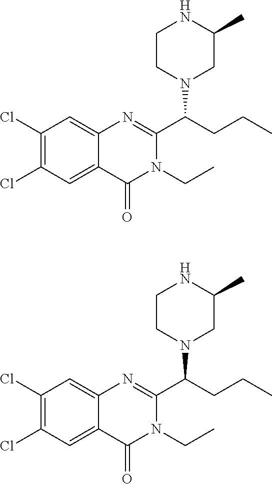 [SMILES: CCC[C@H](c1nc2cc(Cl)c(Cl)cc2c(=O)n1CC)N1CCN[C@@H](C)C1 ; CCC[C@@H](c1nc2cc(Cl)c(Cl)cc2c(=O)n1CC)N1CCN[C@@H](C)C1]

### Examples 49 and 50. 6,7-Dichloro-3-ethyl-2-((R)-1-((S)-3-methylpiperazin-1-yl)butyl)quinazolin-4(3H)-one and 6,7-dichloro-3-ethyl-2-((S)-1-((S)-3-methylpiperazin-1-yl)butyl)quinazolin-4(3H)-one

Starting from 6,7-dichloro-3-ethyl-2-(1-((S)-3-methylpiperazin-1-yl)butyl)quinazolin-4(3H)-one, a chiral preparative HPLC separation [column: Chiralpak AD-H, temperature: ambient; flow: 0.8 mL/min, eluent n-Heptane/(EtOH+0.33% DEA) 90/10 v/v; Rt₁: 6.5′, Rt₂: 16.9′] was carried out to give the title compounds.

HPLC-MS (A) Rt, 2.34 min; ESI+-MS m/z: 397.2 (M+1).

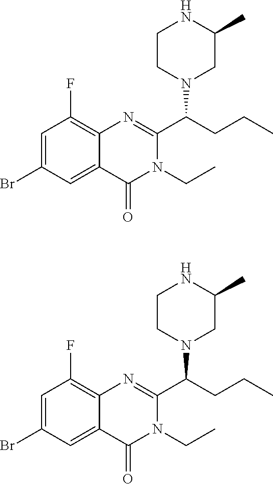 [SMILES: CCC[C@H](c1nc2c(F)cc(Br)cc2c(=O)n1CC)N1CCN[C@@H](C)C1 ; CCC[C@@H](c1nc2c(F)cc(Br)cc2c(=O)n1CC)N1CCN[C@@H](C)C1]

### Examples 51 and 52. 6-Bromo-3-ethyl-8-fluoro-2-((R)-1-((S)-3-methylpiperazin-1-yl)butyl)quinazolin-4(3H)-one and 6-bromo-3-ethyl-8-fluoro-2-((S)-1-((S)-3-methylpiperazin-1-yl)butyl)quinazolin-4(3H)-one

Starting from 6-bromo-3-ethyl-8-fluoro-2-(1-((S)-3-methylpiperazin-1-yl)butyl)quinazolin-4(3H)-one, a chiral preparative HPLC separation [column: Chiralpak IC, temperature: ambient; flow: 11 mL/min, eluent n-Heptane/(IPA+0.33% DEA) 70/30 v/v; Rt₁: 12.4′, Rt₂: 15.5′] was carried out to give the title compounds.

HPLC-MS (A) Rt, 2.18 min; ESI+-MS m/z: 425.2 (M+1).

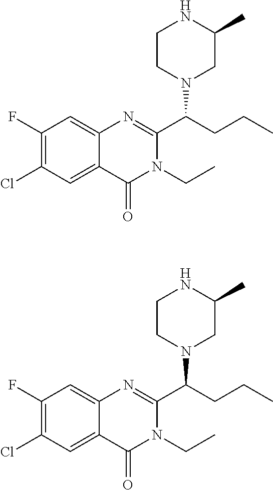 [SMILES: CCC[C@H](c1nc2cc(F)c(Cl)cc2c(=O)n1CC)N1CCN[C@@H](C)C1 ; CCC[C@@H](c1nc2cc(F)c(Cl)cc2c(=O)n1CC)N1CCN[C@@H](C)C1]

### Examples 53 and 54. 6-Chloro-3-ethyl-7-fluoro-2-((R)-1-((S)-3-methylpiperazin-1-yl)butyl)quinazolin-4(3H)-one and 6-chloro-3-ethyl-7-fluoro-2-((S)-1-((S)-3-methylpiperazin-1-yl)butyl)quinazolin-4(3H)-one

Starting from 6-chloro-3-ethyl-7-fluoro-2-(1-((S)-3-methylpiperazin-1-yl)butyl)quinazolin-4(3H)-one, a chiral preparative SFC separation [column: Lux A1 (21.2 mm×250 mm, 5 um), temperature: ambient; flow: 21 mL/min, eluent EtOH (0.2% v/v NH₃)] was carried out to give the title compounds.

HPLC-MS (A) Rt, 2.14 min; ESI+-MS m/z: 381.2 (M+1).

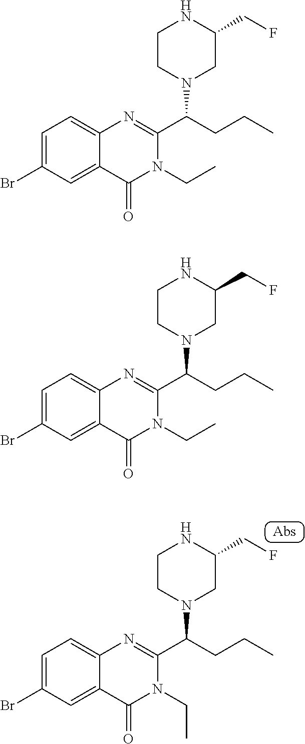 [SMILES: CCC[C@H](c1nc2ccc(Br)cc2c(=O)n1CC)N1CCN[C@H](CF)C1 ; CCC[C@@H](c1nc2ccc(Br)cc2c(=O)n1CC)N1CCN[C@@H](CF)C1 ; CCC[C@@H](c1nc2ccc(Br)cc2c(=O)n1CC)N1CCN[C@H](CF)C1]

### Examples 55, 56 and 57. 6-Bromo-3-ethyl-2-((R)-1-((S)-3-(fluoromethyl)piperazin-1-yl)butyl)quinazolin-4(3H)-one and 6-bromo-3-ethyl-2-((S)-1-((R)-3-(fluoromethyl)piperazin-1-yl)butyl)quinazolin-4(3H)-one and 6-bromo-3-ethyl-2-((S)-1-((S)-3-(fluoromethyl)piperazin-1-yl)butyl)quinazolin-4(3H)-one

Starting from 6-bromo-3-ethyl-2-(1-(3-(fluoromethyl)piperazin-1-yl)butyl)quinazolin-4(3H)-one, a chiral preparative HPLC separation [column: Chiralpak IG, temperature: ambient; flow: 15 mL/min, eluent n-Heptane/(EtOH+0.33% DEA) 70/30 v/v; Rt₁: 9.3′, Rt₂: 11.0′, Rt₃: 11.0′, Rt₄:15.7′] was carried out to give the title compounds.

HPLC-MS (A) Rt, 1.35 min; ESI+-MS m/z: 425.0 (M+1).

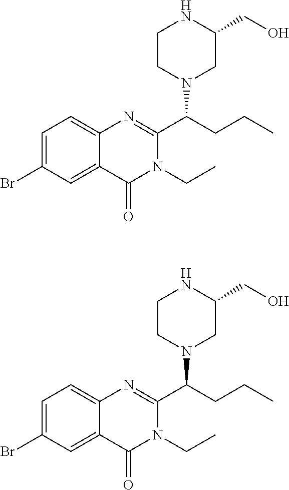 [SMILES: CCC[C@H](c1nc2ccc(Br)cc2c(=O)n1CC)N1CCN[C@H](CO)C1 ; CCC[C@@H](c1nc2ccc(Br)cc2c(=O)n1CC)N1CCN[C@H](CO)C1]

### Examples 58 and 59. 6-Bromo-3-ethyl-2-((R)-1-((S)-3-(hydroxymethyl)piperazin-1-yl)butyl)quinazolin-4(3H)-one and 6-bromo-3-ethyl-2-((S)-1-((S)-3-(hydroxymethyl)piperazin-1-yl)butyl)quinazolin-4(3H)-one

Starting from 6-bromo-3-ethyl-2-(1-((S)-3-(hydroxymethyl)piperazin-1-yl)butyl)quinazolin-4(3H)-one, a chiral preparative HPLC separation [column: Chiralpak AD-H, temperature: ambient; flow: 11 mL/min, eluent n-Heptane/(EtOH+0.33% DEA) 70/30 v/v; Rt₁: 9.4′, Rt₂: 15.8′] was carried out to give the title compounds.

HPLC-MS (A) Rt, 1.94 min; ESI+-MS m/z: 423.1 (M+1).

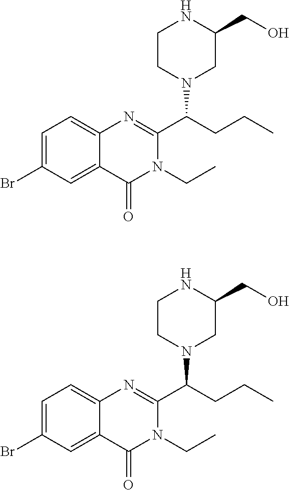 [SMILES: CCC[C@H](c1nc2ccc(Br)cc2c(=O)n1CC)N1CCN[C@@H](CO)C1 ; CCC[C@@H](c1nc2ccc(Br)cc2c(=O)n1CC)N1CCN[C@@H](CO)C1]

### Examples 60 and 61. 6-bromo-3-ethyl-2-((R)-1-((R)-3-(hydroxymethyl)piperazin-1-yl)butyl)quinazolin-4(3H)-one and 6-bromo-3-ethyl-2-((S)-1-((R)-3-(hydroxymethyl)piperazin-1-yl)butyl)quinazolin-4(3H)-one

Starting from 6-bromo-3-ethyl-2-(1-((R)-3-(hydroxymethyl)piperazin-1-yl)butyl)quinazolin-4(3H)-one, a chiral preparative HPLC separation [column: Chiralpak AD-H, temperature: ambient; flow: 12 mL/min, eluent n-Heptane/(IPA+0.33% DEA) 80/20 v/v; Rt₁: 12.5′, Rt₂: 17.6′] was carried out to give the title compounds.

HPLC-MS (A) Rt, 1.94 min; ESI+-MS m/z: 423.1 (M+1).

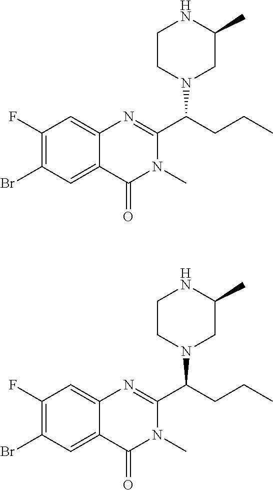 [SMILES: CCC[C@H](c1nc2cc(F)c(Br)cc2c(=O)n1C)N1CCN[C@@H](C)C1 ; CCC[C@@H](c1nc2cc(F)c(Br)cc2c(=O)n1C)N1CCN[C@@H](C)C1]

### Examples 62 and 63. 6-Bromo-7-fluoro-3-methyl-2-((R)-1-((S)-3-methylpiperazin-1-yl)butyl)quinazolin-4(3H)-one and 6-bromo-7-fluoro-3-methyl-2-((S)-1-((S)-3-methylpiperazin-1-yl)butyl)quinazolin-4(3H)-one

Starting from 6-bromo-7-fluoro-3-methyl-2-(1-((S)-3-methylpiperazin-1-yl)butyl)quinazolin-4(3H)-one, a chiral preparative HPLC separation [column: Chiralpak AD-H, temperature: ambient; flow: 13 mL/min, eluent n-Heptane/(EtOH+0.33% DEA) 95/5 v/v; Rt₁: 14.5′, Rt₂: 23.32′] was carried out to give the title compounds.

HPLC-MS (A) Rt, 1.98 min; ESI+-MS m/z: 411.2 (M+1).

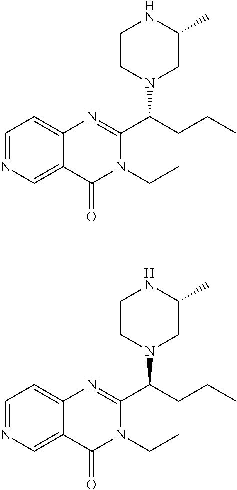 [SMILES: CCC[C@H](c1nc2ccncc2c(=O)n1CC)N1CCN[C@H](C)C1 ; CCC[C@@H](c1nc2ccncc2c(=O)n1CC)N1CCN[C@H](C)C1]

### Examples 64 and 65. 3-Ethyl-2-((R)-1-((R)-3-methylpiperazin-1-yl)butyl)pyrido[4,3-d]pyrimidin-4(3H)-one and 3-ethyl-2-((S)-1-((R)-3-methylpiperazin-1-yl)butyl)pyrido[4,3-d]pyrimidin-4(3H)-one

Starting from 3-methyl-2-(1-((R)-3-methylpiperazin-1-yl)butyl)pyrido[4,3-d]pyrimidin-4(3H)-one, a chiral preparative HPLC separation [column: Chiralpak IA, temperature: ambient; flow: 14 mL/min, eluent n-Heptane/(EtOH+0.33% DEA) 80/20 v/v; Rttr₁: 8.7′, Rt₂: 15.1′] was carried out to give the title compounds.

HPLC-MS (A) Rt, 1.35 min; ESI+-MS m/z: 330.2 (M+1).

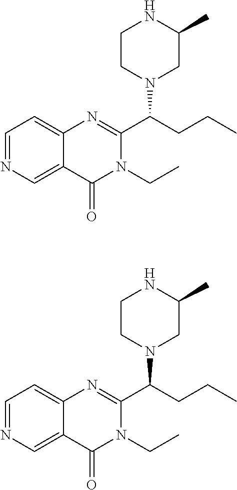 [SMILES: CCC[C@H](c1nc2ccncc2c(=O)n1CC)N1CCN[C@@H](C)C1 ; CCC[C@@H](c1nc2ccncc2c(=O)n1CC)N1CCN[C@@H](C)C1]

### Examples 66 and 67. 3-Ethyl-2-((R)-1-((S)-3-methylpiperazin-1-yl)butyl)pyrido[4,3-d]pyrimidin-4(3H)-one and 3-ethyl-2-((S)-1-((S)-3-methylpiperazin-1-yl)butyl)pyrido[4,3-d]pyrimidin-4(3H)-one

Starting from 3-ethyl-2-(1-((S)-3-methylpiperazin-1-yl)butyl)pyrido[4,3-d]pyrimidin-4(3H)-one, a chiral preparative HPLC separation [(column: Chiralpak AD-H, temperature: ambient; flow: 12 mL/min, eluent n-Heptane/(EtOH+0.33% DEA) 70/30 v/v; Rt₁: 7.1′, Rt₂: 13.9′] was carried out to give the title compounds.

HPLC-MS (A) Rt, 1.39 min; ESI+-MS m/z: 330.2 (M+1).

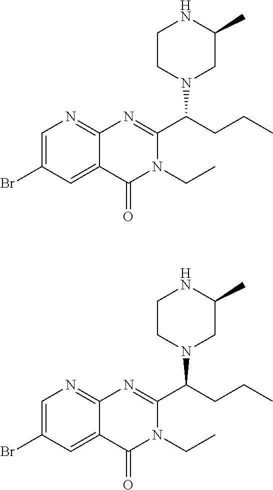 [SMILES: CCC[C@H](c1nc2ncc(Br)cc2c(=O)n1CC)N1CCN[C@@H](C)C1 ; CCC[C@@H](c1nc2ncc(Br)cc2c(=O)n1CC)N1CCN[C@@H](C)C1]

### Examples 68 and 69. 6-Bromo-3-ethyl-2-((R)-1-((S)-3-methylpiperazin-1-yl)butyl)pyrido[2,3-d]pyrimidin-4(3H)-one and 6-bromo-3-ethyl-2-((S)-1-((S)-3-methylpiperazin-1-yl)butyl)pyrido[2,3-d]pyrimidin-4(3H)-one

Starting from 6-bromo-3-ethyl-2-(1-((S)-3-methylpiperazin-1-yl)butyl)pyrido[2,3-d]pyrimidin-4(3H)-one, a chiral preparative SCF separation [column: Chiralpak IG, (20 mm×250 mm, 5 um), temperature: ambient; flow: 50 mL/min, Isocratic Conditions 25:75 MeOH:CO₂ (0.5% v/v DEA)] was carried out to give the title compounds.

HPLC-MS (A) Rt, 1.66 min; ESI+-MS m/z: 408.2 (M+1).

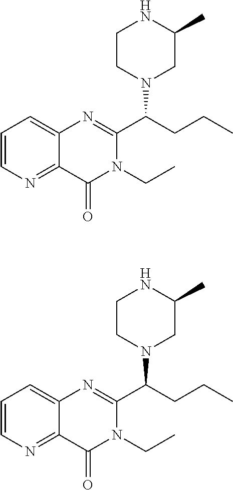 [SMILES: CCC[C@H](c1nc2cccnc2c(=O)n1CC)N1CCN[C@@H](C)C1 ; CCC[C@@H](c1nc2cccnc2c(=O)n1CC)N1CCN[C@@H](C)C1]

### Examples 70 and 71. 3-Ethyl-2-((R)-1-((S)-3-methylpiperazin-1-yl)butyl)pyrido[3,2-d]pyrimidin-4(3H)-one and 3-ethyl-2-((S)-1-((S)-3-methylpiperazin-1-yl)butyl)pyrido[3,2-d]pyrimidin-4(3H)-one

Starting from 3-ethyl-2-(1-((S)-3-methylpiperazin-1-yl)butyl)pyrido[3,2-d]pyrimidin-4(3H)-one, a chiral preparative HPLC separation [column: Chiralpak AD-H, temperature: ambient; flow: 12 mL/min, eluent n-Heptane/(EtOH+0.33% DEA) 80/20 v/v; Rt₁: 7.6′, Rt₂: 10.2′] was carried out to give the title compounds.

HPLC-MS (A) Rt, 1.31 min; ESI+-MS m/z: 330.2 (M+1).

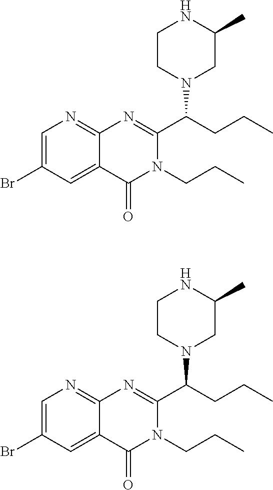 [SMILES: CCC[C@H](c1nc2ncc(Br)cc2c(=O)n1CCC)N1CCN[C@@H](C)C1 ; CCC[C@@H](c1nc2ncc(Br)cc2c(=O)n1CCC)N1CCN[C@@H](C)C1]

### Examples 72 and 73. 6-Bromo-2-((R)-1-((S)-3-methylpiperazin-1-yl)butyl)-3-propylpyrido[2,3-d]pyrimidin-4(3H)-one and 6-bromo-2-((S)-1-((S)-3-methylpiperazin-1-yl)butyl)-3-propylpyrido[2,3-d]pyrimidin-4(3H)-one

Starting from 6-bromo-2-(1-((S)-3-methylpiperazin-1-yl)butyl)-3-propylpyrido[2,3-d]pyrimidin-4(3H)-one, a chiral preparative HPLC separation [column: Chiralpak AD-H, temperature: ambient; flow: 14 mL/min, eluent n-Heptane/(EtOH+0.33% DEA) 90/10 v/v; Rt₁: 8.9′, Rt₂: 10.6′] was carried out to give the title compounds.

HPLC-MS (A) Rt, 1.83 min; ESI+-MS m/z: 422.2 (M+1).

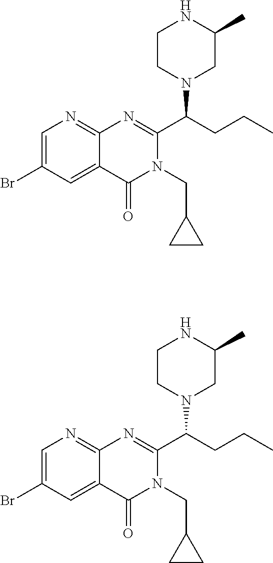 [SMILES: CCC[C@@H](c1nc2ncc(Br)cc2c(=O)n1CC1CC1)N1CCN[C@@H](C)C1 ; CCC[C@H](c1nc2ncc(Br)cc2c(=O)n1CC1CC1)N1CCN[C@@H](C)C1]

### Examples 74 and 75. 6-Bromo-3-(cyclopropylmethyl)-2-((S)-1-((S)-3-methylpiperazin-1-yl)butyl)pyrido[2,3-d]pyrimidin-4(3H)-one and 6-bromo-3-(cyclopropylmethyl)-2-((R)-1-((S)-3-methylpiperazin-1-yl)butyl)pyrido[2,3-d]pyrimidin-4(3H)-one

Starting from 6-bromo-3-(cyclopropylmethyl)-2-(1-((S)-3-methylpiperazin-1-yl)butyl)pyrido[2,3-d]pyrimidin-4(3H)-one, a chiral preparative HPLC separation [column: Chiralpak AD-H, temperature: ambient; flow: 13 mL/min, eluent n-Heptane/(IPA+0.33% DEA) 90/10 v/v; Rt₁: 11.2′, Rt₂: 12.8′] was carried out to give the title compounds.

HPLC-MS (B) Rt, 1.90 min; ESI+-MS m/z: 434.2 (M+1).

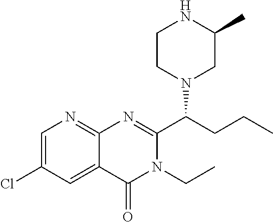 [SMILES: CCC[C@H](c1nc2ncc(Cl)cc2c(=O)n1CC)N1CCN[C@@H](C)C1]

### Example 76. 6-Chloro-3-ethyl-2-((R)-1-((S)-3-methylpiperazin-1-yl)butyl)pyrido[2,3-d]pyrimidin-4(3H)-one

Starting from 6-chloro-3-ethyl-2-(1-((S)-3-methylpiperazin-1-yl)butyl)pyrido[2,3-d]pyrimidin-4(3H)-one, a chiral preparative HPLC separation [column: Chiralpak AS-H, temperature: ambient; flow: 14 mL/min, eluent n-Heptane/(EtOH+0.33% DEA) 95/5 v/v; Rt₁: 7.5′] was carried out to give the title compound, while its diastereoisomer was not isolated.

HPLC-MS (A) Rt, 1.63 min; ESI+-MS m/z: 364.2 (M+1).

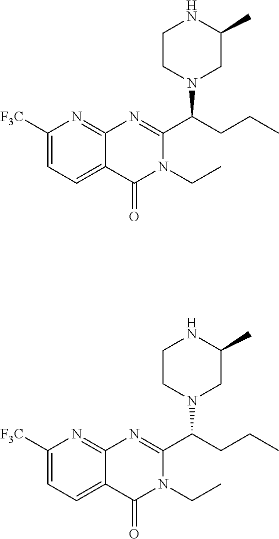 [SMILES: CCC[C@@H](c1nc2nc(C(F)(F)F)ccc2c(=O)n1CC)N1CCN[C@@H](C)C1 ; CCC[C@H](c1nc2nc(C(F)(F)F)ccc2c(=O)n1CC)N1CCN[C@@H](C)C1]

### Examples 77 and 78. 3-Ethyl-2-((S)-1-((S)-3-methylpiperazin-1-yl)butyl)-7-(trifluoromethyl)pyrido[2,3-d]pyrimidin-4(3H)-one and 3-ethyl-2-((R)-1-((S)-3-methylpiperazin-1-yl)butyl)-7-(trifluoromethyl)pyrido[2,3-d]pyrimidin-4(3H)-one

Starting from 3-ethyl-2-(1-((S)-3-methylpiperazin-1-yl)butyl)-7-(trifluoromethyl)pyrido[2,3-d]pyrimidin-4(3H)-one, a chiral preparative HPLC separation [column: Chiralpak IC, temperature: ambient; flow: 13 mL/min, eluent n-Heptane/(EtOH+0.33% DEA) 95/5 v/v; Rt₁: 10.3′, Rt₂: 11.8′] was carried out to give the title compounds.

HPLC-MS (A) Rt, 1.78 min; ESI+-MS m/z: 398.1 (M+1).

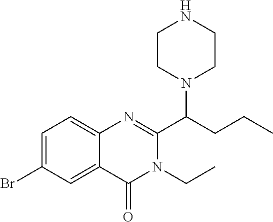 [SMILES: CCCC(c1nc2ccc(Br)cc2c(=O)n1CC)N1CCNCC1]

### Example 79. 6-Bromo-3-ethyl-2-(1-(piperazin-1-yl)butyl)quinazolin-4(3H)-one

### Step a. tert-Butyl 4-(1-(6-bromo-3-ethyl-4-oxo-3,4-dihydroquinazolin-2-yl)butyl)piperazine-1-carboxylate

Starting from the product obtained in step d of example 1 and following the procedure described in step e of example 1, the title compound was obtained (174 mg, Yield: 34%).

### Step b. Title Compound

To a solution of the compound obtained in step a (30 mg, 0.06 mmol) in anh DCM (2 mL), TFA (0.5 mL) was added and the mixture was stirred at r.t. overnight. The reaction mixture was neutralized with aq NaHCO₃ sat sol, and the organic layer was dried over anh Na₂SO₄, filtered, and concentrated to dryness to give the title compound (18 mg, Yield: 76%).

HPLC-MS (F) Rt, 1.85 min; ESI+-MS m/z: 393.1 (M+1).

This method was used for the preparation of examples 80-94 using suitable starting materials:

| | | CHEMICAL | Rt | MS | HPLC |
|---|---|---|---|---|---|
| STRUCTURE | EX | NAME | (min) | (M + H) | Method |
| 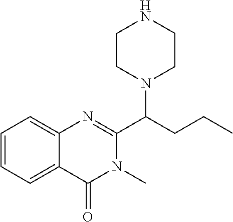 [SMILES: CCCC(c1nc2ccccc2c(=O)n1C)N1CCNCC1] | 80 | 3-methyl-2-(1- (piperazin-1- yl)butyl)quinazolin- 4(3H)-one | 1.34 | 301.1 | C |
| 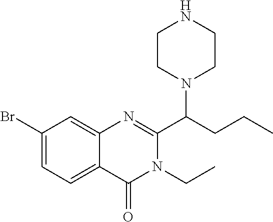 [SMILES: CCCC(c1nc2cc(Br)ccc2c(=O)n1CC)N1CCNCC1] | 81 | 7-bromo-3-ethyl-2-(1- (piperazin-1- yl)butyl)quinazolin- 4(3H)-one | 3.51 | 393.1 | D |
| 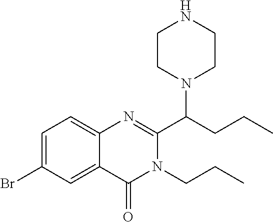 [SMILES: CCCC(c1nc2ccc(Br)cc2c(=O)n1CCC)N1CCNCC1] | 82 | 6-bromo-2-(1- (piperazin-1-yl)butyl)- 3-propylquinazolin- 4(3H)-one | 2.12 | 47.1 | A |
| 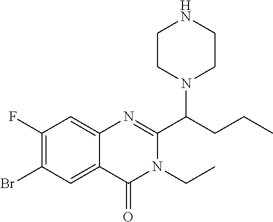 [SMILES: CCCC(c1nc2cc(F)c(Br)cc2c(=O)n1CC)N1CCNCC1] | 83 | 6-bromo-3-ethyl-7- fluoro-2-(1-(piperazin- 1-yl)butyl)quinazolin- 4(3H)-one | 2.04 | 411.1 | A |
| 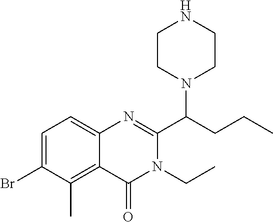 [SMILES: CCCC(c1nc2ccc(Br)c(C)c2c(=O)n1CC)N1CCNCC1] | 84 | 6-bromo-3-ethyl-5- methyl-2-(1-(piperazin- 1-yl)butyl)quinazolin- 4(3H)-one | 2.26 | 407.1 | A |
| 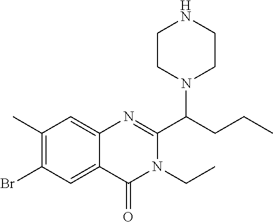 [SMILES: CCCC(c1nc2cc(C)c(Br)cc2c(=O)n1CC)N1CCNCC1] | 85 | 6-bromo-3-ethyl-7- methyl-2-(1-(piperazin- 1-yl)butyl)quinazolin- 4(3H)-one | 2.02 | 375.2 | A |
| 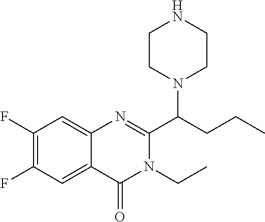 [SMILES: CCCC(c1nc2cc(F)c(F)cc2c(=O)n1CC)N1CCNCC1] | 86 | 3-ethyl-6,7-difluoro-2- (1-(piperazin-1- yl)butyl)quinazolin- 4(3H)-one | 1.92 | 351.1 | A |
| 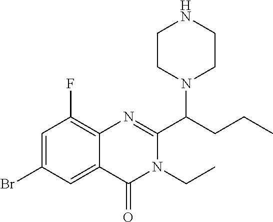 [SMILES: CCCC(c1nc2c(F)cc(Br)cc2c(=O)n1CC)N1CCNCC1] | 87 | 6-bromo-3-ethyl-8- fluoro-2-(1-(piperazin- 1-yl)butyl)quinazolin- 4(3H)-one | 2.12 | 411.1 | A |
| 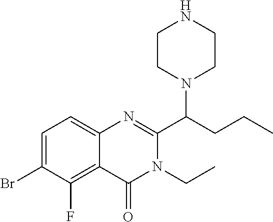 [SMILES: CCCC(c1nc2ccc(Br)c(F)c2c(=O)n1CC)N1CCNCC1] | 88 | 6-bromo-3-ethyl-5- fluoro-2-(1-(piperazin- 1-yl)butyl)quinazolin- 4(3H)-one | 1.99 | 411.1 | A |
| 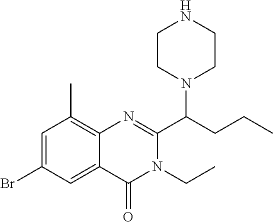 [SMILES: CCCC(c1nc2c(C)cc(Br)cc2c(=O)n1CC)N1CCNCC1] | 89 | 6-bromo-3-ethyl-8- methyl-2-(1-(piperazin- 1-yl)butyl)quinazolin- 4(3H)-one | 2.31 | 407.1 | A |
| 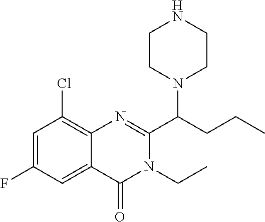 [SMILES: CCCC(c1nc2c(Cl)cc(F)cc2c(=O)n1CC)N1CCNCC1] | 90 | 8-chloro-3-ethyl-6- fluoro-2-(1-(piperazin- 1-yl)butyl)quinazolin- 4(3H)-one | 2.12 | 367.1 | A |
| 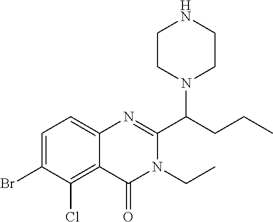 [SMILES: CCCC(c1nc2ccc(Br)c(Cl)c2c(=O)n1CC)N1CCNCC1] | 91 | 6-bromo-5-chloro-3- ethyl-2-(1-(piperazin- 1-yl)butyl)quinazolin- 4(3H)-one | 2.08 | 427.1 | A |
| 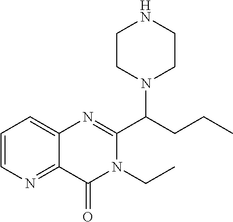 [SMILES: CCCC(c1nc2cccnc2c(=O)n1CC)N1CCNCC1] | 92 | 3-ethyl-2-(1- (piperazin-1- yl)butyl)pyrido[3,2- d]pyrimidin-4(3H)-one | 1.20 | 316.2 | A |
| 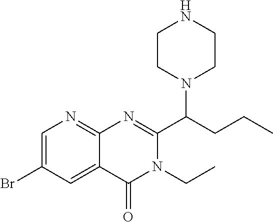 [SMILES: CCCC(c1nc2ncc(Br)cc2c(=O)n1CC)N1CCNCC1] | 93 | 6-bromo-3-ethyl-2-(1- (piperazin-1- yl)butyl)pyrido[2,3- d]pyrimidin-4(3H)-one | 1.56 | 394.2 | A |
| 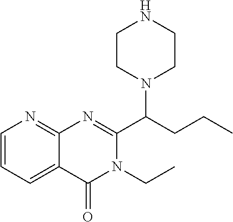 [SMILES: CCCC(c1nc2ncccc2c(=O)n1CC)N1CCNCC1] | 94 | 3-ethyl-2-(1- (piperazin-1- yl)butyl)pyrido[2,3- d]pyrimidin-4(3H)-one | 1.25 | 316.2 | A |

### Examples 95 and 96. (S)-6-bromo-3-ethyl-8-fluoro-2-(1-(piperazin-1-yl)butyl)quinazolin-4(3H)-one and (R)-6-bromo-3-ethyl-8-fluoro-2-(1-(piperazin-1-yl)butyl)quinazolin-4(3H)-one

Starting from the compound obtained in example 87, a chiral preparative HPLC separation [column: Chiralpak AD-H, temperature: ambient; flow: 12 mL/min, eluent n-Heptane/(EtOH+0.33% DEA) 70/30 v/v; Rt₁: 7.1′, Rt₂: 14.5′] was carried out to give the title compounds.

The following compounds were obtained using the same method described in Example 79, but directly separating the enantiomeric or diastereomeric mixtures using chiral HPLC:

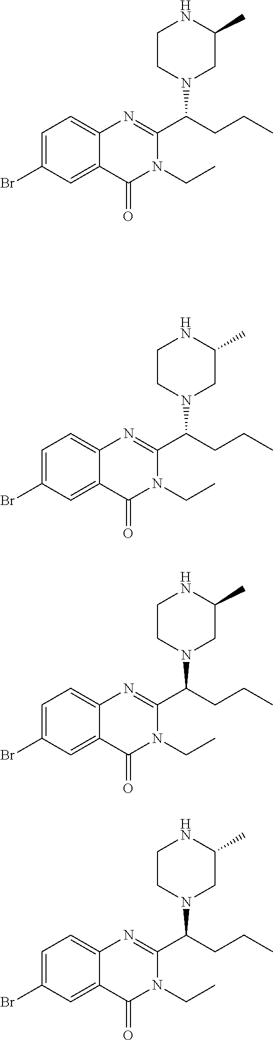 [SMILES: CCC[C@H](c1nc2ccc(Br)cc2c(=O)n1CC)N1CCN[C@@H](C)C1 ; CCC[C@H](c1nc2ccc(Br)cc2c(=O)n1CC)N1CCN[C@H](C)C1 ; CCC[C@@H](c1nc2ccc(Br)cc2c(=O)n1CC)N1CCN[C@@H](C)C1 ; CCC[C@@H](c1nc2ccc(Br)cc2c(=O)n1CC)N1CCN[C@H](C)C1]

### Examples 97, 98, 99 and 100. 6-Bromo-3-ethyl-2-((R)-1-((S)-3-methylpiperazin-1-yl)butyl)quinazolin-4(3H)-one, 6-bromo-3-ethyl-2-((R)-1-((R)-3-methylpiperazin-1-yl)butyl)quinazolin-4(3H)-one, 6-bromo-3-ethyl-2-((S)-1-((S)-3-methylpiperazin-1-yl)butyl)quinazolin-4(3H)-one and 6-bromo-3-ethyl-2-((S)-1-((R)-3-methylpiperazin-1-yl)butyl)quinazolin-4(3H)-one

Starting from 6-bromo-3-ethyl-2-(1-(3-methylpiperazin-1-yl)butyl)quinazolin-4(3H)-one, a chiral preparative HPLC separation [column: Chiralpak AD-H, temperature: ambient; flow: 13 mL/min, eluent n-Heptane/(IPA+0.33% DEA) 95/5 v/v; Rt₁: 12.2′, Rt₂: 15.9′, Rt₃: 18.8′, Rt₄: 22.1′] was carried out to give the title compounds.

HPLC-MS (A) Rt, 2.15 min; ESI+-MS m/z: 407.1 (M+1).

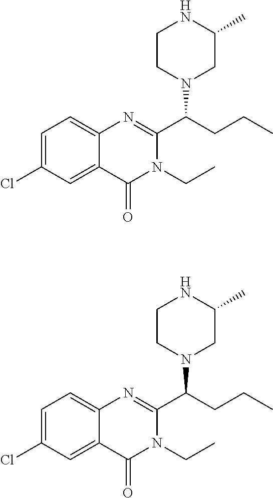 [SMILES: CCC[C@H](c1nc2ccc(Cl)cc2c(=O)n1CC)N1CCN[C@H](C)C1 ; CCC[C@@H](c1nc2ccc(Cl)cc2c(=O)n1CC)N1CCN[C@H](C)C1]

### Examples 101 and 102. 6-Chloro-3-ethyl-2-((R)-1-((R)-3-methylpiperazin-1-yl)butyl)quinazolin-4(3H)-one and 6-chloro-3-ethyl-2-((S)-1-((R)-3-methylpiperazin-1-yl)butyl)quinazolin-4(3H)-one

Starting from 6-chloro-3-ethyl-2-(1-((R)-3-methylpiperazin-1-yl)butyl)quinazolin-4(3H)-one a chiral preparative HPLC separation [column: Chiralpak AD-H, temperature: ambient; flow: 0.8 mL/min, eluent n-Heptane/(EtOH+0.33% DEA) 70/30 v/v; Rt₁: 5.6′, Rt₂: 7.2′] was carried out to give the title compounds.

HPLC-MS (A) Rt, 2.0 min; ESI+-MS m/z: 363.2 (M+1).

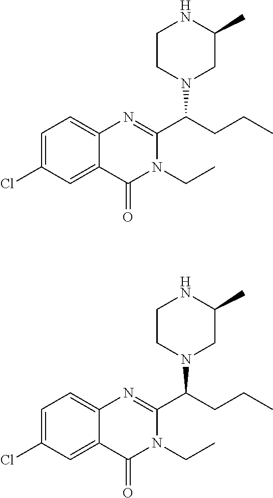 [SMILES: CCC[C@H](c1nc2ccc(Cl)cc2c(=O)n1CC)N1CCN[C@@H](C)C1 ; CCC[C@@H](c1nc2ccc(Cl)cc2c(=O)n1CC)N1CCN[C@@H](C)C1]

### Examples 103 and 104. 6-Chloro-3-ethyl-2-((R)-1-((S)-3-methylpiperazin-1-yl)butyl)quinazolin-4(3H)-one and 6-chloro-3-ethyl-2-((S)-1-((S)-3-methylpiperazin-1-yl)butyl)quinazolin-4(3H)-one

Starting from 6-chloro-3-ethyl-2-(1-((S)-3-methylpiperazin-1-yl)butyl)quinazolin-4(3H)-one, a chiral preparative HPLC separation [column: Chiralpak AD-H, temperature: ambient; flow: 11 mL/min, eluent n-Heptane/(EtOH+0.33% DEA) 70/30 v/v; Rt₁: 6.3′, Rt₂: 11.9′] was carried out to give the title compounds.

HPLC-MS (A) Rt, 2.0 min; ESI+-MS m/z: 363.2 (M+1).

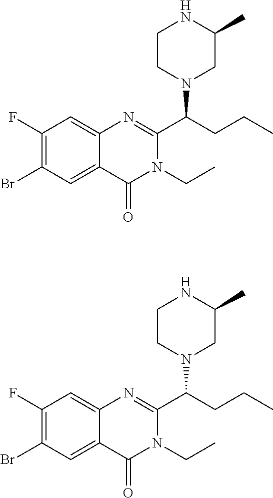 [SMILES: CCC[C@@H](c1nc2cc(F)c(Br)cc2c(=O)n1CC)N1CCN[C@@H](C)C1 ; CCC[C@H](c1nc2cc(F)c(Br)cc2c(=O)n1CC)N1CCN[C@@H](C)C1]

### Examples 105 and 106. 6-Bromo-3-ethyl-7-fluoro-2-((S)-1-((S)-3-methylpiperazin-1-yl)butyl)quinazolin-4(3H)-one and 6-bromo-3-ethyl-7-fluoro-2-((R)-1-((S)-3-methylpiperazin-1-yl)butyl)quinazolin-4(3H)-one

Starting from 6-bromo-3-ethyl-7-fluoro-2-(1-((S)-3-methylpiperazin-1-yl)butyl)quinazolin-4(3H)-one, a chiral preparative HPLC separation [column: Chiralpak AD-H, temperature: ambient; flow: 12 mL/min, eluent n-Heptane/(EtOH+0.33% DEA) 75/25 v/v; Rt₁: 5.6′, Rt₂: 12.4′] was carried out to give the title compounds.

HPLC-MS (A) Rt, 2.12 min; ESI+-MS m/z: 425.2 (M+1).

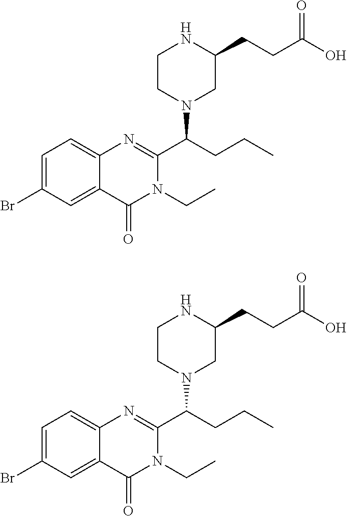 [SMILES: CCC[C@@H](c1nc2ccc(Br)cc2c(=O)n1CC)N1CCN[C@@H](CCC(=O)O)C1 ; CCC[C@H](c1nc2ccc(Br)cc2c(=O)n1CC)N1CCN[C@@H](CCC(=O)O)C1]

### Examples 107 and 108. 3-((S)-4-((S)-1-(6-Bromo-3-ethyl-4-oxo-3,4-dihydroquinazolin-2-yl)butyl)piperazin-2-yl)propanoic acid and 3-((S)-4-((R)-1-(6-bromo-3-ethyl-4-oxo-3,4-dihydroquinazolin-2-yl)butyl)piperazin-2-yl)propanoic acid

Starting from 3-((2S)-4-(1-(6-bromo-3-ethyl-4-oxo-3,4-dihydroquinazolin-2-yl)butyl)piperazin-2-yl)propanoic acid, a chiral preparative HPLC separation [column: Chiralpak AD-H, temperature: ambient; flow: 13 mL/min, eluent n-Heptane/(IPA+0.33% DEA) 85/15 v/v; Rt₁: 7.6.′, Rt₂: 9.4′] was carried out to give the title compounds.

HPLC-MS (A) Rt, 1.73 min; ESI+-MS m/z: 465.2 (M+1).

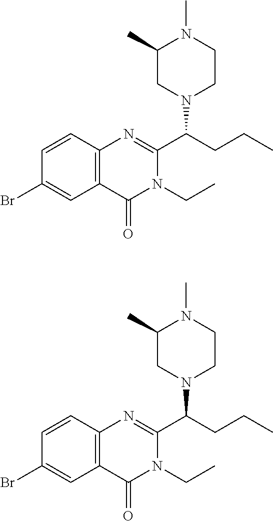 [SMILES: CCC[C@H](c1nc2ccc(Br)cc2c(=O)n1CC)N1CCN(C)[C@H](C)C1 ; CCC[C@@H](c1nc2ccc(Br)cc2c(=O)n1CC)N1CCN(C)[C@H](C)C1]

### Examples 109 and 110. 6-Bromo-2-((R)-1-((R)-3,4-dimethylpiperazin-1-yl)butyl)-3-ethylquinazolin-4(3H)-one and 6-bromo-2-((S)-1-((R)-3,4-dimethylpiperazin-1-yl)butyl)-3-ethylquinazolin-4(3H)-one

Starting from 6-bromo-2-(1-((R)-3,4-dimethylpiperazin-1-yl)butyl)-3-ethylquinazolin-4(3H)-one a chiral preparative HPLC separation [column: Chiralpak IA, temperature: ambient; flow: 13 mL/min, eluent n-Heptane/(IPA+0.33% DEA) 70/30 v/v; Rt₁:5.2.′, Rt₂: 7.2′] was carried out to give the title compounds.

HPLC-MS (A) Rt, 2.52 min; ESI+-MS m/z: 421.2 (M+1).

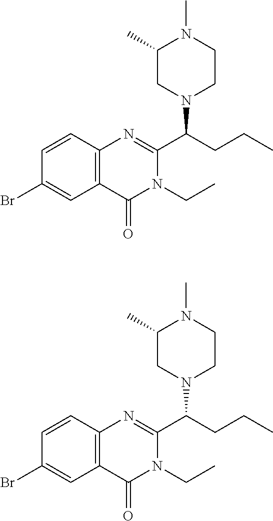 [SMILES: CCC[C@@H](c1nc2ccc(Br)cc2c(=O)n1CC)N1CCN(C)[C@@H](C)C1 ; CCC[C@H](c1nc2ccc(Br)cc2c(=O)n1CC)N1CCN(C)[C@@H](C)C1]

### Examples 111 and 112. 6-Bromo-2-((S)-1-((S)-3,4-dimethylpiperazin-1-yl)butyl)-3-ethylquinazolin-4(3H)-one and 6-bromo-2-((R)-1-((S)-3,4-dimethylpiperazin-1-yl)butyl)-3-ethylquinazolin-4(3H)-one

Starting from 6-bromo-2-(1-((S)-3,4-dimethylpiperazin-1-yl)butyl)-3-ethylquinazolin-4(3H)-one a chiral separation [column: SFC Lux C2 (21.2 mm×250 mm, 5 um), temperature: ambient; flow: 50 mL/min, isocratic Conditions 35:65 MeOH:CO₂ (0.2% v/v NH₃] was carried out to give the title compounds.

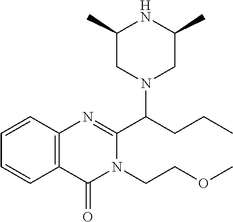 [SMILES: CCCC(c1nc2ccccc2c(=O)n1CCOC)N1C[C@@H](C)N[C@@H](C)C1]

### Example 113. 2-(1-((3S,5R)-3,5-Dimethylpiperazin-1-yl)butyl)-3-(2-methoxyethyl)quinazolin-4(3H)-one

### Step a. 2-Amino-N-(2-methoxyethyl)benzamide

To a solution of 1H-benzo[d][1,3]oxazine-2,4-dione (2 g, 12.3 mmol) in anh ACN (30 mL), 2-methoxyethanamine (1.2 mL, 13.5 mmol) was added and the mixture was stirred at r.t. for 2 h and at 50° C. for 16 h. The solvent was removed under vacuum to give the title product (1.7 g, Yield: 72%).

### Step b. 2-Butyl-3-(2-methoxyethyl)quinazolin-4(3H)-one

To a solution of the compound obtained in step a (0.25 g, 1.3 mmol) in glacial acetic acid (5 mL), pentanoyl chloride (0.2 mL, 1.8 mmol) was added drop wise and the mixture was refluxed overnight. The solvent was removed under vacuum, the residue was neutralized with NaOH 10% aq sol, the product was extracted with EtOAc and washed with brine. The combined organic layers were dried over Na₂SO₄, filtered and concentrated to dryness. The crude product was purified by flash chromatography, silica gel, gradient Chx to Chx:EtOAc (8:2) to give the title compound (55 mg, Yield: 16%).

### Step c. 2-(1-Bromobutyl)-3-(2-methoxyethyl)quinazolin-4(3H)-one

Starting from the compound obtained in step b (55 mg, 0.2 mmol) and following the procedure described in step d of example 1, the title compound was obtained (71 mg, Yield: 99%).

### Step d. Title Compound

Starting from the compound obtained in step c (71 mg, 0.2 mmol) and following the procedure described in step e of example 1, the title compound was obtained (24 mg, Yield: 44%).

HPLC-MS (C) Rt, 1.86 min; ESI+-MS m/z: 373.4 (M+1).

This method was used for the preparation of examples 114-122 using suitable starting materials:

| | | CHEMICAL | Rt | MS | HPLC |
|---|---|---|---|---|---|
| STRUCTURE | EX | NAME | (min) | (M + H) | Method |
| 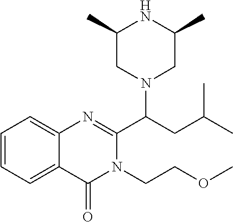 [SMILES: COCCn1c(C(CC(C)C)N2C[C@@H](C)N[C@@H](C)C2)nc2ccccc2c1=O] | 114 | 2-(1-((3S,5R)-3,5- dimethylpiperazin-1- yl)-3-methylbutyl)-3-(2- methoxyethyl) quinazolin-4(3H)-one | 2.09 | 387.4 | C |
| 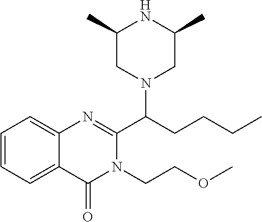 [SMILES: CCCCC(c1nc2ccccc2c(=O)n1CCOC)N1C[C@@H](C)N[C@@H](C)C1] | 115 | 2-(1-((3S,5R)-3,5- dimethylpiperazin-1- yl)pentyl)-3-(2- methoxyethyl) quinazolin-4(3H)-one | 2.06 | 387.4 | C |
| 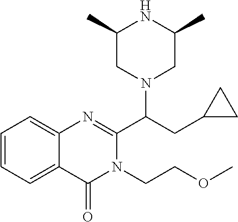 [SMILES: COCCn1c(C(CC2CC2)N2C[C@@H](C)N[C@@H](C)C2)nc2ccccc2c1=O] | 116 | 2-(2-cyclopropyl-1- ((3S,5R)-3,5- dimethylpiperazin-1- yl)ethyl)-3-(2- methoxyethyl) quinazolin-4(3H)-one | 1.87 | 385.4 | C |
| 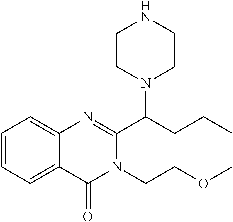 [SMILES: CCCC(c1nc2ccccc2c(=O)n1CCOC)N1CCNCC1] | 117 | 3-(2-methoxyethyl)-2- (1-(piperazin-1- yl)butyl)quinazolin- 4(3H)-one | 1.65 | 345.3 | C |
| 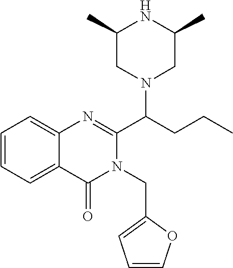 [SMILES: CCCC(c1nc2ccccc2c(=O)n1Cc1ccco1)N1C[C@@H](C)N[C@@H](C)C1] | 118 | 2-(1-((3S,5R)-3,5- dimethylpiperazin-1- yl)butyl)-3-(furan-2- ylmethyl)quinazolin- 4(3H)-one | 1.91 | 395.3 | C |
| 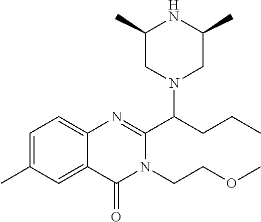 | 119 | 2-(1-((3S,5R)-3,5- dimethylpiperazin-1- yl)butyl)-3-(2- methoxyethyl)-6- methylquinazolin- 4(3H)-one | 1.93 | 387.3 | C |
| 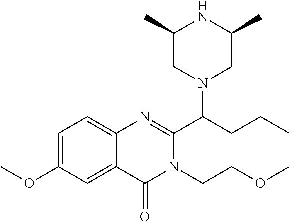 | 120 | 2-(1-((3S,5R)-3,5- dimethylpiperazin-1- yl)butyl)-6-methoxy-3- (2- methoxyethyl) quinazolin-4(3H)-one | 1.82 | 403.3 | C |
| 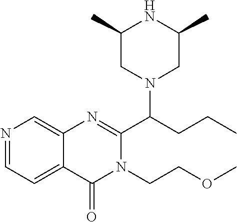 [SMILES: CCCC(c1nc2cnccc2c(=O)n1CCOC)N1C[C@@H](C)N[C@@H](C)C1] | 121 | 2-(1-((3S,5R)-3,5- dimethylpiperazin-1- yl)butyl)-3-(2- methoxyethyl)pyrido[3,4- d]pyrimidin-4(3H)- one | 1.47 | 374.3 | C |
| 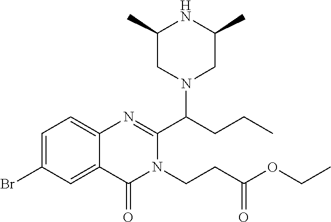 [SMILES: CCCC(c1nc2ccc(Br)cc2c(=O)n1CCC(=O)OCC)N1C[C@@H](C)N[C@@H](C)C1] | 122 | ethyl 3-(6-bromo-2-(1- ((3S,5R)-3,5- dimethylpiperazin-1- yl)butyl)-4- oxoquinazolin-3(4H)- yl)propanoate | 2.12 | 47.1 | A |

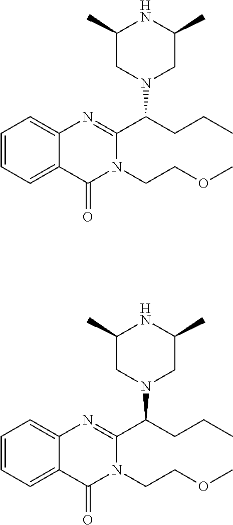 [SMILES: CCC[C@H](c1nc2ccccc2c(=O)n1CCOC)N1C[C@@H](C)N[C@@H](C)C1 ; CCC[C@@H](c1nc2ccccc2c(=O)n1CCOC)N1C[C@@H](C)N[C@@H](C)C1]

### Examples 123 and 124. 2-((R)-1-((3S,5R)-3,5-Dimethylpiperazin-1-yl)butyl)-3-(2-methoxyethyl)quinazolin-4(3H)-one and 2-((S)-1-((3S,5R)-3,5-dimethylpiperazin-1-yl)butyl)-3-(2-methoxyethyl)quinazolin-4(3H)-one

Starting from the compound obtained in example 113 a chiral preparative HPLC separation (column: Chiralpak AD-H, temperature: ambient; flow: 13 mL/min, eluent n-Heptane/(IPA+0.33% DEA) 90/10 v/v; Rt₁: 8.1′, Rt₂: 14.9′) was carried out to give the title compounds.

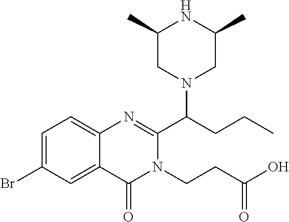 [SMILES: CCCC(c1nc2ccc(Br)cc2c(=O)n1CCC(=O)O)N1C[C@@H](C)N[C@@H](C)C1]

### Example 125. 3-(6-Bromo-2-(1-((3S,5R)-3,5-dimethylpiperazin-1-yl)butyl)-4-oxoquinazolin-3(4H)-yl)propanoic acid

To a solution of the compound obtained in example 122 (54 mg, 0.1 mmol) in MeOH (3 mL) at 0° C., LiOH (8 mg, 0.33 mmol) dissolved in MeOH (1 mL) was added, and the mixture was heated at 75° C. overnight. The solvent was removed under vacuum and the residue was dissolved in MeOH, passed through an ionic column SCX with a gradient of MeOH to NH₃ 2M in MeOH, to afford the title product (16 mg, Yield: 32%).

HPLC-MS (A) Rt, 1.49 min; ESI+-MS m/z: (465.2).

This method was used for the preparation of example 126 using suitable starting materials:

| | | CHEMICAL | Rt | MS | HPLC |
|---|---|---|---|---|---|
| STRUCTURE | EX | NAME | (min) | (M + H) | Method |
| 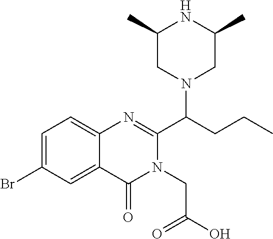 [SMILES: CCCC(c1nc2ccc(Br)cc2c(=O)n1CC(=O)O)N1C[C@@H](C)N[C@@H](C)C1] | 126 | 2-(6-bromo-2-(1- ((3S,5R)-3,5- dimethylpiperazin-1- yl)butyl)-4- oxoquinazolin-3(4H)- yl)acetic acid | 1.46 | 451.1 | A |

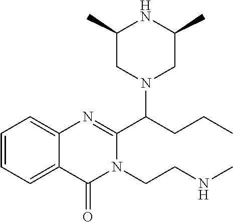 [SMILES: CCCC(c1nc2ccccc2c(=O)n1CCNC)N1C[C@@H](C)N[C@@H](C)C1]

### Example 127. 2-(1-((3S,5R)-3,5-Dimethylpiperazin-1-yl)butyl)-3-(2-(methylamino)ethyl)quinazolin-4(3H)-one

### Step a. tert-Butyl (2-(2-aminobenzamido)ethyl)(methyl)carbamate

Following the procedure described in step a of example 113, but using N-methylethylenediamine the title product was obtained (Yield: 80%).

### Step b. tert-Butyl methyl(2-(2-pentanamidobenzamido)ethyl)carbamate

Starting from the product obtained in step a (1.5 g, 5.1 mmol) and following the procedure described in step b of example 1, the title product was obtained (1.75 g, Yield: 91%).

### Step c. tert-Butyl (2-(2-butyl-4-oxoquinazolin-3(4H)-yl)ethyl)(methyl)carbamate

To a solution of the product obtained in step b (1.75 g, 4.6 mmol) in ethylene glycol (20 mL), lithium hydroxide monohydrate (0.22 g, 9.3 mmol) was added and the mixture was heated, in a sealed tube, at 130° C., overnight. The reaction mixture was cooled to r.t., diluted with DCM and washed with water. The organic layer was dried over anh Na₂SO₄, filtered and concentrated to dryness to give the title compound (1.3 g, Yield: 80%).

### Step d. tert-Butyl (2-(2-(1-bromobutyl)-4-oxoquinazolin-3(4H)-yl)ethyl)(methyl)carbamate

Starting from the product obtained in step c (50 mg, 0.14 mmol) and following the procedure described in step d of example 1, the title product was obtained (30 mg, Yield: 49%).

### Step e. tert-Butyl (2-(2-(1-((3S,5R)-3,5-dimethylpiperazin-1-yl)butyl)-4-oxoquinazolin-3(4H)-yl)ethyl)(methyl)carbamate

Starting from the product obtained in step d (0.79 g, 1.8 mmol) and following the procedure described in step e of example 1, the title product was obtained (0.35 g, Yield: 42%).

### Step f. Title Compound

Starting from the product obtained in step e (190 mg, 0.4 mmol) and following the procedure described in step b of example 79, the title compound was obtained (128 mg, Yield: 86%).

HPLC-MS (C) Rt, 1.54 min; ESI+-MS m/z: 372.3 (M+1).

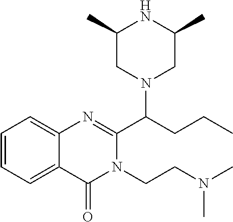 [SMILES: CCCC(c1nc2ccccc2c(=O)n1CCN(C)C)N1C[C@@H](C)N[C@@H](C)C1]

### Example 128. 3-(2-(Dimethylamino)ethyl)-2-(1-((3S,5R)-3,5-dimethylpiperazin-1-yl)butyl)quinazolin-4(3H)-one

To a solution of the compound obtained in example 127 (20 mg, 0.05 mmol) in MeOH (3 mL), K₂CO₃ (19 mg, 0.14 mmol) was added, and the mixture was stirred at r.t. for 10 min. Formaldehyde sol 37% wt in H₂O (16 μL, 0.2 mmol) was added and the reaction mixture was stirred at r.t. overnight. NaBH₄ (9 mg, 0.2 mmol) was added and the reaction mixture was stirred at r.t. for 16 h more. The solvent was removed under vacuum and the residue was dissolved in water and extracted with DCM. The combined organic layers were dried over anh Na₂SO₄, filtered and concentrated to dryness to give the title compound (9 mg, Yield: 24%).

HPLC-MS (C) Rt, 1.7 min; ESI+-MS m/z: 386.3 (M+1).

This method was used for the preparation of examples 129-130 using suitable starting materials:

| | | CHEMICAL | Rt | MS | HPLC |
|---|---|---|---|---|---|
| STRUCTURE | EX | NAME | (min) | (M + H) | Method |
| 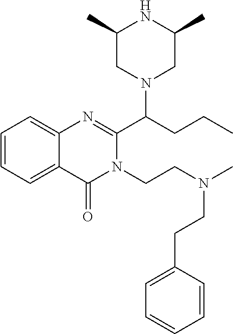 [SMILES: CCCC(c1nc2ccccc2c(=O)n1CCN(C)CCc1ccccc1)N1C[C@@H](C)N[C@@H](C)C1] | 129 | 2-(1-((3S,5R)-3,5- dimethylpiperazin-1- yl)butyl)-3-(2- (methyl(phenethyl) amino)ethyl)quinazolin- 4(3H)-one | 2.26 | 476.3 | C |
| 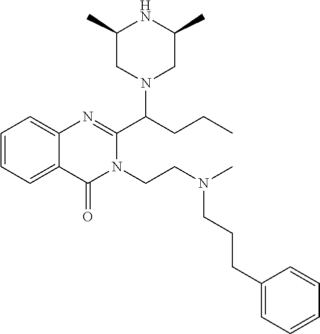 [SMILES: CCCC(c1nc2ccccc2c(=O)n1CCN(C)CCCc1ccccc1)N1C[C@@H](C)N[C@@H](C)C1] | 130 | 2-(1-((3S,5R)-3,5- dimethylpiperazin-1- yl)butyl)-3-(2- (methyl(3- phenylpropyl)amino) ethyl)quinazolin-4(3H)- one | 2.39 | 490.4 | C |

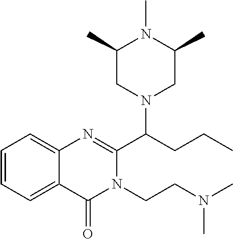 [SMILES: CCCC(c1nc2ccccc2c(=O)n1CCN(C)C)N1C[C@@H](C)N(C)[C@@H](C)C1]

### Example 131. 3-(2-(Dimethylamino)ethyl)-2-(1-((3S,5R)-3,4,5-trimethylpiperazine-1-yl)butyl)quinazolin-4(3H)-one

Starting from the product obtained in example 127 (20 mg, 0.05 mmol) and following the procedure described in example 128, the title compound was obtained as a side product (5.2 mg, Yield: 13%).

HPLC-MS (C) Rt, 1.93 min; ESI+-MS m/z: 400.1 (M+1).

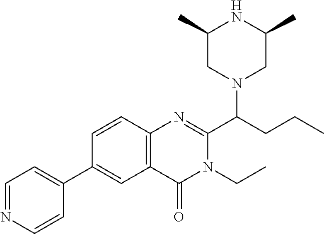 [SMILES: CCCC(c1nc2ccc(-c3ccncc3)cc2c(=O)n1CC)N1C[C@@H](C)N[C@@H](C)C1]

### Example 132. 2-(1-((3S,5R)-3,5-Dimethylpiperazin-1-yl)butyl)-3-ethyl-6-(pyridin-4-yl)quinazolin-4(3H)-one

### Step a. (2S,6R)-tert-Butyl 4-(1-(6-bromo-3-ethyl-4-oxo-3,4-dihydroquinazolin-2-yl)butyl)-2,6-dimethylpiperazine-1-carboxylate

To a solution of the product obtained in example 1 (1.5 g, 3.6 mmol) in anh DCM (60 mL) under Ar atmosphere, TEA (1 mL, 7.1 mmol) and di-tert-butyl dicarbonate (1.7 g, 7.8 mmol) were added and the mixture was stirred at r.t. overnight. The reaction mixture was washed with Na₂CO₃ sat sol, water and brine. The organic layer was dried over Na₂SO₄, filtered and concentrated to dryness to give the title compound (1.6 g, Yield: 82%).

### Step b. (2S,6R)-tert-Butyl 4-(1-(3-ethyl-4-oxo-6-(pyridin-4-yl)-3,4-dihydroquinazolin-2-yl)butyl)-2,6-dimethylpiperazine-1-carboxylate

A MW tube was charged with a solution of the product obtained in step a (65 mg, 0.13 mmol) in DME:H₂O (3 mL). Pyridin-4-ylboronic acid (23 mg, 0.19 mmol), K₂CO₃ (35 mg, 0.25 mmol) and Pd(PPh₃)₄(8 mg, 0.007 mmol) were added and the mixture was heated under MW irradiation (150 W) at 130° C. for 20 min. The solvent was removed under vacuum. The residue was dissolved in EtOAc, washed with aq NaHCO₃ sat sol and the organic layer was dried over Na₂SO₄, filtered and concentrated to dryness to give the title compound (24 mg, Yield: 36%).

### Step c. Title Compound

Starting from the product obtained in step b (24 mg, 0.05 mmol) and following the procedure described in step e of example 79 the title compound was obtained (19 mg, Yield: quant).

HPLC-MS (C) Rt, 1.65 min; ESI+-MS m/z: 420.3 (M+1).

This method was used for the preparation of examples 133-136 using suitable starting materials:

| | | CHEMICAL | Rt | MS | HPLC |
|---|---|---|---|---|---|
| STRUCTURE | EX | NAME | (min) | (M + H) | Method |
| 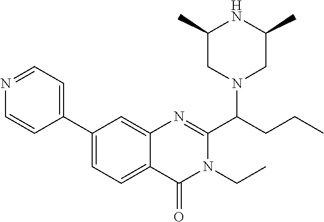 [SMILES: CCCC(c1nc2cc(-c3ccncc3)ccc2c(=O)n1CC)N1C[C@@H](C)N[C@@H](C)C1] | 133 | 2-(1-((3S,5R)-3,5- dimethylpiperazin-1- yl)butyl)-3-ethyl-7- (pyridin-4- yl)quinazolin-4(3H)- one | 1.66 | 420.2 | C |
| 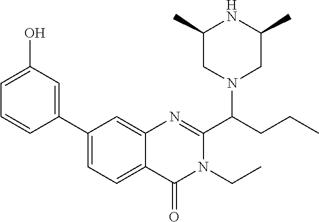 [SMILES: CCCC(c1nc2cc(-c3cccc(O)c3)ccc2c(=O)n1CC)N1C[C@@H](C)N[C@@H](C)C1] | 134 | 2-(1-((3S,5R)-3,5- dimethylpiperazin-1- yl)butyl)-3-ethyl-7-(3- hydroxyphenyl) quinazolin-4(3H)-one | 1.74 | 435.3 | C |
| 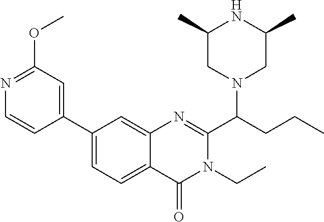 [SMILES: CCCC(c1nc2cc(-c3ccnc(OC)c3)ccc2c(=O)n1CC)N1C[C@@H](C)N[C@@H](C)C1] | 135 | 2-(1-((3S,5R)-3,5- dimethylpiperazin-1- yl)butyl)-3-ethyl-7-(2- methoxypyridin-4- yl)quinazolin-4(3H)- one | 1.98 | 450.3 | C |
| 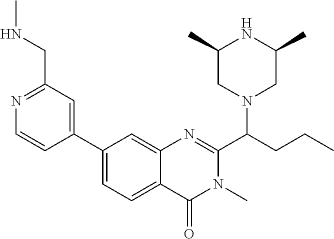 [SMILES: CCCC(c1nc2cc(-c3ccnc(CNC)c3)ccc2c(=O)n1C)N1C[C@@H](C)N[C@@H](C)C1] | 136 | 2-(1-((3S,5R)-3,5- dimethylpiperazin-1- yl)butyl)-3-methyl-7-(2- ((methylamino)methyl) pyridin-4-yl)quinazolin- 4(3H)-one | 1.51 | 449.6 | E |

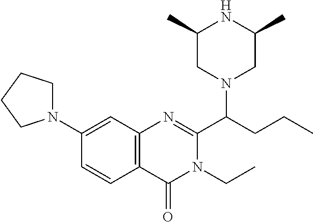 [SMILES: CCCC(c1nc2cc(N3CCCC3)ccc2c(=O)n1CC)N1C[C@@H](C)N[C@@H](C)C1]

### Example 137. 2-(1-((3S,5R)-3,5-Dimethylpiperazin-1-yl)butyl)-3-ethyl-7-(pyrrolidin-1-yl)quinazolin-4(3H)-one

### Step a. (2S,6R)-tert-Butyl 4-(1-(3-ethyl-4-oxo-7-(pyrrolidin-1-yl)-3,4-dihydroquinazolin-2-yl)butyl)-2,6-dimethylpiperazine-1-carboxylate

A schlenk flask was charged with (2S,6R)-tert-butyl 4-(1-(7-bromo-3-ethyl-4-oxo-3,4-dihydroquinazolin-2-yl)butyl)-2,6-dimethylpiperazine-1-carboxylate (obtained as described in example 1, 50 mg, 0.1 mmol), XPhos (5 mg, 0.1 mmol), Pd₂dba₃ (4 mg, 0.005 mmol) and K₂CO₃ (40 mg, 0.3 mmol) and it was evacuated and backfilled with argon. Tert-butanol (4 mL), degassed by means of bubbling argon to the solution for 5 min and pyrrolidine (16 μL, 0.2 mmol) were added and the reaction mixture was heated at 100° C. overnight. The suspension was filtered through celite, washed with EtOAc and the solvent was removed under vacuum. The crude product was purified by flash chromatography, silica gel, gradient Chx to EtOAc (100%) to give the title compound (25 mg, Yield: 51%).

### Step b. Title Compound

Starting from the product obtained in step a (25 mg, 0.05 mmol) and following the procedure described in step b of example 79, the title product was obtained (19 mg, Yield: 94%).

HPLC-MS (C) Rt, 1.89 min; ESI+-MS m/z: 412.3 (M+1).

This method was used for the preparation of examples 138-151 using suitable starting materials:

| | | CHEMICAL | Rt | MS | HPLC |
|---|---|---|---|---|---|
| STRUCTURE | EX | NAME | (min) | (M + H) | Method |
| 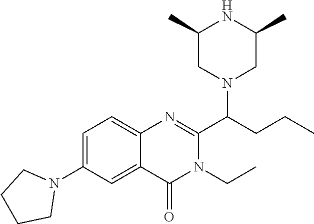 [SMILES: CCCC(c1nc2ccc(N3CCCC3)cc2c(=O)n1CC)N1C[C@@H](C)N[C@@H](C)C1] | 138 | 2-(1-((3S,5R)-3,5- dimethylpiperazin-1- yl)butyl)-3-ethyl-6- (pyrrolidin-1- yl)quinazolin-4(3H)- one | 2.03 | 412.4 | C |
| 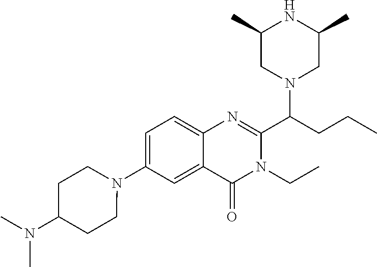 [SMILES: CCCC(c1nc2ccc(N3CCC(N(C)C)CC3)cc2c(=O)n1CC)N1C[C@@H](C)N[C@@H](C)C1] | 139 | 6-(4- (dimethylamino)piperidin- 1-yl)-2-(1-((3S,5R)- 3,5-dimethylpiperazin- 1-yl)butyl)-3- ethylquinazolin-4(3H)- one | 1.65 | 469.4 | F |
| 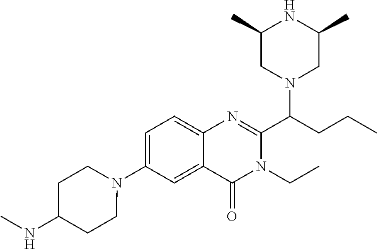 [SMILES: CCCC(c1nc2ccc(N3CCC(NC)CC3)cc2c(=O)n1CC)N1C[C@@H](C)N[C@@H](C)C1] | 140 | 2-(1-((3S,5R)-3,5- dimethylpiperazin-1- yl)butyl)-3-ethyl-6-(4- (methylamino)piperidin- 1-yl)quinazolin-4(3H)- one | 1.53 | 455.3 | F |
| 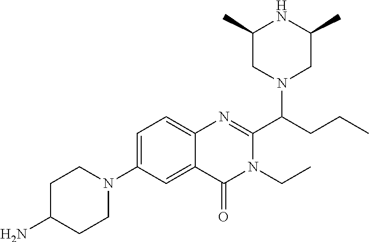 [SMILES: CCCC(c1nc2ccc(N3CCC(N)CC3)cc2c(=O)n1CC)N1C[C@@H](C)N[C@@H](C)C1] | 141 | 6-(4-aminopiperidin-1- yl)-2-(1-((3S,5R)-3,5- dimethylpiperazin-1- yl)butyl)-3- ethylquinazolin-4(3H)- one | 1.40 | 441.3 | F |
| 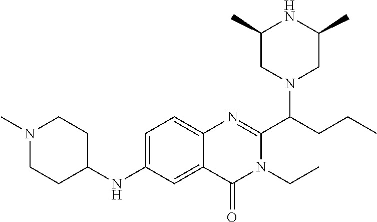 [SMILES: CCCC(c1nc2ccc(NC3CCN(C)CC3)cc2c(=O)n1CC)N1C[C@@H](C)N[C@@H](C)C1] | 142 | 2-(1-((3S,5R)-3,5- dimethylpiperazin-1- yl)butyl)-3-ethyl-6-((1- methylpiperidin-4- yl)amino)quinazolin- 4(3H)-one | 1.48 | 455.3 | F |
| 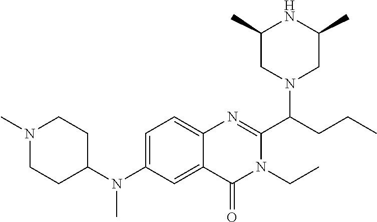 [SMILES: CCCC(c1nc2ccc(N(C)C3CCN(C)CC3)cc2c(=O)n1CC)N1C[C@@H](C)N[C@@H](C)C1] | 143 | 2-(1-((3S,5R)-3,5- dimethylpiperazin-1- yl)butyl)-3-ethyl-6- (methyl(1- methylpiperidin-4- yl)amino)quinazolin- 4(3H)-one | 1.61 | 469.4 | F |
| 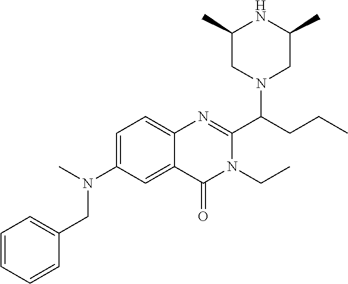 [SMILES: CCCC(c1nc2ccc(N(C)Cc3ccccc3)cc2c(=O)n1CC)N1C[C@@H](C)N[C@@H](C)C1] | 144 | 6- (benzyl(methyl)amino)- 2-(1-((3S,5R)-3,5- dimethylpiperazin-1- yl)butyl)-3- ethylquinazolin-4(3H)- one | 2.77 | 462.2 | G |
| 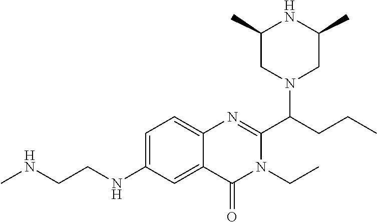 [SMILES: CCCC(c1nc2ccc(NCCNC)cc2c(=O)n1CC)N1C[C@@H](C)N[C@@H](C)C1] | 145 | 2-(1-((3S,5R)-3,5- dimethylpiperazin-1- yl)butyl)-3-ethyl-6-((2- (methylamino)ethyl) amino)quinazolin- 4(3H)-one | 1.28 | 415.5 | E |
| 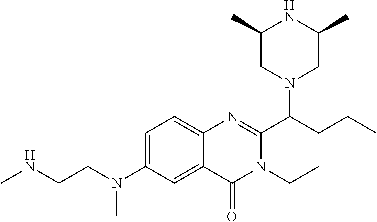 [SMILES: CCCC(c1nc2ccc(N(C)CCNC)cc2c(=O)n1CC)N1C[C@@H](C)N[C@@H](C)C1] | 146 | 2-(1-((3S,5R)-3,5- dimethylpiperazin-1- yl)butyl)-3-ethyl-6- (methyl(2- (methylamino)ethyl) amino)quinazolin- 4(3H)-one | 1.46 | 429.5 | A |
| 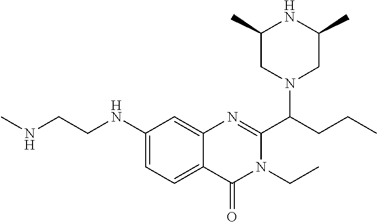 [SMILES: CCCC(c1nc2cc(NCCNC)ccc2c(=O)n1CC)N1C[C@@H](C)N[C@@H](C)C1] | 147 | 2-(1-((3S,5R)-3,5- dimethylpiperazin-1- yl)butyl)-3-ethyl-7-((2- (methylamino)ethyl) amino)quinazolin- 4(3H)-one | 1.19 | 415.3 | E |
| 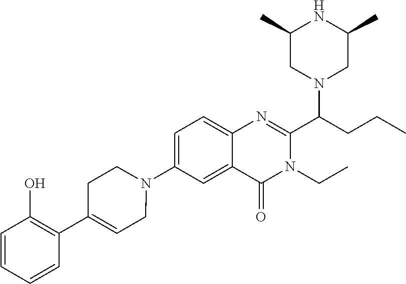 [SMILES: CCCC(c1nc2ccc(N3CC=C(c4ccccc4O)CC3)cc2c(=O)n1CC)N1C[C@@H](C)N[C@@H](C)C1] | 148 | 2-(1-((3S,5R)-3,5- dimethylpiperazin-1- yl)butyl)-3-ethyl-6-(4- (2-hydroxyphenyl)-5,6- dihydropyridin-1(2H)- yl)quinazolin-4(3H)- one | 2.31 | 516.3 | A |
| 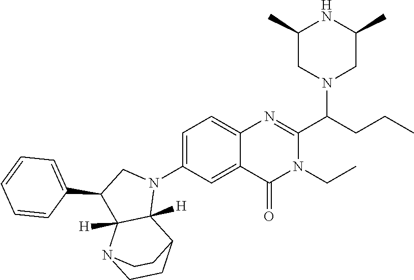 [SMILES: CCCC(c1nc2ccc(N3C[C@H](c4ccccc4)[C@@H]4[C@H]3C3CCN4CC3)cc2c(=O)n1CC)N1C[C@@H](C)N[C@@H](C)C1] | 149 | 2-(1-((3S,5R)-3,5- dimethylpiperazin-1- yl)butyl)-3-ethyl-6- ((3S,3aR,7aR)-3- phenylhexahydro-4,7- ethanopyrrolo[3,2- b]pyridin-1(2H)- yl)quinazolin-4(3H)- one | 3.00 | 569.3 | I |
| 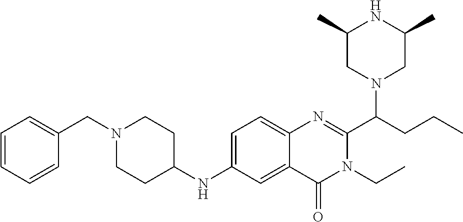 [SMILES: CCCC(c1nc2ccc(NC3CCN(Cc4ccccc4)CC3)cc2c(=O)n1CC)N1C[C@@H](C)N[C@@H](C)C1] | 150 | 6-((1-benzylpiperidin- 4-yl)amino)-2-(1- ((3S,5R)-3,5- dimethylpiperazin-1- yl)butyl)-3- ethylquinazolin-4(3H)- one | 2.88 | 531.3 | D |
| 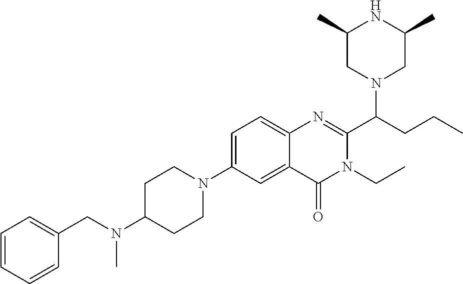 [SMILES: CCCC(c1nc2ccc(N3CCC(N(C)Cc4ccccc4)CC3)cc2c(=O)n1CC)N1C[C@@H](C)N[C@@H](C)C1] | 151 | 6-(4- (benzyl(methyl)amino) piperidin-1-yl)-2-(1- ((3S,5R)-3,5- dimethylpiperazin-1- yl)butyl)-3- ethylquinazolin-4(3H)- one | 2.53 | 545.3 | A |

The following compounds were obtained using the same method described in Example 137, but directly separating the enantiomeric or diastereomeric mixtures using chiral HPLC:

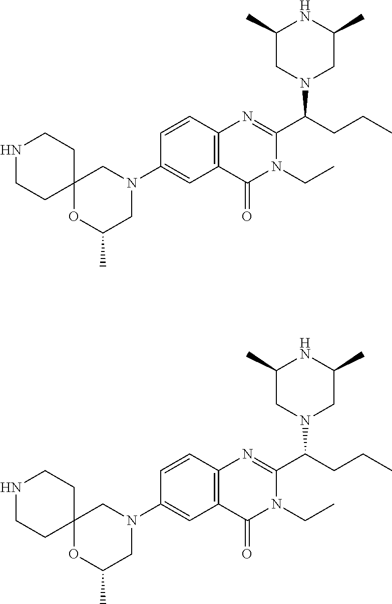 [SMILES: CCC[C@@H](c1nc2ccc(N3C[C@H](C)OC4(CCNCC4)C3)cc2c(=O)n1CC)N1C[C@@H](C)N[C@@H](C)C1 ; CCC[C@H](c1nc2ccc(N3C[C@H](C)OC4(CCNCC4)C3)cc2c(=O)n1CC)N1C[C@@H](C)N[C@@H](C)C1]

### Examples 152 and 153. 2-((S)-1-((3S,5R)-3,5-Dimethylpiperazin-1-yl)butyl)-3-ethyl-6-((S)-2-methyl-1-oxa-4,9-diazaspiro[5.5]undecan-4-yl)quinazolin-4(3H)-one and 2-((R)-1-((3S,5R)-3,5-dimethylpiperazin-1-yl)butyl)-3-ethyl-6-((S)-2-methyl-1-oxa-4,9-diazaspiro[5.5]undecan-4-yl)quinazolin-4(3H)-one

Starting from 2-(1-((3S,5R)-3,5-dimethylpiperazin-1-yl)butyl)-3-ethyl-6-((S)-2-methyl-1-oxa-4,9-diazaspiro[5.5]undecan-4-yl)quinazolin-4(3H)-one, a chiral preparative HPLC separation [column: Chiralpak AD-H, temperature: ambient; flow: 11 mL/min, eluent n-Heptane/(IPA+0.33% DEA) 75/25 v/v; Rt₁: 16.1.′, Rt₂: 23.3] was carried out to give the title compounds.

HPLC-MS (F) Rt, 1.61/1.66 min; ESI+-MS m/z: 511.4 (M+1).

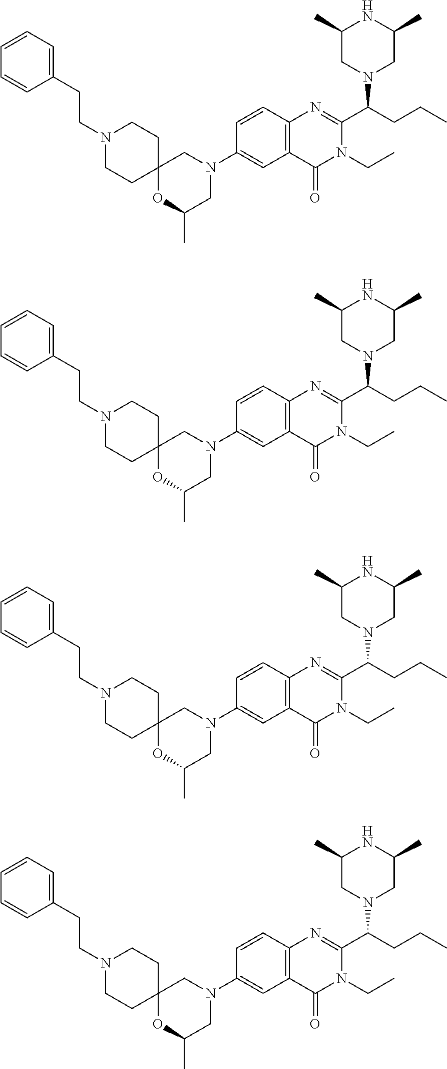 [SMILES: CCC[C@@H](c1nc2ccc(N3C[C@@H](C)OC4(CCN(CCc5ccccc5)CC4)C3)cc2c(=O)n1CC)N1C[C@@H](C)N[C@@H](C)C1 ; CCC[C@@H](c1nc2ccc(N3C[C@H](C)OC4(CCN(CCc5ccccc5)CC4)C3)cc2c(=O)n1CC)N1C[C@@H](C)N[C@@H](C)C1 ; CCC[C@H](c1nc2ccc(N3C[C@H](C)OC4(CCN(CCc5ccccc5)CC4)C3)cc2c(=O)n1CC)N1C[C@@H](C)N[C@@H](C)C1 ; CCC[C@H](c1nc2ccc(N3C[C@@H](C)OC4(CCN(CCc5ccccc5)CC4)C3)cc2c(=O)n1CC)N1C[C@@H](C)N[C@@H](C)C1]

### Examples 154, 155, 156 and 157. 2-((S)-1-((3S,5R)-3,5-Dimethylpiperazin-1-yl)butyl)-3-ethyl-6-((R)-2-methyl-9-phenethyl-1-oxa-4,9-diazaspiro[5.5]undecan-4-yl)quinazolin-4(3H)-one, 2-((S)-1-((3S,5R)-3,5-dimethylpiperazin-1-yl)butyl)-3-ethyl-6-((S)-2-methyl-9-phenethyl-1-oxa-4,9-diazaspiro[5.5]undecan-4-yl)quinazolin-4(3H)-one, 2-((R)-1-((3S,5R)-3,5-dimethylpiperazin-1-yl)butyl)-3-ethyl-6-((S)-2-methyl-9-phenethyl-1-oxa-4,9-diazaspiro[5.5]undecan-4-yl)quinazolin-4(3H)-one, 2-((R)-1-((3S,5R)-3,5-dimethylpiperazin-1-yl)butyl)-3-ethyl-6-((R)-2-methyl-9-phenethyl-1-oxa-4,9-diazaspiro[5.5]undecan-4-yl)quinazolin-4(3H)-one

Starting from 2-(1-((3S,5R)-3,5-dimethylpiperazin-1-yl)butyl)-3-ethyl-6-(2-methyl-9-phenethyl-1-oxa-4,9-diazaspiro[5.5]undecan-4-yl)quinazolin-4(3H)-one, a chiral preparative HPLC separation [column: Chiralpak AD-H, temperature: ambient; flow: 12 mL/min, eluent n-Heptane/(IPA+0.33% DEA) 75/25 v/v; Rt₁: 8.2′, Rt₂: 12.5′, Rt₃: 16.0′, Rt₄: 30.7′] was carried out to give the title compounds.

HPLC-MS (D) Rt, 3.06/3.08/3.13/3.17 min; ESI+-MS m/z: 615.3 (M+1).

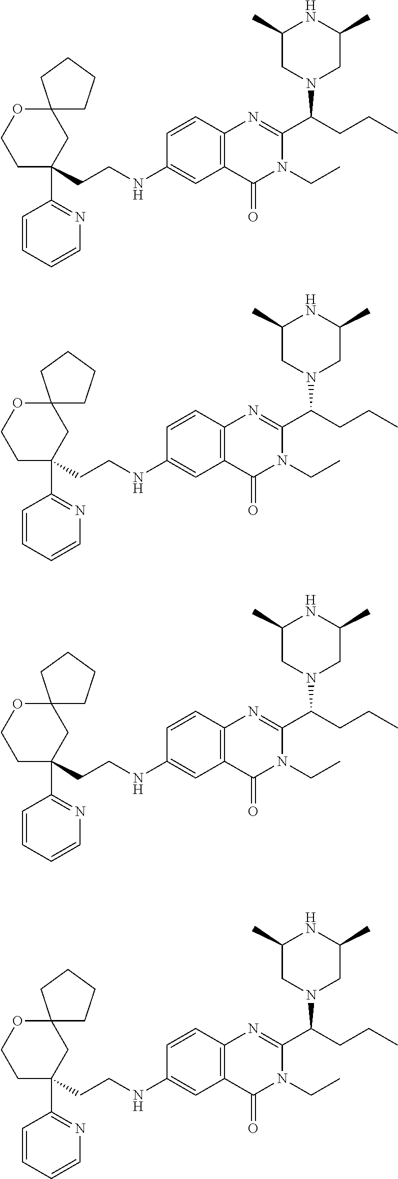 [SMILES: CCC[C@@H](c1nc2ccc(NCC[C@@]3(c4ccccn4)CCOC4(CCCC4)C3)cc2c(=O)n1CC)N1C[C@@H](C)N[C@@H](C)C1 ; CCC[C@H](c1nc2ccc(NCC[C@]3(c4ccccn4)CCOC4(CCCC4)C3)cc2c(=O)n1CC)N1C[C@@H](C)N[C@@H](C)C1 ; CCC[C@H](c1nc2ccc(NCC[C@@]3(c4ccccn4)CCOC4(CCCC4)C3)cc2c(=O)n1CC)N1C[C@@H](C)N[C@@H](C)C1 ; CCC[C@@H](c1nc2ccc(NCC[C@]3(c4ccccn4)CCOC4(CCCC4)C3)cc2c(=O)n1CC)N1C[C@@H](C)N[C@@H](C)C1]

### Examples 158, 159, 160 and 161. 2-((S)-1-((3S,5R)-3,5-Dimethylpiperazin-1-yl)butyl)-3-ethyl-6-((2-((R)-9-(pyridin-2-yl)-6-oxaspiro[4.5]decan-9-yl)ethyl)amino)quinazolin-4(3H)-one, 2-((R)-1-((3S,5R)-3,5-dimethylpiperazin-1-yl)butyl)-3-ethyl-6-((2-((S)-9-(pyridin-2-yl)-6-oxaspiro[4.5]decan-9-yl)ethyl)amino)quinazolin-4(3H)-one, 2-((R)-1-((3S,5R)-3,5-dimethylpiperazin-1-yl)butyl)-3-ethyl-6-((2-((R)-9-(pyridin-2-yl)-6-oxaspiro[4.5]decan-9-yl)ethyl)amino)quinazolin-4(3H)-one, 2-((S)-1-((3S,5R)-3,5-dimethylpiperazin-1-yl)butyl)-3-ethyl-6-((2-((S)-9-(pyridin-2-yl)-6-oxaspiro[4.5]decan-9-yl)ethyl)amino)quinazolin-4(3H)-one

Starting from 2-(1-((3S,5R)-3,5-dimethylpiperazin-1-yl)butyl)-3-ethyl-6-((2-(9-(pyridin-2-yl)-6-oxaspiro[4.5]decan-9-yl)ethyl)amino)quinazolin-4(3H)-one a chiral preparative HPLC separation [column: Chiralpak AD-H, temperature: ambient; flow: 10 mL/min, eluent n-Heptane/(IPA+0.33% DEA) 90/10 v/v and then a column: Chiralpak AD-H, temperature: ambient; flow: 10 mL/min, eluent n-Heptane/(EtOH+0.33% DEA) 90/10 v/v] was carried out to give the title compounds.

HPLC-MS (F) Rt, 2.25 min; ESI+-MS m/z: 601.5 (M+1).

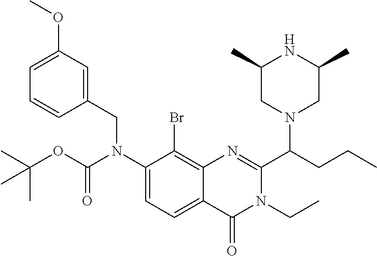 [SMILES: CCCC(c1nc2c(Br)c(N(Cc3cccc(OC)c3)C(=O)OC(C)(C)C)ccc2c(=O)n1CC)N1C[C@@H](C)N[C@@H](C)C1]

### Example 162. tert-Butyl (8-bromo-2-(1-((3S,5R)-3,5-dimethylpiperazin-1-yl)butyl)-3-ethyl-4-oxo-3,4-dihydroquinazolin-7-yl)(3-methoxybenzyl)carbamate

### Step a. 4-Nitro-2-pentanamidobenzoic acid

Starting from 2-amino-4-nitrobenzoic acid (4 g, 22 mmol) and following the procedure described in step b of example 1 the title compound was obtained (5.8 g, Yield: 99%).

### Step b. N-Ethyl-4-nitro-2-pentanamidobenzamide

Starting from the product obtained in step a (4 g, 15 mmol) and following the procedure described in step a of example 1 the title compound was obtained (1.2 g, Yield: 27%).

### Step c. 2-Butyl-3-ethyl-7-nitroquinazolin-4(3H)-one

Starting from the product obtained in step b (1.1 g, 3.5 mmol) and following the procedure described in step c of example 1 the title compound was obtained (0.2 g, Yield: 20%).

### Step d. 7-Amino-2-butyl-3-ethylquinazolin-4(3H)-one

To a solution of SnCl₂ in MeOH:HCl (21.5 mL, 20:1.5), the compound obtained in step c (1 g, 3.6 mmol) was added at −10° C. The mixture was allowed to reach r.t. and stirred overnight. Na₂CO₃ 10% sol was added, and the product was extracted with DCM. The combined organic layers were dried over anh Na₂SO₄, filtered and concentrated to dryness to give the title compound (0.73 g, Yield: 81%).

### Step e. tert-Butyl (2-butyl-3-ethyl-4-oxo-3,4-dihydroquinazolin-7-yl)carbamate

Starting from the compound obtained in step d (0.73 g, 3 mmol) and following the procedure described in step a of example 132 the title compound was obtained (0.21 g, Yield: 20%).

### Step f. tert-Butyl (2-butyl-3-ethyl-4-oxo-3,4-dihydroquinazolin-7-yl)(3-methoxybenzyl)carbamate

To a solution of the compound obtained in step e (78 mg, 0.22 mmol) in anh DMF (4 mL) cooled at 0° C., NaH (60% dispersion in mineral oil, 23 mg, 0.6 mmol) was added portion wise and the mixture was stirred at r.t for 30 min. 1-(Bromomethyl)-3-methoxybenzene (91 mg, 0.5 mmol) was added and the reaction mixture was heated at 65° C. overnight. NaHCO₃ sat sol was added and the product was extracted with EtOAc:Et₂O (1:1). The combined organic layers were washed with NaCl sat sol, dried over Na₂SO₄ and the crude product was purified by flash chromatography, silica gel, gradient DCM to MeOH (100%) to give the title compound (106 mg, Yield: quant).

### Step g. tert-Butyl (2-(1-bromobutyl)-3-ethyl-4-oxo-3,4-dihydroquinazolin-7-yl)(3-methoxybenzyl)carbamate

Starting from the compound obtained in step f (52 mg, 0.11 mmol) and following the procedure described in step d of example 1 the title compound was obtained (53 mg, Yield: 87%).

### Step h. Title Compound

Starting from the compound obtained in step g (53 mg, 0.1 mmol) and following the procedure described in step e of example 1 the title compound was obtained (46 mg, Yield: 82%).

HPLC-MS (C) Rt, 2.60 min; ESI+-MS m/z: 656.3 (M+1).

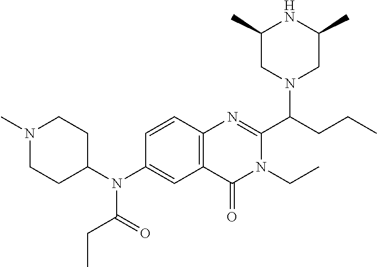 [SMILES: CCCC(c1nc2ccc(N(C(=O)CC)C3CCN(C)CC3)cc2c(=O)n1CC)N1C[C@@H](C)N[C@@H](C)C1]

### Example 163. N-(2-(1-((3S,5R)-3,5-Dimethylpiperazin-1-yl)butyl)-3-ethyl-4-oxo-3,4-dihydroquinazolin-6-yl)-N-(1-methylpiperidin-4-yl)propionamide

### Step a. (2S,6R)-tert-Butyl 4-(1-(3-ethyl-6-((1-methylpiperidin-4-yl)amino)-4-oxo-3,4-dihydroquinazolin-2-yl)butyl)-2,6-dimethylpiperazine-1-carboxylate

Starting from the compound obtained in step a of example 132 (0.3 g, 0.6 mmol) and following the procedure described in step a of example 137 the title compound was obtained (110 mg, Yield: 34%).

### Step b. (2S,6R)-tert-Butyl 4-(1-(3-ethyl-6-(N-(1-methylpiperidin-4-yl)propionamido)-4-oxo-3,4-dihydroquinazolin-2-yl)butyl)-2,6-dimethylpiperazine-1-carboxylate

To a solution of the compound obtained in step a (70 mg, 0.1 mmol) in anh DCM (3 mL), TEA (26 μL, 0.2 mmol) was added and the mixture was stirred at room temperature for 10 min. Then, it was cooled at 0° C., propionyl chloride (12 μL, 0.14 mmol) was added and the reaction mixture was allowed to reach r.t. and was stirred overnight. The resulting mixture was diluted with DCM, washed with aq NaHCO₃ sat sol and NaCl sat sol. The combined organic layers were dried over anh Na₂SO₄ and evaporated under vacuum to give the title compound (48 mg, Yield: 77%).

### Step c. Title Compound

Starting from the compound obtained in step b (48 mg, 0.08 mmol) and following the procedure described in step b of example 79 the title compound was obtained (33 mg, Yield: 83%).

HPLC-MS (F) Rt, 1.56 min; ESI+-MS m/z: 511.4 (M+1).

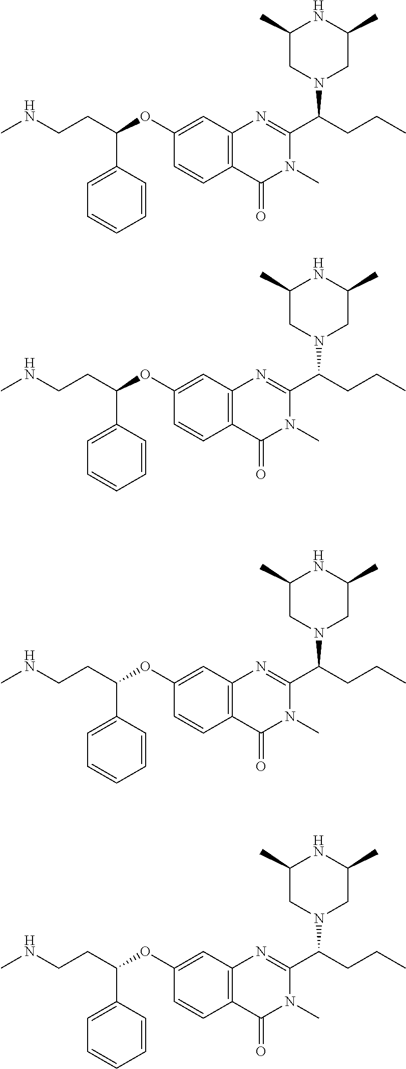

### Example 164, 165, 166 and 167. 2-((S)-1-((3S,5R)-3,5-Dimethylpiperazin-1-yl)butyl)-3-methyl-7-((R)-3-(methylamino)-1-phenylpropoxy)quinazolin-4(3H)-one, 2-((R)-1-((3S,5R)-3,5-dimethylpiperazin-1-yl)butyl)-3-methyl-7-((R)-3-(methylamino)-1-phenylpropoxy)quinazolin-4(3H)-one, 2-((S)-1-((3S,5R)-3,5-dimethylpiperazin-1-yl)butyl)-3-methyl-7-((S)-3-(methylamino)-1-phenylpropoxy)quinazolin-4(3H)-one and 2-((R)-1-((3S,5R)-3,5-dimethylpiperazin-1-yl)butyl)-3-methyl-7-((S)-3-(methylamino)-1-phenylpropoxy)quinazolin-4(3H)-one

### Step a. (2S,6R)-tert-Butyl 4-(1-(7-hydroxy-3-methyl-4-oxo-3,4-dihydroquinazolin-2-yl)butyl)-2,6-dimethylpiperazine-1-carboxylate

Starting from the compound obtained in example 10 (0.5 g, 1.5 mmol) and following the procedure described in step a of example 132 the title compound was obtained (0.52 g, Yield: 80%).

### Step b. (2S,6R)-tert-Butyl 4-(1-(7-(3-((tert-butoxycarbonyl)(methyl)amino)-1-phenylpropoxy)-3-methyl-4-oxo-3,4-dihydroquinazolin-2-yl)butyl)-2,6-dimethylpiperazine-1-carboxylate

Starting from the compound obtained in step a (40 mg, 0.1 mmol) and following the procedure described in step f of example 162 the title compound was obtained (45 mg, Yield: 72%).

### Step c. 2-(1-((3S,5R)-3,5-Dimethylpiperazin-1-yl)butyl)-3-methyl-7-(3-(methylamino)-1-phenylpropoxy)quinazolin-4(3H)-one

Starting from the compound obtained is step b (111 mg, 0.16 mmol) and following the procedure described in step b of example 79 the title compound was obtained (18 mg, Yield: 23%).

### Step d. Title Compounds

Starting from the compound obtained in step c, a chiral preparative HPLC separation [column: Chiralpak AD-H, temperature: ambient; flow: 13 mL/min, eluent n-Heptane/(IPA+0.33% DEA) 90/10 v/v, Rt₁: 11.9′, Rt₂: 14.4′, Rt₃: 18.5′ and a column: Chiralpak IG, temperature: ambient; flow: 13 mL/min eluent n-Heptane/(EtOH+0.33% DEA) 90/10 v/v Rt₃: 20.6, Rt₄: 22.8′] was carried out to give the title compounds.

HPLC-MS (F) Rt, 1.49 min; ESI+-MS m/z: 492.4 (M+1).

This method was used for the preparation of examples 168-171 using suitable starting materials:

| | | CHEMICAL | Rt | MS | HPLC |
|---|---|---|---|---|---|
| STRUCTURE | EX | NAME | (min) | (M + H) | Method |
| 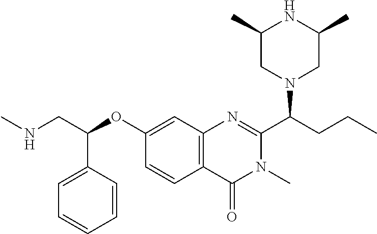 [SMILES: CCC[C@@H](c1nc2cc(O[C@H](CNC)c3ccccc3)ccc2c(=O)n1C)N1C[C@@H](C)N[C@@H](C)C1] | 168 | 2-((S)-1-((3S,5R)-3,5- dimethylpiperazin-1- yl)butyl)-3-methyl-7- ((S)-2-(methylamino)- 1- phenylethoxy)quinazolin- 4(3H)-one | 1.61 | 478.4 | F |
| 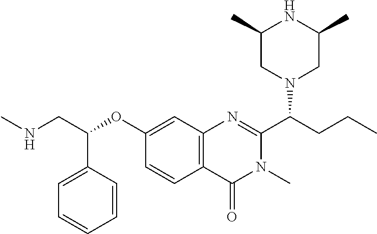 [SMILES: CCC[C@H](c1nc2cc(O[C@@H](CNC)c3ccccc3)ccc2c(=O)n1C)N1C[C@@H](C)N[C@@H](C)C1] | 169 | 2-((R)-1-((3S,5R)-3,5- dimethylpiperazin-1- yl)butyl)-3-methyl-7- ((R)-2-(methylamino)- 1- phenylethoxy)quinazolin- 4(3H)-one | 1.61 | 478.4 | F |
| 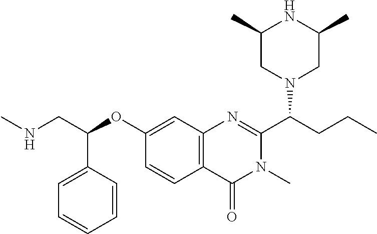 [SMILES: CCC[C@H](c1nc2cc(O[C@H](CNC)c3ccccc3)ccc2c(=O)n1C)N1C[C@@H](C)N[C@@H](C)C1] | 170 | 2-((R)-1-((3S,5R)-3,5- dimethylpiperazin-1- yl)butyl)-3-methyl-7- ((S)-2-(methylamino)- 1- phenylethoxy)quinazolin- 4(3H)-one | 1.59 | 478.7 | F |
| 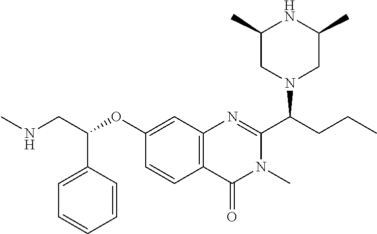 [SMILES: CCC[C@@H](c1nc2cc(O[C@@H](CNC)c3ccccc3)ccc2c(=O)n1C)N1C[C@@H](C)N[C@@H](C)C1] | 171 | 2-((S)-1-((3S,5R)-3,5- dimethylpiperazin-1- yl)butyl)-3-methyl-7- ((R)-2-(methylamino)- 1- phenylethoxy)quinazolin- 4(3H)-one | 1.59 | 478.4 | F |

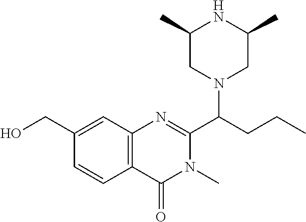 [SMILES: CCCC(c1nc2cc(CO)ccc2c(=O)n1C)N1C[C@@H](C)N[C@@H](C)C1]

### Example 172. 2-(1-((3S,5R)-3,5-Dimethylpiperazin-1-yl)butyl)-7-(hydroxymethyl)-3-methylquinazolin-4(3H)-one

### Step a. Methyl 2-(1-((3S,5R)-4-(tert-butoxycarbonyl)-3,5-dimethylpiperazin-1-yl)butyl)-3-methyl-4-oxo-3,4-dihydroquinazoline-7-carboxylate

Starting from the compound obtained in example 8 (70 mg, 0.19 mmol) and following the procedure described in step a of example 132 the title compound was obtained (80 mg, Yield: 87%).

### Step b. (2S,6R)-tert-Butyl 4-(1-(7-(hydroxymethyl)-3-methyl-4-oxo-3,4-dihydroquinazolin-2-yl)butyl)-2,6-dimethylpiperazine-1-carboxylate

To a solution of the compound obtained in step a (80 mg, 0.16 mmol) in THF:MeOH (5:1, 6 mL), LiBH₄ (2 M in THF, 495 μL, 1 mmol) was added dropwise and the reaction mixture was stirred at r.t. for 1.5 h. The mixture was poured into H₂O, HCl 10% aq sol was slowly added until pH=7 and the product was extracted with EtOAc. The combined organic layers were dried over anh Na₂SO₄, filtered and concentrated to dryness to give the title compound (57 mg, Yield: 76%).

### Step c. Title Compound

Starting from the compound obtained in step b (57 mg, 0.12 mmol) and following the procedure described in step b of example 79 the title compound was obtained (25 mg, Yield: 56%).

HPLC-MS (F) Rt, 1.26 min; ESI+-MS m/z: 359.2 (M+1).

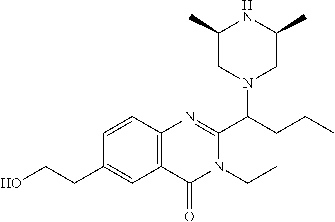 [SMILES: CCCC(c1nc2ccc(CCO)cc2c(=O)n1CC)N1C[C@@H](C)N[C@@H](C)C1]

### Example 173. 2-(1-((3S,5R)-3,5-Dimethylpiperazin-1-yl)butyl)-3-ethyl-6-(2-hydroxyethyl)quinazolin-4(3H)-one

### Step a. 2-(1-((3S,5R)-3,5-Dimethylpiperazin-1-yl)butyl)-3-ethyl-6-(2-((tetrahydro-2H-pyran-2-yl)oxy)ethyl)quinazolin-4(3H)-one

A schlenk flask was charged with the compound obtained in example 1 (0.56 g, 1.3 mmol), bis(di-tert-butyl(4-dimethylaminophenyl)phosphine)dichloropalladium(II) (94 mg, 0.13 mmol), potassium trifluoro(2-((tetrahydro-2H-pyran-2-yl)oxy)ethyl)borate (0.34 g, 1.6 mmol), and Cs₂CO₃ (1.7 g, 5.3 mmol) and it was evacuated and backfilled with argon. Toluene:H₂O (4:1, 10 mL) was added and the reaction mixture was heated at 100° C. overnight. H₂O was added and the product was extracted with EtOAc. The combined organic layers were dried over Na₂SO₄, filtered and concentrated to dryness to give the title compound (0.62 g, Yield: 99%).

### Step b. Title Compound

To a solution of the compound obtained in step a (0.24 g, 0.5 mmol) in EtOAc (20 mL), HCl (2 M in Et₂O, 2.5 mL, 5 mmol) was added and the mixture was stirred at r.t. overnight. The suspension was cooled to 0° C., the solid was filtered, washed with EtOAc and dried under vacuum to give the title compound (181 mg, Yield: 79%).

HPLC-MS (D) Rt, 2.05 min; ESI+-MS m/z: 387.6 (M+1).

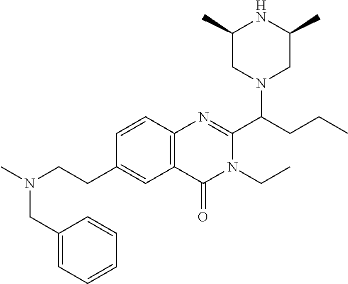 [SMILES: CCCC(c1nc2ccc(CCN(C)Cc3ccccc3)cc2c(=O)n1CC)N1C[C@@H](C)N[C@@H](C)C1]

### Example 174. 6-(2-(Benzyl(methyl)amino)ethyl)-2-(1-((3S,5R)-3,5-dimethylpiperazin-1-yl)butyl)-3-ethylquinazolin-4(3H)-one

### Step a. 6-(2-Bromoethyl)-2-(1-((3S,5R)-3,5-dimethylpiperazin-1-yl)butyl)-3-ethylquinazolin-4(3H)-one

A solution of the compound obtained in example 173 (118 mg, 0.26 mmol) in HBr (48% in water, 10 mL) was heated at 110° C. for 5 h. The solvent was removed under vacuum to give the title compound (134 mg, Yield: quant).

### Step b. Title Compound

A solution of the compound obtained in step a (46 mg, 0.09) in anh ACN (2 mL), TEA (74 μL, 0.5 mmol), KI (1 mg, 0.01 mmol) and N-methyl-1-phenylmethanamine (16 μL, 0.13 mmol) were added and the reaction mixture was heated in a sealed tube at 65° C. overnight. The mixture was diluted with EtOAc and washed with water. The organic layer was dried over Na₂SO₄, filtered and concentrated to dryness. The crude product was purified by flash chromatography, neutral Al₂O₃, gradient Chx to EtOAc (100%) to give the title compound (5 mg, Yield: 12%).

HPLC-MS (A): Rt, 2.46 min; ESI+-MS m/z: 490.3 (M+1)

This method was used for the preparation of example 175 using suitable starting materials:

| | | CHEMICAL | Rt | MS | HPLC |
|---|---|---|---|---|---|
| STRUCTURE | EX | NAME | (min) | (M + H) | Method |
| 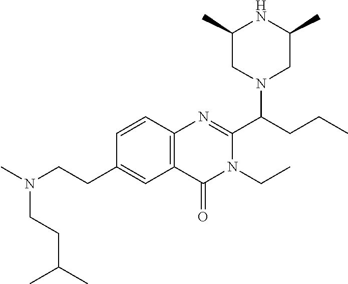 [SMILES: CCCC(c1nc2ccc(CCN(C)CCC(C)C)cc2c(=O)n1CC)N1C[C@@H](C)N[C@@H](C)C1] | 175 | 2-(1-((3S,5R)-3,5- dimethylpiperazin-1- yl)butyl)-3-ethyl-6-(2- (isopentyl(methyl)amino) ethyl)quinazolin- 4(3H)-one | 2.34 | 470.3 | A |

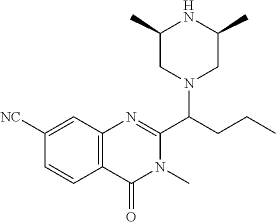 [SMILES: CCCC(c1nc2cc(C#N)ccc2c(=O)n1C)N1C[C@@H](C)N[C@@H](C)C1]

### Example 176. 2-(1-((3S,5R)-3,5-Dimethylpiperazin-1-yl)butyl)-3-methyl-4-oxo-3,4-dihydroquinazoline-7-carbonitrile

### Step a. (2S,6R)-tert-Butyl 4-(1-(7-bromo-3-methyl-4-oxo-3,4-dihydroquinazolin-2-yl)butyl)-2,6-dimethylpiperazine-1-carboxylate

Starting from the compound obtained in example 17 (9.2 g, 22.6 mmol) and following the procedure described in step a of example 132 the title compound was obtained (7 g, Yield: 61%).

### Step b. (2S,6R)-tert-Butyl 4-(1-(7-cyano-3-methyl-4-oxo-3,4-dihydroquinazolin-2-yl)butyl)-2,6-dimethylpiperazine-1-carboxylate

To a solution of the compound obtained in step a (2 g, 4 mmol) in DMF (20 mL), Zn(CN)₂ (0.5 g, 4 mmol) and Pd(PPh₃)₄(0.45 g, 0.4 mmol) were added under Ar atmosphere. The mixture was heated at 110° C. for 45 min under MW irradiation (150 W). The mixture was diluted with EtOAc, washed with NaCl sat sol and water. The combined organic layers were dried over anh Na₂SO₄, filtered and concentrated to dryness. The crude product thus obtained was purified by flash chromatography, silica gel, gradient Chx to EtOAc (100%) to give the title compound (0.97 g, Yield: 53%).

### Step c. Title Compound

Starting from the product obtained in step b (40 mg, 0.1 mmol) and following the procedure described in step b of example 79 the title compound was obtained (26 mg, Yield: 83%).

HPLC-MS (C) Rt, 1.59 min; ESI+-MS m/z: 354.3 (M+1).

This method was used for the preparation of example 177 using suitable starting materials:

| | | CHEMICAL | Rt | MS | HPLC |
|---|---|---|---|---|---|
| STRUCTURE | EX | NAME | (min) | (M + H) | Method |
| 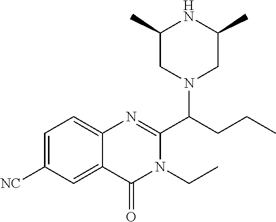 [SMILES: CCCC(c1nc2ccc(C#N)cc2c(=O)n1CC)N1C[C@@H](C)N[C@@H](C)C1] | 177 | 2-(1-((3S,5R)-3,5- dimethylpiperazin-1- yl)butyl)-3-ethyl-4-oxo- 3,4- dihydroquinazoline-6- carbonitrile | 1.75 | 368.2 | C |

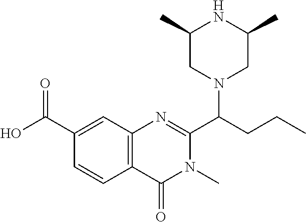 [SMILES: CCCC(c1nc2cc(C(=O)O)ccc2c(=O)n1C)N1C[C@@H](C)N[C@@H](C)C1]

### Example 178. 2-(1-((3S,5R)-3,5-Dimethylpiperazin-1-yl)butyl)-3-methyl-4-oxo-3,4-dihydroquinazoline-7-carboxylic acid

The product obtained in step b of example 176 (0.95 g, 2.1 mmol) was cooled at 0° C., then concentrated HCl (10 mL) was added drop wise and the reaction mixture was heated at 100° C. for 1 h. The solvent was removed under vacuum followed by co-evaporation with toluene, to give the title compound (0.8 g, Yield: 94%).

HPLC-MS (C) Rt, 0.97 min; ESI+-MS m/z: 373.3 (M+1).

This method was used for the preparation of example 179 using suitable starting materials:

| | | CHEMICAL | Rt | MS | HPLC |
|---|---|---|---|---|---|
| STRUCTURE | EX | NAME | (min) | (M + H) | Method |
| 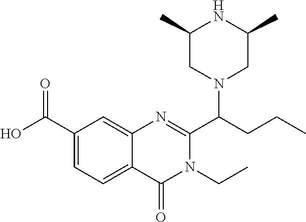 [SMILES: CCCC(c1nc2cc(C(=O)O)ccc2c(=O)n1CC)N1C[C@@H](C)N[C@@H](C)C1] | 179 | 2-(1-((3S,5R)-3,5- dimethylpiperazin-1- yl)butyl)-3-ethyl-4-oxo- 3,4- dihydroquinazoline-7- carboxylic acid | 1.31 | 387.4 | E |

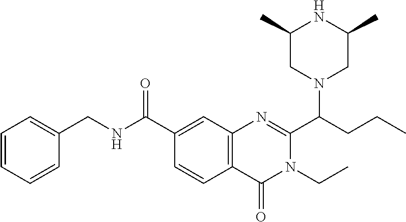 [SMILES: CCCC(c1nc2cc(C(=O)NCc3ccccc3)ccc2c(=O)n1CC)N1C[C@@H](C)N[C@@H](C)C1]

### Example 180. N-Benzyl-2-(1-((3S,5R)-3,5-dimethylpiperazin-1-yl)butyl)-3-ethyl-4-oxo-3,4-dihydroquinazoline-7-carboxamide

### Step a. 2-(1-((3S,5R)-4-(tert-Butoxycarbonyl)-3,5-dimethylpiperazin-1-yl)butyl)-3-ethyl-4-oxo-3,4-dihydroquinazoline-7-carboxylic acid

Starting from the compound obtained in example 179 (0.78 g, 2 mmol) and following the procedure described in step a of example 132 the title compound was obtained (0.98 g, Yield: quant).

### Step b. (2S,6R)-tert-Butyl 4-(1-(7-(benzylcarbamoyl)-3-ethyl-4-oxo-3,4-dihydroquinazolin-2-yl)butyl)-2,6-dimethylpiperazine-1-carboxylate

To a solution of the compound obtained in step a (78 mg, 0.16 mmol), under Ar atmosphere, in DMF (4 mL), HATU (76 mg, 0.2 mmol), TEA (45 μL, 0.3 mmol) and phenylmethanamine (26 μL, 0.24 mmol) were added and the reaction mixture was stirred at r.t. overnight. The reaction crude was diluted with EtOAc:Et₂O (1:1) and washed with NaHCO₃ and NaCl. The combined organic layers were joined and dried over anh Na₂SO₄. The solvent was removed under vacuum and the crude product was purified by flash chromatography, silica gel, gradient Chx to EtOAc (100%), to give the title compound (28 mg, Yield: 30%).

### Step c. Title Compound

Starting from the compound obtained in step b (28 mg, 0.05 mmol) and following the procedure described in step b of example 79, the title compound was obtained (17 mg, Yield: 72%).

HPLC-MS (C) Rt, 1.81 min; ESI+-MS m/z: 476.3 (M+1).

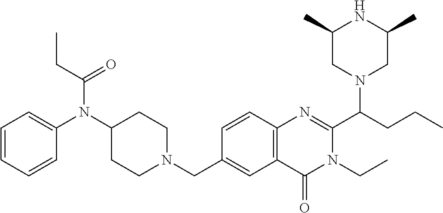 [SMILES: CCCC(c1nc2ccc(CN3CCC(N(C(=O)CC)c4ccccc4)CC3)cc2c(=O)n1CC)N1C[C@@H](C)N[C@@H](C)C1]

### Example 181. N-(1-((2-(1-((3S,5R)-3,5-Dimethylpiperazin-1-yl)butyl)-3-ethyl-4-oxo-3,4-dihydroquinazolin-6-yl)methyl)piperidin-4-yl)-N-phenylpropionamide

### Step a. (2S,6R)-tert-Butyl 4-(1-(3-ethyl-4-oxo-6-((4-(N-phenylpropionamido)piperidin-1-yl)methyl)-3,4-dihydroquinazolin-2-yl)butyl)-2,6-dimethylpiperazine-1-carboxylate

A sealed tube charged with the compound obtained in step a of example 132 (120 mg, 0.22 mmol), potassium trifluoro((4-(N-phenylpropionamido)piperidin-1-yl)methyl)borate (146 mg, 0.4 mmol), Pd(OAc)₂ (3 mg, 0.01 mmol), XPhos (13 mg, 0.03 mmol) and Cs₂CO₃ (225 mg, 0.7 mmol), was evacuated and backfilled with argon. Dioxane:H₂O (9:1, 4 mL), degassed by means of bubbling argon to the solution for 5 min, was added and the reaction mixture was stirred at 110° C. overnight. The solvent was removed under vacuum, the residue was dissolved in EtOAc and washed with aq NaHCO₃ sat sol. The combined organic layers were dried over anh Na₂SO₄, filtered and concentrated under vacuum. The crude product was purified by flash chromatography, silica gel, gradient DCM to DCM:MeOH (9:1) to give the title compound (130 mg, Yield: 82%).

### Step b. Title Compound

Starting from the compound obtained in step a (132 mg, 0.2 mmol) and following the procedure described in step b of example 79, the title compound was obtained (86 mg, Yield: 76%).

HPLC-MS (Method F): Rt, 2.16 min; ESI+-MS m/z: 587.4 (M+1).

This method was used for the preparation of examples 182-186 using suitable starting materials:

| | | CHEMICAL | Rt | MS | HPLC |
|---|---|---|---|---|---|
| STRUCTURE | EX | NAME | (min) | (M + H) | Method |
| 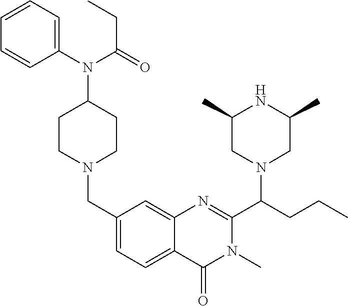 [SMILES: CCCC(c1nc2cc(CN3CCC(N(C(=O)CC)c4ccccc4)CC3)ccc2c(=O)n1C)N1C[C@@H](C)N[C@@H](C)C1] | 182 | N-(1-((2-(1-((3S,5R)- 3,5-dimethylpiperazin- 1-yl)butyl)-3-methyl-4- oxo-3,4- dihydroquinazolin-7- yl)methyl)piperidin-4- yl)-N- phenylpropionamide | 2.02 | 573.4 | F |
| 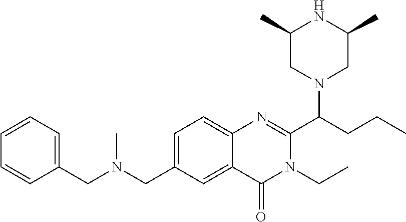 [SMILES: CCCC(c1nc2ccc(CN(C)Cc3ccccc3)cc2c(=O)n1CC)N1C[C@@H](C)N[C@@H](C)C1] | 183 | 6- ((benzyl(methyl)amino) methyl)-2-(1-((3S,5R)- 3,5-dimethylpiperazin- 1-yl)butyl)-3- ethylquinazolin-4(3H)- one | 2.73 | 476.2 | G |
| 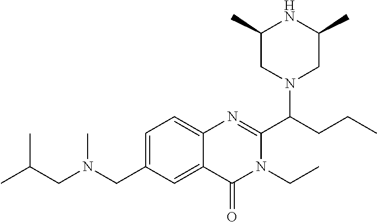 [SMILES: CCCC(c1nc2ccc(CN(C)CC(C)C)cc2c(=O)n1CC)N1C[C@@H](C)N[C@@H](C)C1] | 184 | 2-(1-((3S,5R)-3,5- dimethylpiperazin-1- yl)butyl)-3-ethyl-6- ((isobutyl(methyl)amino) methyl)quinazolin- 4(3H)-one | 2.97 | 442.3 | G |
| 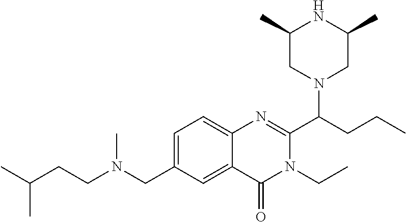 [SMILES: CCCC(c1nc2ccc(CN(C)CCC(C)C)cc2c(=O)n1CC)N1C[C@@H](C)N[C@@H](C)C1] | 185 | 2-(1-((3S,5R)-3,5- dimethylpiperazin-1- yl)butyl)-3-ethyl-6- ((isopentyl(methyl)amino) methyl)quinazolin- 4(3H)-one | 3.57 | 456.3 | D |
| 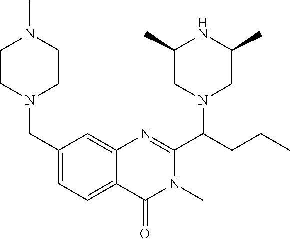 [SMILES: CCCC(c1nc2cc(CN3CCN(C)CC3)ccc2c(=O)n1C)N1C[C@@H](C)N[C@@H](C)C1] | 186 | 2-(1-((3S,5R)-3,5- dimethylpiperazin-1- yl)butyl)-3-methyl-7- ((4-methylpiperazin-1- yl)methyl)quinazolin- 4(3H)-one | 2.07 | 455.4 | G |

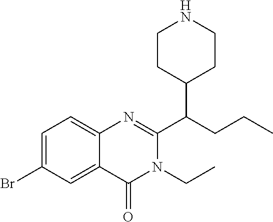 [SMILES: CCCC(c1nc2ccc(Br)cc2c(=O)n1CC)C1CCNCC1]

### Example 187. 6-Bromo-3-ethyl-2-(1-(piperidin-4-yl)butyl)quinazolin-4(3H)-one

### Step a. tert-Butyl 4-(2-((4-bromo-2-(ethylcarbamoyl)phenyl)amino)-2-oxoethyl)piperidine-1-carboxylate

To a solution of 2-(1-(tert-butoxycarbonyl)piperidin-4-yl)acetic acid (3.0 g, 12.3 mmol) in anh DMF (25 mL) under Ar atmosphere, TEA (2.3 mL, 16.5 mmol), HATU (3.7 g, 10 mmol) and 2-amino-5-bromo-N-ethylbenzamide (2.0 g, 8.2 mol) were added and the mixture was stirred at r.t. overnight. The reaction mixture was diluted with DCM, washed with NaHCO₃, and brine. The combined organic layers were dried over Na₂SO₄, filtered and the solvent was removed under vacuum. The crude product was purified by flash chromatography, silica gel, gradient Chx to AcOEt (100%) to give the title compound (3.4 g, Yield: 88%).

### Step b. 6-Bromo-3-ethyl-2-(piperidin-4-ylmethyl)quinazolin-4(3H)-one

To a solution of the compound obtained in step a (3.4 g, 7.3 mmol) and iodine (3.7 g, 14.6 mmol) in DCM (50 mL), HMDS (6.1 mL, 29.2 mmol) was added dropwise and the reaction mixture was stirred at r.t. overnight. The reaction mixture was diluted with DCM, washed with 5% Na₂S₂O₃ aq sol, water and brine. The organic layer was dried over Na₂SO₄ and the solvent was removed under vacuum. The crude product was purified by flash chromatography, silica gel, gradient DCM (100%) to MeOH (100%) to give the title compound (2.2 g, Yield: 87%).

### Step c. tert-Butyl 4-((6-bromo-3-ethyl-4-oxo-3,4-dihydroquinazolin-2-yl)methyl)piperidine-1-carboxylate

Starting from the compound obtained in step b (2.0 g, 5.7 mmol) and following the procedure described in step a of example 132, the title compound was obtained (2.7 g, Yield: quant).

### Step d. tert-Butyl 4-(1-(6-bromo-3-ethyl-4-oxo-3,4-dihydroquinazolin-2-yl)butyl)piperidine-1-carboxylate

To a solution of the compound obtained in step c (2.7 g, 6 mmol) in THE (50 mL) under Ar atmosphere, LiHMDS (15.1 mL, 15.1 mmol) was added and the mixture was stirred for 45 min at −78° C. 1-lodopropane was added and the reaction mixture was stirred at at −78° C. for 1 h and then was allowed to reach r.t. and stirred overnight. The reaction mixture was diluted with EtOAc and NH₄Cl, and the organic layer was washed with water, Na₂SO₃ and brine. The organic layer was dried over anh Na₂SO₄ and the solvent was removed under vacuum. The crude product was purified by flash chromatography, silica gel, gradient Chx to EtOAc (100%) to give the title compound (2.7 g, Yield: 90%).

### Step e. Title Compound

Starting from the compound obtained in step d (45 mg, 0.1 mmol) and following the procedure described in step b of example 79 the title compound was obtained (30 mg, Yield: 84%).

HPLC-MS (A): Rt, 2.02 min; ESI+-MS m/z: 390.1 (M+1).

This method was used for the preparation of examples 188-189 using suitable starting materials:

| | | CHEMICAL | Rt | MS | HPLC |
|---|---|---|---|---|---|
| STRUCTURE | EX | NAME | (min) | (M + H) | Method |
| 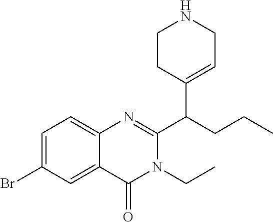 [SMILES: CCCC(C1=CCNCC1)c1nc2ccc(Br)cc2c(=O)n1CC] | 188 | 6-bromo-3-ethyl-2-(1- (1,2,3,6- tetrahydropyridin-4- yl)butyl)quinazolin- 4(3H)-one | 2.03 | 390.0 | A |
| 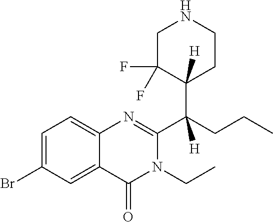 | 189 | 6-bromo-2-((S)-1-((R)- 3,3-difluoropiperidin-4- yl)butyl)-3- ethylquinazolin-4(3H)- one | 2.42 | 428.2 | A |

The following compounds were obtained using the same method described in Example 187, but directly separating the diastereomeric mixtures using chiral HPLC:

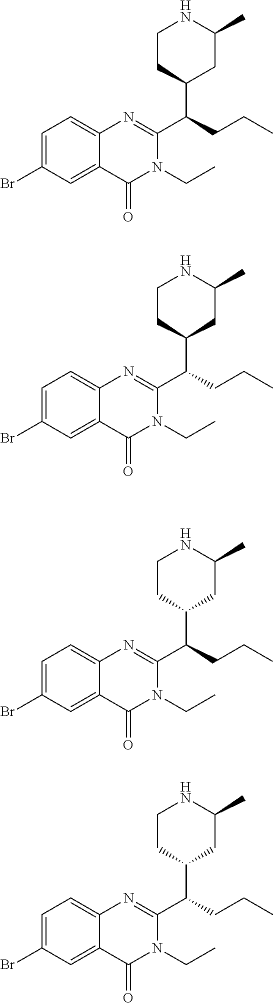 [SMILES: CCC[C@@H](c1nc2ccc(Br)cc2c(=O)n1CC)[C@H]1CCN[C@@H](C)C1 ; CCC[C@H](c1nc2ccc(Br)cc2c(=O)n1CC)[C@H]1CCN[C@@H](C)C1 ; CCC[C@@H](c1nc2ccc(Br)cc2c(=O)n1CC)[C@@H]1CCN[C@@H](C)C1 ; CCC[C@H](c1nc2ccc(Br)cc2c(=O)n1CC)[C@@H]1CCN[C@@H](C)C1]

### Examples 190, 191, 192 and 193. 6-Bromo-3-ethyl-2-((R)-1-((2S,4S)-2-methylpiperidin-4-yl)butyl)quinazolin-4(3H)-one, 6-bromo-3-ethyl-2-((S)-1-((2S,4S)-2-methylpiperidin-4-yl)butyl)quinazolin-4(3H)-one, 6-bromo-3-ethyl-2-((R)-1-((2S,4R)-2-methylpiperidin-4-yl)butyl)quinazolin-4(3H)-one, 6-bromo-3-ethyl-2-((S)-1-((2S,4R)-2-methylpiperidin-4-yl)butyl)quinazolin-4(3H)-one

Starting from 6-bromo-3-ethyl-2-(1-((2S)-2-methylpiperidin-4-yl)butyl)quinazolin-4(3H)-one, two chiral preparative HPLC separations [column: Chiralpak AD-H, temperature: ambient; flow: 13 mL/min, eluent n-Heptane/(IPA+0.33% DEA) 90/10 v/v; Rt1:11.9′, tr Rt2: 14.4′, Rt3:18.5′+column: Chiralpak IG, temperature: ambient; flow: 13 mL/min, eluent n-Heptane/(EtOH+0.33% DEA) 90/10 v/v; Rt3:20.6.′, Rt4: 22.8′] were carried out to give the title compounds.

HPLC-MS (B): Rt, 1.95/1.98/1.99/2.01 min; ESI+-MS m/z: 406.2 (M+1).

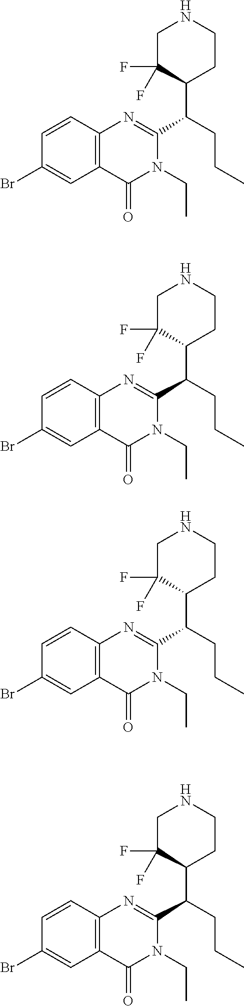 [SMILES: CCC[C@H](c1nc2ccc(Br)cc2c(=O)n1CC)[C@@H]1CCNCC1(F)F ; CCC[C@@H](c1nc2ccc(Br)cc2c(=O)n1CC)[C@H]1CCNCC1(F)F ; CCC[C@H](c1nc2ccc(Br)cc2c(=O)n1CC)[C@H]1CCNCC1(F)F ; CCC[C@@H](c1nc2ccc(Br)cc2c(=O)n1CC)[C@@H]1CCNCC1(F)F]

### Examples 194, 195, 196 and 197. 6-Bromo-2-((S)-1-((S)-3,3-difluoropiperidin-4-yl)butyl)-3-ethylquinazolin-4(3H)-one, 6-bromo-2-((R)-1-((R)-3,3-difluoropiperidin-4-yl)butyl)-3-ethylquinazolin-4(3H)-one, 6-bromo-2-((S)-1-((R)-3,3-difluoropiperidin-4-yl)butyl)-3-ethylquinazolin-4(3H)-one, 6-bromo-2-((R)-1-((S)-3,3-difluoropiperidin-4-yl)butyl)-3-ethylquinazolin-4(3H)-one

Starting from the compound obtained in example 189 a chiral preparative HPLC separation [column: Chiralpak AD-H, temperature: ambient; flow: 13 mL/min, eluent n-Heptane/(EtOH+0.33% DEA) 95/5 v/v; Rt₁: 18.3.′, Rt₂: 21.2′, Rt₃: 24.2′, tr Rt₄: 34.8′] was carried out to give the title compounds.

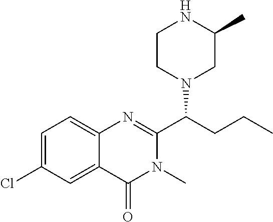 [SMILES: CCC[C@H](c1nc2ccc(Cl)cc2c(=O)n1C)N1CCN[C@@H](C)C1]

### Example 198. Chloro-3-methyl-2-((R)-1-((S)-3-methylpiperazin-1-yl)butyl)quinazolin-4(3H)-one

### Step a. (S)-5-Chloro-2-(2-hydroxypentanamido)-N-methylbenzamide

Starting from 2-amino-5-chloro-N-methylbenzamide (1.7 g, 7.8 mmol) and following the procedure described in step a of example 1 the title compound was obtained (1.2 g, Yield: 53%). ALC-0401

### Step b. (S)-6-Chloro-3-methyl-2-(1-((trimethylsilyl)oxy)butyl)quinazolin-4(3H)-one

To a solution of the compound obtained in step a (1.2 g, 4.1 mmol) in anh DCM (50 mL), iodine (2 g, 8.3 mmol) was added portion wise and the mixture was stirred until the iodine was totally soluble. HMDS (3.5 mL, 16.6 mmol) was added and the reaction mixture was stirred at r.t. overnight. The mixture was diluted with DCM, washed with sat sol Na₂S₂O₃ and NaCl sat sol. The organic layer was dried over anhydrous Na₂SO₄, filtered and concentrated under reduced vacuum to give the title compound (1.3 g, Yield: 89%).

### Step c. (S)-6-Chloro-2-(1-hydroxybutyl)-3-methylquinazolin-4(3H)-one

To a solution of the compound obtained in step b (1.3 g, 3.7 mmol) in anh THE (60 mL), TBAF sol 1M in THE (4 mL, 1 g) was added and the reaction mixture was stirred 30 min at 0° C. The mixture was diluted with EtOAC, washed with H₂O, and sat sol NaCl. The organic layer was dried over anhydrous Na₂SO₄, filtered and concentrated under reduced vacuum. The crude product was purified by flash chromatography, silica gel, gradient Chx (100%) to EtOAc (100%) to give the title compound (0.8 g, Yield: 84%).

### Step d. Title Compound

To a solution of the compound obtained in step c (50 mg, 0.2 mmol) in anh DCM (3 mL) at −78° C., 2,6-lutidine (87 μL, 0.7 mmol) and Tf₂O (1M in DCM, 0.24 mL, 0.24 mmol) were added and the mixture was stirred at −78° C. for 2 h. A solution of (S)-2-methylpiperazine (75 mg, 0.8 mmol) in DMF:DCM (1:1, 0.6 mL) was added and the mixture was allowed to reach r.t. slowly for 4 h. NaHCO₃ was added and the product was extracted with EtOAc. The combined organic layers were washed with NaCl sat sol, dried over anh Na₂SO₄, filtered and concentrated under reduced vacuum. The crude product was purified by flash chromatography, silica gel, gradient DCM (100%) to MeOH (100%) to give the title compound (55 mg, Yield: 84%).

HPLC-MS (B) Rt, 1.89 min; ESI+-MS m/z: 349.2 (M+1).

This method was used for the preparation of examples 199-226 using suitable starting materials:

| | | CHEMICAL | Rt | MS | HPLC |
|---|---|---|---|---|---|
| STRUCTURE | EX | NAME | (min) | (M + H) | Method |
| 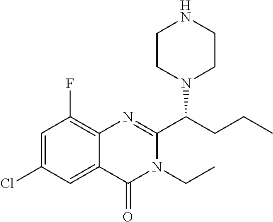 [SMILES: CCC[C@H](c1nc2c(F)cc(Cl)cc2c(=O)n1CC)N1CCNCC1] | 199 | (R)-6-chloro-3-ethyl-8- fluoro-2-(1-(piperazin- 1-yl)butyl)quinazolin- 4(3H)-one | 2.01 | 367.2 | A |
| 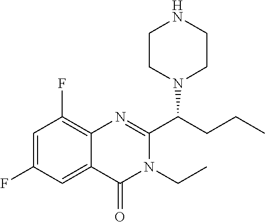 [SMILES: CCC[C@H](c1nc2c(F)cc(F)cc2c(=O)n1CC)N1CCNCC1] | 200 | (R)-3-ethyl-6,8- difluoro-2-(1- (piperazin-1- yl)butyl)quinazolin- 4(3H)-one | 1.85 | 351.0 | A |
| 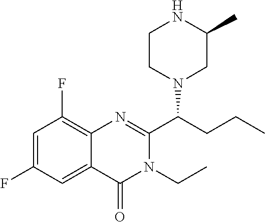 [SMILES: CCC[C@H](c1nc2c(F)cc(F)cc2c(=O)n1CC)N1CCN[C@@H](C)C1] | 201 | 3-ethyl-6,8-difluoro-2- ((R)-1-((S)-3- methylpiperazin-1- yl)butyl)quinazolin- 4(3H)-one | 1.97 | 365.0 | A |
| 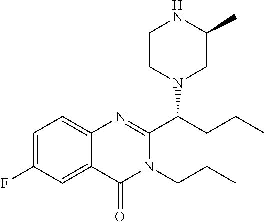 [SMILES: CCC[C@H](c1nc2ccc(F)cc2c(=O)n1CCC)N1CCN[C@@H](C)C1] | 202 | 6-fluoro-2-((R)-1-((S)- 3-methylpiperazin-1- yl)butyl)-3- propylquinazolin- 4(3H)-one | 1.89 | 434.2 | B |
| 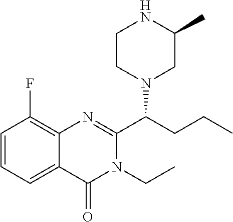 [SMILES: CCC[C@H](c1nc2c(F)cccc2c(=O)n1CC)N1CCN[C@@H](C)C1] | 203 | 3-ethyl-8-fluoro-2-((R)- 1-((S)-3- methylpiperazin-1- yl)butyl)quinazolin- 4(3H)-one | 1.81 | 347.0 | A |
| 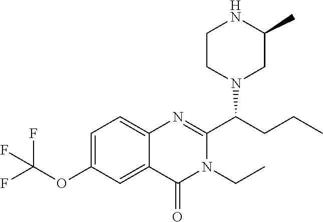 [SMILES: CCC[C@H](c1nc2ccc(OC(F)(F)F)cc2c(=O)n1CC)N1CCN[C@@H](C)C1] | 204 | 3-ethyl-2-((R)-1-((S)-3- methylpiperazin-1- yl)butyl)-6- (trifluoromethoxy) quinazolin-4(3H)-one | 2.22 | 413.1 | A |
| 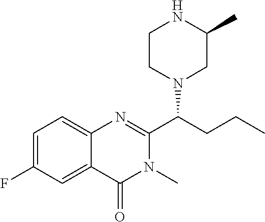 [SMILES: CCC[C@H](c1nc2ccc(F)cc2c(=O)n1C)N1CCN[C@@H](C)C1] | 205 | 6-fluoro-3-methyl-2- ((R)-1-((S)-3- methylpiperazin-1- yl)butyl)quinazolin- 4(3H)-one | 1.70 | 333.0 | A |
| 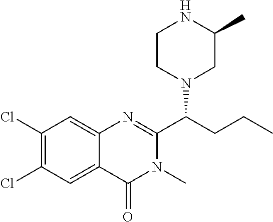 [SMILES: CCC[C@H](c1nc2cc(Cl)c(Cl)cc2c(=O)n1C)N1CCN[C@@H](C)C1] | 206 | 6,7-dichloro-3-methyl- 2-((R)-1-((S)-3- methylpiperazin-1- yl)butyl)quinazolin- 4(3H)-one | 2.23 | 383.2 | B |
| 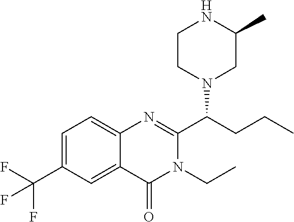 [SMILES: CCC[C@H](c1nc2ccc(C(F)(F)F)cc2c(=O)n1CC)N1CCN[C@@H](C)C1] | 207 | 3-ethyl-2-((R)-1-((S)-3- methylpiperazin-1- yl)butyl)-6- (trifluoromethyl) quinazolin-4(3H)-one | 2.18 | 397.2 | B |
| 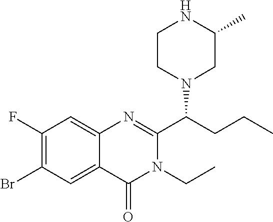 [SMILES: CCC[C@H](c1nc2cc(F)c(Br)cc2c(=O)n1CC)N1CCN[C@H](C)C1] | 208 | 6-bromo-3-ethyl-7- fluoro-2-((R)-1-((R)-3- methylpiperazin-1- yl)butyl)quinazolin- 4(3H)-one | 2.16 | 425.1 | A |
| 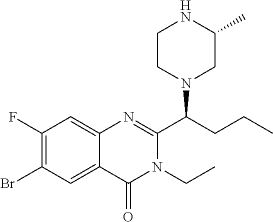 [SMILES: CCC[C@@H](c1nc2cc(F)c(Br)cc2c(=O)n1CC)N1CCN[C@H](C)C1] | 209 | 6-bromo-3-ethyl-7- fluoro-2-((S)-1-((R)-3- methylpiperazin-1- yl)butyl)quinazolin- 4(3H)-one | 2.19 | 425.1 | A |
| 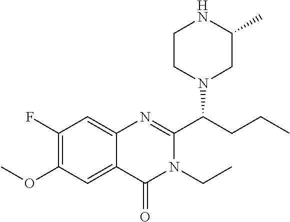 [SMILES: CCC[C@H](c1nc2cc(F)c(OC)cc2c(=O)n1CC)N1CCN[C@H](C)C1] | 210 | 3-ethyl-7-fluoro-6- methoxy-2-((R)-1-((R)- 3-methylpiperazin-1- yl)butyl)quinazolin- 4(3H)-one | 1.84 | 377.2 | A |
| 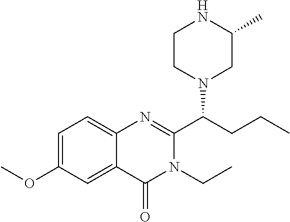 [SMILES: CCC[C@H](c1nc2ccc(OC)cc2c(=O)n1CC)N1CCN[C@H](C)C1] | 211 | 3-ethyl-6-methoxy-2- ((R)-1-((R)-3- methylpiperazin-1- yl)butyl)quinazolin- 4(3H)-one | 1.75 | 359.2 | A |
| 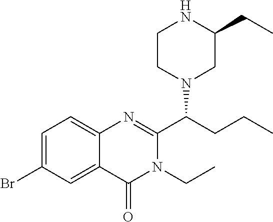 [SMILES: CCC[C@H](c1nc2ccc(Br)cc2c(=O)n1CC)N1CCN[C@@H](CC)C1] | 212 | 6-bromo-3-ethyl-2- ((R)-1-((S)-3- ethylpiperazin-1- yl)butyl)quinazolin- 4(3H)-one | 2.22 | 421.2 | A |
| 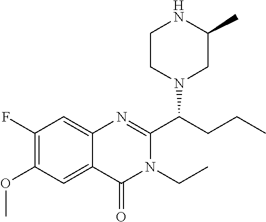 [SMILES: CCC[C@H](c1nc2cc(F)c(OC)cc2c(=O)n1CC)N1CCN[C@@H](C)C1] | 213 | 3-ethyl-7-fluoro-6- methoxy-2-((R)-1-((S)- 3-methylpiperazin-1- yl)butyl)quinazolin- 4(3H)-one | 1.83 | 377.3 | A |
| 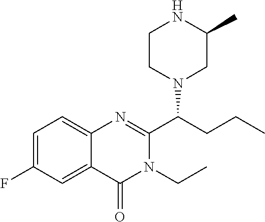 [SMILES: CCC[C@H](c1nc2ccc(F)cc2c(=O)n1CC)N1CCN[C@@H](C)C1] | 214 | 3-ethyl-6-fluoro-2-((R)- 1-((S)-3- methylpiperazin-1- yl)butyl)quinazolin- 4(3H)-one | 1.82 | 347.2 | A |
| 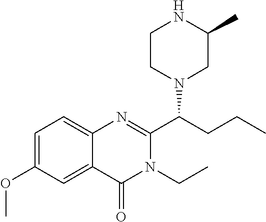 [SMILES: CCC[C@H](c1nc2ccc(OC)cc2c(=O)n1CC)N1CCN[C@@H](C)C1] | 215 | 3-ethyl-6-methoxy-2- ((R)-1-((S)-3- methylpiperazin-1- yl)butyl)quinazolin- 4(3H)-one | 1.76 | 359.2 | A |
| 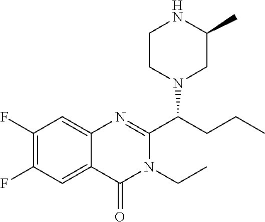 [SMILES: CCC[C@H](c1nc2cc(F)c(F)cc2c(=O)n1CC)N1CCN[C@@H](C)C1] | 216 | 3-ethyl-6,7-difluoro-2- ((R)-1-((S)-3- methylpiperazin-1- yl)butyl)quinazolin- 4(3H)-one | 1.99 | 365.2 | A |
| 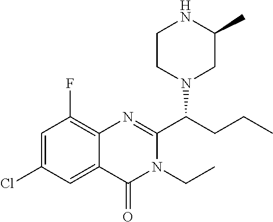 [SMILES: CCC[C@H](c1nc2c(F)cc(Cl)cc2c(=O)n1CC)N1CCN[C@@H](C)C1] | 217 | 6-chloro-3-ethyl-8- fluoro-2-((R)-1-((S)-3- methylpiperazin-1- yl)butyl)quinazolin- 4(3H)-one | 2.12 | 381.2 | A |
| 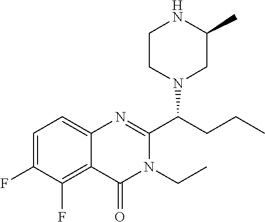 [SMILES: CCC[C@H](c1nc2ccc(F)c(F)c2c(=O)n1CC)N1CCN[C@@H](C)C1] | 218 | 3-ethyl-5,6-difluoro-2- ((R)-1-((S)-3- methylpiperazin-1- yl)butyl)quinazolin- 4(3H)-one | 1.84 | 365.2 | A |
| 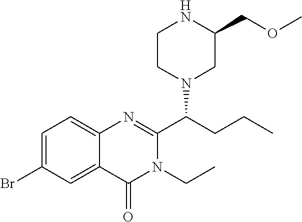 [SMILES: CCC[C@H](c1nc2ccc(Br)cc2c(=O)n1CC)N1CCN[C@@H](COC)C1] | 219 | 6-bromo-3-ethyl-2- ((R)-1-((R)-3- (methoxymethyl) piperazin-1- yl)butyl)quinazolin- 4(3H)-one | 2.25 | 437.2 | A |
| 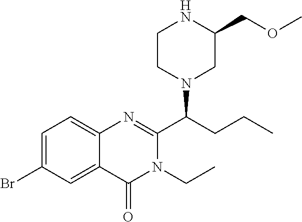 [SMILES: CCC[C@@H](c1nc2ccc(Br)cc2c(=O)n1CC)N1CCN[C@@H](COC)C1] | 220 | 6-bromo-3-ethyl-2- ((S)-1-((R)-3- (methoxymethyl) piperazin-1- yl)butyl)quinazolin- 4(3H)-one | 2.25 | 437.2 | A |
| 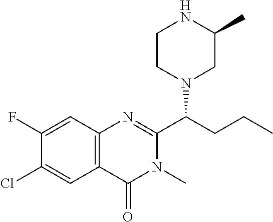 [SMILES: CCC[C@H](c1nc2cc(F)c(Cl)cc2c(=O)n1C)N1CCN[C@@H](C)C1] | 221 | 6-chloro-7-fluoro-3- methyl-2-((R)-1-((S)-3- methylpiperazin-1- yl)butyl)quinazolin- 4(3H)-one | 1.98 | 367.0 | A |
| 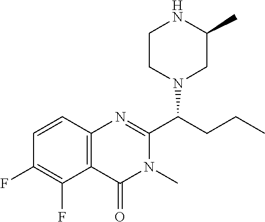 [SMILES: CCC[C@H](c1nc2ccc(F)c(F)c2c(=O)n1C)N1CCN[C@@H](C)C1] | 222 | 5,6-difluoro-3-methyl- 2-((R)-1-((S)-3- methylpiperazin-1- yl)butyl)quinazolin- 4(3H)-one | 1.67 | 351.2 | B |
| 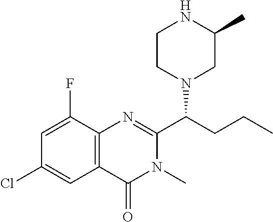 [SMILES: CCC[C@H](c1nc2c(F)cc(Cl)cc2c(=O)n1C)N1CCN[C@@H](C)C1] | 223 | 6-chloro-8-fluoro-3- methyl-2-((R)-1-((S)-3- methylpiperazin-1- yl)butyl)quinazolin- 4(3H)-one | 1.93 | 367.0 | A |
| 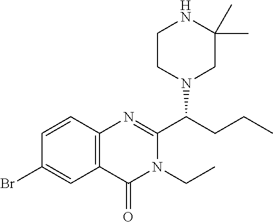 [SMILES: CCC[C@H](c1nc2ccc(Br)cc2c(=O)n1CC)N1CCNC(C)(C)C1] | 224 | (R)-6-bromo-2-(1-(3,3- dimethylpiperazin-1- yl)butyl)-3- ethylquinazolin-4(3H)- one | 2.1 | 421.2 | B |
| 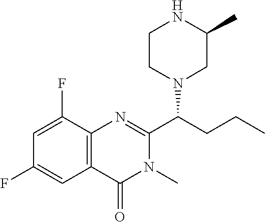 [SMILES: CCC[C@H](c1nc2c(F)cc(F)cc2c(=O)n1C)N1CCN[C@@H](C)C1] | 225 | 6,8-difluoro-3-methyl- 2-((R)-1-((S)-3- methylpiperazin-1- yl)butyl)quinazolin- 4(3H)-one | 1.75 | 351.2 | B |
| 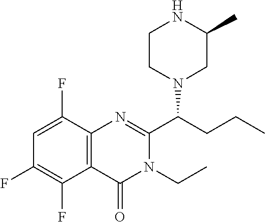 [SMILES: CCC[C@H](c1nc2c(F)cc(F)c(F)c2c(=O)n1CC)N1CCN[C@@H](C)C1] | 226 | 3-ethyl-5,6,8-trifluoro- 2-((R)-1-((S)-3- methylpiperazin-1- yl)butyl)quinazolin- 4(3H)-one | 1.88 | 383.3 | A |

The following compounds were obtained using the same method described in Example 1, but directly separating the enantiomeric or diastereomeric mixtures using chiral HPLC.

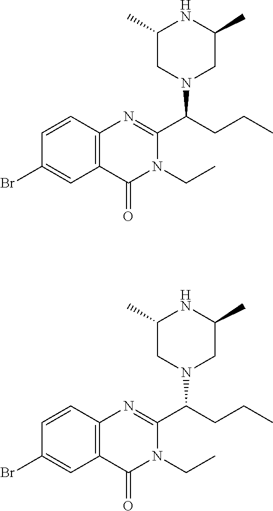 [SMILES: CCC[C@@H](c1nc2ccc(Br)cc2c(=O)n1CC)N1C[C@H](C)N[C@@H](C)C1 ; CCC[C@H](c1nc2ccc(Br)cc2c(=O)n1CC)N1C[C@H](C)N[C@@H](C)C1]

### Examples 227 and 228. 6-Bromo-2-((S)-1-((3S,5S)-3,5-dimethylpiperazin-1-yl)butyl)-3-ethylquinazolin-4(3H)-one and 6-Bromo-2-((R)-1-((3S,5S)-3,5-dimethylpiperazin-1-yl)butyl)-3-ethylquinazolin-4(3H)-one

Starting from 6-bromo-2-(1-((3S,5S)-3,5-dimethylpiperazin-1-yl)butyl)-3-ethylquinazolin-4(3H)-one, a chiral preparative HPLC separation [column: Chiralpak IC, temperature: ambient; flow: 13 mL/min, eluent n-Heptane/(IPA+0.33% DEA) 70/30 v/v; Rt₁: 9.4, Rt₂: 11.3] was carried out to give the title compounds.

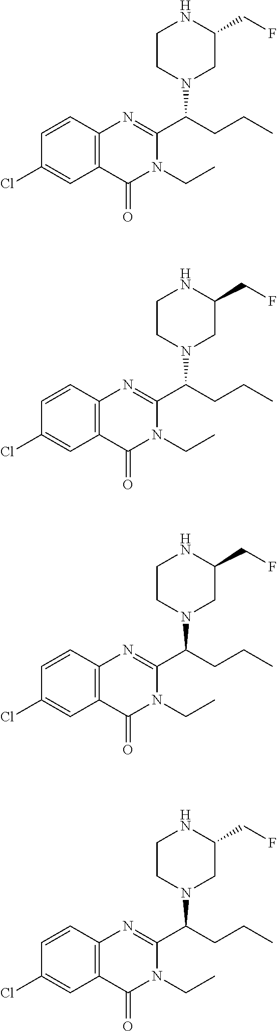 [SMILES: CCC[C@H](c1nc2ccc(Cl)cc2c(=O)n1CC)N1CCN[C@H](CF)C1 ; CCC[C@H](c1nc2ccc(Cl)cc2c(=O)n1CC)N1CCN[C@@H](CF)C1 ; CCC[C@@H](c1nc2ccc(Cl)cc2c(=O)n1CC)N1CCN[C@@H](CF)C1 ; CCC[C@@H](c1nc2ccc(Cl)cc2c(=O)n1CC)N1CCN[C@H](CF)C1]

### Examples 229, 230, 231 and 232. 6-Chloro-3-ethyl-2-((R)-1-((S)-3-(fluoromethyl)piperazin-1-yl)butyl)quinazolin-4(3H)-one, 6-chloro-3-ethyl-2-((R)-1-((R)-3-(fluoromethyl)piperazin-1-yl)butyl)quinazolin-4(3H)-one, 6-chloro-3-ethyl-2-((S)-1-((R)-3-(fluoromethyl)piperazin-1-yl)butyl)quinazolin-4(3H)-one and 6-chloro-3-ethyl-2-((S)-1-((S)-3-(fluoromethyl)piperazin-1-yl)butyl)quinazolin-4(3H)-one

Starting from 6-chloro-3-ethyl-2-(1-(3-(fluoromethyl)piperazin-1-yl)butyl)quinazolin-4(3H)-one, a chiral preparative HPLC separation [column: Chiralpak IG, temperature: ambient; flow: 12 mL/min, eluent n-Heptane/(IPA+0.33% DEA) 75/25 v/v; Rt₁: 12.7, Rt₂: 13.7′, Rt₃: 15.2′, Rt₄: 30.7′] was carried out to give the title compounds.

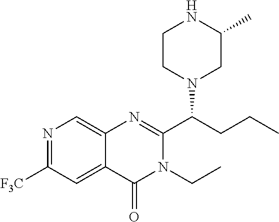 [SMILES: CCC[C@H](c1nc2cnc(C(F)(F)F)cc2c(=O)n1CC)N1CCN[C@H](C)C1]

### Example 233. 3-Ethyl-2-((R)-1-((R)-3-methylpiperazin-1-yl)butyl)-6-(trifluoromethyl)pyrido[3,4-d]pyrimidin-4(3H)-one

### Step a. 5-Amino-2-bromo-N-ethylisonicotinamide

Starting from 5-amino-2-bromoisonicotinic acid (1 g, 4.61 mmol) and following the procedure described in step a of example 1, the title compound was obtained (1.21 g, Yield: quant).

### Step b. (S)-1-((6-Bromo-4-(ethylcarbamoyl)pyridin-3-yl)amino)-1-oxopentan-2-yl acetate

To an ice-cold solution of (S)-2-acetoxypentanoic acid (1.12 g, 6.99 mmol) in anh DCM (20 mL), SOCl₂ (1.12 g, 24.47 mmol) was added and the reaction mixture was stirred at r.t for 4 h. The solvent was removed under vacuum and the crude product was washed twice with DCM and evaporated under vacuum to give (S)-1-chloro-1-oxopentan-2-yl acetate (1.23 g, Yield: 98%).

To a solution of compound obtained in step a (1.21 g, 4.59 mmol) in anh DCM (40 mL), DIPEA (4 mL, 13.77 mmol) and freshly prepared (S)-1-chloro-1-oxopentan-2-yl acetate (1.23, 6.89 mmol) were added under Ar atmosphere and the reaction mixture was stirred at r.t overnight. The reaction crude was washed with H₂O and the crude product was extracted with DCM, dried over anh Na₂SO₄, filtered and the solvent was removed under vacuum. The crude product was purified by flash chromatography, silica gel, gradient Chx to Chx:EtOAc (1:1) to give the title compound (1.63 g, Yield: 92%).

### Step c. (S)-6-Bromo-3-ethyl-2-(1-hydroxybutyl)pyrido[3,4-d]pyrimidin-4(3H)-one

To a solution of the compound obtained in step b (1.63 g, 4.23 mmol) in anh ACN (35 mL), ZnCl₂ (2.31 g, 16.9 mmol) and LiHMDS (3.55 mL, 16.9 mmol) were added and the resulting reaction mixture was heated at 80° C. overnight. The solvent was removed under vacuum and the crude product was redissolved with EtOAc, brine was added and the product was extracted with EtOAc. The organic phases were dried over anh Na₂SO₄, filtered and evaporated under vacuum to give a mixture of (S)-1-(6-bromo-3-ethyl-4-oxo-3,4-dihydropyrido[3,4-d]pyrimidin-2-yl)butyl acetate and (S)-6-bromo-3-ethyl-2-(1-hydroxybutyl)pyrido[3,4-d]pyrimidin-4(3H)-one (1.69 g).

To a solution of the previous product mixture in MeOH (40 mL), K₂CO₃ (292 mg, 2.12 mmol) was added at −70° C. and the reaction mixture was stirred at −20° C. for 4 h. The mixture was diluted with brine and the product was extracted with EtOAc. The organic phases were dried over anh Na₂SO₄, filtered and evaporated under vacuum. The crude product was purified by flash chromatography, silica gel, gradient Chx to Chx:EtOAc (7:3) to give the title compound (1.05 g, Yield: 75%).

### Step d. 6-Bromo-3-ethyl-2-((R)-1-((R)-3-methylpiperazin-1-yl)butyl)pyrido[3,4-d]pyrimidin-4(3H)-one

Starting from the compound obtained in step c (150 mg, 0.46 mmol) and following the procedure described in step b of example 198, the title compound was obtained (133 mg, Yield: 71%).

### Step e. 3-Ethyl-6-iodo-2-((R)-1-((R)-3-methylpiperazin-1-yl)butyl)pyrido[3,4-d]pyrimidin-4(3H)-one

To a solution of the compound obtained in step d (106 mg, 0.26 mmol) in anh Dioxane (4 mL), NaI (77.8 mg, 0.52 mmol), N¹,N²-dimethylethane-1,2-diamine (11 μL, 0.10 mmol) and CuI (9.9 mg, 0.052 mmol) were added under argon atmosphere and the reaction mixture was heated at 120° C. for 20 h. The reaction was quenched by the addition of sat aq NH₄OH solution and the product was extracted with EtOAc. The organic phases were dried over anh Na₂SO₄, filtered and evaporated under vacuum to give the title compound (100 mg, Yield: 81%).

### Step f. (R)-tert-Butyl 4-((R)-1-(3-ethyl-6-iodo-4-oxo-3,4-dihydropyrido[3,4-d]pyrimidin-2-yl)butyl)-2-methylpiperazine-1-carboxylate

To a solution of the compound obtained in step e (66 mg, 0.15 mmol) in anh DCM (5 mL), TEA (40 μL, 0.29 mmol) and di-tert-butyl dicarbonate (40 mg, 0.18 mmol) were added under argon atmosphere and the reaction was stirred at r.t. overnight. The mixture was diluted with EtOAc, washed with sat. aq. NaHCO₃ solution and the again extracted with EtOAc. The combined organic phases were dried over anh Na₂SO₄, filtered and evaporated under vacuum to give the title compound (86 mg, Yield: quant).

### Step g. (R)-tert-Butyl 4-((R)-1-(3-ethyl-4-oxo-6-(trifluoromethyl)-3,4-dihydropyrido[3,4-d]pyrimidin-2-yl)butyl)-2-methylpiperazine-1-carboxylate

To a solution of the compound obtained in step f (86 mg, 0.16 mmol) in DMF (1 mL), methyl 2,2-difluoro-2-(fluorosulfonyl)acetate (59.7 mg, 0.31 mmol) dissolved in DMF (0.5 mL) was added under argon atmosphere and the reaction was heated at 100° C. overnight. The mixture was quenched by the addition of H₂O and the product was extracted with EtOAc. The organic phases were dried over anh Na₂SO₄, filtered and evaporated under vacuum and the crude product was purified by flash chromatography, silica gel, gradient Chx to Chx:EtOAc (85:15) to give the title compound (25 mg, Yield: 32%).

### Step h. Title Compound

Starting from the compound obtained in step g (25 mg, 0.05 mmol) and following the procedure described in step b of example 79, the title compound was obtained (18 mg, Yield: 90%).

HPLC-MS (A) Rt 1.95 min; ESI+-MS m/z: 398.3 (M+1).

This method was used for the preparation of examples 234-235 using suitable starting materials:

| | | CHEMICAL | Rt | MS | HPLC |
|---|---|---|---|---|---|
| STRUCTURE | EX | NAME | (min) | (M + H) | Method |
| 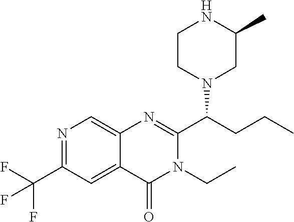 [SMILES: CCC[C@H](c1nc2cnc(C(F)(F)F)cc2c(=O)n1CC)N1CCN[C@@H](C)C1] | 234 | 3-ethyl-2-((R)-1-((S)-3- methylpiperazin-1- yl)butyl)-6- (trifluoromethyl)pyrido [3,4-d]pyrimidin-4(3H)- one | 1.96 | 398.1 | A |
| 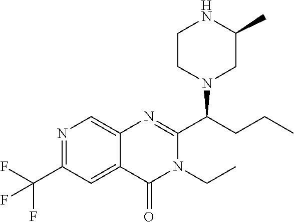 [SMILES: CCC[C@@H](c1nc2cnc(C(F)(F)F)cc2c(=O)n1CC)N1CCN[C@@H](C)C1] | 235 | 3-ethyl-2-((S)-1-((S)-3- methylpiperazin-1- yl)butyl)-6- (trifluoromethyl)pyrido [3,4-d]pyrimidin-4(3H)- one | 1.96 | 398.1 | A |

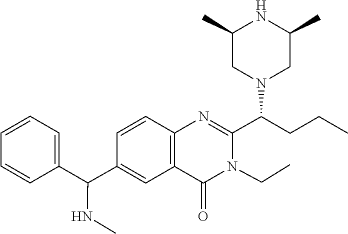 [SMILES: CCC[C@H](c1nc2ccc(C(NC)c3ccccc3)cc2c(=O)n1CC)N1C[C@@H](C)N[C@@H](C)C1]

### Example 236. 2-((R)-1-((3S,5R)-3,5-Dimethylpiperazin-1-yl)butyl)-3-ethyl-6-((methylamino)(phenyl)methyl)quinazolin-4(3H)-one

### Step a. (2S,6R)-tert-Butyl 4-((R)-1-(6-bromo-3-ethyl-4-oxo-3,4-dihydroquinazolin-2-yl)butyl)-2,6-dimethylpiperazine-1-carboxylate

Starting from the compound obtained in example 29 (1.05 g, 2.5 mmol) and following the procedure described in step f of example 233, the title compound was obtained (960 mg, Yield: 74%).

### Step b. (2S,6R)-tert-Butyl 4-((R)-1-(3-ethyl-4-oxo-6-(4,4,5,5-tetramethyl-1,3,2-dioxaborolan-2-yl)-3,4-dihydroquinazolin-2-yl)butyl)-2,6-dimethylpiperazine-1-carboxylate

To a solution of the compound obtained in step a (500 mg, 0.96 mmol) in anh dioxane (14 mL), 4,4,4′,4′,5,5,5′,5′-octamethyl-2,2′-bi(1,3,2-dioxaborolane) (365 mg, 1.43 mmol), potassium acetate (282 mg, 2.9 mmol), Pd(dppf)FeCl₂ (42 mg, 0.058 mmol) were added under argon atmosphere and the reaction mixture was heated at 95° C. 20 h. The crude was diluted with EtOAc and the organic layer was washed with H₂O and the crude product was extracted with EtOAc. The organic phases were dried over anh Na₂SO₄, filtered and evaporated under vacuum to give the title compound (542 mg, Yield: quant).

### Step c. (2-((R)-1-((3S,5R)-4-(tert-Butoxycarbonyl)-3,5-dimethylpiperazin-1-yl)butyl)-3-ethyl-4-oxo-3,4-dihydroquinazolin-6-yl)boronic acid

To a solution of the compound obtained in step b (542 mg, 0.96 mmol) in acetone:H₂O (2:1, 20 mL), ammonium acetate (2.2 g, 28.6 mmol) and sodium (meta)periodate (612.7 mg, 2.9 mmol) were added and the reaction mixture was stirred at r.t overnight. The solvent was removed under vacuum, the crude was dissolved with EtOAc, washed with H₂O, dried over anh Na₂SO₄, filtered and evaporated under vacuum to give the title compound (504 mg, Yield: quant).

### Step d. (2S,6R)-tert-Butyl 4-((1R)-1-(3-ethyl-6-((4-methylphenylsulfonamido)(phenyl)methyl)-4-oxo-3,4-dihydroquinazolin-2-yl)butyl)-2,6-dimethylpiperazine-1-carboxylate

To a solution of the compound obtained in step c (336 mg, 0.69 mmol) in nitrometane (6 mL), N-benzylidene-4-methylbenzenesulfonamide (538 mg, 2.1 mmol), PdCl₂(bpy) (46 mg, 0.14 mmol) and silver nitrate (47 mg, 0.28 mmol) were added and the reaction mixture was heated at 100° C. for 24 h. The solvent was removed under vacuum and the crude product was purified by flash chromatography, silica gel, gradient Chx to Chx:EtOAc (4:6) to give the title compound (310 mg, Yield: 45%).

### Step e. (2S,6R)-tert-Butyl 4-((1R)-1-(6-((N,4-dimethylphenylsulfonamido)(phenyl)methyl)-3-ethyl-4-oxo-3,4-dihydroquinazolin-2-yl)butyl)-2,6-dimethylpiperazine-1-carboxylate

To a solution of the compound obtained in step d (310 mg, 0.44 mmol) in ACN (3 mL), potassium carbonate (610 mg, 4.4 mmol) and methyl iodide (277 μL, 4.4 mmol) were added and the reaction was heated at 80° C. overnight. Then, sat. aq NaHCO₃ solution was added and the product was extracted with EtOAc. The organic phases were dried over anh Na₂SO₄, filtered and evaporated under vacuum. The crude product was purified by flash chromatography, silica gel, gradient Chx to Chx:EtOAc (1:3) to give the title compound (143 mg, Yield: 45%).

### Step f. Title Compound

To a solution of the compound obtained in step e (50 mg, 0.07 mmol) in anh THE (1.5 mL), a freshly prepared solution of Na (8 mg, 0.35 mmol) and naphtalene (8 g, 0.35 mmol) in and THE (0.7 mL) was added at −78° C. and the reaction mixture was stirred at this temperature for 1 h. The reaction was quenched by the addition of aq NH₄Cl solution and the product was extracted with EtOAc. The organic phases were dried over anh Na₂SO₄, filtered and evaporated under vacuum.

The resulting crude product was dissolved in anh DCM (5 mL) and TFA (109 μL, 1.42 mmol) was added drop wise at r.t. The reaction mixture was stirred at r.t. for 20 h. The crude mixture was diluted with DCM, washed with sat. aq NaHCO₃ and the organic layer was dried over anh Na₂SO₄, filtered and evaporated under vacuum. The crude product was purified by flash chromatography, silica gel, gradient DCM to DCM:MeOH (8:2) to give the title compound (3.6 mg, Yield: 11%).

HPLC-MS (A) Rt 2.06 min; ESI+-MS m/z: 462.1 (M+1).

Table of Examples with Binding to the μ-Opioid Receptor and the α₂δ-1 Subunit of the Voltage-Gated Calcium Channel:

Biological Activity

Pharmacological Study

Human α₂δ-1 subunit of Caᵥ2.2 calcium channel assay Human α2δ-1 enriched membranes (2.5 μg) were incubated with 15 nM of radiolabeled [3H]-Gabapentin in assay buffer containing Hepes-KOH 10 mM, pH 7.4. NSB (non specific binding) was measured by adding 10 μM pregabalin. The binding of the test compound was measured at either one concentration (% inhibition at 1 or 10 μM) or five different concentrations to determine affinity values (Ki). After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

Human μ-Opioid Receptor Radioligand Assay

Transfected CHO-K1 cell membranes (20 μg) were incubated with 1 nM of [³H]-DAMGO in assay buffer containing Tris-HCl 50 mM, MgCl2 5 mM at pH 7.4. NBS (non-specific binding) was measured by adding 10 μM Naloxone. The binding of the test compound was measured at either one concentration (% inhibition at 1 or 10 μM) or five different concentrations to determine affinity values (Ki). Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

Results:

As this invention is aimed at providing a compound or a chemically related series of compounds which act as ligands of the α2δ subunit of voltage-gated calcium channels. It is a very preferred embodiment in which the compounds are selected which act as dual ligands of the α2δ subunit of voltage-gated calcium channels and the μ-opioid receptor and especially compounds which have a binding expressed as Kᵢ responding to the following scales:
Kᵢ(μ) is preferably <1000 nM, more preferably <500 nM, even more preferably <100 nM.

Kᵢ(α₂δ-1) is preferably <10000 nM, more preferably <5000 nM, or even more preferably <500 nM.

The following scale has been adopted for representing the binding to μ-opioid receptor expressed as Kᵢ:
+Kᵢ (μ)>=500 nM
++100 nM<=Kᵢ(μ)<500 nM
+++Kᵢ(μ)<100 nM

Preferably, when Kᵢ (μ)>500 nM, the following scale has been adopted for representing the binding to the μ-receptor:
+Kᵢ (μ)>500 nM or inhibition ranges between 1% and 50%.

The following scale has been adopted for representing the binding to the α₂δ-1 subunit of voltage-gated calcium channels expressed as Kᵢ:
+Kᵢ(α₂δ-1)>=5000 nM
++500 nM<=Kᵢ((α₂δ-1)<5000 nM
+++Kᵢ(α₂δ-1)<500 nM

Preferably, when Kᵢ(α₂δ-1)>5000 nM, the following scale has been adopted for representing the binding to the α₂δ-1 subunit of voltage-gated calcium channels: +Kᵢ(α₂δ-1)>5000 nM or inhibition ranges between 1% and 50%.

All compounds prepared in the present application exhibit binding to the α₂δ-1 subunit of voltage-gated calcium channels or binding to the α₂δ-1 subunit of voltage-gated calcium channels and the μ-opioid receptor, in particular the following binding results are shown:

| | μ- | α2δ-1 |
|---|---|---|
| Example | binding | binding |
| 1 | + | +++ |
| 2 | + | +++ |
| 3 | + | +++ |
| 4 | + | +++ |
| 5 | + | ++ |
| 6 | + | +++ |
| 7 | ++ | +++ |
| 9 | + | +++ |
| 10 | + | +++ |
| 11 | + | +++ |
| 12 | + | +++ |
| 13 | + | + |
| 14 | + | +++ |
| 15 | + | +++ |
| 16 | + | +++ |
| 17 | + | +++ |
| 18 | + | +++ |
| 19 | + | +++ |
| 20 | + | + |
| 21 | + | +++ |
| 22 | + | +++ |
| 23 | + | +++ |
| 24 | + | +++ |
| 25 | + | +++ |
| 26 | + | ++ |
| 27 | + | +++ |
| 28 | + | +++ |
| 29 | + | +++ |
| 30 | + | ++ |
| 31 | + | ++ |
| 32 | + | +++ |
| 33 | + | ++ |
| 34 | + | +++ |
| 35 | + | +++ |
| 36 | + | ++ |
| 37 | + | + |
| 38 | ++ | +++ |
| 39 | + | + |
| 40 | + | ++ |
| 41 | ++ | +++ |
| 42 | + | + |
| 43 | + | +++ |
| 44 | + | + |
| 45 | + | + |
| 46 | ++ | +++ |
| 47 | + | +++ |
| 48 | + | + |
| 49 | ++ | +++ |
| 50 | + | + |
| 51 | ++ | +++ |
| 52 | + | + |
| 53 | ++ | +++ |
| 54 | + | + |
| 55 | + | +++ |
| 56 | + | + |
| 57 | + | +++ |
| 58 | + | +++ |
| 59 | + | + |
| 60 | + | +++ |
| 61 | + | + |
| 62 | ++ | +++ |
| 63 | + | + |
| 64 | + | +++ |
| 65 | + | + |
| 66 | + | +++ |
| 67 | + | + |
| 68 | + | +++ |
| 69 | + | + |
| 70 | + | +++ |
| 71 | + | + |
| 72 | + | +++ |
| 73 | + | + |
| 74 | + | + |
| 75 | + | +++ |
| 76 | ++ | +++ |
| 77 | + | + |
| 78 | + | +++ |
| 79 | + | +++ |
| 80 | + | ++ |
| 81 | + | +++ |
| 82 | ++ | +++ |
| 83 | ++ | +++ |
| 84 | ++ | ++ |
| 85 | + | +++ |
| 86 | + | +++ |
| 87 | ++ | ++ |
| 88 | + | +++ |
| 89 | +++ | + |
| 90 | ++ | + |
| 91 | ++ | +++ |
| 92 | + | +++ |
| 93 | + | +++ |
| 94 | + | +++ |
| 95 | + | + |
| 96 | ++ | +++ |
| 97 | ++ | +++ |
| 98 | + | +++ |
| 99 | + | +++ |
| 100 | + | + |
| 101 | + | +++ |
| 102 | + | + |
| 103 | +++ | +++ |
| 104 | + | + |
| 105 | + | +++ |
| 106 | +++ | +++ |
| 107 | + | + |
| 108 | + | + |
| 109 | ++ | +++ |
| 110 | + | + |
| 111 | + | + |
| 112 | +++ | ++ |
| 113 | + | +++ |
| 114 | + | + |
| 115 | + | ++ |
| 116 | + | ++ |
| 117 | + | ++ |
| 118 | + | +++ |
| 119 | + | +++ |
| 120 | + | +++ |
| 121 | + | +++ |
| 122 | + | ++ |
| 123 | + | +++ |
| 124 | + | ++ |
| 125 | ++ | ++ |
| 126 | + | + |
| 127 | + | ++ |
| 128 | + | ++ |
| 129 | + | ++ |
| 130 | + | ++ |
| 131 | + | + |
| 132 | + | +++ |
| 133 | + | +++ |
| 134 | + | +++ |
| 135 | + | +++ |
| 136 | + | ++ |
| 137 | + | +++ |
| 138 | + | +++ |
| 139 | + | +++ |
| 140 | + | +++ |
| 141 | + | +++ |
| 142 | + | +++ |
| 143 | + | +++ |
| 144 | + | +++ |
| 145 | + | +++ |
| 146 | + | +++ |
| 147 | + | +++ |
| 148 | + | +++ |
| 149 | + | +++ |
| 150 | + | +++ |
| 151 | + | +++ |
| 152 | + | ++ |
| 153 | + | ++ |
| 154 | + | +++ |
| 155 | + | +++ |
| 156 | + | ++ |
| 157 | + | ++ |
| 158 | + | +++ |
| 159 | + | ++ |
| 160 | + | +++ |
| 161 | + | + |
| 162 | + | ++ |
| 163 | + | +++ |
| 164 | + | ++ |
| 165 | + | ++ |
| 166 | + | +++ |
| 167 | + | +++ |
| 168 | + | ++ |
| 169 | + | ++ |
| 170 | + | +++ |
| 171 | + | ++ |
| 172 | + | +++ |
| 173 | + | +++ |
| 174 | + | +++ |
| 175 | + | +++ |
| 176 | + | +++ |
| 177 | + | +++ |
| 178 | + | ++ |
| 179 | + | ++ |
| 180 | + | +++ |
| 181 | + | +++ |
| 182 | + | +++ |
| 183 | + | +++ |
| 184 | + | +++ |
| 185 | + | +++ |
| 186 | + | +++ |
| 187 | + | ++ |
| 188 | + | ++ |
| 189 | + | +++ |
| 190 | + | + |
| 191 | ++ | +++ |
| 192 | + | + |
| 193 | + | + |
| 194 | + | + |
| 195 | + | +++ |
| 196 | + | + |
| 197 | + | ++ |
| 198 | ++ | +++ |
| 199 | ++ | +++ |
| 200 | ++ | +++ |
| 201 | +++ | +++ |
| 202 | + | +++ |
| 203 | + | +++ |
| 204 | + | +++ |
| 205 | ++ | +++ |
| 206 | ++ | +++ |
| 207 | ++ | +++ |
| 208 | + | +++ |
| 209 | ++ | +++ |
| 210 | + | +++ |
| 211 | + | +++ |
| 212 | + | +++ |
| 213 | + | +++ |
| 214 | ++ | +++ |
| 215 | + | +++ |
| 216 | ++ | +++ |
| 217 | +++ | +++ |
| 218 | ++ | +++ |
| 219 | + | +++ |
| 220 | + | + |
| 221 | +++ | ++ |
| 222 | + | ++ |
| 223 | +++ | + |
| 224 | +++ | + |
| 225 | ++ | ++ |
| 226 | ++ | ++ |
| 227 | + | + |
| 228 | +++ | + |
| 229 | + | +++ |
| 230 | + | ++ |
| 231 | + | + |
| 232 | + | + |
| 233 | + | +++ |
| 234 | + | +++ |
| 235 | + | + |
| 236 | + | +++ |

1-12. (canceled)

13. A compound of the formula: 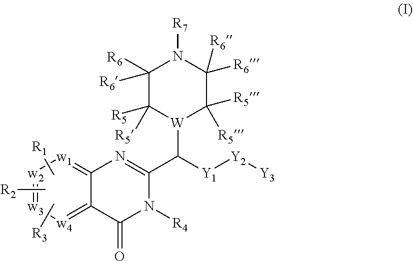
or a salt thereof, or a solvate thereof;
wherein
Y₁ is —C(R_{y}R_{y}′)—,
wherein
R_{y} and R_{y}′ are independently selected from the group consisting of hydrogen, substituted or unsubstituted C₁₋₆ alkyl, substituted or unsubstituted C₂₋₆ alkenyl and substituted or unsubstituted C₂₋₆ alkynyl; or
R_{y} and R_{y}′ together with the carbon atom to which they are attached form a substituted or unsubstituted cycloalkyl;
Y₂ is —C(R_{y}″R_{y}′″)—,
wherein
R_{y}″ and R_{y}′″ are independently selected from the group consisting of hydrogen, substituted or unsubstituted C₁₋₆ alkyl, substituted or unsubstituted C₂₋₆ alkenyl and substituted or unsubstituted C₂₋₆ alkynyl; or
R_{y}″ and R_{y}′″ together with the carbon atom to which they are attached form a substituted or unsubstituted cycloalkyl;
Y₃ is —CH₃ or —CH₂CH₃; or
Y₂ and Y₃ together form a substituted or unsubstituted cycloalkyl;
W is nitrogen or —CR_{w}—,
wherein
R_{w} is hydrogen or halogen; or
R_{w} and one of R₅, R₅′, R₅″ or R₅′″ together form a double bond;
w1, w2, w3 and w4 are independently selected from the group consisting of nitrogen and carbon;
R₁ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₈, —(CH₂)ₙNR₈R₈′, —CH(phenyl)-NR₈R₈′, —NR₈C(O)R₈′, —NR₈C(O)OR₈′, —C(O)NR₈R_{8′}, —C(O)OR₈, —OCHR₈R₈′, haloalkyl, haloalkoxy, —CN, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
n is 0, 1, 2, 3, 4 or 5;
R₈ and R₈′ are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted heterocyclyl, substituted or unsubstituted aryl, substituted or unsubstituted alkylcycloalkyl, substituted or unsubstituted alkylheterocyclyl and substituted or unsubstituted alkylaryl;
R₂ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₂₁, —NO₂, —NR₂₁R₂₁′, —NR₂₁C(O)R₂₁′, —NR₂₁S(O)₂R₂₁′, —S(O)₂NR₂₁R₂₁′, —NR₂₁C(O)NR₂₁′R₂₁″, —SR₂₁, —S(O)R₂₁, —S(O)₂R₂₁, —CN, haloalkyl, haloalkoxy, —C(O)OR₂₁, —C(O)NR₂₁R₂₁′, —NR₂₁S(O)₂NR₂₁′R₂₁″ and —C(CH₃)₂OR₂₁,
wherein
R₂₁, R₂₁′ and R₂₁″ are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
R₃ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, —OR₃₁, —NO₃, —NR₃₁R₃₁′, —NR₃₁C(O)R₃₁′, —NR₃₁S(O)₃R₃₁′, —S(O)₃NR₃₁R₃₁′, —NR₃₁C(O)NR₃₁′R₃₁″, —SR₃₁, —S(O)R₃₁, —S(O)₃R₃₁, —CN, haloalkyl, haloalkoxy, —C(O)OR₃₁, —C(O)NR₃₁R₃₁′, —NR₃₁S(O)₃NR₃₁′R₃₁″ and —C(CH₃)₃OR₃₁,
wherein
R₃₁, R₃₁′ and R₃₁″ are independently selected from the group consisting of hydrogen, substituted or unsubstituted C₁₋₆ alkyl, substituted or unsubstituted C₃₋₆ alkenyl and substituted or unsubstituted C₃₋₆ alkynyl;
R₄ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted alkylheterocyclyl, substituted or unsubstituted alkylaryl and substituted or unsubstituted alkylcycloalkyl;
R₅, R₅′, R₅″ and R₅′″ are independently selected from the group consisting of hydrogen, halogen, substituted or unsubstituted C₁₋₆ alkyl, substituted or unsubstituted C₂₋₆ alkenyl and substituted or unsubstituted C₂₋₆ alkynyl; or
R₅ and R₅′ and/or R₅″ and R₅′″ together with the carbon atom to which they are attached form a carbonyl group;
R₆, R₆′. R₆″ and R₆′″ are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
R₇ is selected from the group consisting of hydrogen, substituted or unsubstituted C₁₋₆ alkyl, substituted or unsubstituted C₂₋₆ alkenyl and substituted or unsubstituted C₂₋₆ alkynyl;
with the proviso that when R₇ is not hydrogen, then one of R₆, R₆′, R₆″ or R₆′″ is not hydrogen.

14. The compound according to claim 13 of the Formula: 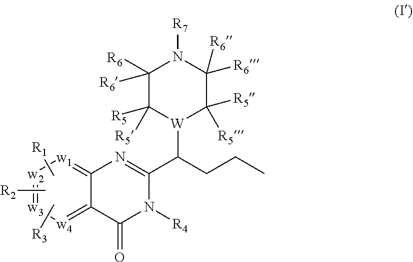
wherein R₁, R₂, R₃, R₄, R₅, R₅′, R₅″, R₅′″, R₆, R₆′, R₆″, R₆′″, R₇, W, w₁, w₂, w₃ and w₄ are as defined in claim 13.

15. The compound according to claim 13 of the Formula: 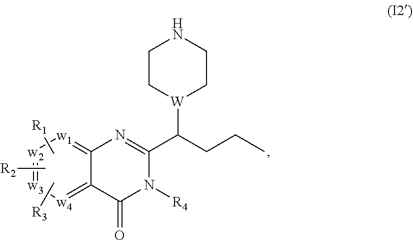
wherein R₁, R₂, R₃, R₄, W, w₁, w₂, w₃ and w₄ are as defined in claim 13.

16. The compound according to claim 13 of the Formula: 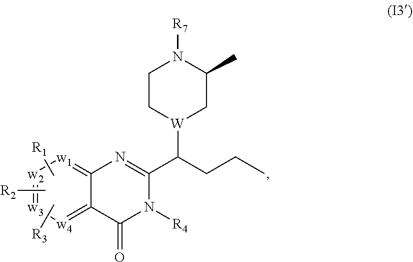
wherein R₁, R₂, R₃, R₄, R₇, W, w₁, w₂, w₃ and w₄ are as defined in claim 13.

17. The compound according to claim 13 of the Formula: 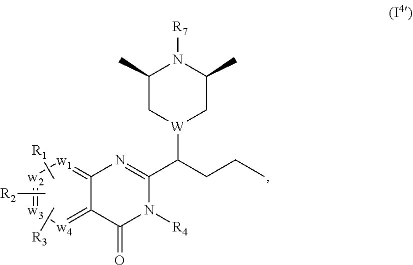
wherein R₁, R₂, R₃, R₄, R₇, W, w₁, w₂, w₃ and w₄ are as defined in claim 13.

18. The compound according to claim 13 of the Formula: 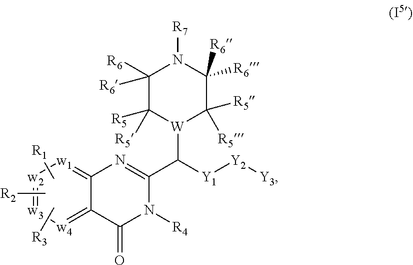
wherein R₁, R₂, R₃, R₄, R₅, R₅′, R₅″, R₅′″, R₆, R₆′, R₆″, R₆′″, R₇, Y₁, Y₂, Y₃, W, w₁, w₂, w₃ and w₄ are as defined in claim 13.

19. The compound according to claim 13, wherein said compound is selected from the group consisting of:

| | |
|---|---|
| 1 | 6-bromo-2-(1-((3S,5R)-3,5-dimethylpiperazin-1-yl)butyl)-3-ethylquinazolin-4(3H)-one |
| 2 | 2-(1-((3S,5R)-3,5-dimethylpiperazin-1-yl)butyl)-3-methylquinazolin-4(3H)-one |
| 3 | 2-(1-((3S,5R)-3,5-dimethylpiperazin-1-yl)butyl)-3-(2-(methylamino)ethyl)quinazolin- |
| | 4(3H)-one |
| 4 | 2-(1-((3S,5R)-3,5-dimethylpiperazin-1-yl)butyl)-3-ethyl-7-methylquinazolin-4(3H)-one |
| 5 | 8-bromo-2-(1-((3S,5R)-3,5-dimethylpiperazin-1-yl)butyl)-3-ethyl-7-methoxyquinazolin- |
| | 4(3H)-one |
| 6 | 2-(1-((3S,5R)-3,5-dimethylpiperazm-1-yl)butyl)-3-ethyl-7-methoxyquinazolin-4(3H)-one |
| 7 | methyl 2-(1-((3S,5R)-3,5-dimethylpiperazin-1-yl)butyl)-3-ethyl-4-oxo-3,4- |
| | dihydroquinazoline-7-carboxylate |
| 8 | methyl 2-(1-((3S,5R)-3,5-dimethylpiperazin-1-yl)butyl)-3-methyl-4-oxo-3,4- |
| | dihydroquinazoline-7-carboxylate |
| 9 | 7-bromo-2-(1-((3S,5R)-3,5-dimethylpiperazin-1-yl)butyl)-3-ethylquinazolin-4(3H)-one |
| 10 | 2-(1-((3S,5R)-3,5-dimethylpiperazin-1-yl)butyl)-7-hydroxy-3-methylquinazolin-4(3H)-one |
| 11 | 2-(1-((3S,5R)-3,5-dimethylpiperazin-1-yl)butyl)-3-ethyl-7-fluoroquinazolin-4(3H)-one |
| 12 | 2-(1-((3S,5R)-3,5-dimethylpiperazin-1-yl)butyl)-3-ethyl-6-hydroxyquinazolin-4(3H)-one |
| 13 | 8-bromo-2-(1-((3S,5R)-3,5-dimethylpiperazin-1-yl)butyl)-3-ethylquinazolin-4(3H)-one |
| 14 | 5-bromo-2-(1-((3S,5R)-3,5-dimethylpiperazin-1-yl)butyl)-3-ethylquinazolin-4(3H)-one |
| 15 | 2-(1-((3S,5R)-3,5-dimethylpiperazin-1-yl)butyl)-3-ethyl-5-hydroxyquinazolin-4(3H)-one |
| 16 | 6-bromo-2-(1-((3S,5R)-3,5-dimethylpiperazin-1-yl)butyl)-3-methylquinazolin-4(3H)-one |
| 17 | 7-bromo-2-(1-((3S,5R)-3,5-dimethylpiperazin-1-yl)butyl)-3-methylquinazolin-4(3H)-one |
| 18 | 6-bromo-2-(1-((3S,5R)-3,5-dimethylpiperazin-1-yl)butyl)-3-ethyl-7-fluoroquinazolin- |
| | 4(3H)-one |
| 19 | 6-bromo-2-(1-((3S,5R)-3,5-dimethylpiperazin-1-yl)butyl)-3-ethyl-7-methylquinazolin- |
| | 4(3H)-one |
| 20 | 6-bromo-2-(1-((3S,5R)-3,5-dimethylpiperazin-1-yl)butyl)-3-ethyl-8-methylquinazolin- |
| | 4(3H)-one |
| 21 | 6-bromo-2-(1-((3S,5R)-3,5-dimethylpiperazin-1-yl)butyl)-3-ethyl-5-methylquinazolin- |
| | 4(3H)-one |
| 22 | 6-bromo-2-(1-((3S,5R)-3,5-dimethylpiperazin-1-yl)butyl)-3-ethyl-5-fluoroquinazolin- |
| | 4(3H)-one |
| 23 | 6-bromo-5-chloro-2-(1-((3S,5R)-3,5-dimethylpiperazin-1-yl)butyl)-3-ethylquinazolin- |
| | 4(3H)-one |
| 24 | 2-(1-((3S,5R)-3,5-dimethylpiperazin-1-yl)butyl)-3-ethyl-6-(ethylamino)quinazolin-4(3H)-one |
| 25 | 2-(1-((3S,5R)-3,5-dimethylpiperazin-1-yl)butyl)-3-ethyl-7-(ethylamino)quinazolin-4(3H)-one |
| 26 | tert-butyl (2-(1-((3S,5R)-3,5-dimethylpiperazin-1-yl)butyl)-3-ethyl-4-oxo-3,4- |
| | dihydroquinazolin-7-yl)(ethyl)carbamate |
| 27 | 2-(1-((3S,5R)-3,5-dimethylpiperazin-1-yl)butyl)-3-ethylpyrido[3,2-d]pyrimidin-4(3H)-one |
| 28 | 6-bromo-2-(1-((3S,5R)-3,5-dimethylpiperazin-1-yl)butyl)-3-ethylpyrido[2,3-d]pyrimidin- |
| | 4(3H)-one |
| 29 | 6-bromo-2-((R)-1-((3S,5R)-3,5-diniethylpiperazin-1-yl)butyl)-3-ethylquinazolm-4(3H)-one |
| 30 | 6-bromo-2-((S)-1-((3S,5R)-3,5-dimethylpiperazin-1-yl)butyl)-3-ethylquinazolin-4(3H)-one |
| 31 | -((S)-1-((3S,5R)-3,5-dimethylpiperazin-1-yl)butyl)-3-methylquinazolin-4(3H)-one |
| 32 | 2-((R)-1-((3S,5R)-3,5-dimethylpiperazin-1-yl)butyl)-3-methylquinazolin-4(3H)-one |
| 33 | 2-((S)-1-((3S,5R)-3,5-dimethylpiperazin-1-yl)butyl)-3-methylquinazolin-4(3H)-one |
| 34 | 2-((R)-1-((3S,5R)-3,5-dimethylpiperazin-1-yl)butyl)-3-ethylquinazolin-4(3H)-one |
| 35 | 2-((R)-1-((3S,5R)-3,5-dimethylpiperazin-1-yl)butyl)-3-ethyl-6-hydroxyquinazolin-4(3H)-one |
| 36 | 2-((S)-1-((3S,5R)-3,5-dirnethylpiperazin-1-yl)butyl)-3-ethyl-6-hydroxyquinazolin-4(3H)-one |
| 37 | (S)-3-ethyl-8-fluoro-6-methoxy-2-(1-(piperazin-1-yl)butyl)quinazolin-4(3H)-one |
| 38 | (R)-3-ethyl-8-fluoro-6-methoxy-2-(1-(piperazin-1-yl)butyl)quinazolin-4(3H)-one |
| 39 | (S)-5-bromo-3-ethyl-8-fluoro-6-methoxy-2-(1-(piperazin-1-yl)butyl)quinazolin-4(3H)-one |
| 40 | (R)-5-bromo-3-ethyl-8-fluoro-6-methoxy-2-(1-(piperazin-1-yl)butyl)quinazolin-4(3H)-one |
| 41 | 6-bromo-3-methyl-2-((R)-1-((S)-3-methylpiperazin-1-yl)butyl)quinazolin-4(3H)-one |
| 42 | 6-bromo-3-methyl-2-((S)-1-((S)-3-methylpiperazin-1-yl)butyl)quinazolin-4(3H)-one |
| 43 | 3-ethyl-2-((R)-1-((S)-3-methylpiperazin-1-yl)butyl)quinazolin-4(3H)-one |
| 44 | 3-ethyl-2-((S)-1-((S)-3-methylpiperazin-1-yl)butyl)quinazolin-4(3H)-one |
| 45 | 3-ethyl-8-fluoro-6-methoxy-2-((S)-1-((S)-3-methylpiperazin-1-yl)butyl)quinazolin- |
| | 4(3H)-one |
| 46 | 3-ethyl-8-fluoro-6-methoxy-2-((R)-1-((S)-3-methylpiperazin-1-yl)butyl)quinazolin- |
| | 4(3H)-one |
| 47 | 6-bromo-3-ethyl-8-fluoro-2-((R)-1-((R)-3-methylpiperazin-1-yl)butyl)quinazolin-4(3H)-one |
| 48 | 6-bromo-3-ethyl-8-fluoro-2-((S)-1-((R)-3-methylpiperazin-1-yl)butyl)quinazolin-4(3H)-one |
| 49 | 6,7-dichloro-3-ethyl-2-((R)-1-((S)-3-methylpiperazin-1-yl)butyl)quinazolin-4(3H)-one |
| 50 | 6,7-dichloro-3-ethyl-2-((S)-1-((S)-3-methylpiperazin-1-yl)butyl)quinazolin-4(3H)-one |
| 51 | 6-bromo-3-ethyl-8-fluoro-2-((R)-1-((S)-3-methylpiperazin-1-yl)butyl)quinazolin-4(3H)-one |
| 52 | 6-bromo-3-ethyl-8-fluoro-2-((S)-1-((S)-3-methylpiperazin-1-yl)butyl)quinazolin-4(3H)-one |
| 53 | 6-chloro-3-ethyl-7-fluoro-2-((R)-1-((S)-3-methylpiperazin-1-yl)butyl)quinazolin-4(3H)-one |
| 54 | 6-chloro-3-ethyl-7-fluoro-2-((S)-1-((S)-3-methylpiperazin-1-yl)butyl)quinazolin-4(3H)-one |
| 55 | 6-bromo-3-ethyl-2-((R)-1-((S)-3-(fluoromethyl)piperazin-1-yl)butyl)quinazolin-4(3H)-one |
| 56 | 6-bromo-3-ethyl-2-((S)-1-((R)-3-(fluoromethyl)piperazin-1-yl)butyl)quinazolin-4(3H)-one |
| 57 | 6-bromo-3-ethyl-2-((S)-1-((S)-3-(fluoromethyl)piperazin-1-yl)butyl)quinazolin-4(3H)-one |
| 58 | 6-bromo-3-ethyl-2-((R)-1-((S)-3-(hydroxymethyl)piperazin-1-yl)butyl)quinazolin-4(3H)-one |
| 59 | 6-bromo-3-ethyl-2-((S)-1-((S)-3-(hydroxymethyl)piperazin-1-yl)butyl)quinazolin-4(3H)-one |
| 60 | 6-bromo-3-ethyl-2-((R)-1-((R)-3-(hydroxymethyl)piperazin-1-yl)butyl)quinazolin-4(3H)-one |
| 61 | 6-bromo-3-ethyl-2-((S)-1-((R)-3-(hydroxymethyl)piperazin-1-yl)butyl)quinazolin-4(3H)-one |
| 62 | 6-bromo-7-fluoro-3-methyl-2-((R)-1-((S)-3-methylpiperazin-1-yl)butyl)quinazolin- |
| | 4(3H)-one |
| 63 | 6-bromo-7-fluoro-3-methyl-2-((S)-1-((S)-3-methylpiperazin-1-yl)butyl)quinazolin-4(3H)-one |
| 64 | 3-ethyl-2-((R)-1-((R)-3-methylpiperazin-1-yl)butyl)pyrido[4,3-d]pyrimidin-4(3H)-one |
| 65 | 3-ethyl-2-((S)-1-((R)-3-methylpiperazin-1-yl)butyl)pyrido[4,3-d]pyrimidin-4(3H)-one |
| 66 | 3-ethyl-2-((R)-1-((S)-3-methylpiperazin-1-yl)butyl)pyrido[4,3-d]pyrimidin-4(3H)-one |
| 67 | 3-ethyl-2-((S)-1-((S)-3-methylpiperazin-1-yl)butyl)pyrido[4,3-d]pyrimidin-4(3H)-one |
| 68 | 6-bromo-3-ethyl-2-((R)-1-((S)-3-methylpiperazin-1-yl)butyl)pyrido[2,3-d]pyrimidin- |
| | 4(3H)-one |
| 69 | 6-bromo-3-ethyl-2-((S)-1-((S)-3-methylpiperazin-1-yl)butyl)pyrido[2,3-d]pyrimidin- |
| | 4(3H)-one |
| 70 | 3-ethyl-2-((R)-1-((S)-3-methylpiperazin-1-yl)butyl)pyrido[3,2-d]pyrimidin-4(3H)-one |
| 71 | 3-ethyl-2-((S)-1-((S)-3-methylpiperazin-1-yl)butyl)pyrido[3,2-d]pyrimidin-4(3H)-one |
| 72 | 6-bromo-2-((R)-1-((S)-3-methylpiperazin-1-yl)butyl)-3-propylpyrido[2,3-d]pyrimidin- |
| | 4(3H)-one |
| 73 | 6-bromo-2-((S)-1-((S)-3-methylpiperazin-1-yl)butyl)-3-propylpyrido[2,3-d]pyrimidin- |
| | 4(3H)-one |
| 74 | 6-bromo-3-(cyclopropylmethyl)-2-((S)-1-((S)-3-methylpiperazin-1-yl)butyl)pyrido[2,3- |
| | d]pyrimidin-4(3H)-one |
| 75 | 6-bromo-3-(cyclopropylmethyl)-2-((R)-1-((S)-3-methylpiperazin-1-yl)butyl)pyrido[2,3- |
| | d]pyrimidin-4(3H)-one |
| 76 | 6-chloro-3-ethyl-2-((R)-1-((S)-3-methylpiperazin-1-yl)butyl)pyrido[2,3-d]pyrimidin- |
| | 4(3H)-one |
| 77 | 3-ethyl-2-((S)-1-((S)-3-methylpiperazin-1-yl)butyl)-7-(trifluoromethyl)pyrido[2,3- |
| | d]pyrimidin-4(3H)-one |
| 78 | 3-ethyl-2-((R)-1-((S)-3-methylpiperazin-1-yl)butyl)-7-(trifluoromethyl)pyrido[2,3- |
| | d]pyrimidin-4(3H)-one |
| 79 | 6-bromo-3-ethyl-2-(1-(piperazin-1-yl)butyl)quinazolin-4(3H)-one. |
| | Step a. tert-Butyl 4-(1-(6-bromo-3-ethyl-4-oxo-3,4-dihydroquinazolin-2- |
| | yl)butyl)piperazine-1-carboxylate |
| 80 | 3-methyl-2-(1-(piperazin-1-yl)butyl)quinazolin-4(3H)-one |
| 81 | 7-bromo-3-ethyl-2-(1-(piperazin-1-yl)butyl)quinazolin-4(3H)-one |
| 82 | 6-bromo-2-(1-(piperazin-1-yl)butyl)-3-propylquinazolin-4(3H)-one |
| 83 | 6-bromo-3-ethyl-7-fluoro-2-(1-(piperazin-1-yl)butyl)quinazolin-4(3H)-one |
| 84 | 6-bromo-3-ethyl-5-methyl-2-(1-(piperazin-1-yl)butyl)quinazolin-4(3H)-one |
| 85 | 6-bromo-3-ethyl-7-methyl-2-(1-(piperazin-1-yl)butyl)quinazolin-4(3H)-one |
| 86 | 3-ethyl-6,7-difluoro-2-(1-(piperazin-1-yl)butyl)quinazolin-4(3H)-one |
| 87 | 6-bromo-3-ethyl-8-fluoro-2-(1-(piperazin-1-yl)butyl)quinazolin-4(3H)-one |
| 88 | 6-bromo-3-ethyl-5-fluoro-2-(1-(piperazin-1-yl)butyl)quinazolin-4(3H)-one |
| 89 | 6-bromo-3-ethyl-8-methyl-2-(1-(piperazin-1-yl)butyl)quinazolin-4(3H)-one |
| 90 | 8-chloro-3-ethyl-6-fluoro-2-(1-(piperazin-1-yl)butyl)quinazolin-4(3H)-one |
| 91 | 6-bromo-5-chloro-3-ethyl-2-(1-(piperazin-1-yl)butyl)quinazolin-4(3H)-one |
| 92 | 3-ethyl-2-(1-(piperazin-1-yl)butyl)pyrido[3,2-d]pyrimidin-4(3H)-one |
| 93 | 6-bromo-3-ethyl-2-(1-(piperazin-1-yl)butyl)pyrido[2,3-d]pyrimidin-4(3H)-one |
| 94 | 3-ethyl-2-(1-(piperazin-1-yl)butyl)pyrido[2,3-d]pyrimidin-4(3H)-one |
| 95 | (S)-6-bromo-3-ethyl-8-fluoro-2-(1-(piperazin-1-yl)butyl)quinazolin-4(3H)-one |
| 96 | (R)-6-bromo-3-ethyl-8-fluoro-2-(1-(piperazin-1-yl)butyl)quinazolin-4(3H)-one |
| 97 | 6-bromo-3-ethyl-2-((R)-1-((S)-3-methylpiperazin-1-yl)butyl)quinazolin-4(3H)-one |
| 98 | 6-bromo-3-ethyl-2-((R)-1-((R)-3-methylpiperazin-1-yl)butyl)quinazolin-4(3H)-one |
| 99 | 6-bromo-3-ethyl-2-((S)-1-((S)-3-methylpiperazin-1-yl)butyl)quinazolin-4(3H)-one |
| 100 | 6-bromo-3-ethyl-2-((S)-1-((R)-3-methylpiperazin-1-yl)butyl)quinazolin-4(3H)-one |
| 101 | 6-chloro-3-ethyl-2-((R)-1-((R)-3-methylpiperazin-1-yl)butyl)quinazolin-4(3H)-one |
| 102 | 6-chloro-3-ethyl-2-((S)-1-((R)-3-methylpiperazin-1-yl)butyl)quinazolin-4(3H)-one |
| 103 | 6-chloro-3-ethyl-2-((R)-1-((S)-3-methylpiperazin-1-yl)butyl)quinazolin-4(3H)-one |
| 104 | 6-chloro-3-ethyl-2-((S)-1-((S)-3-methylpiperazin-1-yl)butyl)quinazolin-4(3H)-one |
| 105 | 6-bromo-3-ethyl-7-fluoro-2-((S)-1-((S)-3-methylpiperazin-1-yl)butyl)quinazolin-4(3H)-one |
| 106 | 6-bromo-3-ethyl-7-fluoro-2-((R)-1-((S)-3-methylpiperazin-1-yl)butyl)quinazolin-4(3H)-one |
| 107 | 3-((S)-4-((S)-1-(6-bromo-3-ethyl-4-oxo-3,4-dihydroquinazolin-2-yl)butyl)piperazin-2- |
| | yl)propanoic acid |
| 108 | 3-((S)-4-((R)-1-(6-bromo-3-ethyl-4-oxo-3,4-dihydroquinazolin-2-yl)butyl)piperazin-2- |
| | yl)propanoic acid |
| 109 | 6-bromo-2-((R)-1-((R)-3,4-dimethylpiperazin-1-yl)butyl)-3-ethylquinazolin-4(3H)-one |
| 110 | 6-bromo-2-((S)-1-((R)-3,4-dimethylpiperazin-1-yl)butyl)-3-ethylquinazolin-4(3H)-one |
| 111 | 6-bromo-2-((S)-1-((S)-3,4-dimethylpiperazin-1-yl)butyl)-3-ethylquinazolin-4(3H)-one |
| 112 | 6-bromo-2-((R)-1-((S)-3,4-dimethylpiperazin-1-yl)butyl)-3-ethylquinazolin-4(3H)-one |
| 113 | 2-(1-((3S,5R)-3,5-dimethylpiperazin-1-yl)butyl)-3-(2-methoxyethyl)quinazolin-4(3H)-one |
| 114 | 2-(1-((3S,5R)-3,5-dimethylpiperazin-1-yl)-3-methylbutyl)-3-(2-methoxyethyl)quinazolin- |
| | 4(3H)-one |
| 115 | 2-(1-((3S,5R)-3,5-dimethylpiperazin-1-yl)pentyl)-3-(2-methoxyethyl)quinazolin-4(3H)-one |
| 116 | 2-(2-cyclopropyl-1-((3S,5R)-3,5-dimethylpiperazin-1-yl)ethyl)-3-(2- |
| | methoxyethyl)quinazolin-4(3H)-one |
| 117 | 3-(2-methoxyethyl)-2-(1-(piperazin-1-yl)butyl)quinazolin-4(3H)-one |
| 118 | 2-(1-((3S,5R)-3,5-dimethylpiperazin-1-yl)butyl)-3-(furan-2-ylmethyl)quinazolin-4(3H)-one |
| 119 | 2-(1-((3S,5R)-3,5-dimethylpiperazin-1-yl)butyl)-3-(2-methoxyethyl)-6-methylquinazolin- |
| | 4(3H)-one |
| 120 | 2-(1-((3S,5R)-3,5-dimethylpiperazin-1-yl)butyl)-6-methoxy-3-(2- |
| | methoxyethyl)quinazolin-4(3H)-one |
| 121 | 2-(1-((3S,5R)-3,5-dimethylpiperazin-1-yl)butyl)-3-(2-methoxyethyl)pyrido[3,4- |
| | d]pyrimidin-4(3H)-one |
| 122 | ethyl 3-(6-bromo-2-(1-((3S,5R)-3,5-dimethylpiperazin-1-yl)butyl)-4-oxoquinazolin- |
| | 3(4H)-yl)propanoate |
| 123 | 2-((R)-1-((3S,5R)-3,5-dimethylpiperazin-1-yl)butyl)-3-(2-methoxyethyl)quinazolin- |
| | 4(3H)-one |
| 124 | 2-((S)-1-((3S,5R)-3,5-dimethylpiperazin-1-yl)butyl)-3-(2-methoxyethyl)quinazolin- |
| | 4(3H)-one |
| 125 | 3-(6-bromo-2-(1-((3S,5R)-3,5-dimethylpiperazin-1-yl)butyl)-4-oxoquinazolin-3(4H)- |
| | yl)propanoic acid |
| 126 | 2-(6-bromo-2-(1-((3S,5R)-3,5-dimethylpiperazin-1-yl)butyl)-4-oxoquinazolin-3(4H)- |
| | yl)acetic acid |
| 127 | 2-(1-((3S,5R)-3,5-dimethylpiperazin-1-yl)butyl)-3-(2-(methylamino)ethyl)quinazolin- |
| | 4(3H)-one |
| 128 | 3-(2-(dimethylamino)ethyl)-2-(1-((3S,5R)-3,5-dimethylpiperazin-1-yl)butyl)quinazolin- |
| | 4(3H)-one |
| 129 | 2-(1-((3S,5R)-3,5-dimethylpiperazin-1-yl)butyl)-3-(2- |
| | (methyl(phenethyl)amino)ethyl)quinazolin-4(3H)-one |
| 130 | 2-(1-((3S,5R)-3,5-dimethylpiperazin-1-yl)butyl)-3-(2-(methyl(3- |
| | phenylpropyl)amino)ethyl)quinazolin-4(3H)-one |
| 131 | 3-(2-(dimethylamino)ethyl)-2-(1-((3S,5R)-3,4,5-trimethylpiperazin-1- |
| | yl)butyl)quinazolin-4(3H)-one |
| 132 | 2-(1-((3S,5R)-3,5-dimethylpiperazin-1-yl)butyl)-3-ethyl-6-(pyridin-4-yl)quinazolin- |
| | 4(3H)-one |
| 133 | 2-(1-((3S,5R)-3,5-dimethylpiperazin-1-yl)butyl)-3-ethyl-7-(pyridin-4-yl)quinazolin- |
| | 4(3H)-one |
| 134 | 2-(1-((3S,5R)-3,5-dimethylpiperazin-1-yl)butyl)-3-ethyl-7-(3-hydroxyphenyl)quinazolin- |
| | 4(3H)-one |
| 135 | 2-(1-((3S,5R)-3,5-dimethylpiperazin-1-yl)butyl)-3-ethyl-7-(2-methoxypyridin-4- |
| | yl)quinazolin-4(3H)-one |
| 136 | 2-(1-((3S,5R)-3,5-dimethylpiperazin-1-yl)butyl)-3-methyl-7-(2- |
| | ((methylamino)methyl)pyridin-4-yl)quinazolin-4(3H)-one |
| 137 | 2-(1-((3S,5R)-3,5-dimethylpiperazin-1-yl)butyl)-3-ethyl-7-(pyrrolidin-1-yl)quinazolin- |
| | 4(3H)-one |
| 138 | 2-(1-((3S,5R)-3,5-dimethylpiperazin-1-yl)butyl)-3-ethyl-6-(pyrrolidin-1-yl)quinazolin- |
| | 4(3H)-one |
| 139 | 6-(4-(dimethylamino)piperidin-1-yl)-2-(1-((3S,5R)-3,5-dimethylpiperazin-1-yl)butyl)-3- |
| | ethylquinazolin-4(3H)-one |
| 140 | 2-(1-((3S,5R)-3,5-dimethylpiperazin-1-yl)butyl)-3-ethyl-6-(4-(methylamino)piperidin-1- |
| | yl)quinazolin-4(3H)-one |
| 141 | 6-(4-aminopiperidin-1-yl)-2-(1-((3S,5R)-3,5-dimethylpiperazin-1-yl)butyl)-3- |
| | ethylquinazolin-4(3H)-one |
| 142 | 2-(1-((3S,5R)-3,5-dimethylpiperazin-1-yl)butyl)-3-ethyl-6-((1-methylpiperidin-4- |
| | yl)amino)quinazolin-4(3H)-one |
| 143 | 2-(1-((3S,5R)-3,5-dimethylpiperazin-1-yl)butyl)-3-ethyl-6-(methyl(1-methylpiperidin-4- |
| | yl)amino)quinazolin-4(3H)-one |
| 144 | 6-(benzyl(methyl)amino)-2-(1-((3S,5R)-3,5-dimethylpiperazin-1-yl)butyl)-3- |
| | ethylquinazolin-4(3H)-one |
| 145 | 2-(1-((3S,5R)-3,5-dimethylpiperazin-1-yl)butyl)-3-ethyl-6-((2- |
| | (methylamino)ethyl)amino)quinazolin-4(3H)-one |
| 146 | 2-(1-((3S,5R)-3,5-dimethylpiperazin-1-yl)butyl)-3-ethyl-6-(methyl(2- |
| | (methylamino)ethyl)amino)quinazolin-4(3H)-one |
| 147 | 2-(1-((3S,5R)-3,5-dimethylpiperazin-1-yl)butyl)-3-ethyl-7-((2- |
| | (methylamino)ethyl)amino)quinazolin-4(3H)-one |
| 148 | 2-(1-((3S,5R)-3,5-dimethylpiperazin-1-yl)butyl)-3-ethyl-6-(4-(2-hydroxyphenyl)-5,6- |
| | dihydropyridin-1(2H)-yl)quinazolin-4(3H)-one |
| 149 | 2-(1-((3S,5R)-3,5-dimethylpiperazin-1-yl)butyl)-3-ethyl-6-((3S,3aR,7aR)-3- |
| | phenylhexahydro-4,7-ethanopyrrolo[3,2-b]pyridin-1(2H)-yl)quinazolin-4(3H)-one |
| 150 | 6-((1-benzylpiperidin-4-yl)amino)-2-(1-((3S,5R)-3,5-dimethylpiperazin-1-yl)butyl)-3- |
| | ethylquinazolin-4(3H)-one |
| 151 | 6-(4-(benzyl(methyl)amino)piperidin-1-yl)-2-(1-((3S,5R)-3,5-dimethylpiperazin-1- |
| | yl)butyl)-3-ethylquinazolin-4(3H)-one |
| 152 | 2-((S)-1-((3S,5R)-3,5-dimethylpiperazin-1-yl)butyl)-3-ethyl-6-((S)-2-methyl-1-oxa-4,9- |
| | diazaspiro[5.5]undecan-4-yl)quinazolin-4(3H)-one |
| 153 | 2-((R)-1-((3S,5R)-3,5-dimethylpiperazin-1-yl)butyl)-3-ethyl-6-((S)-2-methyl-1-oxa-4,9- |
| | diazaspiro[5.5]undecan-4-yl)quinazolin-4(3H)-one |
| 154 | 2-((S)-1-((3S,5R)-3,5-dimethylpiperazin-1-yl)butyl)-3-ethyl-6-((R)-2-methyl-9- |
| | phenethyl-1-oxa-4,9-diazaspiro[5.5]undecan-4-yl)quinazolin-4(3H)-one |
| 155 | 2-((S)-1-((3S,5R)-3,5-dimethylpiperazin-1-yl)butyl)-3-ethyl-6-((S)-2-methyl-9-phenethyl- |
| | 1-oxa-4,9-diazaspiro[5.5]undecan-4-yl)quinazolin-4(3H)-one |
| 156 | 2-((R)-1-((3S,5R)-3,5-dimethylpiperazin-1-yl)butyl)-3-ethyl-6-((S)-2-methyl-9- |
| | phenethyl-1-oxa-4,9-diazaspiro[5.5]undecan-4-yl)quinazolin-4(3H)-one |
| 157 | 2-((R)-1-((3S,5R)-3,5-dimethylpiperazin-1-yl)butyl)-3-ethyl-6-((R)-2-methyl-9- |
| | phenethyl-1-oxa-4,9-diazaspiro[5.5]undecan-4-yl)quinazolin-4(3H)-one |
| 158 | 2-((S)-1-((3S,5R)-3,5-dimethylpiperazin-1-yl)butyl)-3-ethyl-6-((2-((R)-9-(pyridin-2-yl)-6- |
| | oxaspiro[4.5]decan-9-yl)ethyl)amino)quinazolin-4(3H)-one |
| 159 | 2-((R)-1-((3S,5R)-3,5-dimethylpiperazin-1-yl)butyl)-3-ethyl-6-((2-((S)-9-(pyridin-2-yl)-6- |
| | oxaspiro[4.5]decan-9-yl)ethyl)amino)quinazolin-4(3H)-one |
| 160 | 2-((R)-1-((3S,5R)-3,5-dimethylpiperazin-1-yl)butyl)-3-ethyl-6-((2-((R)-9-(pyridin-2-yl)- |
| | 6-oxaspiro[4.5]decan-9-yl)ethyl)amino)quinazolin-4(3H)-one |
| 161 | 2-((S)-1-((3S,5R)-3,5-dimethylpiperazin-1-yl)butyl)-3-ethyl-6-((2-((S)-9-(pyridin-2-yl)-6- |
| | oxaspiro[4.5]decan-9-yl)ethyl)amino)quinazolin-4(3H)-one |
| 162 | tert-Butyl (8-bromo-2-(1-((3S,5R)-3,5-dimethylpiperazin-1-yl)butyl)-3-ethyl-4-oxo-3,4- |
| | dihydroquinazolin-7-yl)(3-methoxybenzyl)carbamate |
| 163 | N-(2-(1-((3S,5R)-3,5-dimethylpiperazin-1-yl)butyl)-3-ethyl-4-oxo-3,4- |
| | dihydroquinazolin-6-yl)-N-(1-methylpiperidin-4-yl)propionamide. |
| 164 | 2-((S)-1-((3S,5R)-3,5-dimethylpiperazin-1-yl)butyl)-3-methyl-7-((R)-3-(methylamino)-1- |
| | phenylpropoxy)quinazolin-4(3H)-one |
| 165 | 2-((R)-1-((3S,5R)-3,5-dimethylpiperazin-1-yl)butyl)-3-methyl-7-((R)-3-(methylamino)-1- |
| | phenylpropoxy)quinazolin-4(3H)-one |
| 166 | 2-((S)-1-((3S,5R)-3,5-dimethylpiperazin-1-yl)butyl)-3-methyl-7-((S)-3-(methylamino)-1- |
| | phenylpropoxy)quinazolin-4(3H)-one |
| 167 | 2-((R)-1-((3S,5R)-3,5-dimethylpiperazin-1-yl)butyl)-3-methyl-7-((S)-3-(methylamino)-1- |
| | phenylpropoxy)quinazolin-4(3H)-one |
| 168 | 2-((S)-1-((3S,5R)-3,5-dimethylpiperazin-1-yl)butyl)-3-methyl-7-((S)-2-(methylamino)-1- |
| | phenylethoxy)quinazolin-4(3H)-one |
| 169 | 2-((R)-1-((3S,5R)-3,5-dimethylpiperazin-1-yl)butyl)-3-methyl-7-((R)-2-(methylamino)-1- |
| | phenylethoxy)quinazolin-4(3H)-one |
| 170 | 2-((R)-1-((3S,5R)-3,5-dimethylpiperazin-1-yl)butyl)-3-methyl-7-((S)-2-(methylamino)-1- |
| | phenylethoxy)quinazolin-4(3H)-one |
| 171 | 2-((S)-1-((3S,5R)-3,5-dimethylpiperazin-1-yl)butyl)-3-methyl-7-((R)-2-(methylamino)-1- |
| | phenylethoxy)quinazolin-4(3H)-one |
| 172 | 2-(1-((3S,5R)-3,5-dimethylpiperazin-1-yl)butyl)-7-(hydroxymethyl)-3-methylquinazolin- |
| | 4(3H)-one |
| 173 | 2-(1-((3S,5R)-3,5-dimethylpiperazin-1-yl)butyl)-3-ethyl-6-(2-hydroxyethyl)quinazolin- |
| | 4(3H)-one |
| 174 | 6-(2-(benzyl(methyl)amino)ethyl)-2-(1-((3S,5R)-3,5-dimethylpiperazin-1-yl)butyl)-3- |
| | ethylquinazolin-4(3H)-one |
| 175 | 2-(1-((3S,5R)-3,5-dimethylpiperazin-1-yl)butyl)-3-ethyl-6-(2- |
| | (isopentyl(methyl)amino)ethyl)quinazolin-4(3H)-one |
| 176 | 2-(1-((3S,5R)-3,5-dimethylpiperazin-1-yl)butyl)-3-methyl-4-oxo-3,4-dihydroquinazoline- |
| | 7-carbonitrile |
| 177 | 2-(1-((3S,5R)-3,5-dimethylpiperazin-1-yl)butyl)-3-ethyl-4-oxo-3,4-dihydroquinazoline-6- |
| | carbonitrile |
| 178 | 2-(1-((3S,5R)-3,5-dimethylpiperazin-1-yl)butyl)-3-methyl-4-oxo-3,4-dihydroquinazoline- |
| | 7-carboxylic acid |
| 179 | 2-(1-((3S,5R)-3,5-dimethylpiperazin-1-yl)butyl)-3-ethyl-4-oxo-3,4-dihydroquinazoline-7- |
| | carboxylic acid |
| 180 | N-benzyl-2-(1-((3S,5R)-3,5-dimethylpiperazin-1-yl)butyl)-3-ethyl-4-oxo-3,4- |
| | dihydroquinazoline-7-carboxamide |
| 181 | N-(1-((2-(1-((3S,5R)-3,5-dimethylpiperazin-1-yl)butyl)-3-ethyl-4-oxo-3,4- |
| | dihydroquinazolin-6-yl)methyl)piperidin-4-yl)-N-phenylpropionamide |
| 182 | N-(1-((2-(1-((3S,5R)-3,5-dimethylpiperazin-1-yl)butyl)-3-methyl-4-oxo-3,4- |
| | dihydroquinazolin-7-yl)methyl)piperidin-4-yl)-N-phenylpropionamide |
| 183 | 6-((benzyl(methyl)amino)methyl)-2-(1-((3S,5R)-3,5-dimethylpiperazin-1-yl)butyl)-3- |
| | ethylquinazolin-4(3H)-one |
| 184 | 2-(1-((3S,5R)-3,5-dimethylpiperazin-1-yl)butyl)-3-ethyl-6- |
| | ((isobutyl(methyl)amino)methyl)quinazolin-4(3H)-one |
| 185 | 2-(1-((3S,5R)-3,5-dimethylpiperazin-1-yl)butyl)-3-ethyl-6- |
| | ((isopentyl(methyl)amino)methyl)quinazolin-4(3H)-one |
| 186 | 2-(1-((3S,5R)-3,5-dimethylpiperazin-1-yl)butyl)-3-methyl-7-((4-methylpiperazin-1- |
| | yl)methyl)quinazolin-4(3H)-one |
| 187 | 6-bromo-3-ethyl-2-(1-(piperidin-4-yl)butyl)quinazolin-4(3H)-one |
| 188 | 6-bromo-3-ethyl-2-(1-(l,2,3,6-tetrahydropyridin-4-yl)butyl)quinazolin-4(3H)-one |
| 189 | 6-bromo-2-((S)-1-((R)-3,3-difluoropiperidin-4-yl)butyl)-3-ethylquinazolin-4(3H)-one |
| 190 | 6-bromo-3-ethyl-2-((R)-1-((2S,4S)-2-methylpiperidin-4-yl)butyl)quinazolin-4(3H)-one |
| 191 | 6-bromo-3-ethyl-2-((S)-1-((2S,4S)-2-methylpiperidin-4-yl)butyl)quinazolin-4(3H)-one |
| 192 | 6-bromo-3-ethyl-2-((R)-1-((2S,4S)-2-methylpiperidin-4-yl)buty])quinazolin-4(3H)-one |
| 193 | 6-bromo-3-ethyl-2-((S)-1-((2S,4R)-2-methylpiperidin-4-yl)butyl)quinazolin-4(3H)-one |
| 194 | 6-bromo-2-((S)-1-((S)-3,3-difluoropiperidin-4-yl)butyl)-3-ethylquinazolin-4(3H)-one |
| 195 | 6-bromo-2-((R)-1-((R)-3,3-difluoropiperidin-4-yl)butyl)-3-ethylquinazolin-4(3H)-one |
| 196 | 6-bromo-2-((S)-1-((R)-3,3-difluoropiperidin-4-yl)butyl)-3-ethylquinazolin-4(3H)-one |
| 197 | 6-bromo-2-((R)-1-((S)-3,3-difluoropiperidin-4-yl)butyl)-3-ethylquinazolin-4(3H)-one |
| 198 | 6-chloro-3-methyl-2-((R)-1-((S)-3-methylpiperazin-1-yl)butyl)quinazolin-4(3H)-one |
| 199 | (R)-6-chloro-3-ethyl-8-fluoro-2-(1-(piperazin-1-yl)butyl)quinazolin-4(3H)-one |
| 200 | (R)-3-ethyl-6,8-difluoro-2-(1-(piperazin-1-yl)butyl)quinazolin-4(3H)-one |
| 201 | 3-ethyl-6,8-difluoro-2-((R)-1-((S)-3-methylpiperazin-1-yl)butyl)quinazolin-4(3H)-one |
| 202 | 6-fluoro-2-((R)-1-((S)-3-methylpiperazin-1-yl)butyl)-3-propylquinazolin-4(3H)-one |
| 203 | 3-ethyl-8-fluoro-2-((R)-1-((S)-3-methylpiperazin-1-yl)butyl)quinazolin-4(3H)-one |
| 204 | 3-ethyl-2-((R)-1-((S)-3-methylpiperazin-1-yl)butyl)-6-(trifluoromethoxy)quinazolin- |
| | 4(3H)-one |
| 205 | 6-fluoro-3-methyl-2-((R)-1-((S)-3-methylpiperazin-1-yl)butyl)quinazolin-4(3H)-one |
| 206 | 6,7-dichloro-3-methyl-2-((R)-1-((S)-3-methylpiperazin-1-yl)butyl)quinazolin-4(3H)-one |
| 207 | 3-ethyl-2-((R)-1-((S)-3-methylpiperazin-1-yl)butyl)-6-(trifluoromethyl)quinazolin-4(3H)-one |
| 208 | 6-bromo-3-ethyl-7-fluoro-2-((R)-1-((R)-3-methylpiperazin-1-yl)butyl)quinazolin-4(3H)-one |
| 209 | 6-bromo-3-ethyl-7-fluoro-2-((S)-1-((R)-3-methylpiperazin-1-yl)butyl)quinazolin-4(3H)-one |
| 210 | 3-ethyl-7-fluoro-6-methoxy-2-((R)-1-((R)-3-methylpiperazin-1-yl)butyl)quinazolin- |
| | 4(3H)-one |
| 211 | 3-ethyl-6-methoxy-2-((R)-1-((R)-3-methylpiperazin-1-yl)butyl)quinazolin-4(3H)-one |
| 212 | 6-bromo-3-ethyl-2-((R)-1-((S)-3-ethylpiperazin-1-yl)butyl)quinazolin-4(3H)-one |
| 213 | 3-ethyl-7-fluoro-6-methoxy-2-((R)-1-((S)-3-methylpiperazin-1-yl)butyl)quinazolin- |
| | 4(3H)-one |
| 214 | 3-ethyl-6-fluoro-2-((R)-1-((S)-3-methylpiperazin-1-yl)butyl)quinazolin-4(3H)-one |
| 215 | 3-ethyl-6-methoxy-2-((R)-1-((S)-3-methylpiperazin-1-yl)butyl)quinazolin-4(3H)-one |
| 216 | 3-ethyl-6,7-difluoro-2-((R)-1-((S)-3-methylpiperazin-1-yl)butyl)quinazolin-4(3H)-one |
| 217 | 6-chloro-3-ethyl-8-fluoro-2-((R)-1-((S)-3-methylpiperazin-1-yl)butyl)quinazolin-4(3H)-one |
| 218 | 3-ethyl-5,6-difluoro-2-((R)-1-((S)-3-methylpiperazin-1-yl)butyl)quinazolin-4(3H)-one |
| 219 | 6-bromo-3-ethyl-2-((R)-1-((R)-3-(methoxymethyl)piperazin-1-yl)butyl)quinazolin-4(3H)-one |
| 220 | 6-bromo-3-ethyl-2-((S)-1-((R)-3-(methoxymethyl)piperazin-1-yl)butyl)quinazolin-4(3H)-one |
| and | |
| 221 | 6-chloro-7-fluoro-3-methyl-2-((R)-1-((S)-3-methylpiperazin-1-yl)butyl)quinazolin- |
| | 4(3H)-one |
| 222 | 5,6-difluoro-3-methyl-2-((R)-1-((S)-3-methylpiperazin-1-yl)butyl)quinazolin-4(3H)-one |
| 223 | 6-chloro-8-fluoro-3-methyl-2-((R)-1-((S)-3-methylpiperazin-1-yl)butyl)quinazolin- |
| | 4(3H)-one |
| 224 | (R)-6-bromo-2-(1-(3,3-dimethylpiperazin-1-yl)butyl)-3-ethylquinazolin-4(3H)-one |
| 225 | 6,8-difluoro-3-methyl-2-((R)-1-((S)-3-methylpiperazin-1-yl)butyl)quinazolin-4(3H)-one |
| 226 | 3-ethyl-5,6,8-trifluoro-2-((R)-1-((S)-3-methylpiperazin-1-yl)butyl)quinazolin-4(3H)-one |
| 227 | 6-Bromo-2-((S)-1-((3S,5S)-3,5-dimethylpiperazin-1-yl)butyl)-3-ethylquinazolin-4(3H)-one |
| 228 | 6-Bromo-2-((R)-1-((3S,5S)-3,5-dimethylpiperazin-1-yl)butyl)-3-ethylquinazolin-4(3H)-one |
| 229 | 6-Chloro-3-ethyl-2-((R)-1-((S)-3-(fluoromethyl)piperazin-1-yl)butyl)quinazolin-4(3H)-one |
| 230 | 6-chloro-3-ethyl-2-((R)-1-((R)-3-(fluoromethyl)piperazin-1-yl)butyl)quinazolin-4(3H)-one |
| 231 | 6-chloro-3-ethyl-2-((S)-1-((R)-3-(fluoromethyl)piperazin-1-yl)butyl)quinazolin-4(3H)-one |
| 232 | 6-chloro-3-ethyl-2-((S)-1-((S)-3-(fluoromethyl)piperazin-1-yl)butyl)quinazolin-4(3H)-one. |
| 233 | 3-Ethyl-2-((R)-1-((R)-3-methylpiperazin-1-yl)butyl)-6-(trifluoromethyl)pyrido[3,4- |
| | d]pyrimidin-4(3H)-one |
| 234 | 3-ethyl-2-((R)-1-((S)-3-methylpiperazin-1-yl)butyl)-6-(trifluoromethyl)pyrido[3,4- |
| | d]pyrimidin-4(3H)-one |
| 235 | 3-ethyl-2-((S)-1-((S)-3-methylpiperazin-1-yl)butyl)-6-(trifluoromethyl)pyrido[3,4- |
| | d]pyrimidin-4(3H)-one |
| 236 | 2-((R)-1-((3S,5R)-3,5-Dimethylpiperazin-1-yl)butyl)-3-ethyl-6- |
| | ((methylamino)(phenyl)methyl)quinazolin-4(3H)-one |

20. A process for the preparation of a compound of Formula (I) as defined in claim 1:
(A) when W is —CH— said process comprising alkylating a compound of the Formula: 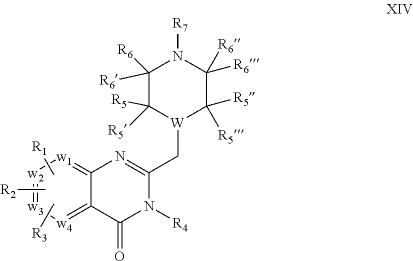
with an alkylating compound of the Formula: 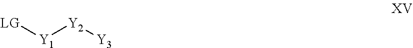
in the presence of a base under conditions sufficient to produce said compound of Formula I where W is —CH—,
wherein
R₁, R₂, R₃, R₄, R₅, R₅′, R₅″, R₅′″, R₆, R₆′, R₆″, R₆′″, R₇, Y₁, Y₂, Y₃, w₁, w₂, w₃ and w₄ are as defined in claim 13, and
LG is a leaving group,
or
(B) when W is nitrogen, said process comprising reacting a compound of the Formula: 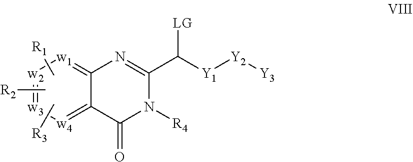
with an amine compound of the Formula: 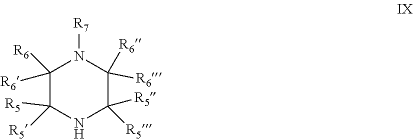
in the presence of a base under conditions sufficient to produce said compound of Formula I, where W is nitrogen,
wherein
R₁, R₂, R₃, R₄, R₅, R₅′, R₅″, R₅′″, R₆, R₆′, R₆″, R₆′″, R₇, Y₁, Y₂, Y₃, wt, w₂, w₃ and w₄ are as defined in claim 13; and
LG is a leaving group.

21. A pharmaceutical composition comprising a compound of Formula (I) according to claim 13 or a pharmaceutically acceptable salt thereof, and a pharmaceutically acceptable carrier, adjuvant or vehicle.

22. The pharmaceutical composition of claim 21, wherein said composition comprises an enantimerically enriched isomer of said compound of Formula (I).

23. The pharmaceutical composition of claim 21, wherein said composition comprises a diastereomerically enriched isomer of said compound of Formula (I).

24. The pharmaceutical composition of claim 21, wherein said composition comprises a racemic mixture of said compound of Formula (I).

25. A method for treating pain in a subject, said method comprising administering to a subject in need of pain treatment a therapeutically effective amount of a compound of claim 13.

26. The method of claim 25, wherein said pain is selected from the group consisting of visceral pain, chronic pain, cancer pain, migraine, inflammatory pain, acute pain, neuropathic pain, allodynia, and hyperalgesia.
